(19) United States
(12) Patent Application Publication
(10) Pub. No.: US 2016/0074480 A1
(43) Pub. Date: Mar. 17, 2016

(54) INNOVATIVE METHODS OF TREATMENTING TUBERCULOSIS

(71) Applicant: Totada R. SHANTHA, Stone Mountain, GA (US)

(72) Inventor: Totada R. SHANTHA, Stone Mountain, GA (US)

(21) Appl. No.: 14/951,367

(22) Filed: Nov. 24, 2015

(51) Int. Cl. A61K 38/28; A61K 31/375; A61K 45/06; A61K 31/366; A61K 31/44; A61K 31/7048; A61K 9/00; A61K 31/496

(52) U.S. Cl. CPC A61K 38/28; A61K 9/0019; A61K 31/375; A61K 31/496; A61K 31/366; A61K 31/44; A61K 31/7048; A61K 45/06

(57) ABSTRACT

A Safe and effective treatment methods to cure and curtail tuberculosis affliction described using high dose Vitamin C, and other known anti-mycobacterium tuberculosis drugs especially rifampicin administered intravenously for 6 weeks instead of 6 to 24 months of conventional treatment. Insulin is administered to induce moderate hypoglycemia to augment and add effectiveness of anti-tuberculosis drugs and Vitamin C. Invention also delivers the drugs directly to a tuberculin lesion through a catheter. An embodiment of the invention uses a nebulizer and other methods of administration of Vitamin C with anti-tuberculosis drugs, interferon Y⁻, Coley's vaccine, dinitrophenol hyperthermia, ozone therapy, Hydrogen peroxide therapy and artemisinin, combined with oxygen supplementation including autohemotherapy with ozone, hyperbaric therapy, and hypertheramia to increase the respiration of the M. tuberculosis bacteria which has a killing effect.

### TECHNICAL FIELD

This invention relates to the methods for curtailing and curing mycobacterium tuberculosis bacterial infection of mammals, in particular humans, which infects millions of people and results in millions of deaths every year all over the world.

### BACKGROUND OF THE INVENTION

Tuberculosis is one of the oldest recognized diseases, described in Indian Rig-Veda and Atharvaveda as “YAKSHMA” between 1500-1000 BC (or even older i.e. 3500 BC) and is the most prevalent chronic bacterial diseases of mankind besides leprosy. The Greco—Romans called it PHTHISIS (Greek: a dwindling or wasting away—archaic name for TB) and TB is also known as CONSUMPTION for the same reason. Evidence of spinal tuberculosis has been found in the some of the thousands of years old Egyptian mummies (1550 BC, diagnosed by PCR). Aristotle (354-322 BC) stated that when one comes near consumptives, one does contact the disease, due to pernicious disease producing air breathed by these patients.

Tuberculosis (TB) is the single leading cause of death from an infectious disease in the world, and a primary public health threat especially in Africa, India, and other overcrowded countries. When we say the person has tuberculosis, it is taken as “suffering from TB of the lungs” though the other parts of the body (3% vertebral) could have the infection other than the lungs. 40% of the pulmonary TB cases are associated with lymphadenitis (mediastinal, posterior cervical and supraclavicular areas). Other membrane and non-membrane structures in the body such as pleura, peritoneum, pericardium, meninges; bones and joints; digestive system, genitourinary tract, etc. can also have TB infection. No tissue and organ in our body is immune to M. tuberculosis bacterial infection.

Tuberculosis is a major public health threat, and even now it continues as a global endo-epidemic. A relapse in areas in which TB is endemic is largely due to reinfection (Narayanan et. al. 2004). TB is winning the war by accumulating resistance alleles before an effective chemotherapy can be developed. Countless millions of people have died and continue to die due to this chronic infectious disease caused by the tubercle bacillus. Over 2 billion people harbor latent TB infection and more than 9.2 million new TB cases are reported (Maartens G, Wilkinson R J 2007), of who 500,000 are multidrug-resistant (MDR) and nearly 1.7 million deaths (some estimate as high as 3 million US 2014/0050776 A1) occur each year which is very high for curable disease. In 2004 nearly 14.6 million people had active tuberculosis and 75% of these cases affect the lungs. That means, one-third of the world is infected with latent mycobacterium tuberculosis (TB) and 5000 (to 8219?) people die of TB every day. It is predicted that over 150 million people will contract active TB, and about 36 million people will die over the next 20 years all over the world unless TB control and curative treatments are developed and are widely used. There is 50% death if TB untreated; hence the innovative treatment using new modalities as described herein is needed. In spite of all these gloomy statistics, there is high success in the treatment protocol due to WHO program ascribed to the Directly Observed Therapy Short course (DOTS) strategy, which closely monitors patient adherence to chemotherapeutic regimens, because the compliance is s problem due to prolonged therapy, and due to poor socioeconomic conditions. Need to be adopted all over world without second thought.

Tuberculosis in India: Facts and Figures:
a) TB kills two people every 3 minute in India, 330,000 people a year and 900 people a day;
b) Nearly 2 million new cases reported annually, together with 0.8 million new sputum smear positive cases annually, including estimated 75 sputum smear positive pulmonary TB per 100,000 population;
c) An estimated 5% of TB patients are HIV infected in India;
d) Indirect cost to society is estimated to be $3 billion dollars annually (after Dr. C. V. Rao: Power point slide 2013 on Tuberculosis), and
e) An estimated 40% of the populations of India are infected (exposed)—i.e. an estimated ⅕th of the world populations of TB patients are in India with 6 million radiologically proven cases.
f) It is projected that 4 million people in India will die of tuberculosis in the next decade.
g) India has the highest burden of both drug-susceptible and drug-resistant tuberculosis (TB) in the world, with an estimated 3 million prevalent TB cases, of which 75,000 are multidrug-resistant TB (MDR TB).
h) Massive population growth; vast overcrowding of streets, transportation system, and dwellings; lack of proper sanitary system besides lack of proper nutrition, and many such factors contribute to endo-epidemic (widespread in the population and spreading unabated) of tuberculosis in India.

Evolution of the Effective Treatment of Tuberculosis (TB):

Mitchison and Davies (2012) describe the evolution of chemotherapy of TB and explain its past, present and the future, whose studies we have incorporated herein. It started in 1946, with the introduction of streptomycin (SM, S) undertaken by the Tuberculosis Research Unit (under Director Philip Hart) of the British Medical Research Council (BMRC), SM given alone caused a dramatic reduction in immediate mortality and striking improvements in chest radiology and bacteriology, (Medical Research Council Streptomycin treatment of pulmonary tuberculosis. BMJ. 1948; 2:769-782). But the 5-year assessment showed that the patients who received SM eventually died in almost the same proportion and speed as those who did not receive it, due to the frequent emergence of SM resistance M. tuberculosis bacteria (Fox W, Sutherland I, and Daniels M. A five-year assessment of patients in a controlled trial of streptomycin in pulmonary tuberculosis. Q J Med. 1954; 23: 347-366). A second BMRC clinical trial then showed that combined treatment with SM and para-aminosalicylic acid (PAS, P) greatly reduced the incidence of SM resistance. In 1952, isoniazid (INH, H) was introduced as a new wonder drug. Its efficacy stemmed from its low minimum inhibitory concentration (MIC) against Tuberculosis bacteria and its low toxicity. In 1960, the classic study at the Tuberculosis Chemotherapy Centre, Madras (now renamed as Chennai), India, under the direction of Fox, showed that domiciliary chemotherapy could be as effective as treatment in expensive hospitals or sanatoria (Tuberculosis Chemotherapy Centre, Madras. A concurrent comparison of home and sanatorium treatment of pulmonary tuberculosis in South India. Bull World Health Organ. 1959; 21:51-144. Andrews R H, Devadatta S, Fox W, Radhakrishna S, Ramakrishnan C V, Velu S. Prevalence of tuberculosis among close family contacts of tuberculous patients in South India, and influence of segregation of the patient on the early attack rate. Bull World Health Organ. 1960; 23:463-510). John Crofton explored a regimen starting with the three drugs, SM, PAS and INH (SPH/PH) so that two would be available for almost any resistant strain in the community, followed by a continuation phase of the two oral drugs, PAS and INH (Crofton J. Chemotherapy of pulmonary tuberculosis. BMJ. 1959; 1: 1610-1614. Crofton J. Sputum conversion and the metabolism of isoniazid. Am Rev Tuberc. 1958; 77: 869-871). Crofton's experience led to a clinical trial under the auspices of the International Union against Tuberculosis to assess this regimen (International Union against Tuberculosis An international investigation of the efficacy of chemotherapy in previously untreated patients with pulmonary tuberculosis. Bull Int Union Tuberc. 1964; 34(2):83-191). However, it required at least one year of treatment in hospital with very expensive drug bills due to the large amounts of PAS, this meant that the regimen could not be used widely in any but the richer countries. This gradually led to the home therapy with multiple drug combination with new anti-TB drugs development, which ultimately leads many years later to the evolution of “Directly Observed Treatment Short course” (DOTS) is the internationally recommended and adopted strategy for TB control that has been recognized as a highly efficient and cost-effective strategy. In spite of all these developments and DOTS efforts by WHO, and state governments; tuberculosis infection is number one infectious disease killer though it is curable.

The WHO Principles of DOTS: It Comprises of Five Components to be Successful:
1. Sustained political and financial commitment. TB can be cured and the epidemic reversed if adequate resources and administrative support for TB control are provided
2. Diagnosis by quality ensured sputum-smear microscopy. Chest symptomatic examined by this way, combined with Chest X ray and fluoroscopy if possible, helps to reliably find infectious patients,
3. Standardized short-course anti-TB treatment (SCC) given under direct and supportive observation (DOT). Helps to ensure the right drugs are taken at the right time for the full duration of treatment.
4. A regular, uninterrupted supply of high quality anti-TB drugs. Ensures that a credible national TB program does not have to turn anyone away.
5. Standardized recording and reporting. Helps to keep track of each individual patient and to monitor overall program performance.

With TB in near endo-epidemic proportions in some parts of the world and with the rapid increase in multidrug-resistant strains of Mycobacterium tuberculosis in spite of DOTS, new methods of curing and curtailing this epidemic is needed. The World Health Organization declared TB to be a global public health emergency (Raviglione. 1995) and March 24th each year is observed as world TB day to bring its awareness and treatments. World TB Day is observed on March 24. This annual event commemorates the date in 1882 when Dr. Robert Koch announced his discovery of Mycobacterium tuberculosis, the bacillus that causes tuberculosis (TB). World TB Day provides an opportunity to raise awareness and knowledge about TB-related problems and solutions and to support worldwide TB control efforts. For 2014, CDC selected the theme “Find TB. Treat TB. Working together to eliminate TB.” Health officials in local and state TB programs are encouraged to reach out to their communities to raise awareness about TB and partner with others who are caring for those most at risk for TB. Every person has a role in ensuring that one day TB will be eliminated and it can be achieved only with education about this disease and participation of every one with isolation of open cases from contact with the healthy. With 2 billion people exposed to M. tuberculosis infection, many with hidden bacteria in the body waiting to emerge, it may take at least 75 years before we can eradicate and/or reduced to accountable number the menace of the M. tuberculosis bacterial infection of human. Further, new drugs and methods with potent anti-tuberculosis activity, especially against non-multiplying persisters are needed to shorten the duration of treatment for TB, thus facilitate the global implementation of directly observed therapy (DOTS). We describe such a method in this invention.

The numbers of individuals succumbing to tuberculosis have vastly increased as a result of the HIV/AIDS pandemic, and increased mobility owing to global travel has increased the transfer of virulent and multidrug resistant tuberculosis. Prolonged antibiotic treatment is also required bringing noncompliance by some of the afflicted, as the bacterium can enter a dormant, anti-tuberculosis drugs resistant phase.

Besides the morbidity and mortality due to tuberculosis, the incidence of lung cancer in these pulmonary tuberculosis patients is said to be greater than people without tuberculosis (about 11 times?) (www.sciencedaily.com/releases/2011/01/110102202238.htm. Evidence of increased lung cancer risk among tuberculosis patients. Science Daily, 2 Jan. 2011. Researchers at China Medical University and Hospital in Taiwan-). These patients also suffer from chronic obstructive pulmonary disease (COPD) and other lung infections besides development of anxiety and depression in some of these patients (Aydin and Aylin Uluahin 2001. Szulakowski P, et. al. 2006).

Apart from very young children, a small number of individuals become sick immediately after tuberculosis bacteria enter the body. M. tuberculosis bacteria that enter the lungs are immediately killed by the body's immune system. TB bacteria that survive are trapped (phagocytosed) inside macrophages; remain in a dormant state for many years as latent TB (Persister). In 90 to 95% of cases, the TB bacteria cause no further problems. But in 5 to 10% of infected people, the TB bacteria start to multiply (active TB). It is in this active TB phase that an infected person becomes sick and can spread the disease to those in close contact. It is important to note that reactivation of latent TB for most of the time begins at the apex of the lungs, where the oxygen tension (PO2) is highest because the M. tuberculosis is a strict obligate aerobe.

Tuberculosis is a highly contagious disease and the individuals contract the infection by inhaling tuberculosis bacilli-droplet infection (Mycobacterium tuberculosis hominis) in the air from the infected patients; the bacterium released through coughing, sneezing, singing, breathing, and talking (pulmonary tuberculosis-FIG. 5, FIG. 8 flow chart) or drinking raw milk (Mycobacterium tuberculosis bovis) causing gastrointestinal tract tuberculosis. WHO has estimated that less than 50 percent of all cases globally are currently diagnosed and treated (WHO 2010), since each person with undiagnosed and untreated active TB will survive an average of 2 years, it has been estimated that they will infect 20 others (Rieder H L1999). We propose to treat all active, latent TB by means of persister cell with our therapy and thus prevent the later development of active TB and spreading of disease.

Each inhaled droplet nuclei produced while coughing (FIG. 1a)) which is main mode of tuberculosis bacterial infection and is capable of transporting 1-3 bacilli into the alveoli of the lungs (Dannenberg and Rook 1994). Other mycobacteria such as Mycobacterium Africanum, Canetti, and Microti can also cause tuberculosis, but these Mycobacterium species do not usually infect healthy adults. Once the M. tuberculosis lands in the alveoli, the following events follow:
1. TB bacteria phagocytosed by macrophage of the alveoli and survive inside the cell and multiply.
2. This inflammation response in the alveoli brings more defensive white blood cells to the area of infection in the alveoli resulting in cytokine production that damages the alveolar wall.
3. This results in tubercle formation with dead macrophages releasing the bacteria surrounded by living lymphocytes surrounding the primary lesion.
4. The final drama comes to play in the battle inside the alveoli with rupture of the tubercle with spread of the bacteria to other part of the lungs and other organs. It is at this phage that coughing will transmit TB bacteria in droplets to those in close contact by air.

M. tuberculosis bacteria can remain dormant (latent TB— persister-individual infected with TB, but not suffering from TB disease) in the body after infection for years, concealed in the phagocytosed cells, and never develop into the disease. There are indications that bone marrow Mesenchymal stem cell may provide a place for dormant M. tuberculosis (Das et. al. 2013). It is estimated that 5 to 10% of latent cases will progress to be infectious or to Tuberculosis disease due let down guard by impaired immune system and due any number of reasons including being on corticosteroids, debilitating diseases, HIV infection etc. When a child is infected, they have fewer bacteria in their lungs than an adult with the disease; hence it is infrequent for children to infect other individuals with tuberculosis.

Symptoms: The classic symptoms of TB are: a chronic cough with blood tinged sputum, late day fever, night sweats, weight loss, lymph node enlargement, chest pain, and spread to other organs with or without the development of cold abscess and other symptoms.

Diagnosis: The diagnosis relies on microscopic examination of sputum, tuberculin skin test, blood tests, radiology (chest X-rays, MRI scans), biopsy of suspected lesions and cold abscess contents, examination of trachea-bronchial-alveolar lavage, and microbiological culture of bodily fluids including sputum.

M. tuberculosis Pathogen's General Characteristics:

Mycobacterium tuberculosis (M. tuberculosis) is non-motile, slowly growing and rod shaped (2-4 μm in length and 0.2-0.5 μm in width) takes about 15-20 hours to multiply in culture (FIG. 1). We have developed a new method how to grow M. tuberculosis bacteria rapidly in culture which is under study and results are promising. The M. tuberculosis bacteria is a gram-positive, an obligate aerobe and requires a host for growth and reproduction, and does not from spores. Also M. tuberculosis is considered a facultative intracellular parasite that is transferred through the air, which is why tuberculosis appears in the upper lungs first. If the tuberculosis bacterium develops resistance to treatment, it may be prolonged from 6 months to 24 months or more. This long duration of treatment is necessary due to poor efficacy of available antibiotics, including the main drugs isoniazid and rifampin, against dormant M. tuberculosis bacteria that persist in particular environments such as granuloma, caseous material, cavitary TB, and the walls of the cold abscess. It is interesting to note that a rapidly growing culture of M. tuberculosis is killed within days by the antibacterial drugs, but it takes 6 months to complete the sterilization of tuberculous lesions in the lungs of patients. This is attributed due to the presence of slowly growing, at different stages in development of the same population, or non-multiplying populations of persister bacilli, in particular those in the stationary phase of growth or existing under anaerobic conditions (Michison and Selkon 1956).

Many attempts have been made for identifying antibacterial substances having bactericidal properties against Mycobacterium tuberculosis cells in the dormant state. Hu et al. (2006) described vitro activity of pyrazinamide, a sterilizing substance, against M. tuberculosis conditions mimicking cells in dormant state. Murphy et al, (2007) have determined the targets for drug discovery included in several regulatory elements (devR/devS, relA, mprAB) enzymes involved in redox balance and respiration, sulfur transport and fixation, pantothenate, isoprene and NAD biosynthesis. However, there is still a need for novel anti M. tuberculosis bactericidal substances, active against the dormant state of M. tuberculosis bacteria cells, as well as techniques for the screening and selection of such bactericidal substances.

Histological Characteristics of Tuberculosis Bacteria:

The Tubercle bacilli are slender, beaded, non-motile (though it has flagellum, may be just stationary mobility, wiggling), with waxy cell wall (due to high content of mycolic acid—FIG. 1) and are responsible for the staining pattern of poor absorption followed by high retention that confer acid-fastness (retention of carbol fuchsin after rinsing with acid alcohol so also bacillus Leprae). It is a gram-positive bacillus, strict, obligate aerobe that proliferates within phagocytes in the body. It is surrounded by two cell membranes, and the cytoplasm contains ribosome, plasmid, chromosomal DNA, and proteosomes. The bacteria are provided with flagellum which is said to help in their motility (just wiggle locally). It is also endowed with multiple hairs like appendage named pili (pilus) on the outer surface. Though we do not know the function of these pili in this bacteria cell wall, it is thought to be used to protect the cell's surface and helps in adhesion to other surfaces. May be they helps to latch on to the surface of the air passages, alveolar surface and macrophage in the lung alveoli. They grow and multiply (by binary fission) slowly in culture and require 3 or more weeks to develop colonies. It is endowed with a very thick cell wall (FIGS. 1, 3, 4), resists heat and disinfectants but is killed quickly by ultraviolet light. That is why it takes several months of anti TB drugs to break through and penetrate the thick cell walls and kill the bacteria (FIG. 4). We plan to develop methods to grow the organism rapidly in the laboratory.

Genetic Makeup of Tuberculosis Bacteria:

The complete genome sequence of the best-characterized strain of Mycobacterium tuberculosis. The Mycobacterium tuberculosis H37Rv genome is 4.41 Million by long and contains approximately 4047 predicted genes. H37Rv has been determined to improve our understanding of the biology of this slow-growing pathogen and to help the conception and development of new prophylactic and therapeutic interventions. The genome comprises 4,411,529 base pairs, contains around 4047 genes, and has a very high guanine+cytosine content that is reflected in the biased amino-acid content of the proteins. M. tuberculosis differs from other bacteria in that a very large portion of its coding capacity is devoted to the production of enzymes involved in lipogenesis and lipolysis, and to two new families of glycine-rich proteins with a repetitive structure that may represent a source of antigenic variation (Cole et al. 1998). It is also endowed with large amount of proteosomes unlike most other bacteria; which is being explored to develop therapeutic agents which attack proteosomes. A proteasome is a protein degradation “machine” within the cell that can digest a variety of proteins into short polypeptides and amino acids. The proteasome is itself made up of proteins and requires ATP to function. Structurally, it is hollow and has openings at both ends to allow entry of the protein to be digested and extruded.

M. tuberculosis Bacterial Cell Wall Structure (FIGS. 1, 3, 4):

The M. tuberculosis has two cell walls that contain peptidoglycan with periplasmic intermembrane space between the cell walls called periplasmic space (FIG. 1 #87). Sixty percent of the cell wall is lipid, in particular mycolic acids are said to determine the virulence of the microbe (FIG. 4). Mycolic acids are hydrophobic and help determine the permeability of the cell surface. The cell also contains the “cord” factor that causes colonies of M. tuberculosis to grow in a serpentine like fashion. The inner lining membranes play a major role in metabolism of the bacteria besides its matrix producing needed ATP energy. Insulin which we incorporate in the treatment of tuberculosis makes these membranes more permeable to anti-tuberculosis therapeutic agents and Vitamin C.

M. tuberculosis Bacterial Reproduction:

M. tuberculosis bacteria can reproduce within the phagocytic cells of humans including dendritic cells and macrophages and lay hidden as latent persisters cell TB (FIG. 1a). Bacteria grow in a warm host (typically human), but can remain alive up to 10 weeks in a dry state in saliva, and are killed by sun's ultraviolet rays. The bacteria initially grow and divide in the lung alveoli, alveolar ducts, and draining lymph nodes in addition to the phagocytic cells (FIG. 1a, 5). TB bacteria like other bacteria replicate by binary fission. A single bacterial cell, called a mother cell, copies the chromosome, and then the cell splits in half, giving each half of the cell a copy of the chromosome. The two new identical daughter cells are essentially clones of the mother cell. After entering a host, M. tuberculosis lays dormant (latent) for years or even decades hiding within the macrophages (persisters). This may be in part due to the immune response of the host.

Let us examine what happens after the bacteria lands in the lungs alveoli and phagocytosed by the macrophages in the alveoli. The BV of the alveoli pour inflammatory cell to the alveolar cavity to attack the free bacteria and phagocytosed macrophages. In 5-10% of the cases, the bacteria overcomes the immune response and emerge as victorious, set up residence in the alveoli and initiate a full blown infection within the host (FIG. 1a) lung alveoli where they reside. The immune response to infection and infected macrophages in the alveoli results in:
a) Formation of non-necrotic granuloma (tubercle— FIGS. 2, 5) consisting of macrophages, giant cells at the periphery, lymphocytes, T cells, B-cells, which may be dotted with patches of caseous necrosis (FIG. 2). Non necrotic granuloma consists of macrophage with TB bacteria, surrounded by lymphocytes mixed with non-infected macrophages, with giant cells positive for bacteria at the periphery of the lesion.
b) As the disease progress, necrosis sets in with necrotic granuloma formation. The necrosis sets in the granuloma center. The lesion consists of caseous necrosis center with transition Zone of non-infected macrophages, surrounded by infected and non-infected macrophages intermingled with massive lymphocyte infiltration surrounding of the caseous center. This lymphocyte mantle is dotted with giant cells with and without M. tuberculosis bacteria.
c) The granuloma may coalesce into a larger yellowish caseous cheese mass and/or the center may breakdown, liquefy, leading to cold abscess formation, made up of liquefaction of granuloma center surrounded by reactive exudates composed of serum, leucocytes, and M. tuberculosis bacilli. It is surrounded by infected and non-infected macrophages dotted with giant cells in the periphery with massive wreath of lymphocytes (FIG. 1a, 2).
d) In the lungs, this necrotic granuloma lesion can breakdown leading to cavitary tuberculosis (FIG. 1a, 2, 5) spewing the infection to other parts of the lungs and body; and spread infection to others people with droplet infection due to coughing.
e) Or persists without further progression, may be even surrounded with fibrous capsule and mottled calcification (Calcified Ghon's focus).

After the host immune response is depressed, the bacteria re-emerge and initiate a full blown infection within the host (FIG. 1a). The infected macrophages form granuloma (tubercle— FIGS. 2, 5) consisting of macrophages, giant cells, lymphocytes, T cells, B-cells, with patches of caseous necrosis, and fibroblasts surrounding the lesions (FIG. 2). These may coalesce into a larger yellowish mass or the center may breakdown leading to cold abscess, made up of liquefaction of granuloma center surrounded by reactive exudates composed of serum, leucocytes, and M. tuberculosis bacilli. In the lungs, the lesion can breakdown leading to cavitary tuberculosis (FIG. 1a, 2).

Tuberculosis Bacterial Metabolism:

Under aerobic condition, M. tuberculosis sustains itself via glycolysis, and oxidative phosphorylation provides the necessary ATP without mitochondria (FIGS. 1, 4). Glycolysis is the metabolic pathway that converts glucose C6H12O6, into pyruvate, C H3COCOO−+H+. The free energy released in this process is used to form the high-energy compounds ATP (adenosine triphosphate) and NADH (reduced nicotinamide adenine dinucleotide. Glycolysis occurs, with variations, in nearly all organisms, both aerobic and anaerobic. It is this energy we want to block by using dinitrophenol and preventing M. tuberculosis bacillus to thrive. It is suggested that M. tuberculosis sustains itself on lipids while it replicates within the macrophage endosome (a microscopic membranous sac inside a living cell that is pinched off from the cell's outer membrane and contains substances ingested by the cell). Through the process of B-oxidation, M. tuberculosis degrades and uses host-cell lipids as the precursors for many of its own metabolic processes. M. tuberculosis usually contains examples of “every known lipid and polypeptide biosynthetic system” (Cole 1998). M. tuberculosis divides every 15-20 hours. We are developing a method to enhance this division and growth rapidly in the culture media.

Different Stages M. tuberculosis Undergoes within the Host after Entry:

Stage 1:

Onset (1-7 days): Bacteria are inhaled through the air and typically engulfed by alveolar macrophages (FIG. 1a). At this instant, disease progression depends on: 1. the virulence of the inhaled strain, 2. the anti-mycobacterial capabilities of the macrophage in question and 3. Immune system of the infected. Tuberculosis begins when the inhaled mycobacteria reach air passage and alveolar macrophages laden with M. tuberculosis bacteria (FIG. 1a) with tubercle formation (Todar 2005).

Stage 2:

Symbiosis (7-21 days): If the initial macrophage does not succeed in killing the bacteria, the bacteria will replicate until the macrophage bursts out of the releasing the bacteria (FIG. 1a). The bacteria are now engulfed by other alveolar macrophages and non-activated macrophages. The macrophages that arrive from the bloodstream, migrate to alveoli, engulf the exposed bacteria in a symbiotic manner—neither the host nor the bacteria is harmed at this stage (Danenberg 1994).

Stage 3:

Caseous Necrosis (14-21 days): The next stage of TB disease development begins when bacterial reproduction slows. Growth slows because as the bacteria reproduce, and kill all the surrounding non activated macrophages at the same time run out of cells to divide within. In addition, the increased number of bacteria produces anoxic conditions and reduces the local pH (Dannenberg 1994). The bacteria can no longer reproduce in this tubercule lesion, but can remain alive for long periods of time at this state. Tubercules are also described as having caseous centers due to their appearance as shown in FIG. 1a, 2, 5, and 7. The host kills its own tissues to prevent the spread of the bacteria. Also at this stage, the host will test positive for tuberculin (Dannenberg 1994). As the granulomas mature, they show more vacularization and develop a capsule which somewhat separated macrophages, granulocytes, and the lymphocytic infiltrate. As the disease progress, the centers of the granulomas lose vacularization and undergo necrosis (hence the name caseous necrosis, FIG. 1a, 2; Russell 2007). At this stage, T-cells begin to recognize presented M. tuberculosis, which causes T-cell activation and production of cytokines including INF-gamma. INF-gamma activates macrophages, increasing their ability to destroy the invading bacteria (Todar 2007).

Stage 4:

Interplay of Tissue-Damaging and Macrophage Activating Immune Response (After 21 days): Macrophages surround the tubercule, some of which may be inactivated. M. tuberculosis uses the inactive macrophages to reproduce, causing the tubercule to grow. The tubercule may break off or spread into the bronchus, then other parts of the lung and the blood vessels of the alveoli carrying the bacteria far and wide (FIG. 1a).

Stage 5:

Liquefaction and Cavitary Formation: At some point the centers of the tubercules liquefy, which produces a very conducive environment for the bacteria and rapid spread of the disease. Only a very small percentage of infected individuals will progress to this cavitary stage, mostly in lungs as shown in FIG. 5 (Todar 2005).

Reactivation—

Latent re-emergence of TB after the primary infection triggered by various factors including HIV/AIDS, poor nutrition, old age, and stress (Russell 2007). In addition, people with HIV are approximately fifty times more likely to contract and develop TB than individuals without HIV (WHO 2007). These effects are apparent in sub-Saharan Africa where since the mid-1980s, the onset of the HIV epidemic, annual reported TB infections have risen approximately 400 percent (Areas 2004).

Immunity and M. tuberculosis Bacteria-why is it so Deadly?

Mycobacterium tuberculosis is the most successful pathogens to date, killing millions of individuals worldwide every year. A hallmark of M. tuberculosis infection is that following phagocytosis the microorganisms resist lysosomal delivery, instead residing within phagosome that do not fuse with lysosomes. Phagolysosomes are equipped with the machinery to generate peptide-MHC II complexes. Inhibition of Phagolysosomes fusion has been proposed to represent a mechanism by which M. tuberculosis escapes efficient antigen presentation by host MHC II complexes. This can occur by fusion and fission of phagosomes with endoplasmic reticulum-derived vesicles containing newly synthesized MHC I molecules. Recently, the inventors (U.S. Pat. No. 8,647,641 B2) reported that a putative mycobacterial zinc met allo-protease, Zmp1, may play an important role in disease pathogenesis by interfering with two pathways of pathogen defense: inflammasome activation and phagosome maturation (Master, et. al. 2008).

Macrophages which are primarily infected with the M. tuberculosis bacteria in the lungs have been shown to act as the principal effectors of Mycobacterium immunity; and T cells are the major inducers of such immunity. The essential role of T cells in protection against Mycobacterium infection is illustrated by the frequent occurrence of Mycobacterium infection in AIDS patients, due to the depletion of CD4+ therapeutic cells associated with human immunodeficiency virus (HIV) infection.

Both CD4+ and CD8+ T-cells play a role in immune response to TB infection, CD4+ cells are more crucial to the success of the immune response. CD4+ cells are further subdivided into Th1 and Th2, (Chan 1994). Th1 cells produce INF-gamma, IL-2, and lymphotoxin increase antimicrobial activity of macrophages and help control the disease progression through INF-gamma. Th2 cells produce IL-4, IL-5, IL-6, and IL-10; support B-cell proliferation and differentiation. But Th2 cells hinder immune response through the production of IL-4 by inhibiting the Th1 growth (Barnes, P. F., Modlin, R. L., and Ellner, J. J. 1994. T-Cell Responses and Cytokines. In: Tuberculosis: Pathogenesis, Protection, and Control (Bloom, B. R.). ASM Press, 417-435). As a result, activation of a Th2 response generally leads to disease progression, whereas activation of a Th1 response leads to protection (WHO 2007)

M. tuberculosis replicates within vacuoles in macrophages, as a result MHC-II recognition of the pathogen via CD4+ cells and the CD4+ Th1 cells release substantial amount of INF-gamma. INF-gamma plays a crucial role in mounting the immune response to M. tuberculosis, because induces anti-mycobacterial activity in macrophages, specifically inducing NOS2 expression (Chan, J., and Kaufmann, S. H. E. 1994. Immune Mechanisms of Protection. In: Tuberculosis Pathogenesis, Protection, and Control (Bloom, B. R.). ASM Press, 389-415). CD4+ T-cells also have the capability to lyse phagocytes displaying mycobacteria on the MHC class II receptors. These cells lyse infected cells through perforin (bores a hole in the infected cells membrane) and granulysin (activates the capase cascade) (Flynn, J. L. and Chan, J. 2001. Immunology of Tuberculosis. Annual Review of Immunology 19:93-129). More effective phagocytes then hopefully take up exposed bacteria. On the other hand target cell lysis causes host cell damage in the form of tissue and organ damage. Therefore, lysis of M. tuberculosis infected cells can be detrimental or beneficial depending on the circumstances (Chan 1994). CD8+ cells have two effector functions in the immune response to tuberculosis: lysis of infected cells and production of cytokines, INF-gamma. Some M. tuberculosis antigens have been presented on MHC-I molecules, but the mechanism by which these antigens gain access to MHC-I is not understood (Flynn 2001).

In addition to αβ T-cells, gamma delta T-cells has also been shown to respond in the mycobacterium infections. In vitro, gamma delta T-cells are activated by mycobacteria and have been shown to accumulate at the site of infection and gamma delta T cells have been reported to secrete INF-gamma as part of the early immune response. Whereas CD4+ cells are vital for protective immunity and memory (Tsukaguchi, K., Balaji, K. N., and Boom, W. H. 1995. CD4+αβ T Cell and gd T Cell Responses to Mycobacterium tuberculosis: Similarities and Differences in Ag Recognition, Cytotoxic Effector Function, and Cytokine Production. Journal of Immunology 154: 1786-1796).

Mycobacterium-reactive CD4+ T cells have been shown to be potent producers of y-interferon (IFN-γ), which trigger the anti-M. Tuberculosis bacterial effects of macrophages in mice. Studies on the role of IFN-γ in humans show that 1,25-dihydroxyvitamin D3, either alone or in combination with IFN-γ or tumor necrosis factor-alpha, activates human macrophages to inhibit M. tuberculosis infection. Also, it is known that IFN-γ stimulates human macrophages to make 1,25-dihydroxy-vitamin D3. Similarly, interleukin-12 (IL-12) has been shown to play a role in stimulating resistance to M. tuberculosis infection. For a review of the immunology of M. tuberculosis bacterial infection, peruse Chan & Kaufmann (2003), and Harrison's Principles of Internal Medicine, (2005). There is a need for effective treatment strategies to prevent infection and prevent reactivation of Mycobacterium tuberculosis bacterial infections, from both active and latent infections. The present inventions described formulate to achieve this objective.

Latent TB and Activation of Hidden M. tuberculosis:

About one-third of the world population has latent TB infection (LTBI), the majority of which is distributed in 22 high-burden countries. Early diagnosis and treatment is top priority LTBI contributes significantly to the pool of active TB cases later on, especially in high-risk groups. Our invention will help in this menace of latent infection. A high prevalence of drug-resistant TB, the HIV epidemic, and delays in the diagnosis of active TB cases are other major concerns in areas of high TB prevalence.

Reactivation—Latent re-emergence of TB after the primary infection triggered by various factors including HIV/AIDS, poor nutrition, old age, and stress (Russell 2007). In addition, people with HIV are approximately fifty times more likely to contract and develop TB than individuals without HIV (WHO 2007). These effects are apparent in sub-Saharan Africa where since the mid-1980s, the onset of the HIV epidemic, annual reported TB infections have risen approximately±400 percent.

After entering the body, in the majority of cases, the bacteria are suppressed by the immune system and do not replicate. Instead they lie dormant; hibernating inside the patient's phagocytotic (persister) cells (FIG. 1a, 3, 5) called as latent tuberculosis which can be detected with immune based tests such as the tuberculin skin test or interferon gamma release assays (IGRA). Therapy for those with positive tests can reduce the subsequent risk of re-activation and development of active TB. Current therapy is isoniazid (INH) reduces the risk of active TB by as much as 90 percent if taken daily for 9 months, but the compliance is problem.

Only about 5-10 percent of these latent TB cases turn into active infections (reemergence of TB after the primary infection), but it is important to treat latent TB in certain groups of people that are at high risk for developing the full-blown disease, triggered in immune-compromised individuals, such as with HIV/AIDS, on monoclonal antibody and corticosteroid therapy, immune suppression therapy, poor nutrition, old age, other debilitating systemic diseases, stress (Russell 2007, DC Supplemental www.pnas.org/cgi/content/full/0711697105/) and children. These latent tuberculosis cases when diagnosed can be treated using high doses of Vitamin C and rifampicin to suppress/kill the M. tuberculosis bacteria and even eliminate them completely from the body as described in this invention instead of INH therapy for 9 months.

When an individual contracts tuberculosis infection, the body natural defense mechanisms unleash to combat the infections. Bio-Chemicals known as cytokines are released to activate macrophages to kill the tuberculosis bacteria. CD4 cells are also very important for the body to fight this type of infection. HIV destroys CD4 cells, which makes it very easy for the HIV positive individual to contract tuberculosis. However, in most TB cases, the immune response is not strong enough, TB lesions (Tubercles) develop in the lungs and the infection moves to the hilar lymph nodes (FIG. 1a, 5) creating symptomatic primary tuberculosis. As the disease advances, the tuberculosis bacterium causes pleural effusions, pneumonia, and lobar collapse especially in children. Depending on the state of the immune system, the tuberculosis can also cause miliary tuberculosis or tuberculous meningitis.

Ghon's Complex—the Pulmonary Site of Primary Tuberculosis:

First Infection with TB bacteria usually results in so-called primary tuberculosis or Ghon's complex in approximately 90% of infected (FIG. 1a, 5A #49). This usually limited infection comprises a focal multiplication of the Mycobacterium in the lung tissue in association with lymphangitis, infection, and vast enlargement of the corresponding hilar draining lymph node. 95% of these individuals, the inflammatory reaction is contained (FIG. 1a #68,69) and in many cases calcifies and persists for the remainder of the TB infection all through the person's life or may be active within 2 years or may take up as long as 5 years after infection as described in patent US 2010/0022504 A1 which is incorporated here. Despite the clinical resolution of Ghon's complex, several M. tuberculosis organisms remain viable for life, a condition known as latent or dormant tuberculosis (FIG. 1a, 5). These viable bacteria contribute to the reactivation of the disease decades later as the conditions permit with rundown immune system due to lowered body defences to any number of reasons. This primary tuberculosis more often than not stimulates strong and long-lasting cellular immune response to M. tuberculosis antigens, detected even years later by the delayed-type hypersensitivity skin test (the purified protein derivative—PPD or Mantoux test).

Anatomic Histology of the Lungs and its Relation to Tuberculosis Exposure for Infection:

Tuberculosis mainly affects the lungs (FIG. 5, FIG. 8 flow chart) because it is the most exposed to droplet infection from other patients whose sputum is positive for mycobacterium tuberculosis. The human lungs have a combined surface area of 50 to 100 m²(1076 sq. ft. compared to 2 square meters or 22 square feet of skin) exposed from within and to the surroundings for multitudes of infections and adverse environmental conditions. If all of the capillaries surrounding the lungs alveoli were unwound and laid end to end, they would extend for about 992 kilometers (616 miles). The lungs weigh approximately 1.3 kilograms (approximately 2.5 lb each to a total of 5 lb, compared to skin about 20 lb., liver 3.21b, brain 3 lb., heart 0.61b), with the right lung weighing more than the left. Both the lungs contain approximately 2,400 kilometers (1,500 miles) of airways and 274-790 million alveoli (estimated by counting their openings at the level of the free septal edges, where they form a two-dimensional network) involved in gas exchange meaning exposed to droplet infection of tuberculosis from the open TB patients and other infections from the inspired air transmitted. One cubic millimeter lung parenchyma contains around 170 alveoli (Ochs, et al. 2004). Terminal bronchiole branches and grape-like clusters of alveolar sacs extend from the terminal bronchioles surrounded by miles of capillaries exposed to air and TB droplets. The respiratory passages with lungs besides the skin are the only organs in the body that are constantly and directly open to the external environment exposing them to epidemiological, environmental (droplet infection from tuberculosis and other viral-bacterial infected person, FIG. 5), occupational, personal and social factors (close congested living) and their related diseases as exemplified by the TB. The lungs are also often affected in multisystem diseases from within besides external environmental afflictions making them more susceptible for droplet TB infection, blood emboli, blood and lymphatic infections, and cancers, heart failure, endocrine dysfunctions, kidney afflictions, emaciation, and the list is endless.

Various TB Bacterial Infection Treatment (FIGS. 8, 8a) and Drug Resistance:

The treatment comprises of Latent infection, active infection and drug resistant infection. According to Sia and Wieland of May (2011) clinic proceedings, the latent infection is treated using the following regimen using Isoniazid:

| Medication | Adult dose (maximum) | Interval and duration |
|---|---|---|
| Preferred regimen | | |
| Isoniazid | 5 mg/kg (300 mg) | Daily for 9 months |
| Alternative regimens | | |
| Isoniazid | 15 mg/kg (900 mg) | Twice weekly for |
| | | 9 months |
| Isoniazid | 5 mg/kg (300 mg) | Daily for 6 months |

Treatment of TB and Drug Resistance M. tuberculosis Bacteria:

The treatment of tuberculosis comprises of two components, i.e. 1. Intensive phase and, 2. Continuation phase. The purpose of the intensive phase is to kill microorganisms which are dividing (growing) and to render the tissues (sputum) negative of organism and prevent its spread. Our invention described here will help in these processes. Our inventive therapies described herein are applicable in the intensive phase of the therapy. If treatment is stopped at this point, then there is a high relapse rate (more than 20%) besides developing drug resistant tuberculosis. This is due to organisms which lie dormant and persist in the tissues (persisters—FIG. 1a). The component of the therapy is the continuation phase to kill persisters (FIG. 1a, #69) and bring the relapse rate as low as possible (less than 5%). Due to longer duration of treatment extending more than six to twenty four months or more, the expenses involved, and compliance is problematic. To improve compliance the therapy is given as a directly observed therapy (DOT). This increases the cost of therapy considerably, but better compliance. Non adherence to treatment is a major problem in TB control. Inadequate treatment can lead to:

1. Treatment failure,

2. Relapse,

3. Ongoing transmission to other healthy people, and

4. Development of drug resistance.

Educating patients about TB disease ensures their successful completion of therapy and DOT can help in the successes. It is important to note that the compliance with the relatively long course of treatment is generally poor. Such non-compliance may well lead to treatment failure resulting in development of drug resistance. That is why we believe that the method described here will lead to treatment successes due to short duration of treatment instead of 6 to 24 months with increased compliance. At present, important antibacterial drugs are: isoniazid, rifampin, and streptomycin and inhibiters of the development of resistance are: isoniazid, rifampin, and ethambutol.

Unfortunately, the results of modern multiple drug therapy for the treatment of TB due to long therapy, cost and compliance failure are discouraging. It takes even a longer time for effective treatment when tuberculosis organisms are resistant to single drug (SDR) or multidrug resistance (MDR-XDR) such as Rifampicin and Isoniazid. Ethionamide (2-ethylthioiso-nicotinamide, 2-ethylpyrimidine-4-carbothioamide), a structural analogue of isoniazid, is currently the last line of defense in the treatment (U.S. Pat. No. 8,912,329 B2) of multi-drug-resistant tuberculosis (MDR-TB) with little cross-resistance with isoniazide. Up to a 1 g/day are required for an acceptable concentration in blood (Holdiness, M. R., Clin Pharmacokinet 1984, 9, 511-44), which is associated with severe side-effects including neurotoxicity and fatal hepatotoxicity. Here again using our hypoglycemic method (IHAT), the dose can be cut down more than 50% and used to treat drug resistant TB with minimum side effects.

It is thought that the resistance can only occur through chromosomal mutation although rarely movement of mobile genetic elements, such as the insertion sequence IS6110, has been associated with new resistance emerging through the inactivation of critical genes. Patients who been never treated before, or treated for less than one month develop drug resistance are called “Primary drug resistance” and those who develop resistance M. tuberculosis bacteria to drugs are defined as “acquired drug resistance” Treatment of MDR tuberculosis are very expensive, toxic, arduous and frequently ineffective. It is here our described therapy can be very effective.

TABLE 1
Summary of the molecular mechanisms of some of the anti-tuberculosis
drug resistance:

| Drug | Associated mutated gene or mutation |
|---|---|
| Rifampin | rpoB |
| Isoniazid | katC, inhA, oxyR, ahpC, furA |
| Streptomycin | rrs, rpsL |
| Pyrazinamide | pncA, IS 6110 insertion |
| Ethambutol | embB |
| Fluoroquinolones | gyrA, gyrB |

Clinical complications such as empyema and extensive lung cavitations at the site of infection permit a large population to develop in a compartment into which drugs may not permeate and penetrate. Our inventive device and therapies (FIGS. 5, 6, 7) facilitates the delivery of therapeutic agents to the site of lesion, where the systemic therapeutic agents may not be able to reach in enough concentrations to kill the M. tuberculosis. Such treatment should be effective to reduce the burden of treatment failure cases, and be effective in management of treatment failure cases including MDR tuberculosis. Our invention will make it possible to convert sputum positive to negative for Mycobacterium tuberculosis rapidly and prevent the spread of the disease from the respiratory system by coughing (droplet method of spread) and close contact, ultimately curing the disease.

The achievements of the World Health Organization's “Stop TB” program have been largely overlooked by the media, common people, as well as care givers, but mortality and incidence rates for clinically diagnosed tuberculosis have fallen for the second year (WHO, 2012a). Besides first time tuberculosis, we have to look into TB relapse. It is a complicated phenomenon influenced by local public health conditions; for instance, countries with low burden may have increases relapse rates due to reactivation of latent TB (Jasmer et. al., 2004), while relapses in areas in which the TB is endemic may be largely due to reinfection (Narayanan et al., 2010). With clinical descriptions of multiple (MDR), extensively (XDR), and totally (TDR) drug resistant strains in the literature (Shah et al., 2011; Udwadia and Amale, 2012), it looks as if the M. tuberculosis bacteria is winning the war by accumulating resistance alleles before an effective anti TB drugs and chemotherapy can be developed.

BCG Vaccination as Prophylaxis Against Tuberculosis-Pros and Cons:

The use of vaccines to prevent tuberculosis in humans has proved to be a tremendous challenge for almost a century now. BCG, derived from M. bovis, is currently the only tuberculosis vaccine in use and is the most widely used vaccine in the world. The development and generalized administration of the BCG vaccine since the beginning, of the 1920s represented a significant advance, with the prospect of being able to eradicate tuberculosis from the world. However, these initial promises were not achieved and, from the results of a large number of efficacy trials, it is clear that the BCG vaccine in its current form is of limited use in controlling the disease, particularly in respiratory forms in adults in third world areas where the disease is endemic. With more knowledge of the virulence of M. tuberculosis and immune response models that lead to the generation of protective immunity, it is possible to develop better vaccines than BCG. The observation that higher protection levels are achieved when the host is vaccinated with BCG suggests that viability and persistence are fundamental properties required for the success of a tuberculosis vaccine. In 2014 U.S. Pat. No. 8,642,011 B2, by Montanes et. al., invention describe the use aM. tuberculosis strain with the inactivated Rv0757 (phoP) gene and a second independent mutation of phoP, which prevents DIM synthesis, as a prototype single dose live vaccine, and show that, as well as being more attenuated than BCG in immune-compromised SCID mice, it provided protection levels comparable to those conferred by BCG in mice and higher protection than BCG in guinea pigs.

BCG (Bacille Calmette-Guerin), the vaccine named for its creators Albert Calmette and Camille Guerin, who began developing a TB vaccine in 1908 by culturing M. bovis. The first vaccine was administered in 1921 since then more than one billion individuals have been inoculated with BCG. This is a widely controversial vaccine because of extremely variant results from no protection to excellent protection (Aeras 2004). The BCG vaccine can be anywhere from 0 to 80% effective in preventing tuberculosis for a duration of 15 years; however, its protective effect appears to vary according to geography and the lab in which the vaccine strain was grown (Venkataswamy, et. al., 2012). A 1994 systematic review found that BCG reduces the risk of getting TB by about 50%. A systematic review and meta-analysis conducted in 2014 demonstrated that the BCG vaccine reduced infections by 19-27% and reduced progression to active TB by 1% (Roy A, et. al. 2014. “Effect of BCG vaccination against Mycobacterium tuberculosis infection in children: systematic review and meta-analysis”. doi:10.1136/bmj.g4643:BMJ 349: g4643). Recent study has shown that natural infection with M. tuberculosis and vaccination with BCG does not differ in their capacity to bring about protective immunity against tuberculosis (Sampson, S. L., Dascher, C. C., Sambandamurthy, V. K. et al. 2004). This raises questions as to whether or not it is possible to improve BCG by rational attenuation of M. tuberculosis bacteria.

One can see by these studies how effective (or ineffective) BCG vaccination is in protecting the exposed to M. tuberculosis bacteria from infection! Currently more vaccines are being developed (U.S. Pat. No. 8,642,011 B2. 2014 by Montanes et. al. U.S. Pat. No. 8,647,641 B2 by Boettger and Sander 2014) and one day we will have an effective vaccination against this dreaded endo-pandemic disease. Invention WO 2014/176498 A1 to Hartman et. al. addresses a need for improved methods of treating M. tuberculosis infection using enhancers of respiration, and compositions for treating tuberculosis as well as assays for identifying novel agents for treating M. tuberculosis infection. We describe in this invention a method to increase TB bacterial respiration by hyperthermia, insulin, Vitamin C, anti-tuberculosis drugs, and oxygen supplementation as well as by use of cysteine, dithiothreitol, penicillamine if need be as proposed by Hartman et. al.

The current recommended antibiotic chemotherapy treatment of pulmonary tuberculosis involves a two month course with daily administration of the front line antibiotics Isoniazid (INH), Rifampicin (RIF), Pyrazinamide (PZA), and Ethambutol (EMB); followed by a four month continuation phase of INH and RIF (WHO, 2012b). Multi drug resistance (MDR) and single drug resistance (SDR) strains require treatment which regularly lasts two years or more. The necessity for such a protracted therapeutic agent's regimen is thought to be due to the presence of persister cells which are insensitive to antibiotic treatment (FIG. 1a). The persister cells are a fraction of the population that do not harbor genetically encoded features which render them permanently resistant; their survival mechanism is temporary, and sensitivity to antibiotics can be restored upon re-growth. Monitoring Treatment and adherence to the protocol is an important component of therapy to cure TB according to the study by Swaminathan, and their group (Swaminathan et. al. 2000, 2010). Phenothiazines such as thioridazine and chlorpromazine are old antipsychotic agents have been known to have anti-tuberculosis effects for several decades (Molnar et. al. 1977); their drawback being cardiac and neurological toxicity (Musuka S. et. al. 2013). New drugs are gradually being brought into use, such as TMC207, nitroimadazoles, PA824 and OPC67683. They will need to be tested in new combination regimens for drug-susceptible and multi- and extensively drug-resistant disease. Michison and Davies (2013) have excellent review on the chemotherapy of the tuberculosis: past, present and future.

There is still a need for novel M. tuberculosis bactericidal therapeutic method against the active as well as to methods for the screening and treating the latent cases (dormant M. tuberculosis bacteria and bacteria containing cells). We describe such a therapeutic methods, a new regimen in this invention to treat active and latent TB, and to reduce or eliminate morbidity and mortality with following major goals in mind:
a) Cure the individual patient of M. tuberculosis bacterial infection rapidly within weeks, not 6 months or years as it takes now,
b) Decrease the risk of death and disability; with proper treatment, and
c) Reduce and possibly eliminate the transmission of M. tuberculosis to other persons by making the sputum negative rapidly, and improving the living conditions which plays an important role in the spread of the disease by droplet infection due to coughing to others in close contact,
d) Eliminate latent TB, and persister cells with M. tuberculosis bacteria form the body to prevent reactivation of the tuberculosis at a later date.
e) Effective prophylaxis to kill the M. tuberculosis (effective vaccine or other methods to prevent infection) when and if the exposure is suspected to open TB patient

### REFERENCES

1. Alabaster, Oliver; et al. Metabolic Modification by Insulin Enhances Methotrexate Cytotoxicity in MCF-7 Human Breast Cancer Cells, Eur J Cancer Clinic; 1981, Vol 17, pp 1223-1228.
2. Allison, K. R., Brynildsen, M. P., and Collins, J. J. (2011). Metabolite-enabled eradication of bacterial persisters by aminoglycosides. Nature 4 73, 216-220.
3. Amann R, Peskar B A. Anti-inflammatory effect of aspirin and sodium salicylate. Eur J Pharmacol 2002; 447:1-9.
4. American Thoracic Society and Centers for Disease Control and Prevention. Diagnostic standards and classification of tuberculosis in adults and children. Am J Respir Crit Care Med 2000; 161: 1376-1395.
5. American Thoracic Society, Centers for Disease Control and Prevention, Infectious Diseases Society of America. Treatment of tuberculosis. Am J Respir Crit Care Med 2003; 167:603-662; reprinted inJ Infect Dis 2007; 196: S35-S45. MMWR Recomm Rep 2003; 52:1-77.
6. Andersen et. al. Proteins Released from Mycobacterium tuberculosis During Growth, Infection and Immunity 1905-1910, 1991.
7. Andrews R H, Devadatta S, Fox W, Radhakrishna S, Ramakrishnan C V, Velu S. Prevalence of tuberculosis among close family contacts of tuberculous patients in South India, and influence of segregation of the patient on the early attack rate. Bull World Health Organ. 1960; 23:463-510).
8. Andries, K., Verhasselt, P., Guillemont, J., Gohlmann, H. W. H., Nlefs, J.-M., H., Van Glestd, 1, Timmerman, P., Zhu, M., Lte, E., et al. (2005). A diarylquinolim drug activity on the ATP synthase of Mycobacterium tuberculosis. Science (New York, N.Y.) 307, 223-227.
9. Asbury et. al. “Trapping of DNA in Nonuniform Oscillating Electric Fields”, Biophysical Journal, February 1998, p. 1024-1030, vol. 74, Seattle, Wash.
10. Aydin Ismail Orhan, Aylin Ulusahin, Depression, anxiety comorbidity, and disability in tuberculosis and chronic obstructive pulmonary disease patients: applicability of GHQ-12. General Hospital Psychiatry, Volume 23, Issue 2, March-April 2001, Pages 77-83
11. Bachynsky, Nicholas and Roy, 2009. Chemically induced intracellular hyperthermia by DNP, U.S. Pat. No. 7,635,722 B1, PCT WO00/06143, Human weight loss inducing method by DNP. U.S. Pat. No. 4,673,691.
12. Balaban, N. Q., Merrin, J., Chait, R., Kowalik, L., and Leibler, S. (2004). Bacterial persistence as a phenotypic switch. Science (New York, N.Y.) 305, 1622-1625.
13. Ballel L, Field R A, Duncan K, Young R J. New small-molecule synthetic antimycobacterials. Antimicrob Agents Chemother. 2005; 49:2153-2163.
14. Barker K, Seger D, Kumar S (2006). “Comment on” Pediatric fatality following ingestion of Dinitrophenol: postmortem identification of a dietary supplement. Clin Toxicol (Phila) 44 (3): 351. Dinitrophenol-Induced Hyperthermia Resolving With Dantrolene Administration. Abstracts of the North American Congress of Clinical Toxicology. Clin Toxicol 2002; 40:599-673.
15. Barry, C. E., Boshon: HJ., Dart.ois, V., Dick, T., Ehrt, S., Flynn, J., Schnappinger, Dilkinson, R. J., and Young, D. (2009). The spectrum of latent tuberculosis: rethinking the biology and intervention strategies. Nature Reviews. Microbiology 7, 845-855.
16. Basu S, Andrews J R, Poolman E M, et al. Prevention of nosocomial transmission of extensively drug-resistant tuberculosis in rural South African district hospitals: an epidemiological modeling study. Lancet. 2007; 370: 1500-1507.
17. Beltan. E. et al. 2000. Secretion of cytokine by human macrophages upon infection by pathogenic and on pathogenic Mycobacteria. Microb. Pathog. 28, 313-318
18. Bemey, M., and Cook, G. M. (2010). Unique flexibility in energy metabolism allows mycobacteria to combat starvation and hypoxia. PloS One 5, e8614.
19. Benator D, Bhattacharya M, Bozeman L, et al. Rifapentine and isoniazid once a week versus rifampicin and isoniazid twice a week for treatment of drug-susceptible pulmonary tuberculosis in HIV-negative patients: a randomized clinical trial. Lancet. 2002; 360: 528-534.
20. Berg et al, “Electric Field Effects on Biological Membranes: Electoincorporation and Electofusion”, Ettore MaJ Inter. Science, 1987, pp. 135-166, vol. 32, Phys. Science, New York.
21. Beste, D. J. V, Espasa, M., Bonde, B., Kierzek, A. M., Stewart, G. R., and McFadden, J. (2009). The genetic requirements for fast and slow growth in mycobacteria. PloS One 4, c5349.
22. Block G, Jensen C, Dietrich M, et al. Plasma C-reactive protein concentrations in active and passive smokers: influence of antioxidant supplementation. J Am Coll Nutr. 2004 April; 23(2):141-7.
23. Bocci V, Valacchi G, Corradeschi F et al., (1998). “Studies on the biological effects of ozone: Generation of reactive oxygen species (ROS) after exposure of human blood to ozone”. J. Biol. Regul. Homeost. Agents 12 (3): 67-75.
24. Bocci, V.; Borrelli, E.; Travagli, V.; Zanardi, I. (2009). “The ozone paradox: Ozone is a strong oxidant as well as a medical drug”. Medicinal Research Reviews 29 (4): 646-682.
25. Bocci, V., “Oxygen-Ozone Therapy: A Critical Evaluation”, Kluwer Academic Publishers, 2002
26. Budavari, Susan(ed); O'Neil, Maryadele J(ed); Heckelman, Patricia E (red). “The Merck Index” Rahway, N J; Merck & Co; 1989. [1900] p.
27. Burdett, Beeler and clip. Distribution of Glucose Transporters in Insulin Receptors in the Plasma Membrane and Transverse Tubules of Skel et al Muscle. Arch Biochem Biopys 253:279-286, 1987.
28. Busch W. Aus der Sitzung der medicinischen Section vom 13 Nov. 1867. Berl Klin Wochenschr. 1868; 5:137.
29. Byrne Sean T., Steven M. Denkin and Ying Zhang. Aspirin and ibuprofen enhance pyrazinamide treatment of murine tuberculosis. Oxford Journals Medicine & Health Journal of Antimicrobial Chemotherapy (2006) Volume 59, Issue 2 Pp. 313-316. Clinical Trials Trial number NCT00910871. ClinicalTrials.gov.
30. Cameron E, Pauling L. Supplemental ascorbate in the supportive treatment of cancer: reevaluation of prolongation of survival times in terminal human cancer. Proceeding of the National Academy of Sciences 75:4538-4542, 1978
31. Cameron E, Pauling L and Leibovitz B (1979) Ascorbic acid and cancer: a review. Cancer Res 39: 663-681
32. Canetti, G. 1955. The tubercle bacillus in the pulmonary lesion of man. Springer Publishing Co., Inc., New York, N. Y
33. Canan, C H, N. S. Gokhale, B. Carruthers, W. P. Lafuse, L. S. Schlesinger, J. B. Torrelles, J. Turner. Characterization of lung inflammation and its impact on macrophage function in aging. Journal of Leukocyte Biology, 2014; 96 (3): 473
34. Carmen, J. C., et. al., 2004. Ultrasonically enhanced vancomycin activity against Staphylococcus epidermidis biofilms in vivo. J. Biomater. Appl. 18:237-245.
35. Carpenter Kenneth J. The history of scurvy & vitamin C. Published by Cambridge University Press 1986.
36. Campbell E A. et. al. Structural Mechanism for Rifampicin Inhibition of Bacterial RNA polymerase. Cell, Mar. 23, 2001, Vol. 104, 901-912.
37. Casciari J J, N H Riordan, T L Schmidt, X L Meng1, J A Jackson and H D Riordan. Cytotoxicity of ascorbate, lipoic acid, and other antioxidants in hollow fiber in vitro tumors. British Journal of Cancer (2001) 84, 1544-1550.
38. Centers for Disease Control and Prevention. 2003. Treatment of tuberculosis. Morb. Mortal. Wkly. Rep. Recomm. Rep. 52:1-77.
39. Centers for Disease Control and Prevention. Guidelines for using the QuantiFERON-TB Gold test for detecting Mycobacterium tuberculosis infection, United States. MMWR 2005; 54 (RR-15).
40. Central T B Division, Directorate General of Health Services, Ministry of Health and Family Welfare. TB India 2008-RNTCP status report. Available from http://www.tbcindia.org/pdfs/TB-India-2008.pdf (accessed Dec. 27, 2009).
41. Centers for Disease Control and Prevention. Notice to readers: Updated guidelines for the use of rifamycins for the treatment of tuberculosis among HIV-infected patients taking protease inhibitors or non-nucleoside reverse transcriptase inhibitors. MMWR 2004; 53(02):37.
42. Centers for Disease Control and Prevention (2006). “Notice to Readers: Revised Definition of Extensively Drug-Resistant Tuberculosis”. JAMA: the Journal of the American Medical Association (American Medical Association) 296 (23): 2792
43. Centers for Disease Control and Prevention. Guidelines for the investigation of contacts of persons with infectious tuberculosis: recommendations from the National Tuberculosis Controllers Association and CDC. MMWR 2005; 54 (No. RR-15).
44. Chan & Kaufmann, Tuberculosis: Pathogenesis, Protection and Control (Bloom ed., 1994), Tuberculosis (2nd ed., Rom and Garay, eds., 2003),
45. Chaisson R E, Clermont H C, Holt E A, Cantave M, Johnson M P, Atkinson J, Davis H, Boulos R, Quinn T C, Halsey N A. Six-month supervised intermittent tuberculosis therapy in Haitian patients with and without HIV. Am J Respir Crit Care Med 1996; 154: 1034-1038.
46. Chopra, P., Singh, A., Koul, A., Ramachandran, S., Drlica, K., Tyagi, A. K., and Singh, Y. 2003. Cytotoxic activity of nucleoside diphosphate kinase secreted from Mycobacterium tuberculosis. European Journal of Biochemistry 270: 625-634.
47. Cole S. T. et al. Deciphering the biology of Mycobacterium tuberculosis from the complete genome sequence. Nature 393, 537-544 (11 Jun. 1998.
48. Coley, William B. “A Preliminary Note on the Treatment of Inoperable Sarcoma by the Toxic Product of Erysipelas.” Post-graduate 8:278-86, 1893
49. Cohen, T, Douglas Wilson—and Megan Murray. Mixed-Strain Mycobacterium tuberculosis Infections among Patients Dying in a Hospital in KwaZulu-Natal, South Africa. J Clin Microbiol 2011, V.49(1), 385-3
50. Condos R, Rom W N, Schluger N W (1997) Treatment of multidrug-resistant pulmonary tuberculosis with interferon-c via aerosol. Lancet 349: 1513-1515.
51. Condos R, Raju B, Canova A, Zhao B Y, Weiden M, et al. (2003) Recombinant gamma interferon stimulates signal transduction and gene expression in alveolar macrophages in vitro and in tuberculosis patients. Infect Immun 71: 2058-2064.
52. Coley, W. B. (1893) “The Treatment of Malignant Tumors by Repeated Inoculations of Erysipelas” Am. Journal Medical Science; 105:487-511. A Preliminary Note on the Treatment of Inoperable Sarcoma by the Toxic Product of Erysipelas. “Post-graduate 8:278-86, 1893. Contribution to the knowledge of sarcoma. Ann Surg. 1891; 14: 199-220.
53. Coley-Nauts H, McLaren J R. Coley Toxins—the first century. Adv Exp Med Biol. 1990; 267:483.
54. Collin, Jonathan, “The H2O2 Crusades,” Townsend Letter for Doctors, Op. Cit., June 1989, p. 322.
55. Cone Jr. Clarence D. U.S. Pat. Nos. 4,724,230, 4,724,234, 4,935,450: Method for Producing Oncolysis, Cancer Therapy System for Effecting Oncolysis of Malignant Neoplasms 1990: DNP to treat cancers.
56. Cox, R., and Cook, G. (2007). Growth regulation in the mycobacterial cell. Current Molecular Medicine 231-245.
57. Chemical Technology Encyclopedia; Barnes & Noble 1968 vol 1 pp 82-3.
58. Chuang C H, Sheu B S, Kao A W, et al. Adjuvant effect of vitamin C on omeprazole-amoxicillin-clarithromycin triple therapy for Helicobacter pylori eradication. Hepato gastro enterology. 2007 January; 54(73):320-4.
59. Crofton J. Chemotherapy of pulmonary tuberculosis. BMJ. 1959; 1:1610-1614. Crofton J. Sputum conversion and the metabolism of isoniazid. Am Rev Tuberc. 1958; 77: 869-87.
60. Cumming J N; Ploypradith P; Posner G H (1997). “Antimalarial activity of artemisinin (qinghaosu) and related trioxanes: mechanism(s) of action”. Adv. Pharmacol. 37: 253-97.
61. Cutting, W. C. et al., “Actions and Uses of Dinitrophenol,” JAMA 101, pp. 193-195 (1933).
62. Dannenberg, A. M., and Rook, G. A. W. 1994. Pathogenesis of Pulmonary Tuberculosis: an Interplay of Tissue-Damaging and Macrophage-Activating Immune Responses—Dual Mechanisms That Control Bacillary Multiplication. In: Tuberculosis: Pathogenesis, Protection, and Control (Bloom, B. R.). ASM Press, 459-484.
63. Das, B., Kashino, S. S., Pulu, 1., Kalita, D., Swaami, V., Yeger, H., Felshcr, D. W., and Campos-Neto, A. (2013). CD271+ Bone Marrow Mesenchymal Stem Cells May Provide a Niche for Dormant Mycobacterium tuberculosis. Science Translational Medicine 5, 170ral3.
64. Davis, J Lucian “Diagnostic accuracy of same-day microscopy versus standard microscopy for pulmonary tuberculosis: a systematic review and meta-analysis”, The Lancet Infectious Diseases 23rd Oct. 2012.
65. Dawson R, Condos R, Tse D, Huie M L, Ress S, et al. (2009) Immunomodulation with Recombinant Interferon-Y⁻ 1b in Pulmonary Tuberculosis. PLoS ONE 4(9): e6984. oi:10.1371/journal.pone.0006984.
66. Deng X R1, Liu Z X, Liu F, Pan L, Yu H P, Jiang J P, Zhang J J, Liu L, Yu J. Holotransferrin enhances selective anticancer activity of artemisinin against human hepatocellular carcinoma cells. J Huazhong Univ Sci Technolog Med Sci. 2013 December; 33(6):862-5. doi: 10.1007/s11596-013-1212-x. Epub 2013 Dec. 13.
67. Denkin S, Byrne S, Jie C, et al. Gene expression profiling analysis of Mycobacterium tuberculosis genes in response to salicylates. Arch Microbiol 2005; 184:152-7
68. Dhar, N., and McKinney, J. D. (2007). Microbial phenotypic heterogeneity and antibiotic tolerance. Current Opinion in Microbiology 10, 30-38. Diacon, Andreas H. et al. (2009)
69. Diarylquinoline TMC207 for Multidrug-Resistant Tuberculosis. N Engl J Med 2009; 360:2397-2405.
70. Dickinson, J. M., and D. A. Mitchison. 1987. In vitro properties of rifapentine (MDL473) relevant to its use in intermittent chemotherapy of tuberculosis. Tubercle 68:113-118.
71. Doggrell S A, New drugs being developed for the treatment of tuberculosis. Expert Opin, Investig Drugs. 2005 July; 14(7):917-20.
72. Douglass, Wm M D., “Hydrogen Peroxide: Medical Miracle”, Rhino Publishing Inc. 2003
73. Dodwad V, Gupta S, Kumar K, Sethi M, Masamatti S. Changing paradigm in pocket therapy-ozone versus conventional irrigation. Int J Public Health Dent. 2011; 2:7-12.
74. Dye, C., S. Scheele, P. Dolin, V. Pathiana, and M. C. Raviglione. 1999. Global burden of tuberculosis: estimated incidence, prevalence and mortality by country. JAMA 282:677-686.
75. Dye C, Espinal M A. Will tuberculosis become resistant to all antibiotics? Proc. R. Soc. 2001; 268:45-52.
76. Dye C, Williams B G. Criteria for the control of drug-resistant tuberculosis. Proc. Natl Acad. Sci. USA. 2000; 97: 8180-8185.
77. Edlin, B. R., J. I. Tokars, M. H. Grieco, J. T. Crawford, J. Williams, E. M. Sordillo, K. R. Ong, J. O.
Kilburn, S. W. Dooley, and K. G. Castro. 1992. An outbreak of multidrug-resistant tuberculosis among hospitalized patients with the acquired immunodeficiency syndrome. N. Engl. J. Med. 326:1514-1521.
78. Efferth T, Dunstan H, Sauerbrey A, Miyachi H, Chitambar C R. The anti-malarial artesunate is also active against cancer. Int J Oncol. 2001 April; 18(4):767-73.
79. Elliott, A. M., S. E. Berning, M. D. Iseman, and C. A. Peloquin. 1995. Failure of drug penetration and acquisition of drug resistance in chronic tuberculous empyema. Tuber. Lung Dis. 76:463-467.
80. Enarson, D. A., and A. Rouillon. 1994. The epidemiological basis of tuberculosis control, p. 19-32. In P. D. O. Davis (ed.), Clinical tuberculosis.
81. Chapman & Hall, London, United Kingdom. Elut, S., Guo, X. V, Hickey, C. M., Ryou, M., Monteleone, M., Riley, L. W., and Schnappinger, D. (2005). Controlling gene expression in mycobacteria with anhydrotetracycline and Tet repressor. Nucleic Acids Research 33, e21.
82. Ernst E. 2001. “A primer of complementary and alternative medicine commonly used by cancer patients”. Med. J. Aust. 174 (2): 88-92
83. Farr, C H: “The Therapeutic Use of Intravenous Hydrogen Peroxide (Monograph). Genesis Medical Center, Oklahoma City, Okla. 73120, January 1987.
84. “Fact Sheets Interferon-Gamma Release Assays—Blood Tests for TB Infection”, CDC www.cdc.gov/tb/publicationsfactsheets/85.
Fenton H. J. H. (1894). “Oxidation of tartaric acid in presence of iron”. J. Chem. Soc., Trans. 65 (65): 899-911
86. Filippia A. The effect of ozonated water on epithelial wound healing. Douch Dent J. 2001; 143: 96-101.
87. Filippini, P., lona, E., Piccaro, G., Peyron, P., Neyrollcs, 0, and Fattorini, L. (2010). Activity of drug combinations against dormant Mycobacterium tuberculosis. Anti microbial Agents and Chemotherapy 54, 2712-2715.
88. Flanagan, S. et. al. (1998) “Increased Flux Of Free Radicals In Cells Subjected To Hyperthermia: Detection By Electron Paramagnetic Resonance Spin Trapping” FEBS 431:285-286.
89. Fischl, M. A., R. B. Uttamchandani, G. L. Daikos, R. B. Poblete, J. N. Moreno, R. R. Reyes, A. M. Boota, L. M. Thompson, T. J. Cleary, and S. Lai. 1992. An outbreak of tuberculosis caused by multiple-drug-resistant tubercle bacilli among patients with HIV infection. Ann. Intern. Med. 117:177-183.
90. Ernst E (January 2001). “A primer of complementary and alternative medicine commonly used by cancer patients”. Med. J. Aust. 174 (2): 88-92
91. Faisal A. Khasawnehl and Roger D. Smalligan. Guidewire-Related Complications during Central Venous Catheter Placement: A Case Report and Review of the Literature. Critical care. Volume 2011 (2011), Article ID 287261, 4 pages http://dx.doi.org/10.1155/2011/287261.
92. Ferreira S T, De Felice F G. Neuroprotective actions of 2,4-dinitrophenol: Friend or foe? Dementia & Neuropsychologia 2007; 1(4):334-338.
93. Fishbane S, Mittal S K, Maesaka J K. Beneficial effects of iron therapy in renal failure patients on hemodialysis. Kidney Int Suppl. 1999 March; 69:S67-70.
94. Flor de Lima, B, and M Tavares. “Risk factors for extensively drug-resistant tuberculosis: a review.” The Clinical Respiratory Journal 8.1 (2013): 11-23.)
95. “Fluorescent light-emitting diode (LED) microscopy for diagnosis of tuberculosis”, WHO, 2011
96. Fox W, Sutherland I, Daniels M. A five-year assessment of patients in a controlled trial of streptomycin in pulmonary tuberculosis. Q J Med. 1954; 23: 347-366.
97. Gandhi, N R; Moll, A; Sturm, A W; Pawinski, Robert; Govender, Thiloshini; Lalloo, Umesh; Zeller, Kimberly; Andrews, Jason; Friedland, Gerald (2006). “Extensively drug-resistant tuberculosis as a cause of death in patients co-infected with tuberculosis and HIV in a rural area of South Africa”. The Lancet 368 (9547): 1575-80.
98. Gasché C, Dejaco C, Waldhoer T, Tillinger W, Reinisch W, Fueger G F, Gangl A, Lochs H. Intravenous iron and erythropoietin for anemia associated with Crohn's disease. A randomized, controlled trial. Ann Intern Med. 1997 May 15; 126(10):782-87.
99. Gaurrand S, Desjardins S, Meyer C, et al. Conformational analysis of R207910, a new candidate for the treatment of tuberculosis, by a combined NMR and molecular modeling approach. Chem. Biol. Drug Design. 2006; 68:77-84.
100. Ghosh et. al., “Post-Metaphase Mitotic Events in Cells Treated with Dinitrophenol,” Indian Journal of Experimental Biology, 27(4): 317-323, 1989.
101. Giladi et. al. Microbial Growth Inhibition by Alternating Electric Fields, Antimicrobial Agents and Chemotherapy, October 2008, p. 3517-3522.
102. Goepp, J. Newly Discovered Health Benefits of Vitamin C. Life Extension Magazine April 2008 Issue.
103. Gomez, J. B., and McKinney, J. D. (2004). Mycobacterium Tuberculosis persistence, latency, and drug tolerance. Tuberculosis 84, 29-44.
104. Grant, S. S., Kaufmann, B. B., Chand, N. S., Haseh˜y, N., Hung, D. T., and Schmidt Grant, S. (2012). Eradication of bacterial persisters with antibiotic-generated hydroxyl radicals. Proceedings of the National Academy of Sciences of the United States of America 109, 12147-12152.
105. Gras J. Bedaquiline for the treatment of pulmonary, multidrug-resistant tuberculosis in adults. Drugs Today (Barc). 2013 June; 49(6):353-61.
106. Grosset, J. 2003. Mycobacterium tuberculosis in the extracellular compartment: an underestimated adversary. Antimicrob. Agents Chemother. 47: 833-836.
107. Grundlingh Johann, Paul I. Dargan, Marwa El-Zanfaly, David M. Wood. 2,4-Dinitrophenol (DNP): A Weight Loss Agent with Significant Acute Toxicity and Risk of Death. J. Med. Toxicol. (2011) 7: 205-212
108. Gurumurthy P, Ramachandran G, Hemanth Kumar A K, Rajasekaran S, Padmapriyadarsini C, Swaminathan S, Bhagavathy S, Venkatesan P, Sekar L, Mahilmaran A, et al. Decreased bioavailability of rifampicin and other anti-tuberculosis drugs in patients with advanced human immunodeficiency virus disease. Antimicrob Agents Chemother 2004; 48:4473-4475.
109. Gutierez M, et al. (2004) Autophagy is a defense mechanism inhibiting BCG and M. tuberculosis survival in infected macrophages. Cell 119: 753-766
110. Gumbo, T., A. Louie, et. al., 2007. Isoniazid's bactericidal activity ceases because of the emergence of resistance, not depletion of Mycobacterium tuberculosis in the log phase of growth. J. Infect. Dis. 195:194-201.
111. Gumbo Tawanda, et. al., Concentration-Dependent Mycobacterium tuberculosis Killing and Prevention of Resistance by Rifampin. 43rd Interscience Conference on Antimicrobial Agents and Chemotherapy, September 2003, Chicago, Ill., abstr. A-1156
112. Gupta, A. K., D. S. Chauhan, K. Srivastava, R. Das, S. Batra, M. Mittal, P. Goswami, N. Singhal, V. D. Sharma, K. Venkatesan, S. E. Hasnain, and V. M. Katoch. 2006. Estimation of efflux mediated multi-drug resistance and its correlation with expression levels of two major efflux pumps in mycobacteria. J. Communicable Dis. 38: 246-254.
113. Haagsma A C, Abdillahi-Ibrahim R, Wagner M J, et al. Selectivity of TMC207 towards mycobacterial ATP synthase compared with that towards the eukaryotic homologue. Antimicrob. Agents Chemother. 2009; 53:1290-1292.
114. Haber F, Weiss J. (1932). “Über die Katalyse des Hydroperoxydes (On the catalysis of hydroperoxide)”. Naturwissenschaften 20 (51): 948-950; Koppenol, W. H. (2001). “The Haber-Weiss cycle—70 years later”. Redox Report 6 (4): 229-234).
115. Hartman, T. et. al. Method of Treating Tuberculosis. WO. 2014/176498 PCT/US2014/035448
116. Harrison's Principles of Internal Medicine, Chapter 150, pp. 953-966 (16th ed., Braunwald, et al., eds., 2005).
117. Haynes, R K; Cheu, K W; N′Da, D; Coghi, P; Monti, D (August 2013). “Considerations on the mechanism of action of artemisinin antimalarials: part 1—the ‘carbon radical’ and ‘heme’ hypotheses.” Infectious disorders drug targets 13 (4): 217-77.
118. Haynes, R. K.; Cheu, K. W.; Li, K. Y.; Tang, M. M. K.; Wong, H. N.; Chen, M. J.; Guo, Z. F.; Guo, Z. H.; Coghi, P.; Monti, D. (2011). “A Partial Convergence in Action of Methylene Blue and Artemisinins: Antagonism with Chloroquine, a Reversal with Verapamil, and an Insight into the Antimalarial Activity of Chloroquine”. ChemMedChem 6 (9): 1603-1615.
119. Heep, M., U. Rieger, D. Beck, and N. Lehn. 2000. Mutations in the beginning of the rpoB gene can induce resistance to rifamycins in both Helicobacter pylori and Mycobacterium tuberculosis. Antimicrob. Agents Chemother. 44:1075-1077.
120. Heidenreich K A, Olefsky J M. Metabolism of Insulin Receptors: Molecular Bases for Insulin Action. Page 163, Plenum Press, New York, 1985
121. Heym, B., B. Saint-Joanis, and S. T. Cole. 1999. The molecular basis of isoniazid resistance in Mycobacterium tuberculosis. Tuber. Lung Dis. 79:267-271.
122. Hoffer L J, Levine M, Assouline S, Melnychuk D, Padayatty S J, Rosadiuk K, Rousseau C, Robitaille L, Miller W H Jr. Phase I clinical trial of I.V. ascorbic acid in advanced malignancy. Ann. Oncol. 2008 November; 19 (11):1969-74.
123. Hofmann et. al “Electronic Genetic-Physical and Biological Aspects of Cellular Electomanipulation”, IEEE Eng. in Med. and Biology Mag., December 1986, p. 6-23, New York.
124. Homer, W. D., “Cataract Following Dinitrophenol Treatment for Obesity,” Archives of Opthalmology 16, pp. 447-461 (1936).
125. Hippocrates described symptoms of scurvy in book 2 of his Prorrheticorum and in his Liber de internisaffectionibus. (Cited by James Lind, A Treatise on the Scurvy, 3rd ed. (London, England: G. Pearch and W. Woodfall, 1772, page 285.)
126. Hu et. al., 2006, International Journal of tuberculosis and lung disease, Vol. [0016] 10 (number): 317-322.
127. Huch, A. D. et. al. (1969) “Oxygen Transport in Anesthetized Dogs in Hypoxia, with Oxygen Uptake Increased by 2,4-Dinitrophenol” Respir. Physiol., 6:187-201.
128. Huitric E, Verhasselt P, Andries K, Hoffner S E. In vitro antimycobacterial spectrum of a diarylquinoline ATP-synthase inhibitor. Antimicrob. Agents Chemother. 2007; 51:4202-4204.
129. Ibrahim M, Andries K, Lounis N, et al. Synergistic activity of R207910 combined with pyrazinamide against murine tuberculosis. Antimicrob. Agents Chemother. 2007; 51:1011-1015.
130. Ibrahim M, Truffot-Pernot C, Andries K, Jarlier V, Veziris N. Sterilizing activity of R207910 (TMC207)—containing regimens in the murine model of tuberculosis. Am. J. Respir. Crit. Care Med. 2009; 180:553-557.
131. Imperiale, B R, N S Morcillo, J C Palomino, P Vandamme, and A Martin. (2014). “Predictive value of direct nitrate reductase assay and its clinical performance in the detection of multi and extensively drug resistant tuberculosis.” J Med Microbiol. 2014; 63(Pt_{—}4): 522.
132. Iseman, M D. A Clinician's Guide to Tuberculosis. Baltimore: Lippincott Williams and Wilkens; 2006
133. Ivanyi J, Zumla A. Nonsteroidal antiinflammatory drugs for adjunctive tuberculosis treatment. J Infect Dis 2013; 208:185-8.
134. Jack, D. B. (1997), One hundred years of aspirin. Lancet, 350, 437-439.
135. Janigro et. al. “Alternating current electrical stimulation enhanced chemotherapy: a novel strategy to bypass multidrug resistance in tumor cells”, BMC Cancer, 2006, 6:72.
136. Janin Y I. Anti-tuberculosis drugs: ten years of research. Bioorg. Med. Chem. 2007; 15: 2479-2513.
137. Jasmer, R. M et al., (2004). Recurrent tuberculosis in the United States and Canada: relapse or reinfection′? American Journal of Respiratory and Critical Care Medicine 1 70, 1360-1366.
138. Jayaram, R., Gaonkar, S., and Kaur, P. (2003). Pharmacokinetics-pharmacodynamics of rifampin in an aerosol infection model of tuberculosis. Antimicrobial Agents 47, 2118-2124.
139. Jayaram, R., and Shandil, R. (2004). Isoniazid pharmacokinetics-pharmacodynamics in an aerosol infection model of tuberculosis. Antimicrobial Agents. 48, 2951-2957.
140. Jyoti P, Veeresh M. Nagathan Subramanian M. Rao, Bhemmappa F. B. 2013, Ozone in dental therapy: an outlook. Int J. of clinical dental science. pp 4-8
141. Kanchan, A. et al. (2011) “Rapid Diagnosis of Extensively Drug-Resistant Tuberculosis by Use of a Reverse Line Blot Hybridization Assay.” J Clin Microbiol 49.7: 2546-2551.
142. Keane J, Gershon S, Wise R P, et al. Tuberculosis associated with infliximab, a tumor necrosis factor alpha neutralizing agent. N Engl J Med 2001; 345:1098-104.
143. Keno, Translocation Hypothesis of Insulin Action on Glucose Transport. Federation Proc 43:2256-2257, 1984).
144. Keren, 1., Kaldalu, N., Spotering, A., Wang, Y., and Lewis, K. (2004). Persister cells and tolerance to antimicrobials. FEMS Microbiology Letters 230, 13-18.
145. Keshavjee, S; Gelmanova, I; Farmer, P; Mishustin, S; Strelis, A; Andreev, Y; Pasechnikov, A; Atwood, S et al. (2008). “Treatment of extensively drug-resistant tuberculosis in Tomsk, Russia: a retrospective cohort study”. The Lancet 372(9647): 1403.
146. Kint, C. I., Verstraeten, N., Fauvart, M., and Michiels, J. (2012). New-found fundamentals of bacterial persistence. Trends in Microbiology 20, 577-585.
147. Kirksey M A, Tischler A D, Simeone R, Hisert K B, Uplekar S, Guilhot C, McKinney J D. 2011. Spontaneous phthiocerol dimycocerosate-deficient variants of Mycobacterium tuberculosis are susceptible to gamma interferon-mediated immunity. Infect Immun. 79:2829-2838.
148. Kirson E D, Gurvich Z, Schneiderman R, et al. Disruption of cancer cell replication by alternating electric fields. Cancer Res. 2004; 64: 3288-3295.
149. Kirson, E. D., et. al. 2007. Alternating electric fields arrest cell proliferation in animal tumor models and human brain tumors. Proc. Natl. Acad. Sci. USA 104:10152-10157.
150. Kirwan, Daniela E “Same-day diagnosis and treatment of tuberculosis”, The Lancet Infectious Diseases 23rd Oct. 2012
151. Klenner, Frederick R. The Use of Vitamin C as an Antibiotic. Journal of Applied Nutrition, 1953, Vol. 6, pp. 274-278
152. Klenner, Frederick R., Observations on the Dose and Administration of Ascorbic Acid When Employed Beyond the Range of A Vitamin In Human Pathology. Journal of Applied Nutrition Vol. 23, No's 3 & 4, Winter 1971. Clinical Guide to the Use of Vitamin C, The Clinical Experiences of Frederick R. Klenner, M. D., summarized by Lendon H. Smith, M. D. 2233 SW Market Street, Portland, Oreg. 97201
153. Klenner F R. Massive doses of vitamin C and the virus diseases. South Med J, 1951, April; 113(4):101-7. Klenner F R. The vitamin and massage treatment for acute poliomyelitis. South Med J, 1952, August; 114(8):194-7. Fatigue, normal and pathological, with special consideration of myasthenia gravis and multiple sclerosis. Southern Medicine and Surgery, 1949, September, Vol 111, No 9. The role of ascorbic acid in therapeutics. (Letter) Tri-State Medical J, 1955, November, p 34.
154. Klenner F R. Poliomyelitis: Case histories. Tri-State Medical J, 1956, September, p 28-31. Klenner F R. An insidious virus. Tri-State Med J, 1957, June. The folly in the continued use of a killed polio virus vaccine. Tri-State Med J, 1959, February, p 1-8. Significance of high daily intake of ascorbic acid in preventive medicine. J Int Acad Prev Med, 1974; 1:1, 45-69. Also: In Williams R J and Kalita D K, editors. A physician's handbook on orthomolecular medicine. 1977. New York: Pergamon, p 51-59. About Diabetes Mellitus (letter). Tri-State Med J, 1955, May.
155. Klenner F R. The protocol of how to effectively treat Multiple Sclerosis. Journal of Applied Nutrition, 1973.
156. Koul A, Vranckx L, Dendouga N, et al. Diarylquinolines are bactericidal for dormant mycobacteria as a result of disturbed ATP homeostasis. J. Biol. Chem. 2008; 83:25273-25280.
157. Koul A, Dendouga N, Vergauwen K, et al. Diarylquinolines target subunit c of mycobacterial ATP synthase. Nat. Chem. Biol. 2007; 3:323-324.
158. Kreis, B., S. Pretet, J. Birenbaum, P. Guibout, J. J. Hazeman, E. Orin, S. Perdrizet, and J. Weil. 1976. Two three-month treatment regimens for pulmonary tuberculosis. Bull. Int. Union Tuberc. 51:71-75
159. Kirson et. al., “Disruption of Cancer Cell Replication by Alternating Electric Fields”, Cancer Research 64, May 2004, p. 3288-3295, Haifa, Israel.
160. Kuldeep Sing Sachdeva in Kelly Morris, “The new face of tuberculosis”, The Lancet Infectious Diseases, Vol 11, October 2011, 736.
161. Kumar, A., Farhana, A., Guidry, L., Saini, V., Hondalus, M., and Steyn, A. J. C. (2011). Redox homeostasis in mycobacteria: the key to tuberculosis control? Expert Reviews in Molecular Medicine 13, e39.
162. Kusunose et al., “Superoxide Dismutase from Mycobacterium tuberculosis”, J. Biochem. 80:1343-1352, 1976.
163. Lai H1, Sasaki T, Singh N P. Targeted treatment of cancer with artemisinin and artemisinin-tagged iron-carrying compounds. Expert Opin Ther Targets. 2005 October; 9(5):995-1007 Leading Edge: The tuberculosis X factor. Lancet Infect. Dis. 2006; 6: 679.
164. Lee Jongsoon and Pilch Paul F. The insulin receptor: structure, function, and signaling. The American Physiological Society, 1994. Invited review, Pages C319-C334
165. Lenaerts A J, Hoff D, Aly S, et al. Location of persisting mycobacteria in guinea pig model of tuberculosis revealed by R207910. Antimicrob. Agents Chemother. 2007; 51:3338-3345.
166. Levy Thomas E. Curing the Incurable: Vitamin C, Infectious Diseases, and Toxins, 3rd Edition cites 1200 such articles supporting the use of Vitamin C.
167. Lewis, K. (2010). Persister cells. Annual Review of Microbiology 64, 357-372.
168. Li J, Munsiff S S, Driver C R, Sackoff J. Relapse and acquired rifampin resistance in HIV-infected patients with tuberculosis treated with rifampin- or rifabutin-based regimens in New York City, 1977-2000. Clin Infect Dis 2005; 41:83-91.
169. Li, Y., and Zhang, Y. (2007). PhoU is a persistence switch involved in persister formation and tolerance to multiple antibiotics and stresses in Escherichia coli. Antimicrobial Agents and Chemotherapy 51, 2092-2099.
170. Lim S A (April 2006). “Ethambutol-associated optic neuropathy” (PDF). Ann. Acad. Med. Singap. 35 (4): 274-8.
171. Lin G. et. al. Inhibitors selective for mycobacterial versus human proteasomes. Nature 461, 621-626 (1 Oct. 2009)
172. Locbcl, R. O., Shorr, E., and Richardson, H. B. (1933). The Influence of Adverse Conditions upon the Respiratory Metabolism and Growth of Human Tubercle Bacilli. Journal of Bacteriology 26, 167-200.
173. Long, E. R. 1935. From pathology to epidemiology in tuberculosis: softening of the caseous tubercle and its results. JAMA 104:1883-1888
174. Louis N, Gevers T, Van den Berg J, Vranchkx L, Andreis K. ATP synthase inhibition of Mycobacterium avium is not bactericidal. Antimicrob. Agents Cheother. 2009; 53:4927-4929.
175. Lounis N, Veziris N, Chauffour A, Truffot-Pernot C, Andries K, Jarlier V. Combinations of R207910 with drugs used to treat multidrug-resistant tuberculosis have the potential to shorten treatment duration. Antimicrob. Agents Chemother. 2006; 50:3543-3547.
176. Lounis N, Guillemont J, Veziris N, Koul A, Jarlier V, Andries K. R207910 (TMC207): a new antibiotic for the treatment of tuberculosis. Med. Mal. Infect. 2009 DOl: 10.1016/J. medmal. 2009.09.007.
177. Lounis N, Veziris N, Chauffour A, Truffot-Pernot C, Andries K, Jarlier V. Combinations of R207910 with drugs used to treat multidrug-resistant tuberculosis have the potential to shorten treatment duration. Antimicrob. Agents Chemother. 2006; 50: 3543-3547.
178. Luis Reinaldo Camacho, et. al., “Identification of a Virulence Gene Cluster of Mycobacterium Tuberculosis by Signature-Tagged Transposon Mutagenesis”, Molecular Microbiology, 1999, pp. 257-267, vol. 34, No. 2.
179. Luis R. Camacho, et al., “Analysis of the Phthiocerol Dimycocerosate Locus of Mycobacterium tuberculosis, Evidence that this Lipid is Involved in the Cell Wall Permeability Barrier”, Journal Biological Chemistry, 2001, pp. 19845-19854, vol. 276, No. 23
180. Ma, C., Sirn, S., Shi, W., Du, L., Xing, D., and Zhang, Y. (2010). Energy production genes sucB and ubiF are involved in persister survival and tolerance to multiple antibiotics and stresses in Escherichia coli. FEMS Microbiology Letters 303, 33-40.
181. Maartens G, Wilkinson R J (2007) Tuberculosis. Lancet 370: 2030-2043.
182. Madhusudana S N, Shamsundar R, Seetharaman. In vitro inactivation of the rabies virus by ascorbic acid. Int J Infect Dis. 2004 January; 8(1):21-5.
183. Master et al., Mycobacterium tuberculosis prevents inflammasome activation. Cell Host Microbe 3, 224-232, 2008.
184. Martineau, et al. (2007) J Clin Invest 117: 1988-1994
185. Matteelli A, Migliori G B, Cirillo D, Centis R, Girardi E, Raviglione R. Multidrug-resistant and extensively drug-resistant Mycobacterium tuberculosis: epidemiology and control. Expert Rev. Anti Infect. Ther. 2007; 5:857-871.
186. Matteelli A1, Carvalho A C, Dooley K E, Kritski A. TMC207: the first compound of a new class of potent anti-tuberculosis drugs. Future Microbiol. 2010 June; 5 (6):849-58.
187. Maximiliano G. Autophagy is a Defense Mechanism Inhibiting BCG and Mycobacterium tuberculosis Survival in Infected Macrophages. Cell, 2004, Volume 119, Issue 6, p 753-766.
188. McKinney, J. D., HonerzuBentmp, K., Muiioz-EHas, E. J., Miczak, a, Chen, B., Chan, W. T., Svvcnson, D., Sacchcttini, J. C., Jacobs, W. R., and Russell, D. O. (2000). Persistence of Mycobacterium tuberculosis in macrophages and mice requires the glyoxylate shunt enzyme isocitratclyase. Nature 406, 735-738.
189. Menzies D, Hamdan A I Jandali, and Badriah A I Otaibi. Recent developments in treatment of latent tuberculosis infection. Indian J Med Res. 2011 March; 133(3): 257-266.
190. Mitchison, D. A. 1998. Basic concepts in the chemotherapy of tuberculosis, p. 15-50. In P. R. J. Gangadharam and P. A. Jenkins (ed.), Mycobacteria II. Chemotherapy. Chapman & Hall, New York, N.Y
191. Migliori, G B; Ortmann, J; Girardi, E. “et al”. (2007). “Extensively drug-resistant tuberculosis, Italy and Germany” 13: 5.
192. Milazzo G. et al., Insulin Receptor Expression and Function in Human Breast Cancer Cell Lines. Cancer Research 1992, 52, 3924-3930.
193. Missouri Department of Health & Senior Services. “Drug Susceptibility Testing (DST). N.p., n.d. January 2014
194. Mitchison D A, Selkon J B. The bactericidal activities of anti-tuberculous drugs. Am Rev Tuberc. 1956; 74(2 Part 2):109-116
195. Michison G, and Davies G. The chemotherapy of tuberculosis: past, present and future. Int J Tuberc Lung Dis. 2012 June; 16(6): 724-732.
196. Mitnick C D, Castro K G, Harrington M, Sacks L V, Burman W. Randomised trials to optimize treatment of multidrug-resistant tuberculosis. PLoS Med. 2007; 4: e292.
197. Molnar, J., I. Beladi, and I. Foldes. 1977. Studies on antituberculotic action of some 482 phenothiazine derivatives in vitro. Zentralbl. Bakteriol. Orig. A 239:521-526.
198. Moyalan, D. J. et. al. (1989) “Hyperthermia-Scientific Basis and Clinical Applications” Therapeutic Radiology, Manfield, C. M. Ed., Elsevier-N.Y. 531-541.
199. Montanes C M, Zaragoza (E S) et. al. Tuberculosis vaccine. U.S. Pat. No. 8,642,011 B2; 2014
200. Murphy et. al., 2007, During Treatment of Tuberculosis: A Theoretical Basis for Shorter Duration; Curative Monotherapy with Congeners. BMC Infectious diseases, Vol. 7:[0018] 84
201. Musuka S et. al., Thioridazine Exhibits Wobbly Pharmacokinetic-Pharmacodynamic Parameters. 2013, Antimicrob. Agents Chemother. doi:10.1128/AAC.00829-13
202. Myal, Shiu, Bhomic, and Baja. Receptor Binding and Growth Promoting Activity of Insulin like Growth Factors and Human Breast Cancer Cells. Cancer Research 44:5486-5490, 1984.
203. Nagabhushanam V, et al. 2003. Innate inhibition of adaptive immunity: M. tuberculosis induced IL-6 inhibits macrophage response to IFN-Y⁻. J. Immunol. 171, 4750-4757.
204. Nakase I1, Lai H, Singh N P, Sasaki T. Anticancer properties of artemisinin derivatives and their targeted delivery by transferrin conjugation. Int J Pharm. 2008 Apr. 16; 354(1-2):28-33. Epub 2007 Sep. 6.
205. Narayanan, S., Swaminathan, S., Supply, P., Shanmugam, S., Narendran, G., Hari, L., Ramachandran, R., Locht, C., Jawahar, M. S., and Narayanan, P. R. (2010). Impact of HIV infection on the recurrence of tuberculosis in South India. The Journal of infectious Diseases 201, 691-703.
206. Nauts, Helen Coley, Bacterial Products in the Treatment of Cancer: Past, Present and Future. Paper read at the International Colloqium on Bacteriology and Cancer, Clogne, Federal Republic of Germany, Mar. 16-18, 1982. Bacterial vaccine therapy of cancer. Dev Biol Stand. 1977; 38487-94. Bacterial pyrogens: beneficial effect on cancer patients. Prog Clin Biol Res. 1982; 107:687-96. Bacteraia and cancer-antagonisms and benefits, Cancer Surv. 1989; 8:713-23.
207. Netherlands Organization for Scientific Research. “Tuberculosis Treatment May Be Shortened.” <www.sciencedaily.com/releases/2009/09/090902122523.htm. Science Daily. 14 Sep. 2009.
208. “New Laboratory Diagnostic Tools for Tuberculosis Control”, Stop TB Partnership, 2009 http://apps.who.int/tdr/svc/publications/non-tdr-publications/
209. Nuermberger E. Once weekly treatment of tuberculosis with the diarylquinoline R207910. Am. J. Respir. Crit. Care Med. 2009; 179: 2-3.
210. Ochs M l, Nyengaard J R, Jung A, Knudsen L, Voigt M, Wahlers T, Richter J, Gundersen H J. The number of alveoli in the human lung. Am J Respir Crit Care Med. 2004 Jan. 1; 169(1):120-4.
211. Odell R H Jr, Sorgnard R E. Anti-inflammatory effects of electronic signal treatment. Pain Physician. 2008; 11 (6):891-907.
212. Odell R H, Sorgnard R E. New technique combines electrical currents and local anesthetic for pain management. Pract Pain Manage. 2011; 11 (6): 52-68.
213. Odell R H, Sorgnard R E. Milne R. Combined electrochemical treatment for peripheral neuropathy. Practice of pain management, October 2015, 47-55.
214. Ozone Treatment of Phthisis: The Internal Administration of Ozone in the Treatment of Phthisis”. Lancet II: Vol 140, #3512, 1180-1181. 1892.
215. Padayatty S J, Sun H, Wang Y, Riordan H D, Hewitt S M, Katz A, Wesley R A, Levine M. Vitamin C pharmacokinetics: implications for oral and intravenous use. Ann. Intern. Med. 2004 April; 140 (7):533-537.
216. Paramasivan C N, Venkataraman P. Drug resistance in tuberculosis in India. Indian J Med Res 2004; 120: 377-386.
217. Pareilleux, A., and N. Sicard. 1970. Lethal effects of electric current on Escherichia coli. Appl. Microbiol. 19:421-424.
218. Park, S. & J. A. Imlay, (2003) High levels of intracellular cysteine promote oxidative DNA damage by driving the Fenton reaction. J. Bacterial. 185: 1942-1950.
219. Pauling L: Vitamin C and the Common Cold. San Francisco: WH Freeman, 1976.
220. Petit S, Coquerel G, Meyer C, Guillemont J. Absolute configuration and structural features of R2071910, a novel anti-tuberculosis agent. J. Mol. Struct. 2007; 837:252-256.
221. Peloquin, C. 2003. What is the ‘right’ dose of rifampin? Int. J. Tuberc. Lung Dis. 7:3-5.
222. Petrella S, Cambau E, Chauffour A, Andries K, Jarlier V, Sougakoff W. Genetic basis for natural and acquired resistance of the diaryquinoline R207910 in mycobacteria. Antimicrob. Agents Chemother. 2006; 50:2853-2856.
223. Peto, H M, Pratt, R. et. al. Epidemiology of extrapulmonary tuberculosis in the United States, 1993-2006. Clin Infect Dis. 2009; 49: 1350-1357
224. Piccaro, G., F. Giannoni, P. Filippini, A. Mustazzolu& L. Fattorini, (2013) Activities of drug combinations against Mycobacterium tuberculosis grown in aerobic and hypoxic acidic conditions. Antimicrob. Agents Chemother. 57: 1428-1433
225. Piddock, L. J. V. (2000). Accumulation of rifampicin by Mycobacterium aurum, Mycobacterium smagmatis and Mycobacterium tuberculosis. Journal of Antimicrobial Chemotherapy 45, 159-165.
226. Popovic Pavelic. Insulin and Glucagon Secretion by Renal Adenocarcinoma. Cancer 48:98-100, 1981
227. Rao S P, Alonso S, Rand L, Dick T, Pethe K. The proton motive force is required for maintaining ATP homeostasis and viability of hypoxic, nonreplicating Mycobacterium tuberculosis. Proc. Natl Acad. Sci. USA. 2008; 105:11945-11950.
228. Ramaswamy, S., and Musser, J. M. (1998). Molecular genetic basis of antimicrobial agent resistance in Mycobacterium tuberculosis: Tuber. Lung Dis. 79, 3-29.
229. Radiologic clinic of North America, Vol 27, Number 3 May 1989, guest editor Dr. Steeves A. R, published by W.B. Saunders.
230. Raviglione, M. C., Snider, D. E., Jr., and Kochi, A. (1995). Global epidemiology of tuberculosis. Morbidity and mortality of a worldwide epidemic. JAMA 273, 220-226.
231. Raviglione M C, Uplekar M. WHO's new Stop TB strategy. Lancet. 2006; 367:952-955.
232. Raynaud et al., “Extracellular Enzyme Activities Potentially Involved in the Pathogenicity of Mycobacterium tuberculosis”, Microbiology 144:577-587, 1998.
233. Rediske, A. M., N. Rapoport, and W. G. Pitt. 1999. Reducing bacterial resistance to antibiotics with ultrasound. Lett. Appl. Microbiol. 28:81-84.
234. Rieder H L. Epidemiologic basis of tuberculosis control. 1st ed. Paris, France: International Union against Tuberculosis and Lung Disease; 1999. pp. 1-162.
235. Rodrigues, C. et al. (2008)“Drug susceptibility testing of Mycobacterium tuberculosis against second-line drugs using the Bactec MGIT 960 System.” Int J Tuberc Lung Dis. 12.12:1449-1455.
236. Rook, G. A. W., and B. R. Bloom. 1994. Mechanisms of pathogenesis in tuberculosis, p. 485-501. In B. R. Bloom (ed.), Tuberculosis: pathogenesis, protection, and control. American Society for Microbiology, Washington, D.C.
237. Rosen. After Insulin Binds. Science 273:1452-1457, 1987
238. Rosenthal, I. M., K. Williams, S. Tyagi, C. A. Peloquin, A. A. Vernon, W. R. Bishai, J. H. Grosset, and E. L. Nuermberger. 2006. Potent twice-weekly rifapentine-containing regimens in murine tuberculosis. Am. J. Respir. Crit. Care Med. 174: 94-101.
239. Rosenthal I M, Zhang M, Williams K N, Peloquin C A, Tyagi S, Vernon A A, Bishai W R, Chaisson R E, Grosset J H, Nuermberger E L. Daily dosing of rifapentine cures tuberculosis in three months or less in the murine model. PLoS Med. 2007 December; 4(12):e344.
240. Rossig L, Hoffmann J, Hugel B, et al. Vitamin C inhibits endothelial cell apoptosis in congestive heart failure. Circulation. 2001 Oct. 30; 104(18):2182-7
241. Roy A, Eisenhut M, Harris R J; et al. (2014). “Effect of BCG vaccination against Mycobacterium tuberculosis infection in children: systematic review and meta-analysis”. doi:10.1136/bmj.g4643:BMJ 349: g4643).
242. Russell, D. G. 2007. Who puts the tubercle in tuberculosis? Nature Reviews Microbiology 5: 39-47.
243. Rustomjee R, Diacon A H, Allen J, et al. Early bactericidal activity and pharmacokinetics of the dairylquinoline TMC207 in treatment of pulmonary tuberculosis. Antimicrob. Agents Chemother. 2008; 52:2831-2835
244. Sabatine Marc S., et. al., Efficacy and Safety of Evolocumab in Reducing Lipids and Cardiovascular Events. Mar. 15, 2015D01: 10.1056/NEJMoa1500858.
245. Sacchetti, J. C., Rubin, E. J., and Freundlich J. S. (2008). Drugs versus bugs: in pursuit of the persistent predator Mycobacterium tuberculosis. Nature Reviews. Microbiology 6, 41-52.
246. Sacks L V, Behrman L E. Developing new drugs for the treatment of drug-resistant tuberculosis: a regulatory perspective. Tuberculosis. 2008; 88(Suppl. 1):S93-S100.
247. Salzberg M, Kirson E, Patti Y, Rochlitz C. A pilot study with very low-intensity, intermediate-frequency electric fields in patients with locally advanced and/or metastatic solid tumors. Onkologie. 2008; 31:362-365.
248. Sambandamurthy, V. K., Wang, X., Chen, B., Russell, R. G., Derrick, S., Collins, F. M., Morris, S. L., and Jacobs, W. R. (2002). A pantothenate auxotroph of Mycobacterium tubrculosisis highly attenuated and protects mice against tuberculosis. Nature Medicine 8, 1171-1174.
249. Sanger Christian et. al. Inununodominant PstSI antigen of Mycobacterium tuberculosis is a potent biological response modifier for the treatment of a bladder cancer, BMC Cancer Nov. 26, 2004, pp. 1-14.
250. Sankpal. V, and Mohan Krishna and others on Tuberculosis of spine—www.slide share.net/drmkreddy78/tuberculosis-of-spine-Jul. 20, 2013.
251. Sampson, S. L., Dascher, C. C., Sambandamurthy, V. K. et al. Protection elicited by a double leucine and pantothenate auxotroph of Mycobacterium tuberculosis in guinea pigs. Infect Immun 2004, 72(5), 3031-3037.
252. Saint Fabien et. al., “Do Prognostic Parameters of Remission versus Relapse after Bacillus Calmette-Guerin (BCG) Inununotherapy Exist? Analysis of a Quarter Century of Literature”, European Urology, vol. 43, 351-361, 2003.
253. Schaller A, et al. Salicylate reduces susceptibility of Mycobacterium tuberculosis to multiple antituberculosis drugs. Antimicrob Agents Chemother 2002; 46:2636-9.
254. Schecterle, L. M. et al (1995) “Hyperthermia as a Potential Treatment in HIV Disease” Alternative Med. J. 2(3): 28-32. (1997) “Hyperthermia Alters HIV Kinetics” Alternative Therapies in Clinical Practice 4(5):162-164.
255. Schilsky, Richard L. and Frederick. S. Ordway. Insulin effects on methotrexatepolyglutamate synthesis and enzyme binding in cultured human breast cancer cells. Cancer Chemother Pharmacol (1985) 15: 272-277
256. Schnetzler, Rubin, and Pilch. Structural Requirements for the Transmembrane Activation of the Insulin Receptor Kinase. J Biol Chern 261:15281-15287, 1986.
257. Schoenmakers, R. et. al. 2014. Composition for treatment of tuberculosis. U.S. Pat. No. 8,912,329 B2.
258. Science Daily, 2 Jan. 2011. Evidence of increased lung cancer risk among tuberculosis patients.” Researchers at China Medical University and Hospital in Taiwan www.sciencedaily.com/releases/2011/01/110102202238.htm.
259. Seaverson K, Tschetter D, Kaur T. Patient guide to oxygen/ozone therapy. Health centered cosmetic dentistry. [Online]. [Cited 2010 Jan. 13]. Available from: URL: http://www.toothbythelake.net/ozone_therapy.html
260. Seldinger 1.S. I., “Catheter replacement of the needle in percutaneous arteriography; a new technique,” Acta Radiologica, vol. 39, no. 5, pp. 368-376, 1953.
261. Shah N S, Wright A, Drobniewski F, et al. (2005). “Extreme drug resistance in tuberculosis (XDR-TB): global survey of supranational reference laboratories for Mycobacterium tuberculosis with resistance to second-line drugs”. Int J Tuberc Lung Dis 9 (Suppl 1): S77.
262. Shah, N. S., Richardson, J., Moodley, P., Moodley, S., Babaria, P., Ramtahal, M., Hcyscll, S. K., Li, X., Moll, A. P., Friedland, G., et al. (2011). Increasing drug resistance in extensively drug-resistant tuberculosis, South Africa. Emerging Infectious Diseases 17, 510-513.
263. Shamas, Dhurandhar, Blackar, Insulin secreating Bronchial Carcinoid Tumor with Widespread Metastases. Am J Med 44:632-637, 1981.
264. Shandilya A, Chacko S, Jayaram B. Ghosh I. (2013). “A plausible mechanism for the antimalarial activity of artemisinin: A computational approach”. Sci. Rep. 4: 2513.
265. Shantha T R and Bourne G H: The Perineural epithelium: and significance. Journal Nature 199, 4893:577-579 (1963).
266. Shantha T R and Bourne G H: Perineural epithelium: A new concept of its role in the integrity of the peripheral nervous system. Science 154:1464-1467 (1966).
267. Shantha T R and Bourne G H: The “Perineural Epithelium”: A new concept. Its role in the integrity of the peripheral nervous system. Structure and Function of Nervous Tissues. Volume I. pp 379-458 (GH Bourne, Ed.) Academic Press, New York. 1969.
268. Shantha T R: Retrograde intubation's using the sub-cricoid region, British Journal of Anesthesia 68:109-112 (1992).
269. Shantha, T R; 1. discovery of insulin and IPT: amazing history, 2. high dose methotrexate therapy using Insulin; 3 local injections of tumors with insulin and cytotoxic drugs; 4. two and three cycle insulin Potentiation therapy (IPT); Effective Treatment of Scleroderma using IPT and chemotherapeutic agents: Presented at 2^{nd} international conference on Insulin Potentiation Therapy held at Cancun, Mexico, Jun. 28-Jul. 1, 2004.
270. Shantha, T R; Treatment of massive peritoneal infection of Candida albicans and other co-infections after gastric bypass surgery with peritoneal hyperthermia with Diflucan continuous irrigation under anesthesia. Presented in Hyperthermia conference in Destin Fla., 2003
271. Shantha, T R; Treatment of tuberculosis and cancer in a patient with high dose vitamin C and chemo therapeutic agents, (work in progress).
272. Shantha, T R; Treatment of Methicillin-resistant Staphylococcus aureus (MRSA) infection with high dose Vitamin C, antibiotics, induced hypoglycemia, and hyperbaric therapy (Under preparation for publication).
273. Shantha, T R; Treatment of MSRA and other “super bug” infections including Ebola virus infection with high dose Vitamin C, auto-haemo-ozone therapy, antibiotics, hyperbaric therapy (under study)
274. Shantha, T. R. Unknown Health Risks of Inhaled Insulin. Life Extension, September 2007 pp 74 79, Post publication comments in September 2008 issue of Life Extension, Pages 24.
275. Shantha, T. R. Shantha. Known Health Risks of Inhalation Insulin for Diabetics: Panacea or Evolving Future Health Risk, February 2015 issue of Life Extension, pages 62-69.
276. Shantha, T R and Shantha, Jessica G. Inhalation Insulin, Oral and Nasal Insulin Sprays for Diabetics: Panacea or Evolving Future Health Disaster. Part I: Townsend Letter Journal: Issue #305, December 2008 pages: 94 98; Part II: Townsend Letter, January, 2009, Issue #306, pages 106-110.
277. Shantha, T R. in US20110052678 age related macular degeneration, US20110217260 enhancing eye lashes growth, US20110294730 for the treatment of glaucoma, US20110081333 US20120003296 for the treatment of dry eye syndrome, US20120101033 retinitis pigmentosa, US20100172865 enhancing hair growth, US20110217260 enhancing eye lashes, US20120128683 for Autism treatment, US20090304776 for diabetes, US20110020279 for rabies cure using Insulin and other therapeutic agents, U.S. Pat. No. 5,792,100 for viral infections, U.S. Pat. No. 7,804,233, WO/2009/026213A1 light bulb, U.S. Pat. No. 7,954,900 leg support, U.S. Pat. No. 8,226,228 cooling glasses for eyes, U.S. Pat. Nos. 5,735,817, 5,792,100, US 2012/0323214 A1, US 2015/0073330 A1, US 2015/0080785 A1 for the treatment of Alzheimer's disease; US20120234331, US20120255561, US20120234332, US20110155143, U.S. Pat. No. 9,072,613, B2 2015 for snoring and sleep apnea, patent pending on the Treatment of Parkinson's disease, hair loss, migraine headache.
278. Shantha, T R. Alzheimer's disease treatment with multiple therapeutic agents including insulin delivered to the olfactory region through a special delivery catheter and iontophoresis. US20120323214, US20140012182, WO 2015/013252 A1 for the treatment of Alzheimers.
279. Shantha T. R. and Yasuo Nakajima. Yerkes Regional Primate Research Center, Emory University, Atlanta, Ga.: Histological and Histochemical Studies on the Rhesus Monkey (Macaca mulatta) Olfactory Mucosa. Z. Zellforsch. 103, 291-319 (1970).
280. Shantha T R: Peri-vascular (Virchow—Robin) space in the peripheral nerves and its role in spread of local anesthetics, ASRA Congress at Tampa, Regional Anesthesia 17 (March-April, 1992).
281. Shantha T. R., Alternative Therapies to Cure and Curtail Ebola Viral Infection: Under preparation for publication 2015.
282. Sharma S K, Liu J J. Progress of DOTS in global tuberculosis control. Lancet. 2006; 367:950-952.
283. Shulman, Jamison and Spiegel. Use of DNP to treat various diseases including non-alcoholic fatty liver. WO 2015/031598 PCT/US2014/053127, WO 2015/031756, PCT/US2014/053406 to
284. Shi L, Sohaskey C D, Kana B D, et al. Changes in energy metabolism of Mycobacterium tuberculosis in mouse lung and in vitro conditions affecting aerobic respiration. Proc. Natl Acad. Sci. USA. 2005; 102:15629-15634.
285. Shingadia D, Novelli V. “Diagnosis and treatment of tuberculosis in children” (2010) Department of Academic Child Health, St Barthlomews and The London Medical and Dental School, Queen Mary, University of London, UK
286. Shinnick, T., ed. (1996). Current Topics in Microbiology and Immunology (New York: Springer-Verlag).
287. Sia I G., Wieland M L. Current Concepts in the Management of Tuberculosis. Mayo Clinic Proceedings. April 2011 Volume 86, Issue 4, Pages 348-361.
288. Siddiqi, N., R. Das, N. Pathak, S. Banerjee, N. Ahmed, V. M. Katoch, and S. E. Hasnain. 2004. Mycobacterium tuberculosis isolate with a distinct genomic identity overexpresses a tap-like efflux pump. Infection 32:109-111.
289. Siddiqi, Kamran “Clinical diagnosis of smear-negative pulmonary tuberculosis in low-income countries: the current evidence”, The Lancet Infectious Diseases, Vol 3, May 2003, 288
290. Sidley, P. (2006). “South Africa acts to curb spread of lethal strain of TB”. Brit Med J 333 (7573): 825.
291. Skerry C, Harper J, Klunk M, Bishai W R, Jain S K. Adjunctive TNF inhibition with standard treatment enhances bacterial clearance in a murine model of necrotic TB granulomas. PLoS One 2012; 7: e39680.
292. Solovieva, M. E., V. V. Soloviev& V. S. Akatov, (2007) Vitamin B12b increases the cytotoxicity of short-time exposure to ascorbic acid, inducing oxidative burst and iron-dependent DNA damage. Eur. J. Phamacol. 566: 206-214.
293. Spigelman M K. New tuberculosis therapeutics: a growing pipeline. J. Infect. Dis. 2007; 196: S28-S34.
294. “Sputum Culture”, WebMD www.webmd.com/lung/sputum-culture
295. “Sputum Gram stain—Overview”, University of Maryland Medical Center www.umm.edu/ency/article
296. Sreevatsan, S., K. E. Stockbauer, X. Pan, B. N. Kreiswirth, S. L. Moghazeh, W. R. Jacobs, Jr., A. Telenti, and J. M. Musser. 1997. Ethambutol resistance in Mycobacterium tuberculosis: critical role of embB mutations. Antimicrob. Agents Chemother. 41:1677-1681
297. Srikanth, A, Manthena Sathish, and Anumolu Venkatanaga Sri Harsha. Application of ozone in the treatment of periodontal disease. J Pharm Bioallied Sci. 2013 June; 5 (Supp) 1): S89-S94. doi: 10.4103/0975-7406.113304.
298. Srinivasa P. S. Rao, Sylvie Alonso, Lucinda Rand, Thomas Dick, Kevin Pethe. The proton motive force is required for maintaining ATP homeostasis and viability of hypoxic, nonreplicating mycobacterium tuberculosis. Current Issue vol. 105 no. 33. www.pnas.org/cgi/content/full/0711697105/DCSupplemental
299. Stadler N, Eggermann J, Voo S, Kranz A, Waltenberger J. Smoking-induced monocyte dysfunction is reversed by vitamin C supplementation in vivo. Arterioscler Thromb Vasc Biol. 2007 January; 27(1):120-6.
300. Stephenson C M, Levin R D, Spector T, Lis C G. Phase I clinical trial to evaluate the safety, tolerability, and pharmacokinetics of high-dose intravenous ascorbic acid in patients with advanced cancer. Cancer Chemother Pharmacol. 2013 July; 7 2(1):139-146.
301. Stoker, George (1916). “The Surgical Uses of Ozone”. Lancet II: 712.
302. Suchkov B P (June 1964). Study of the Ozonization of Drinking Water Containing Pathogenic Bacteria and Viruses, Gig Sanit (in Russian) 29: 22-9. PMID 14235449
303. Sutezolid (PNU-100480, PF-02341272) is an oxazolidinone antibiotic currently in development as a treatment for extensively drug-resistant tuberculosis. N Engl J Med. 2009 Jun. 4; 360(23):2397-405.
304. Swaminathan S, Ramachandran R, Bhaskar R, Ramanathan U, Prabhakar R, Datta M. Risk of development of tuberculosis in HIV infected individuals in India. Int J Tuberc Lung Dis 2000; 4: 839-844.
305. Swaminathan S, et al. Efficacy of a 6-month versus 9-month Intermittent Treatment Regimen in HIV-infected Patients with Tuberculosis. A Randomized Clinical Trial Am J Respir Crit Care Med 2010, Vol 181. pp 743-751.
306. Szulakowski P, et. al., “The Effect of Smoking on the Transcriptional Regulation of Lung Inflammation in Patients with Chronic Obstructive Pulmonary Disease”, American Journal of Respiratory and Critical Care Medicine, Vol. 174, No. 1 (2006), pp. 41-50.
307. Tainter, M. L. et. al (1932) “Actions of Dinitrophenol” Proc. Soc. Exper. Bioi. Med. 29:1268-1275. Tainter, M. L. et. al (1933) “Febrile, Respiratory and Some Other Actions of Dinitrophenol” J. Pharmacal. Exp. Therap., 48:410-418. Tainter, M. L. (1933) “Dinitrophenol as a Metabolic Stimulant” Bull. Calif. Dietetic Assoc., 1:3-6. Tainter, M. L. et. al. (1934) “A Case of Fatal Dinitrophenol Poisoning” J. A. M. A., 102:1147. Tainter, M. L. (1934) “Dinitrophenol in Diet, on Growth and Duration of Life of the White Rat” Proc. Soc. Exper. Bioi. Med., 31:1161-1163. Tainter, M. L. et al., “Dinitrophenol in the Treatment of Obesity,” JAMA 105, pp. 332-336 (1935). Tainter, M. L. (1938) “Growth, Life-Span and Food Intake of White Rats Fed Dinitrophenol throughout Life” J. Pharm. Exp. Ther., 63, No. 1.30. Tainter, M. L. et. al, (1934) “Chronic Toxicity of Dinitrophenol: Renal Function” Proc. Soc. Exper. Bioi. Med., 1:1163-1166. “Dinitrophenol on Liver Function” Calif. and West. Med., 43: No. 5.
308. TB Alliance clinical trials. [29 Mar. 2010].www.tballiance.org/new/portfolio/html-portfolio.php.
309. “TB diagnosis: Improving the yield with fluorescence microscopy”, 2007 www.aidsmap.com/TB-diagnosis-Improving-the-yield-with-fluorescence-microscopy/
310. “TB in India: burden, progress, and needs”, TB diagnostics in India conference August 2011, tbevidence.org
311. “TB Testing & Diagnosis”, CDC www.cdc.gov/tb/topic/testing/
312. Telenti, A., P. Imboden, F. Marchesi, D. Lowrie, S. Cole, M. J. Colston, L. Matter, K. Schopfer, and T. Bodmer. 1993. Detection of rifampicin-resistance mutations in Mycobacterium tuberculosis. Lancet 341:647-650.
313. Thotathil Z, Jameson M B (2007). “Early experience with novel immunomodulators for cancer treatment”. Expert Opinion on Investigational Drugs 16 (9): 1391-403.1).
314. Tischler A D, McKinney J D. 2010. Contrasting persistence strategies in Salmonella and Mycobacterium. Curr. Opin. Microbiol. 13:93-99.
315. Tischler A D, Leistikow, R L, Kirksey M A, Voskuil M I, McKinney J D. 2013. Mycobacterium tuberculosis requires phosphate-responsive gene regulation to resist host immunity. Infect. Immun. 81: 317-328.
316. Todar, Kenneth. 2007. “Tuberculosis.” Todar's Online Textbook of Bacteriology. “Tuberculosis.” Todar's Online Textbook of Bacteriology.
317. Tsukaguchi, K., Balaji, K. N., and Boom, W. H. 1995. CD4+αβ T cell and gd T Cell Responses to Mycobacterium tuberculosis: Similarities and Differences in Ag Recognition, Cytotoxic Effector Function, and Cytokine Production. Journal of Immunology 154: 1786-1796.
318. Tuberculosis Chemotherapy Centre, Madras. A concurrent comparison of home and sanatorium treatment of pulmonary tuberculosis in South India. Bull World Health Organ. 1959; 21:51-144.
319. Udwadia, Z., and Amale, R. (2012). Totally drug-resistant tuberculosis in India. Clinical Infectious. 54, 579-581.
320. Valle, A., E. Zanardini, P. Abbruscato, P. Argenzio, G. Lustrato, G. Ranalli, and C. Sorlini. 2007. Effects of low electric current (LEC) treatment on pure bacterial cultures. J. Appl. Microbiol. 103:1376-1385.
321. van der Wel Nicole et. al., M. tuberculosis and M. leprae Translocate from the Phagolysosome to the Cytosol in Myeloid Cells, Cell 129 p 1287-1298, 2007
322. U.S. Food and Drug Administration. Information for Healthcare Professionals. Erythropoiesis Stimulating Agents (ESAs). Link. Accessed: Jun. 17, 2009.
323. Velayati A A, Masjedi M R, Farnia P, Tabarsi P, Ghanavi J, Ziazarifi A H, Hoffner S E. Emergence of new forms of totally drug-resistant tuberculosis bacilli: super extensively drug-resistant tuberculosis or totally drug-resistant strains in Iran. Chest. 2009; 136(2): 420-425.
324. Venkataswamy, Manjunatha M.; Goldberg, Michael F.; Baena, Andres; Chan, John; Jacobs, W R., Jr.; Porcelli, Steven A. (1 Feb. 2012). “In vitro culture medium influences the vaccine efficacy of Mycobacterium bovis BCG”. (doi:10.1016/j.vaccine.2011.12.044. PMC 3269512) Vaccine 30 (6): 1038-1049.
325. Vernon A, Burman W, Benator D, Khan A, Bozeman L. Acquired rifamycin monoresistance in patients with HIV-related tuberculosis treated with once-weekly rifapentine and isoniazid. Tuberculosis Trials Consortium. Lancet. 1999; 353:1843-1847.
326. Veziris N, Ibrahim M, Lounis N, et al. A once weekly R207910-containing regimen exceeds activity of the standard daily regimen in murine tuberculosis. Am. J. Respir. Crit. Care Med. 2009; 179:75-79.
327. Vilaplana C, Marzo E, Tapia G, Diaz J, Garcia V, Cardona P J. Ibuprofen therapy resulted in significantly decreased tissue bacillary loads and increased survival in a new murine experimental model of active tuberculosis. J Infect Dis. 2013; 208:199-202
328. Vilcheze, C., T. Hartman, B. Weinrick& W. R. Jacobs, Jr., (2013) Mycobacterium tuberculosis is extraordinarily sensitive to killing by a vitamin C-induced Fenton reaction. Nature Communications 4: 1881; doi: 10.1038/ncomms2898.
329. Vilcheze, C., and Jacobs, W. R. (2007). The Mechanism of Isoniazid Killing: Clarity through the Scope of Genetics. Annual Review of Microbiology 61, 35-50.
330. Wallis R S. Reconsidering adjuvant immunotherapy for tuberculosis. Clin Infect Dis 2005; 41: 201-8.
331. Wallis, R S., Patil, S., Cheon, S. H., Edmonds, K., et. al. (1999). Drug tolerance in Mycobacterium tuberculosis. Antimicrobial Agents and Chemotherapy 43, 2600-2606.
332. Ward, S. K., E. A. Hoye, A. M. Talaat, (2008). The global responses of Mycobacterium tuberculosis to physiological levels of copper. J. Bacterial. 190: 2939-2946.
333. Wardman, P. & L. P. Candeias, (1996) Fenton chemistry: an introduction. Radiat. Res. 145: 523-531.
334. Watson Pharma, Inc. Ferrlecit (sodium ferric gluconate complex in sucrose injection) US prescribing information. Link. Accessed: Jun. 17, 2009.
335. Wayne, L. G., and Hayes, L. G. (1996). An in vitro model for sequential study of shift down of Mycobacterium tuberculosis through two stages of non-replicating persistence. Infection and immunity 64, 2062-2069.
336. Wei D Z, Yang J Y, Liu J W, Tong W Y. Inhibition of liver cancer cell proliferation and migration by a combination of (−)-epigallocatechin-3-gallate and ascorbic acid. J Chemother. 2003 December; 15(6):591-5.
337. Weiner M, Burman W, Vernon A, et al. Low isoniazid concentrations and outcome of tuberculosis treatment with once-weekly isoniazid and rifapentine. Am. J. Respir. Crit. Care Med. 2003; 167:1341-1347.
338. Weiner, M., N. Bock, C. A. Peloquin, W. Peloquin, W. J. Burman, A. Khan, A. Vernon, Z. Zhao, S. Weis, T. R. Sterling, K. Hayden, and S. Goldberg. 2004. Pharmacokinetics of rifapentine at 600, 900, and 1,200 mg during once-weekly tuberculosis therapy. Am. J. Respir. Crit. Care Med. 169:1191-1197.
339. Winder, F., and Collins, P. (1970). Inhibition by isoniazid of synthesis of mycolic acids in Mycobacterium tuberculosis. Journal of General Microbiology 41-48
340. Wilson, T. M., G. W. De Lisle, and D. M. Collins. 1995. Effect of inhA and katG on isoniazid resistance and virulence of Mycobacterium bovis. Mol. Microbiol. 15:1009-1015.
341. World Health Organization (WHO-2008). “Countries with XDR-TB confirmed cases as of June 2008”
342. Wong and Holdaway. Insulin Binding by Normal and Neoplastic: Tissue. Int J Cancer 35:335-341, 1985
343. WHO (2012a). Global Tuberculosis Report,
344. WHO. WHO/CDS/STB/2001.16. WHO; Geneva, Switzerland: 2001. An expanded DOTS framework for effective tuberculosis control.
345. WHO Extensively drug-resistant tuberculosis (XDR-TB): recommendations for prevention and control. Wkly Epidemiol. Rec. 2006; 81: 430-432. WHO The Global Plan to Stop TB 2006-2015. [29 Mar. 2010]. http://www.stoptb.org/global/plan/. WHO (2010) Global tuberculosis control. Surveillance, planning, financing. (Report No.: WHO/HTM/TB/2006.362) Geneva: World Health Organization.
346. WHO (2012b). Treatment of tuberculosis guidelines (Geneva, Switzerland).
347. WHO. “Multidrug-resistant tuberculosis (MDR-TB) 2013 Update”. Retrieved 14-6-2013.
348. Winder, F., and Collins, P. (1970). Inhibition by isoniazid of synthesis of mycolic acids in Mycobacterium Tuberculosis. Journal of General Microbiology 41-48.
349. Woollard K J, Loryman C J, Meredith E, et al. Effects of oral vitamin C on monocytes: endothelial cell adhesion in healthy subjects. Bioch Biophys Res Commun. 2002 Jun. 28; 294(5):1161-8.
350. Wu et al., “Identification and Subcellular Localization of a Novel Cu, Zn Superoxide Dismutase of Mycobacterium tuberculosis” FEBS Letters 439:192-196, 1998.
351. www.sciencedaily.com/releases/2011/01/110102202238.htm. Evidence of increased lung cancer risk among tuberculosis patients. Science Daily, 2 Jan. 2011. Researchers at China Medical University and Hospital in Taiwan.
352. Yanase, M. et. al. (1998) “Intracellular Hyperthermia for Cancer Using Magnetite Cationic Liposomes An in vivo Study” Jpn. J. Cancer Res. 89:463-469.
353. Zhang Y, Wade M M, Scorpio A, Zhang H, Sun Z. Mode of action of pyrazinamide: disruption of Mycobacterium tuberculosis membrane transport and energetics by pirazinoic acid. J. Antimicrob. Chemother. 2003; 52: 790-795.

### SUMMARY OF THE INVENTION

The present invention is explained in the following embodiment with illustration and examples. Those examples below should not, however, be considered to limit the scope of the invention, it is contemplated that modifications will readily come to mind to those skilled in the art, which modifications will be within the spirit of the invention and the scope of the appended claims.

The object of the present invention is to provide a new method of treating all forms of tuberculosis comprising intravenous administration of high doses of a pharmaceutical composition containing Vitamin C, combined with different anti-Mycobacterium tuberculosis therapeutic agents by enhancing (augmentation) their effect with use of Insulin to cure and eradicate the disease.

In the present embodiments, the subject is a mammal; the mammal is human.

The present invention provides the method of curing tuberculosis patients comprising intravenous administration of a pharmaceutical composition containing high dose vitamin C to stop spread of the disease to others by droplet infection whose sputum is positive for mycobacterium tuberculosis.

The present invention provides the method of treating tuberculosis comprising intravenous administration of a pharmaceutical composition containing high dose vitamin C along with other anti M. tuberculosis therapeutic agents (drugs) for killing Mycobacterium tuberculosis bacteria within days, not months, or years to cure tuberculosis (TB).

The object of the present invention is to induce mild hypoglycemia by systemic parenteral insulin injection to make the mycobacterium tuberculosis membrane and phagocytes containing the TB bacteria more permeable to Vitamin C and anti-TB therapeutic agents at the same time enhance and augment the therapeutic effectiveness of Vitamin C and anti-tuberculosis drugs many times to kill the TB bacteria by producing reactive oxygen species (ROS=.O2−+H2O2→.OH+OH−+O2) by using parenteral insulin induced hypoglycemia activated therapy (IHAT). The purpose of the present invention is to provide the method of treating and curing tuberculosis comprising administration of a pharmaceutical composition containing streptomycin for faster curing patients suffering from tuberculosis along with other tuberculosis bacterial therapeutic agents and vitamin C with insulin.

The intent of the present invention is to provide the method of treating and curing tuberculosis comprising administration of pharmaceutical composition containing large doses of IV Rifampicin (RIF) for curing patients suffering from tuberculosis infection for use along with other anti-tuberculosis bacterial therapeutic agents with Vitamin C with insulin induced hypoglycemia activated therapy (IHAT).

The intent of the present invention is to provide the method of treating and curing tuberculosis comprising administration of a pharmaceutical composition containing Isoniazid (INH), for curing patients suffering from tuberculosis for use along with other anti-tuberculosis bacterial therapeutic agents with Vitamin C with insulin (IHAT),

An additional aspect of the invention provides methods of treating and curing of treating tuberculosis infection comprising administration of a pharmaceutical composition containing Pyrazinamide (PZA), for curing patients suffering from tuberculosis along with other anti-tuberculosis bacterial therapeutic agents with Vitamin C with insulin,

Yet another object of the present invention is to provide the method of treating and curing tuberculosis comprising administration of a pharmaceutical composition containing Ethambutol (EMB) for curing patients suffering from tuberculosis for use along with other anti M. tuberculosis bacterial therapeutic agents and Vitamin C with insulin (IHAT),

Intention of the present invention is to provide the method of treating and curing tuberculosis comprising administration of a pharmaceutical composition containing multiple anti M. tuberculosis bacterial therapeutic agents along with Vitamin C and insulin for treatment of all categories of tuberculosis including treatment failures, latent TB, as well as single or multiple drug resistant (MDR) tuberculosis,

Intention of the present invention is to provide the method of treating and curing tuberculosis comprising administration of a pharmaceutical composition containing Ethionamide (2-ethylthioiso-nicotinamide, 2-ethylpyrimidine-4-carbothioamide; Trecator® U.S. Pat. No. 8,912,329 B2), a structural analogue of isoniazid, is currently the last line of defense in the treatment of multi-drug-resistant tuberculosis (MDR-TB). Using Ethionamide along with Vitamin C and insulin IHAT reduces the dose of this therapeutic agent's toxicity by reducing the dose that is administered.

The goal of the present invention is to provide the method of treating and curing tuberculosis comprising administration of a pharmaceutical composition containing multiple anti M. tuberculosis bacterial therapeutic agents with Vitamin C and insulin for treatment of all categories of tuberculosis for reduction in duration of treatment of tuberculosis,

The purpose of the present invention is to provide the method of treating and curing tuberculosis comprising administration of a pharmaceutical composition containing multiple effective chemotherapeutic agents selected from the list therapeutic agents effective against drug resistant bacteria with insulin, and Vitamin C for treatment of all drug-resistant M. tuberculosis bacteria. A combination of antibiotics called fluoroquinolones and injectable medications, such as amikacin, kanamycin or capreomycin, are generally used for 20 to 30 months. By our method it can be cut down to a few weeks. Some types of TB which are developing resistance to these medications as well need to be treated with High dose Vitamin C and rifampicin described here in which needs only a shorter duration of treatment,

Yet another object of the present invention is to provide the method of treating and curing tuberculosis comprising administration of a pharmaceutical composition containing multiple anti M. tuberculosis bacterial therapeutic agents and insulin with Vitamin C for treatment of all drug-resistant TB. New drugs are being developed add-on therapy to the current drug-resistant combination treatment including: Bedaquiline, Delamanid, PA-824, Linezolid, Sutezolid, and other future therapeutic agents under development,

Yet another purpose of the present invention is to provide the method of treating and curing tuberculosis comprising administration of a pharmaceutical composition containing multiple anti M. tuberculosis bacterial therapeutic agents combined with the anti-inflammatory NSAID drug aspirin and ibuprofen to reduce the inflammation, diminish the formation of tuberculosis granuloma lesion, and caseation lesions where dormant M. tuberculosis bacilli are thought to persist,

It is the intent of this invention to deliver Vitamin C and anti-tuberculosis drugs including recombinant interferon-Y⁻ 1 b through a nebulizer and/or directly delivered through the described device (FIGS. 5, 6, 7) to the site of granuloma-cavitary tuberculosis,

It is the intent of this invention to provide hyperbaric oxygen (HBO) therapy during the administration of Vitamin C to increase the output of ROS to kill the drug resistant and non-resistant mycobacterium tuberculosis bacteria rapidly,

It is another target of this invention to deliver anti-TB agents with Vitamin C and insulin (IHAT) prophylactically to eradicate latent TB infection in suspected cases (those exposed to open TB cases-latent TB), thus preventing further development into full blown disease at the same time preventing its spread by preventing becoming an open case (meaning M. tuberculosis bacteria present in the sputum),

It is another intention of this invention to deliver anti-TB agents with Vitamin C and insulin directly to the site of the disease through special therapeutic agents delivery device described here in (FIGS. 5-7) with intermittent or continuous delivery of selected anti-tuberculosis drugs through syringe, syringe pump or delivery pump,

It is another object of this invention to deliver anti-TB therapeutic agents with Vitamin C and insulin directly to the site of the TB cold abscess after draining the pus, delivering therapeutic agents for many days through an indwelling catheter (FIG. 7) described herein,

It is another object of this invention for treating pulmonary and extrapulmonary tuberculosis by delivering the relatively weak electrical fields and alternate electric (AC) currents in the range of 10 KHz to 100 to 500 KHz which passes from anterior to posterior chest wall traversing through the infected lungs granuloma, cavitary lesions and infected hilar lymph nodes that will have bactericidal—bacteriostatic effect on the dividing M. tuberculosis bacteria in the infected lungs, thus acting as adjuvant therapy to augment the effect of anti TB drugs,

Another intent of this invention to inject anti TB drugs with insulin and Vitamin C palpable lymph nodes in the neck and other accessible areas of the body.

Another intent of this invention to inject anti TB drugs with insulin and Vitamin C around the vertebra and bones early, into and around the suspected lesions, before it expresses as granuloma and cold abscess.

It is another object of this invention to deliver 2,4, dinitrophenol (DNP) locally to produce heat within M. tuberculosis and phagocytosed cells by collapsing the proton (hydrogen cations) motive force (proton ionophore) and allowing the proton to leak through the inner M. tuberculosis cell membrane which is akin to mitochondrial inner membrane. It induces glycolysis, thus making ATP production less efficient. The production or “phosphorylation” of ATP by ATP synthase gets disconnected or “uncoupled” from oxidation. Instead of producing ATP, the energy of the proton gradient is lost as heat that enhances the production of ROS, which is bactericidal, and the lack of ATP production in the M. tuberculosis results in its death.

It is the intent of this invention to use intravenous Ozone autohemotherapy, to kill the tuberculosis bacteria in the open or latent TB infection. Ozone counteracts the effect of Vitamin C; hence it is used only 12 to 24 hours after the use of high dose Vitamin C (FIGS. 3, 4, 3, 9).

Presently-used drugs to treat TB were developed more than four decades ago, the treatments using these drugs are very long and require up to eight pills a day for longer than six to twenty four months or more, which can become burdensome treatments increasing patient's noncompliance and can lead to an increase in multidrug-resistant tuberculosis (MDR TB). In addition, the presently-used drugs typically do not target latent, nonproliferating TB bacteria hence the present invention will overcome these hurdles.

The invention explained herein further provides treatment to prevent the emergence of additional drug resistance and treat them effectively diagnosing and curing existing MDR- and XDR-TB cases. Our success will depend on the development of or new methods of administration in combination of multiple therapeutic agents already available, and administration of newly developed anti-M. tuberculosis bacterial agents in addition that are designed to achieve four major objectives reached by the invention, described here:
a) Shorten treatment duration to days, not months after months extending to years,
b) Increase adherence by patients by enabling intermittent examination and therapy schedules, education of the patients adhering to the protocol, and by providing guidance of the healthcare workers,
c) incorporate newly introduced agents with novel mechanisms of action to ensure activity against drug-resistant and non-resistant mycobacterium tuberculosis,
d) Decrease incidence and spread of TB by developing safer, shorter duration treatment modalities/regimens for Latent TB infection (LTBI) and converting the acid fast mycobacterium tuberculosis positive to negative sputum.

The foremost goal of this invention for treatment of tuberculosis M. tuberculosis bacterial infection include:
a) Curing the individual patient within a short period of time,
b) Minimizing risk of death and disability with proper treatment goals and follow up of the treatment and make sure the patient follows the treatment as prescribed,
c) Reducing transmission of mycobacterium tuberculosis to other persons by early treatment and isolating the open tuberculosis with proper mask wearing and protecting the care giving members and contacts,
d) Eliminating the latent TB infection so that patients do not develop the full blown disease due to any number of reasons including immune system suppression as seen in AIDS, other chronic debilitating diseases and the patents on immunosuppressive anti-TNF alpha drugs such as infliximab (Remicade®), Etanercept (Enbrel®), and adalimumab (Humira®) (Keane, et al. 2001). Interestingly, the studies also led to suggestions regarding the value of TNF-α blockade, particularly with infliximab, to efficiently block granuloma formation and increase clearance of M. tuberculosis in combination with effective tuberculosis chemotherapy (Wallis 2005, Skerry. et al. 2012).

It is the intent of this invention additionally that provides a method for treating tuberculosis using inexpensive drugs i.e. Vitamin C, rifampicin, Insulin (IHAT) and ibuprofen which with other existing anti M. tuberculosis bacterial drugs; that are widely available now to treat TB instead of waiting for new therapeutic agents which costs more than 100 to 500 million dollars, taking decades to develop each therapeutic agent to curtail and cure this endemic disease which kills millions year after year.

The flow chart 8 and 8a summarizes the principle of treatments of tuberculosis described in this invention.

### B RIEF DESCRIPTION OF THE DRAWINGS

FIG. 1 is the diagrammatic presentation 100 of the structure of the M. tuberculosis, how it gets its energy and how we can use it to eliminate it.

FIG. 1a is the diagrammatic presentation 100a showing the histology of the pulmonary TB bacteria from droplet infection entering the pulmonary air passages and taken up by white blood cell-macrophage.

FIG. 2 is the diagrammatic presentation 200 showing the histology of the pulmonary or lymph node tuberculosis lesion.

FIG. 3 is the diagram of the 300 of the mechanism of action of vitamin C, insulin and therapeutic agents against Mycobacterium tuberculosis.

FIG. 4 is the diagram of the 400 of the mycobacterium tuberculosis showing the mechanism of action of vitamin C, insulin and other therapeutic agents.

FIG. 5 is the diagram of the 500 showing pulmonary tuberculosis lesions and therapeutic agent's delivery catheter to the TB lesion and TB cavitary in the lungs.

FIG. 6 is the diagrammatic presentation 600 of the inventive device 80 to deliver therapeutic agents to the pathologic lesions the bronco pulmonary segment.

FIG. 6a is the diagrammatic presentation 600a of the inventive device 80 to deliver therapeutic agents to the pathologic lesions the bronco pulmonary segment continuously.

FIG. 7 is the diagrammatic presentation of 700 showing the cold abscess and its drainage and treatment with indwelling therapeutic agent's delivery catheter.

FIG. 8 is the diagrammatic presentation 800 showing the flow chart of end result of tuberculosis bacterial infection of a person.

FIG. 8a is the diagrammatic presentation 800a showing the flow chart of treatment outlines described in our invention.

FIG. 9 is the diagrammatic presentation 900 showing the production of zone and it use in our invention to treat tuberculosis.

FIG. 10 is the diagrammatic presentation 1000 showing the effect of insulin and anti-tuberculosis drugs on the cell membrane insulin receptors.

FIG. 11 is diagram of chest wall 1100 with AC electrical current delivery device.

FIG. 12 is the diagram of mitosis of the cell 1200 and how AC electrical field inhibits and disrupts mitosis in TB bacteria

### DETAILED DESCRIPTION OF THE INVENTION

In the following detailed description of the invention, reference is made to the drawings in which reference numerals refer to like elements, and which are intended to show by way of illustration specific embodiments in which the invention may be practiced. It is understood that other embodiments may be utilized and that structural changes may be made without departing from the scope and spirit of the invention.

### DEFINITIONS OF TERMS USED

The terms “Tuberculosis”, also called “TB”, is an infection caused by Mycobacterium tuberculosis bacteria characterized by the formation of “tubercle” a small round mass, projection, nodule, eminence in the lungs and other tissues of the body hence the name “tuberculosis” TB usually affects the lungs, but it can spread to the kidneys, bones, spine, brain, and other parts of the body.

The term “curtail” means controlling or bringing down the active tuberculosis infection and making the sputum negative to prevent its spread to those in close contact.

The term “Cure” or “Cured” refers to elimination of the tuberculosis bacteria from the body without any symptoms and signs; and the bacterial number in the patients to be below the limit of detection effectively curing the tuberculosis.

The term “prophylactic” means the methods to prevent the tuberculosis infection development such as by vaccination and/or administration of therapeutic agents to prevent development of full blown infection after possible suspected exposure to the disease by open known case or prevent infection by M. tuberculosis bacteria vaccinations such as bacillus Calmette-Guérin (BCG), an attenuated M. tuberculosis bacteria.

The term “persister” tuberculosis cells manes that the tuberculosis bacteria is still present in the macrophages in spite of the treatment and may not spread and produce the active tuberculosis. They can be called bacteria that are dormant or in hiding.

The term “latent tuberculosis” (LTB), also called “latent tuberculosis infection” (LTBI) means a patient is infected with Mycobacterium tuberculosis, but the patient does not have active tuberculosis and do not spread the disease. The identification and treatment of people with latent TB is an important part of controlling tuberculosis. Various treatment regimens are in use to treat latent tuberculosis, which generally need to be taken for several months. Our method described in this invention may cut down the time of treatment.

The term “inactive”, “dormant” or “latent infection of M. tuberculosis” refers to a M. tuberculosis infection without disease symptoms.

The term “reactivation” used when a person with latent TB develops active TB (disease), with the symptoms that can lead to delays in seeking care, and results in transmission of the bacteria to others.

The term “tuberculosis reactivation” refers to the later manifestation of disease symptoms in an individual that tests positive in a tuberculin test but does not have apparent disease symptoms. The individual is infected had been treated sufficiently to bring the tuberculosis into an inactive or latent state.

The term “Primary tuberculosis” refers to clinical illness (manifestation of disease symptoms) soon following infection.

The term “Secondary tuberculosis” or “post-primary tuberculosis” refers to the reactivation of a dormant, inactive or latent M. tuberculosis infection.

The term “active infection of M. tuberculosis” refers to tuberculosis bacterial infection with symptoms.

The term “drug resistant” M. tuberculosis infection refers to a M. tuberculosis bacterial infecting strain is not held static or killed and is resistant to “front-line” chemotherapeutic agents e.g., isoniazid, rifampin, ethambutol, streptomycin and pyrazinamide. A number of patients do not complete the prescribed regimen and course of treatment, which can lead to the development of drug resistance.

The term “Contagious” means that the infected patient may be asymptomatic, but that the person is still spewing the tuberculosis bacteria in the droplets from the lungs and extras pulmonary lesion which can infects those in close contacts.

The term “multi-drug resistant” refers to tuberculosis bacterial infection and the infecting strain is resistant to two or more of “front-line” chemotherapeutic agents.

Patients who been never treated before, or treated for less than one month develop drug resistance are defined or termed “Primary drug resistance” and those who develop resistance M. tuberculosis bacteria to drugs are defined or termed as “acquired drug resistance” Treatment of MDR tuberculosis are very expensive, toxic arduous and frequently ineffective. It is here our described therapy can be very effective.

The term “chemotherapeutic agent” and “anti-tuberculosis drugs” or “therapeutic agents” “anti TB drugs” refers to pharmacologic agents used in the treatment of M. tuberculosis infection.

The terms “effective amount,” “pharmaceutically effective amount” and “therapeutically effective amount” refer to a nontoxic but sufficient amount of an agent to provide the desired biological result resulting in reduction and/or alleviation of the signs, symptoms, or causes of a disease, or any other desired alteration of a biological.

The term “disease” is a state of health of an living being that cannot maintain homeostasis, and wherein if the disease is not ameliorated then the animal's health continues to deteriorate.

The term “alleviated” if the harshness of a symptom of the disease or disorder, the frequency with which such a symptom is experienced by a patient, or both, is reduced.

The term “abnormal,” when used in the context of organisms, tissues, cells or components thereof, refers to those organisms, tissues, cells or components thereof that differ in at least one observable or detectable characteristic (e.g., age, treatment, time of day, and the like) from those organisms, tissues, cells or components thereof that display the “normal” (expected) respective characteristic. Characteristics that are normal or expected for one cell or tissue type might be abnormal for a different cell or tissue type.

The terms “patient” and “subject” and “individual” are used interchangeably herein, and refer to any animal, or human amenable to the methods described herein.

The term “potency” refers to the dose needed to produce half the maximal response (ED₅₀), here refers to curing and/or curtailing tuberculosis.

The acronym or shorthand term “IHAT” refers to insulin induced hypoglycemia activated therapy that enhances anti TB drugs uptake and augment their effect. This method of therapy increases the uptake of anti TB therapeutic agents due to increased permeability of cell membrane, and increase their effectiveness many folds. It has also been named as insulin Potentiation therapy “IPT”, insulin-potentiated targeted low dose therapy “IPTLD” All these acronyms carry the same meaning.

In the following detailed description of the inventive device and anti-tuberculosis therapies elaborated with reference is made to the drawings in which reference numerals are intended to show by way of illustration of specific embodiments in which the inventions may be used to treat the tuberculosis. It is understood that other embodiments may be utilized, structural changes may be made and deliver combination of different anti-tuberculosis therapeutic agents without departing from the scope and spirit of the invention.

According to the present invention, tuberculosis disease patients are treated by recognizing patient condition attributable at least in part to lung signs and symptoms or afflictions in other parts of the body. With reference now to the various figures in which identical embodiments are not numbered alike throughout the description of the present invention presented. These diagrams represent and describe this invention individually and describe how the anti-tuberculosis therapeutic agents delivered to the lungs or local lesions using the described combination of TB therapeutic agents and inventive device or by parenteral administration to cure tuberculosis infection of the body. While the preferred embodiment of the present invention has been described, it should be understood that various changes, adaptations, and modifications may be made thereto. It should be understood, therefore, that the invention is not limited to details of the illustrated invention. The inventive device system described herein delivers therapeutic agent described herein to treat tuberculosis to the lungs or in other regions of the body with specific beneficial or curative therapeutic agents to treat this diseases.

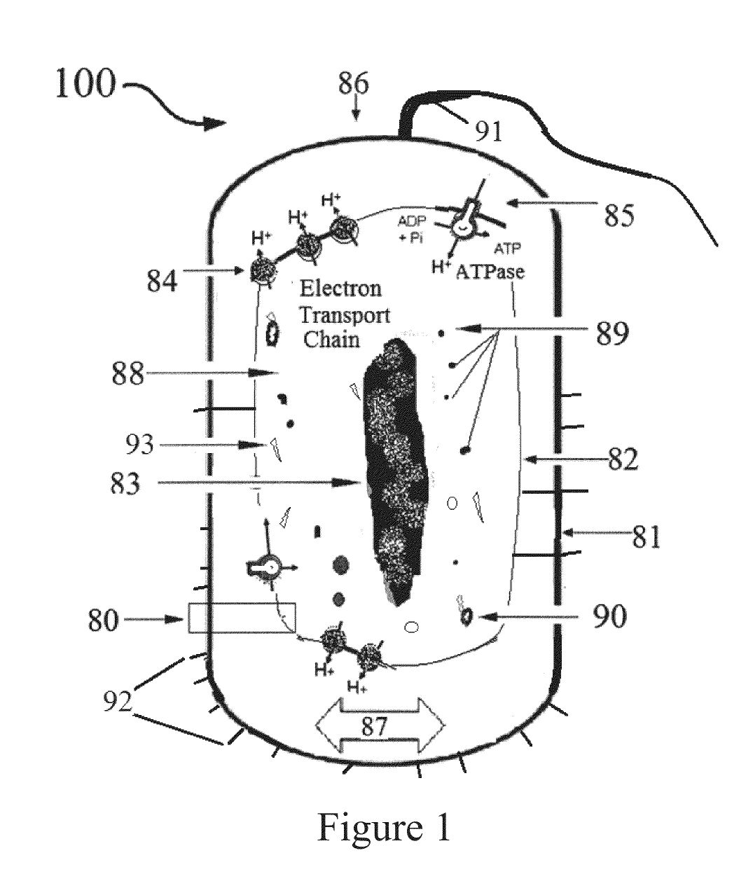

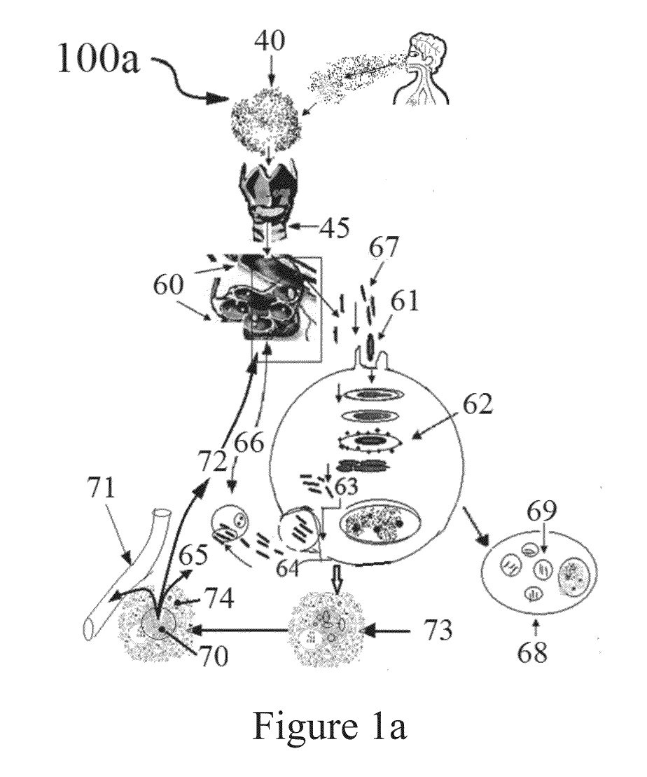

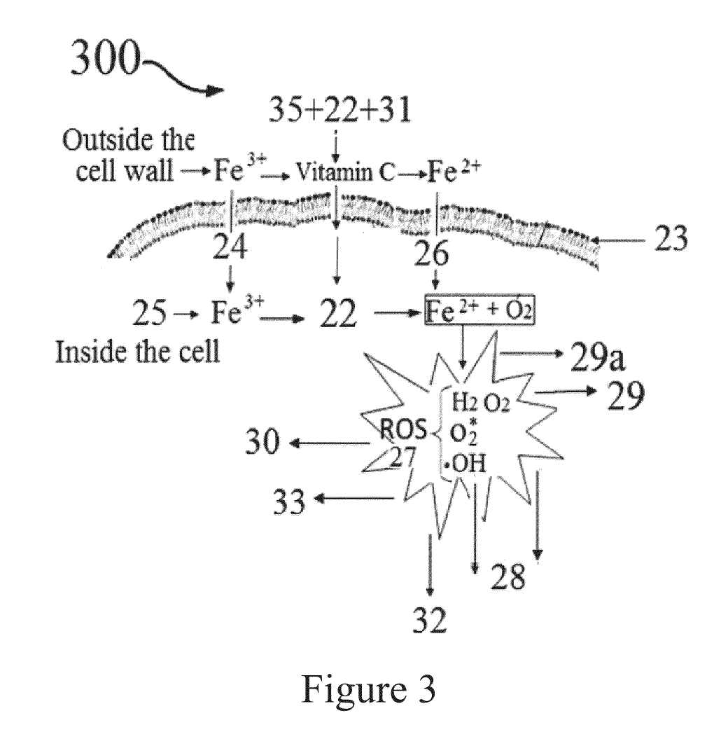

FIG. 1 is the diagrammatic presentation 100 of the structure of the M. tuberculosis, how it gets its energy and how we can use it to eliminate the infection by anti TB therapeutic agents. The diagram shows that M. tuberculosis bacteria are made up of double membrane namely out capsule with cell wall 81, and the inner part of the cell wall lined by plasma membrane 82. These are akin to the mitochondrion cell wall in our body cells which are also made up of two layers. It is shows that it is surrounded by extracellular space 86. It has a cell wall 80 which is made of outer cell membrane 81 (cell wall or capsule), and inner cell membrane or wall 82 with intermembrane space 87 in-between and is a periplasmic space. Any therapeutic agent that enters the heart of the M. tuberculosis bacteria has to traverse through this space 87 into cytoplasm 88. The inner cell has electron transport system 85 which produces ATP for survival and functioning of the cell like the inner membrane of the mitochondrion. Note that the electron transports chain which contains proton pumps 84 plays a major role in production of electrons to produce ATP. The 2, 4, dinitrophenol (DNP), blocks the ATP production 85 contributing to intracellular heat (intracellular hyperthermia) resulting in cell death due to heat, paucity of ATP for energy, and enhanced ROS production. The other uncoupling proteins which increase heat are carbonyl cyanide m-chlorophenylhydrazone (CCCP), salicylic acid, thyroid hormone, nor epinephrine, epinephrine, leptin, usnic acid as such. FIG. 3 show how these membranes play a role in the M. tuberculosis bacterial destruction by anti-tuberculosis drugs.

The bacteria are provided with flagellum 91 which is said to help in their motility (just wiggle locally). It is also endowed with multiple hairs like appendage named pili (pilus) 92 on the outer surface. Though we do not know the function of these pili in this bacteria cell wall, it is thought to be used to protect the cell's surface and helps in adhesion to other surfaces. May be they helps to latch on to the surface of the air passages, alveolar surface and macrophage in the lung alveoli.

The center of the cell (matrix) has a dense mass of nucleiod circular DNA 83. The cytoplasm contains ribosomes 89, plasmid 90 and proteosomes 93. The ribosome is a multi protein complex, and it is the factory where protein synthesis occurs. The structure of the ribosome in bacteria and human cells differs significantly and this difference allows some antibiotics to specifically kill bacteria. Plasmid 90 is a small DNA molecule, physically separated from the chromosomal DNA 83 and can replicate independently. It may play a role in bacterial resistance to therapeutic agents. For instance, many plasmids contain genes that, when expressed, make the host bacterium resistant to an antibiotic (so it won't die when treated with that antibiotic). Plasmids contain genes that help the host to digest unusual substances (anti-bacterial therapeutic agents) or to kill other types of bacteria. Specifically, plasmids are nonessential, extra chromosomal pieces of DNA with up to 100 genes. What exactly does that mean? A plasmid is a short, usually circular, double-stranded segment of DNA that is found in the cytoplasm separate from the main bacterial chromosome. TB bacteria like other bacteria replicate by binary fission. A single bacterial cell, called a mother cell, copies the chromosome, and then the cell splits in half, giving each half of the cell a copy of the chromosome. The two new identical daughter cells are essentially clones of the mother cell. But what about the plasmids? Plasmids carry genes that direct their own replication and additional factors that ensure that the copies get separated into the new daughter cells. This ensures that the plasmids are not lost from the cells during M. tuberculosis bacterial binary fission. Any therapeutic agent that inhibits DNA will also inhibit the activity of plasmid and plays a role in killing the bacteria.

Most antibiotics against non-replicating TB work by preventing the bacteria from building essential proteins, such as those found in its cell wall—an outer layer that protects the microbe against immune attack. A unique feature of tuberculosis bacteria is that it is the only bacterium to have a proteasome 93 structures inside the cell that remove damaged proteins and help the bacteria survive in the dormant state. Proteasomes are ATP-dependent protein degradation machines present in all archaea and eukaryotes, and found in several bacterial species of the order Actinomycetales. Mycobacterium tuberculosis, an Actinomycete, pathogenic to humans, requires proteasome function to cause disease. Proteasomes are protein complexes inside all eukaryotes and in some bacteria. In eukaryotes, they are located in the nucleus and the cytoplasm. The main function of the proteasome is to degrade unwanted or damaged proteins by proteolysis, a chemical reaction that breaks peptide bonds. Enzymes that carry out such reactions are called proteases.

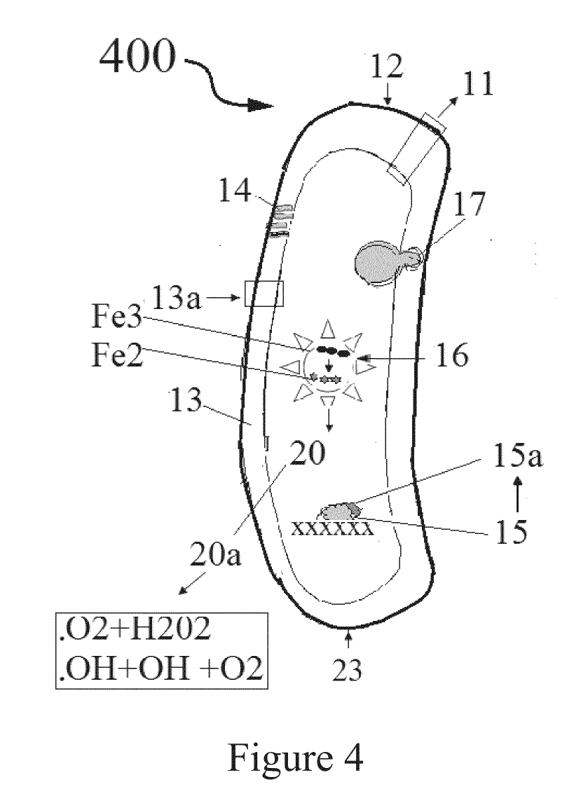

Proteasomes are part of a major mechanism by which cells regulate the concentration of particular proteins and degrade misfolded proteins besides being involved in cell stress response, cellular differentiation. The degradation process yields peptides of about seven to eight amino acids long, which can then be further degraded into shorter amino acid sequences and used in synthesizing new proteins. It is known, that removal of proteasome gene is lethal to the cell. If we could specifically target the proteasome and stop it from working, damaged proteins would accumulate inside the bacteria and cause the microbe's death. The group of compounds called “oxathiazol-2-one,” could effectively block the proteasome activity. Researchers tested the inhibitors on TB bacteria and on monkey and human cells. They found that the inhibitors killed the TB bacteria, but they did not appear to harm mammalian cells, which also have Proteasomes. The researchers also observed that oxathiazol-2-one (derived from the dipeptide backbone of Bortezomib), inhibited the TB proteasome about 1,000 times more effectively than the human proteasome (Lin, G et. al 2009) and kill non-replicating M. tuberculosis. This could be in new TB therapy in the horizon using our method of treatment. Currently, treating latent TB requires six to twenty four months of multi drug therapy—a lengthy process can lead to noncompliance, and that it can lead to antibiotic resistance if the patient stops taking the medication. Because of these drawbacks, researchers would like to find more effective treatments for the disease, which may arise through new approaches to attacking the bacteria by use of oxathiazol-2-one. See FIG. 4 for details on how and where various anti TB drugs, Vitamin C, heat producing uncouplers act on the above described histological structure of M. tuberculosis bacteria to kill them.

Researchers and clinicians visualize treating TB in four to six weeks, and being able to cure it is a desire right now, but drugs that appear to attack targets that the bacteria need to survive even though they're not all replicating would be a real breakthrough. We believe that delivering the existing anti TB therapeutic agents with Vitamin C after insulin induced hypoglycemia activated therapy can achieve such a goal by acting on the genetic material, cell wall, proteosomes, RNA polymerase, plasmids, enzymes inside the cell walls. Our method of treating TB with insulin will augment the division of the bacteria, at which time they become vulnerable for the bactericidal effects of anti TB drugs.

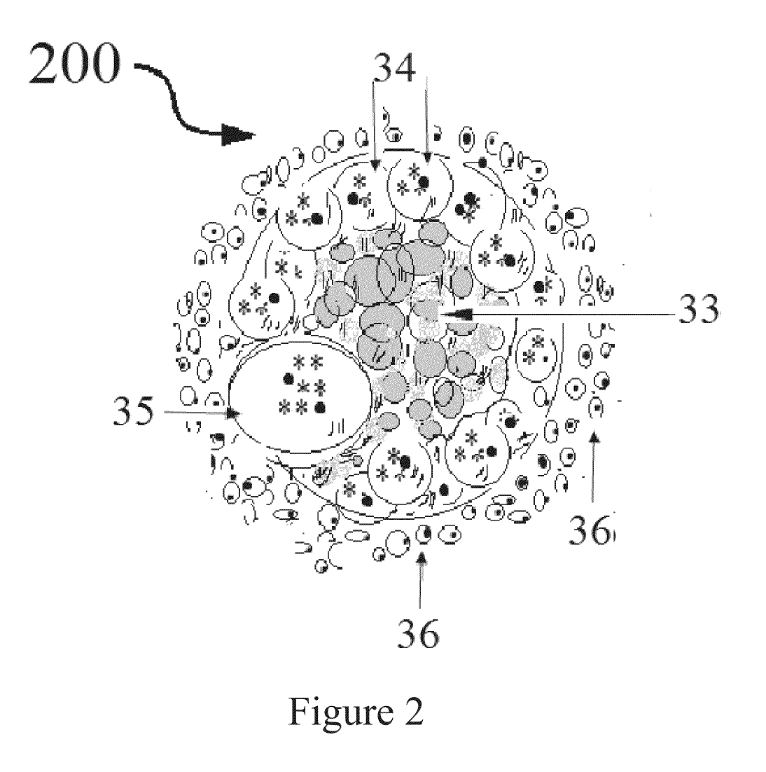

FIG. 1a is the diagrammatic presentation 100a showing life cycle histology of the pulmonary TB bacteria from droplet infection 40 entering the trachea-bronchial-pulmonary air passages 45, and reach alveoli 60 and phagocytosed by white blood cell—macrophages 62. Each inhaled droplet nuclei is capable of transporting 1-3 bacilli into the alveoli of the lungs (Dannenberg and Rook 1994). As the droplets containing the M. tuberculosis bacteria are breathed from the infected person 40, 67, the macrophages in the alveoli phagocytose the TB bacteria 61, 67 and form phagosome 62 within their cytoplasm enveloped by macrophage cell membrane. The bacteria replicates in the alveoli, and in phagosome of macrophages by binary fission. The blood vessels in the wall of the alveoli bring more defensive cells to the alveolar infection site. The alveolar wall is damaged by the migration of leukocytes and cytokines of this inflammatory response to the infection and also due to the development of tubercles. As the bacteria are phagocytosed by macrophages 61, it forms phagosome 62 with a wall surrounding it. The bacterium multiplies within the phagosome. Ultimately the phagosome membrane 62 is breached, and the multiplied bacteria liberated to cytoplasm 63 of the macrophage. From the cytoplasm, the bacteria are extruded to air passages and other surrounding structures (may bacteria come out as droplets while coughing infecting the close contacts) and/or the macrophage succumbs to infection liberating the TB bacteria to surrounding tissue 64 which are picked up again by the other macrophages 65 and cells of the immune system in the air passages and alveoli 66 ultimately forming tuberculosis granulation tissue (granuloma tubercle). Some of these liberated TB bacteria also enter the lymphatic's, spreading to the hilar mediastinal and other lymph nodes directly by lymphatic ducts or carried by the immune system's cells through lymphatic ducts and may be even by hematologic spread. This figure also shows the hiding of the M. tuberculosis bacteria in the macrophages as latent infection 68 within the persister macrophages 69. Ultimately tubercle 73 forms in the alveoli with dying macrophages teaming with multiplying bacteria in the center (see FIG. 2) surrounded by lymphocytes, epithelioid cells and giant cells. In due course, the tubercle granuloma center becomes necrotic 70 with dead macrophages. The wall formed around the tubercle mostly by lymphocytes 74 ruptures (3 bent arrows) liberating the bacteria to the alveoli 72 with more inflammatory response presented as pulmonary tuberculosis case. The caseous tubercle may rupture into the blood vessel 71 in the wall of the alveoli, spreading the infection to rest of the lungs producing miliary tuberculosis as well as far and wide hematogenously away from the lungs.

FIG. 2 is the histological diagram presentation 200 of a typical pulmonary tuberculosis lesion (late stage—tubercle with caseous center), showing a parenchymal focus and hilar lymph node lesion. The detailed section of the diagram shows characteristic features of tuberculous granuloma with different types of immune system cells surrounding the lesion. It has a central caseous necrosis 33 surrounded by epithelioid macrophages 34, multinucleated giant cells 35 present at the edge of lesion, and the entire lesion surrounded by multilayered lymphocytes and white blood cells 36 cells infiltration with some fibroblasts to wall of the lesion. Appearance of epithelioid cells in the TB lesion signals the destruction of bacilli and the limitation of the infection. Hypersensitivity (resistance to infection) is associated with increased phagocytosis of bacilli, conversion of macrophages to epithelioid cells 34, the formation of giant cells 35, and the inhibition of replication of tubercle bacilli. Bacilli are usually found in the caseous centers or in epithelioid cells, histiocytes or giant cells at the perimeter of the lesions. Because the sensitized T cells are lacking, the tubercle bacilli multiply freely and enter the blood stream and lymphatic's; disseminating the bacillus far and wide form the primary focus of inflammation.

Caseation (caseum-cheese like) is the “semisolid” necrosis of the exudative initial alveolar lesion and of the lung tissue surrounding the lesion. It results in alveolar destruction, but the elastic fibers of the alveolar walls and their vessels often persist within the caseous lesion. The persistence of elastic fibers is likely responsible for the hardness or the rubbery consistency of many solid caseous foci (Canetti 1955). A crucial phenomenon happens within the caseous lesion: the death of the majority of the tubercle bacilli. The host locally destroys its own tissue to control the uninhibited intracellular multiplication of bacilli that would otherwise be fatal. In a majority of cases (up to 90% of infected individuals), highly activated macrophages surround the caseous center. The bacillary antigens released by the dead bacilli expand T-cell populations. These T cells release interferon and other lymphokines that activate local macrophages. Such macrophages ingest and destroy the bacilli that escape from the edge of the caseum. In a resistant host, the caseous lesion is surrounded by a capsule. In time, its central part calcifies and even ossifies, especially if the caseous lesion had occurred remotely, for example, during childhood. Caseous lesions of small size can be infiltrated by sclerosis and even reabsorbed. Such lesions are devoid of viable tubercle bacilli (Canetti, 1955). Some caseous lesions of a certain size can persist for long periods of time without a clearly defined capsule or modification of the caseous center. In about 10% of the cases, the softening of the caseum begins and it is one of the most important events of tuberculosis. Because of softening, infection with M. tuberculosis progresses into tuberculosis, the disease. In a majority of cases, the softening of the caseum is associated with emptying of the softened material through a communication with the bronchial tree, resulting in the formation of a lung cavity (cavitary TB), and explosive growth of tubercle bacilli in the newly oxygen-enriched environment. With a cough, the softened caseous material with its high bacillary content is discharged into the bronchi and subsequently to other parts of the lung and to the outside environment (Grosset 2003). Although softening of the caseum is the most serious event in the course of tuberculosis, its mechanism remains largely unknown.

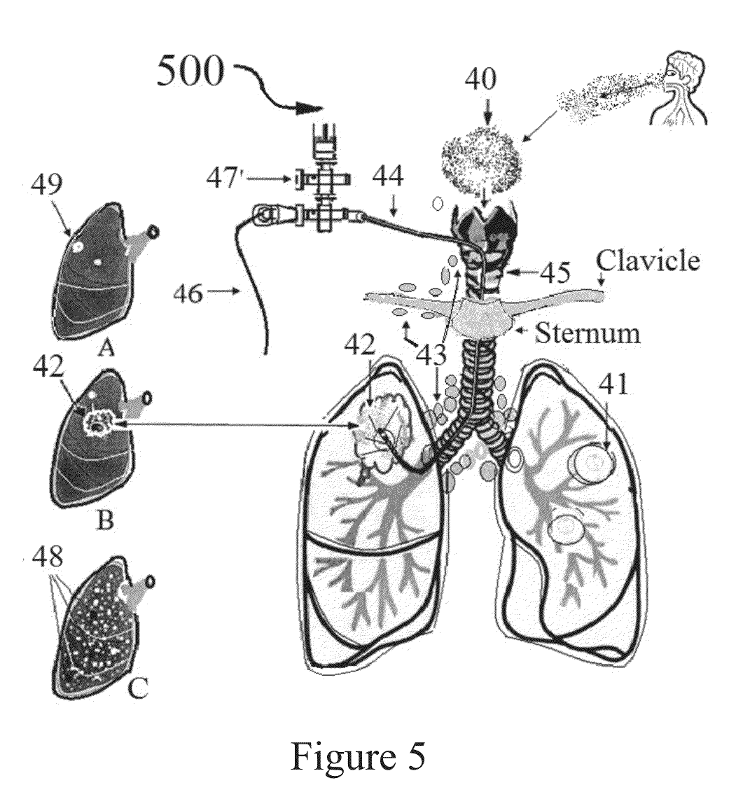

The same type of histological feature akin to primary tuberculosis lesion is also observed in the formation of Ghon's complex (FIG. 5), usually located sub-pleural. When this lesion breaks loose from the surrounding structures, it spills out the bacteria and its caseous material to terminal bronchioles (or to the blood vessels) of the alveoli setting up for active TB development. This acts as foreign material in the air passages, and produces a cough reflex expelling the material in droplet from containing mycobacterium tuberculosis. These active mycobacterium tuberculosis containing droplets are transmitted to those who are in close contact and breathe these droplets spreading the infection.

FIG. 3 is the diagrammatic presentation 300 showing the mechanism of action of vitamin C (possibly ozone, hyperbaric oxygen, hydrogen peroxide, some anti TB drugs), and insulin, against Mycobacterium tuberculosis. Vitamin C 22 enters Mycobacterium tuberculosis bacterial cells wall 23 and the cells infected with the tuberculosis bacteria and reduces ferric ions 24 in cytosol 25 to generate ferrous 26 ions. Cytosol is the part of the cytoplasm inside the cell 25 that is not held by any of the organelles in the cell and the central, granular mass in the cytoplasm is the endoplasm while the surrounding lucid layer is known as the cell cortex or the ectoplasm. It is in the cytosol that all the metabolic chemical reactions of prokaryotes take place. These ferrous ions in the cytosol 25 in the presence of oxygen, generate superoxide (an oxygen .O2-molecule with an extra electron), hydrogen peroxide H2O2 and hydroxyl (the negative ion formed by the attachment of an oxygen atom and a hydrogen atom), —OH radicals (an atom, molecule, or ion that has unpaired valence electrons or an open electron shell) through the Harber-Weiss and Fenton reactions as shown in the diagram. The production of these reactive oxygen species 27 (.O2−+H2O2→.OH+OH−+O2) leads to cells and organelle membrane disruption 33, and the RNA-DNA damage 28, alteration of lipids 30, fatty acid oxidation 32 redox imbalance 29 and NADPH depletion 29a leading to the destruction of the Mycobacterium tuberculosis. Insulin 35 with induction of hypoglycemia makes the mycobacterium tuberculosis membrane 23 more permeable for glucose as well as anti TB therapeutic agents 31 including Vitamin C 22 and enhance the activity of the Vitamin C inside the bacillus and phagocytosed cell containing the tuberculosis (and other microbes). In chemistry, a free radical is an atom, molecule, or ion that has unpaired valence electrons or an open electron shell, and therefore may be seen as having one or more “dangling” covalent bonds. Injection of insulin 35 increases the permeability of the mycobacterium tuberculosis membrane 23 and at the same time increases the production of reactive oxygen species 27 (ROS, chemically reactive molecules containing oxygen) and enhances and augments the therapeutic activity and effectiveness (Alabastor 1981) of anti TB drugs due to presence of insulin. In general, harmful effects of reactive oxygen species produced by Vitamin C enhanced by insulin and other anti TB drugs on the cell and mycobacterium tuberculosis bacteria are:

Damage RNA-DNA: When cellular RNA-DNA (#28) is damaged beyond repair. Therapeutic agents may also cause p53 (an apoptosis transcription factor normally found in the cytosol) be transported to the nucleus where it promotes p53 mediated apoptosis of the affected cells (macrophages) which contain the Mycobacterium tuberculosis, oxidations of polyunsaturated fatty acids in lipids (lipid peroxidation) damaging the cell wall (#32), oxidations of amino acids in proteins disrupting their configuration and reducing their survival mechanisms, oxidatively inactivate specific enzymes by oxidation of co-factors disrupting the bacterial function and growth.

Now, we describe how effective Vitamin C is in killing the mycobacterium tuberculosis by producing ROS 27 by what is called by Fenton reaction. Further, adding other anti TB agents and insulin is like pouring gasoline on the fire with strong anti-bactericidal-killing-effects. The Fenton reaction has importance in biology, because it involves the creation of free radicals by chemicals that are present in vivo. Transition-m et al ions such as iron and copper donate or accept free electrons via intracellular reactions and help in creating free radicals. Most intracellular iron is in ferric (+3 ions) 24 forms and must be reduced to the ferrous (+2 ions or d) 26 form inside the cell 25 to take part in Fenton reaction. Superoxide ions and transition met als act in a synergistic manner in the creation of free radical (ROS=−.O2−+H2O2→.OH+OH−+02) that kills the mycobacterium tuberculosis and the insulin enhances this biochemical and biological reaction within the cell.

FIG. 4 is the diagrammatic presentation 400 of the mycobacterium tuberculosis and its membranes 11 showing the mechanism of action of vitamin C 16, on M. tuberculosis bacteria 23; the effects of insulin induced hypoglycemia activated therapy on anti TB drugs, and various therapeutic agents used against tuberculosis bacteria by Fenton reaction 20 and with ROS 20a production. It shows that the streptomycin 12 damages TB cell membrane 11, Bedaquiline (R207910) 17 inhibits ATP synthase thus blocking the energy needed for the survival and multiplication of the bacteria. Rifampicin 15 inactivates RNA polymerase beta subunit 15a inside the bacteria needed for the survival and multiplication of these tuberculosis bacteria. Isoniazid 14 inhibits mycolic acid in the cell membrane, Pyrazinamide 13 inhibits or disrupts mycobacterium tuberculosis membrane transport system, cyclosporine 13a inhibits the cell wall biosynthesis and Vitamin C 16 damages the mycobacterium tuberculosis cell machinery and inflicts DNA damage by reactive oxygen species (ROS) 20, 20a production as explained in FIGS. 3, 4. Insulin 23 enhances the uptake and augments the effectiveness of all these therapeutic agents (Alabastor 1981, Shantha 2004), against mycobacterium tuberculosis many times. Insulin induced hypoglycemia activated therapy (IHAT) described here augments the uptake and therapeutic activity anti TB drugs many times.

Studies by Hanneke Later-Nijland through the Netherlands Organization for Scientific Research (2009) carried out research within the Indonesian, Tanzanian and Dutch research network showed that it is possible to shorten the duration of treatment by increasing the dose of the important drug rifampicin. After increasing the dose, the concentration of the drug in the blood plasma was higher than expected and did not experience adverse effects at an increased dose than at a standard dose. We want in our studies to increase the dose of rifampicin double the dose, and at the same time lower the sugar level by using the insulin to increase the uptake and effectiveness of rifampicin (and other therapeutic agents—Alabastor 1981) and make it possible to reduce the length of treatment for tuberculosis. Intravenous (I V) Injection of large doses of rifampicin day after day after insulin induced hypoglycemia activated therapy with high dose Vitamin C will eliminate the active, latent, and persisters' cell TB infection rapidly. Our method of treating TB with insulin will also augment the binary fission of the bacteria, at which time they become vulnerable for the bactericidal effects of anti TB drugs.

FIG. 5 is the diagrammatic presentation 500 showing pulmonary tuberculosis lesions and method of delivering therapeutic agents through our inventive device to the site of pathology. The diagram shows the droplets of 40 mycobacterium tuberculosis entering the lungs through the air passage 45 transmitted due to coughing, sneezing, forceful expiration, rapid breathing, and develops into tubercle granuloma known as Ghon's lesion 49 and that can lead to a caseous lesions 41,42. The destruction and coalesation of these lesions in the lungs leads to formation of lung cavitary 42 and may be even bronchiectasis where this inventive device 44 is used to treat these conditions.

The wall of the tuberculosis cavity in the lungs 42 consists of an external zone of collagen, the cavity's capsule, and an internal zone of softening caseum where intersected with elastic tissue and BV. Because of the direct connection with the airways, the high oxygen content favors the rapid multiplication of tubercle bacilli. For the first time during the course of the disease, the bacilli are free to multiply extracellularly (Grosset 2003). Ultimately, it is the softening of the caseous tubercle results in the tuberculous lung cavity (Long E R. 1935), that perpetuates the disease in humans. By coughing, the patient with a lung cavity aerosolizes and disseminates bacilli to the other parts of the lung and to the outside world-open air. Our method of delivery of therapeutic agents with the device described here will eliminate these multiplying bacteria and their spread. Except in rare occasions, the tuberculous cavity does not heal spontaneously without treatment. However, a range of outcomes are possible and they are:

At one extreme the bacilli discharged from the cavity are ingested by non-activated macrophages, in which they temporarily grow until the delayed type hypersensitivity (DTH) or tissue-damaging hypersensitivity process kills the bacillus-containing macrophages and destroys nearby tissues. A new caseous focus is then created, and if the caseation process is repeated, a large part of the lungs is destroyed and the patient eventually dies. Before the antibiotic era, 50% of patients with cavitary lung tuberculosis died within 2 years (Enarson and Rouillon 1994).

At the other extreme, the bacilli discharged from the cavity are also ingested by macrophages. On the other hand, in a host with good cell mediated immunity, immunologically specific T cells and their lymphokines activate macrophages, which are then able to kill the intracellular bacilli without excessive tissue damage. In such a host (25% of tuberculosis patients before the antibiotic era), continuous destruction of host tissue is not necessary to contain the growth of bacilli and the lesions become more or less stable.

Intermediate between these two extreme events, another 25% of untreated patients experienced a chronic waxing-and-waning course of their cavitary tuberculosis.

From a practical point of view, it is the extracellular bacillary population and, as a top priority, those present in the lung cavity that clinicians aim to eliminate. Our method of treatment with direct delivery of therapeutic agents will eliminate these extracellular bacilli. It is this actively dividing population in the cavity, ranging well into the millions of organisms, that is most responsible for the person-to-person transmission of tuberculosis and that provides the reservoir for drug-resistant mutants. Used in combination, these drugs have the ability to kill drug-susceptible organisms and prevent the selection of drug-resistant mutants. Indeed, within the first 1 months of appropriate chemotherapy, the vast majority of bacilli have been killed, virtually eliminating the risk of transmission and the selection of drug-resistant mutants. Provided that appropriate chemotherapy is continued, the major therapeutic challenge remaining is to eliminate the tiny number of viable drug-susceptible bacilli that persist despite several months of effective drug therapy. In that respect, rifampin is undoubtedly the most important drug we incorporate in our invention to treat the cases with high dose Vitamin C. With the incorporation of rifampin into multidrug chemotherapy, we can achieve cure in a majority of patients within 6 weeks instead of 6 months, whereas therapy with isoniazid previously required a minimum of 18 months. Although 6 months of therapy is a great benefit compared to 18 months, 6 months of therapy cannot be considered a short duration. To begin to understand why it takes months to kill a handful of persisters, we must address the issue of the nature, the metabolic status, and the location of these persisters.

The special activity of rifampin against them does not close the debate regarding whether they are located intracellularly or extracellularly because rifampin is as active in mice, in which tubercle bacilli are mainly intracellular, as in humans, in which they are mainly extracellular. Combining high dose Vitamin C with rifampicin will eliminate tuberculosis bacteria whether they are locate extra or intracellular. One is tempted to conclude that the persisters are likely to remain “latent” as intact cells with occasional spurts of metabolism (Mitchison, D A. 1998), taking shelter in tiny areas of semi-solid caseous material. However, some fraction of the bacillary population also persist intra cellular, in unusual forms in some patients (Rook, G. A. W., and B. R. Bloom. 1994). No matter what bacterial form or their location, M. tuberculosis bacteria remain a significant adversary for the infected patients, clinicians and the scientists alike which can be eliminated using the method described herein.

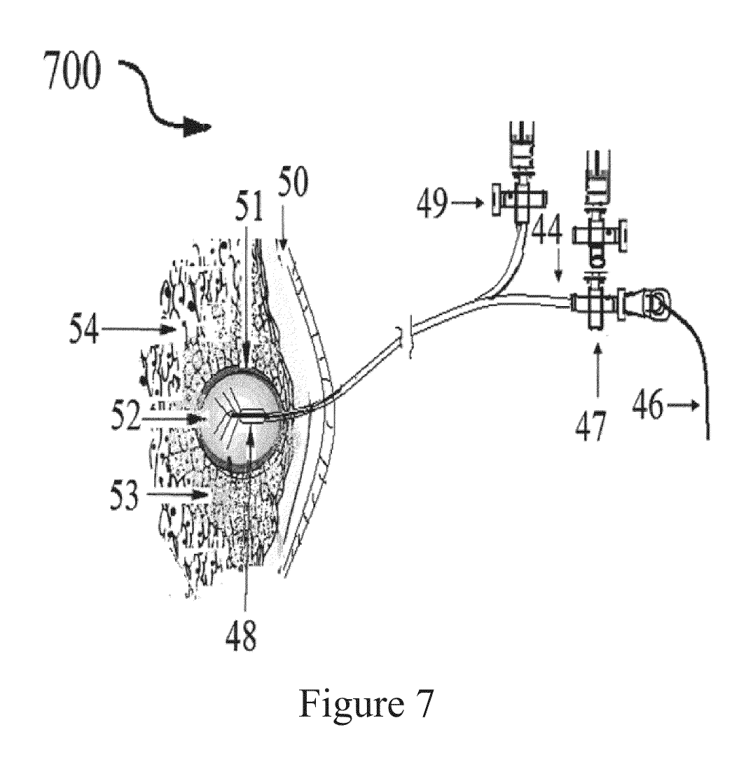

Tuberculosis bacteria from the lungs, the lung lymphatic's transport the mycobacterium tuberculosis bacteria to the lung hilar lymph nodes 43 and may be even to cervical and clavicular lymph node (arrows from 43) infecting them. The enlarged hilar lymph nodes produce hilar shadow in X ray of the chest (FIG. 5, #43). The neck lymph nodes enlargements are felt in the neck on palpation if infected. The distant tissues (bone and other organs) with development of TB lesions that can lead to cold abscesses formation (FIG. 7) are diagnosed by history of signs and symptoms, and radiological examination.

Later on, the resting bacilli in the Ghon's lesion 49 may break out and cause serious tuberculous infection when immune mechanisms wane or fail with clinical manifestation of abrupt high fever and develop low grade fever as the time passes (may be associated with progression to tuberculous pneumonia), pleurisy with effusion, and lymphadenitis. Radiographically, the Ghon's lesions are spherical and cavitary—the so-called coin lesions. A fibrous capsule surrounds the caseous, acellular center, which contains numerous tubercle bacilli. If a branch of pulmonary artery and vein in the wall of TB lesion breaks, it can lead to massive hemoptisis, asphyxiation and even exsanguinations; and spread of the mycobacterium tuberculosis bacteria to other regions of the lugs developing miliary tuberculosis 48, and to distant organs away from the lungs. The lung diagram “A” shows the Ghon's lesion 49, lung diagram “B” shows the cavitary tuberculosis lesion in the upper lobe of the lungs 42, and the lung diagram “C” shows the miliary tuberculosis 48 all over the lobes of the lungs due to hematogenous spread of the M. tuberculosis from the ruptured TB lesion into a blood vessel.

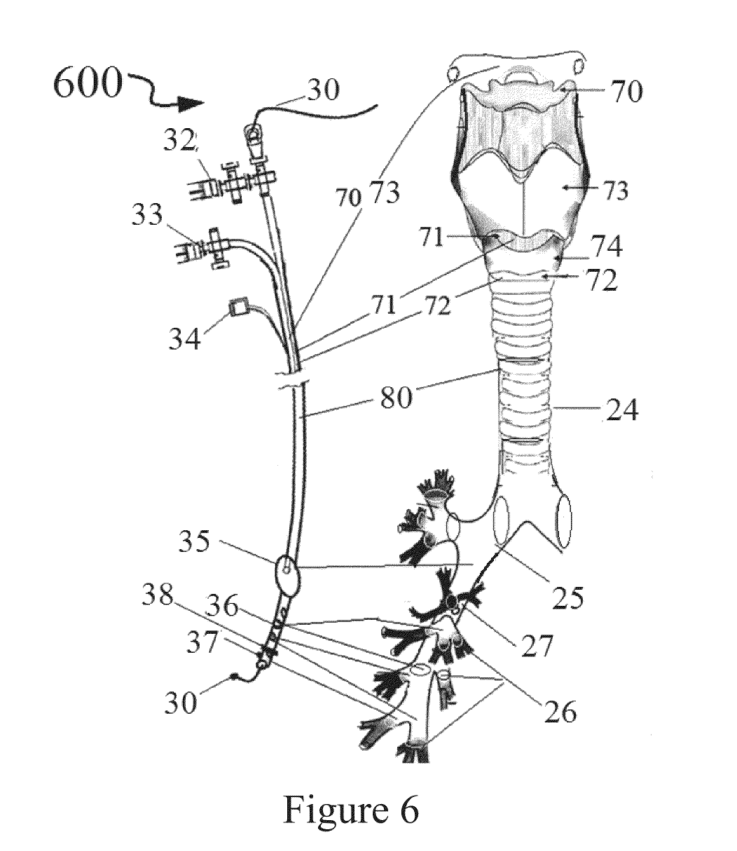

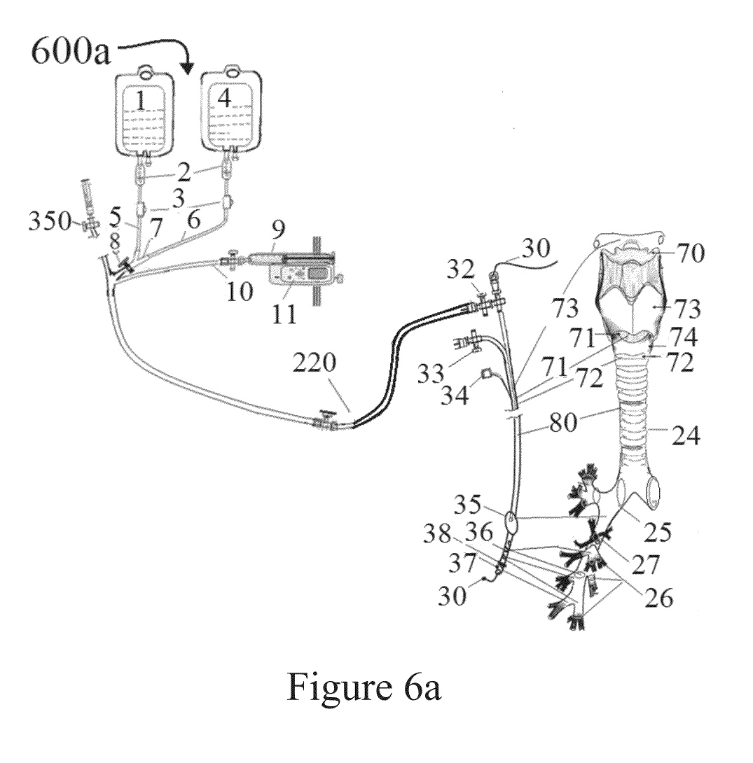

The diagram also shows the therapeutic agents delivery catheter device 44 (see FIG. 6 for details) to treat TB cavitary 42 or localized granulomatous as well as cavitary pulmonary lesions. The catheter is passed through the cricothyroid or subcricoid region 45 (Shantha 1992) and the therapeutic agent's delivery tip positioned in the cavitary or granuloma with the help of stilet 46 using Seldinger technique, and/or with the help of the fiber optic imagery 34 as shown in the FIG. 6 #34. The proximal end of the device has three way stopcock 47 to deliver therapeutic agents through a syringe 47 attached to the stopcock or by an automatic infusion pump (not shown) to deliver Vitamin C and other anti-mycobacterium tuberculosis drugs with insulin into the TB cavitary directly in the lungs or to the site of the lesion. It can be connected to continuous therapeutic agent's delivery infusion bags or automatic delivery micro syringe as described in FIG. 6a. At the distal end, it is provided with balloon, radio opaque tip indicator, and fiber optic image transmitter incorporated as described in FIG. 6 to facilitate the passage and positioning of the therapeutic agents delivery catheter device 44.

FIG. 6 is the diagrammatic presentation 600 of the inventive device 80 to deliver therapeutic agents to the pathologic lesions through the bronco pulmonary alveoli segments 24,25,26,27 (the air passages) to treat TB and other pulmonary lesions including cancers. It is long semi rigid tubing made up of non-reacting, non-allergic silicone-plastic-composite material provided with two tubes at the proximal end-one to inflate the balloon and the other to deliver therapeutic agents to the site of pathology. It is connected by 3-waystop cocks 33 to the distal end balloon 35 located±5 cm from the tip of the device. When inflated from the syringe with open stopcock 33 with air or gel, it expands and blocks the retrograde flow of injectate from the terminal bronchi and bronchioles leading alveoli or from the cold abscesses. It is provided with guide wire 30 (J wire) whose tip is rounded like a pin head to prevent trauma to the tracheal and bronchial lining and alveoli as the device is passed, and has a C shaped curve for ease of introducing and guiding the device as it is inserted thorough the bronchiole openings 25, 26, 27 and placing therapeutic agents delivery device tip to its proper location (site of TB lesion), preferably in the center of the lesion. The terminal 3 cm of the device is provided with multiple pores to deliver the therapeutic agents to the site of pulmonary tissue and TB cavitary and granuloma in the lungs. The proximal end is provided with therapeutic agent's delivery 3-way stopcock with syringe 32 to deliver Vitamin C and anti TB therapeutics through the tubing main body to the tip location at the site of pathology. The distal end in addition is provided with radio opaque detector 38 to locate the position of the tip of the pulmonary therapeutic agent's delivery device in the lungs through imaging x ray. The tip is additionally provided with fiber optic terminal 37 connected to the image capture device 34 from the distal fiber optic tip 37 to view as the device is passed down to the site of pathology. The device is passed through trachea 24 then to right side main bronchus 25 then to the bronchi 27 and bronchioles 26. This device is inserted into the trachea 24 through the larynx 70, 73 or cricothyroid membrane 71 or subcricoid region 72, 74 (Shantha 1992). In 1953 Seldinger described a simple, over a guide wire, approach for catheter insertion (Seldinger, 1.S. I. 1953). Seldinger technique offered considerable advantages over the previously used methods, revolutionizing the field of bedside procedures and it is widely used in the intensive care unit (ICU), operating rooms, interventional radiologists and in cardiac cath labs and to place central venous catheters (CVCs, SG catheters), hemodialysis (HD) catheters, arterial catheters, and chest tubes. The positioning of this delivery device can be achieved by use of the J guide wire using the Seldinger method with ease. First inject 3-4 ml of local anesthetic (2% xylocaine-lidocaine) using a fine needle passing though the cricothyroid or subcricoid membrane into the trachea. It initiates cough reflex and spreads the local anesthetics in the sensitive airways. Wait 3-5 minutes to local anesthetic to take effect, then pass the guide wore through intracath and position the tip at the site of pathology. Remove the intracath, make a nick on the skin to facilitate the insertion of the device, and then slide the delivery device along the guide wire. Once the delivery device is positioned, the J wire is withdrawn, the balloon inflated, and the infusion of anti-tuberculosis therapeutic agents begins.

Continuous infusion of Vitamin C and anti TB drugs is instituted through the syringe 32 or with the help of a continuous infusion pump or micro delivery bag attached to the proximal end of the delivery device 80. The same device is used to treat the cold abscess (see FIG. 7) to drain and sterilize the wall and surroundings tissue of the cold abscess TB cavitary. This device can be used to treat the carcinoma and other lungs pathology besides tuberculosis. The inflated balloon will help to hold the device inside the cold abscess or in the lung bronchioles and alveoli without being slipped out and can stay in the site for many days till the goal of draining and curing the local condition of the mycobacterium tuberculosis infection is achieved. The catheter without the fiber optic embodiment is all that is needed for treating cold abscess. This inventive device described herein will deliver the therapeutic agents against tuberculosis bacteria to the site of pathology where the bacteria are located in large doses instead of therapeutic agents circulating all over the body to reach this site of pathogen and pathology with unwanted systemic effects in healthy organs and tissues.

FIG. 6a is the diagrammatic presentation 600a of the inventive device 80 to deliver therapeutic agents to the pathologic lesions the bronco pulmonary segment is connected continuous delivery of therapeutic agents to the lung lesion. It shows the catheter 220 connected to the device 80 at stopcock 32 to deliver anti-tuberculosis therapeutic agents from the infusion bags 1, 4 and automatic delivery micro syringe 11 to the TB lesions in the lungs. It shows anti-Parkinson's drugs in Primary infusion bag 1, macro- or micro-drip tubing's 2, roller type flow control clamp 3, adjuvant therapeutic agents in secondary infusion bag (piggyback) 4, connected to primary infusion line 5, secondary infusion line 6, by Y-type connector 7 with three way stopcock 8 to the main delivery tubing 220. The diagram also shows the continuous infusion micro delivery syringe pump 11 with therapeutic agent's delivery syringe 9, connected to the device tubing 10, and 220 with 3 way stopcock then to the pulmonary device 80 in the cavitary tuberculosis lesion of the lungs. One can adjust the flow rate from these delivery bags from 0.1 ml to 3 ml per hour (can be increased up to 50 m per hours) delivered to the lungs through our device 80 depending on the severity of the disease and need of the patient. These infusion bags 1, 4 and additional delivery syringes 340 deliver desired therapeutic agents continuously or intermittently at the desired flow rate and desired time.

Instead of therapeutic agent's infusion bags, anti-tuberculosis drugs loaded automatic delivery micro syringe can be connected to the lung delivery device 80. The micro infusion pump 11 has the capacity to hold and deliver from 10 ml to 50 ml in the syringe 9. It has flow rate increment of 0.1 to 1 ml or more per hour connected to lungs delivery device 80. It is operated by battery pack of nickel mega halide, which is rechargeable. The rest of the explanations of device 80 are same as FIG. 6.

FIG. 7 is the diagrammatic presentation of 700 showing the cold abscess (non-pulmonary lesion) as a result of hematologic or lymphatic spread of mycobacterium tuberculosis bacteria from the lungs blood vessels and lymphatic vessels. It shows the bulging of the skin 50 due to expansion of the granuloma and pyogenic cold abscess 52 produced by the inflammatory reaction to M. tuberculosis bacterial infection, surrounded by pyogenic membrane 51. The cold abscess is surrounded by granulation tissue 53 which is projecting above the fatty layer (subcutaneous tissue) of the skin 54. The diagram shows the drainage of the cold abscess by using especially designed catheter 44 introduced through a wide bore introducer (not shown) or by using a wide bore needle using Zig Zag aspiration method. Ultrasound guided pig tail drainage catheter can also be used to drain large retro peritoneal abscess. The J wire or the stilet 46 is provided and it also facilitates the insertion and positioning of the catheter and the three-way stopcock 47 is attached to therapeutic agent's delivery syringe. Open drainage using dependent incision of the cold abscess performed if the aspiration failed to clear the content of the abscess. The drainage catheter is proximally provided with an opening through a three-way stopcock 47 to inject and irrigate the abscess with Vitamin C and other anti TB drugs especially streptomycin and rifampicin through the distal end which has pores to deliver the therapeutic agents. Ozonized water can be used to irrigate the cold abscess after drainage to sterilize the wound and debridement as described by Jyoti et al (2013) in dental practice. A weak solution of hydrogen peroxide can also use immediately after draining the abscess. It is provided with a balloon 48 that can be expanded by the inflating syringe 49 attached to the three way stopcock 49 from outside with air or liquid and it will hold the injection-drainage tubing in position after the procedure is completed. The terminal catheter is provided with openings to deliver therapeutic agents and drain the cold abscess. Once the procedure is complete, the proximal end is attached to a sterile glove or drainage collector container. This device can be used repeatedly for week at a time and replaced if further drainage and injection of therapeutic agents is needed. Every day the cold abscess is irrigated with 10%-15% solutions of Vitamin C and anti TB drugs (1000 mg of streptomycin and/or 600 mg rifampicin) and other anti TB therapeutic agents are left in the drained abscess cavitary and process repeated daily, every other day, and twice a week, till the cold abscess begins to heal with minimum or no inflammatory productive material.

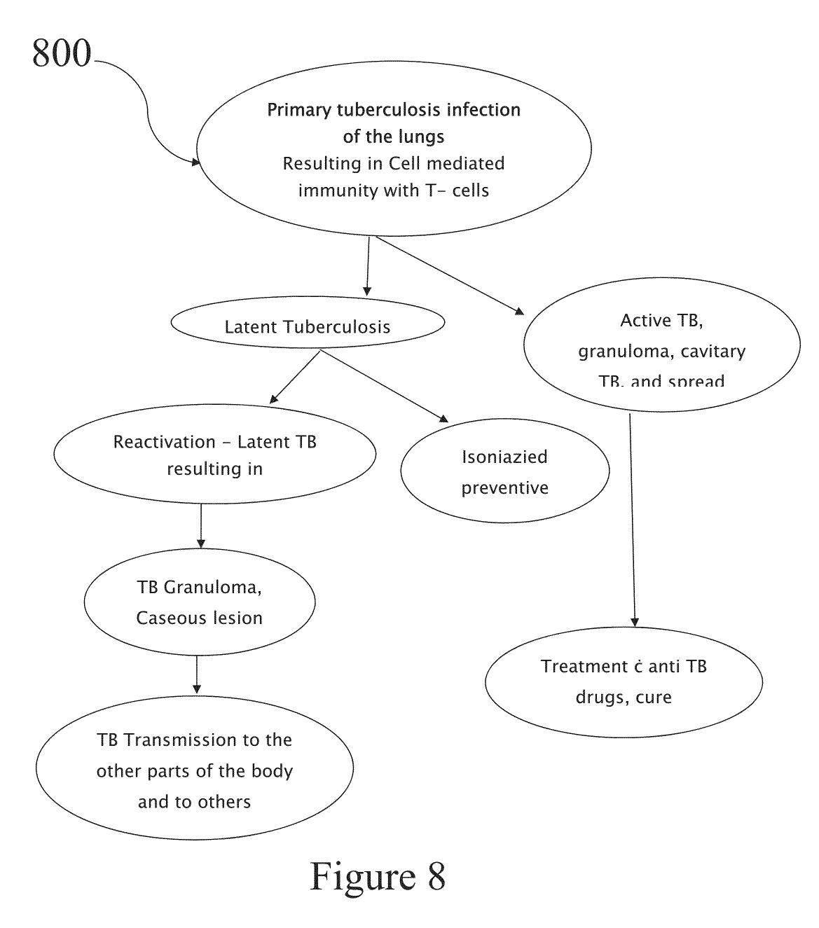

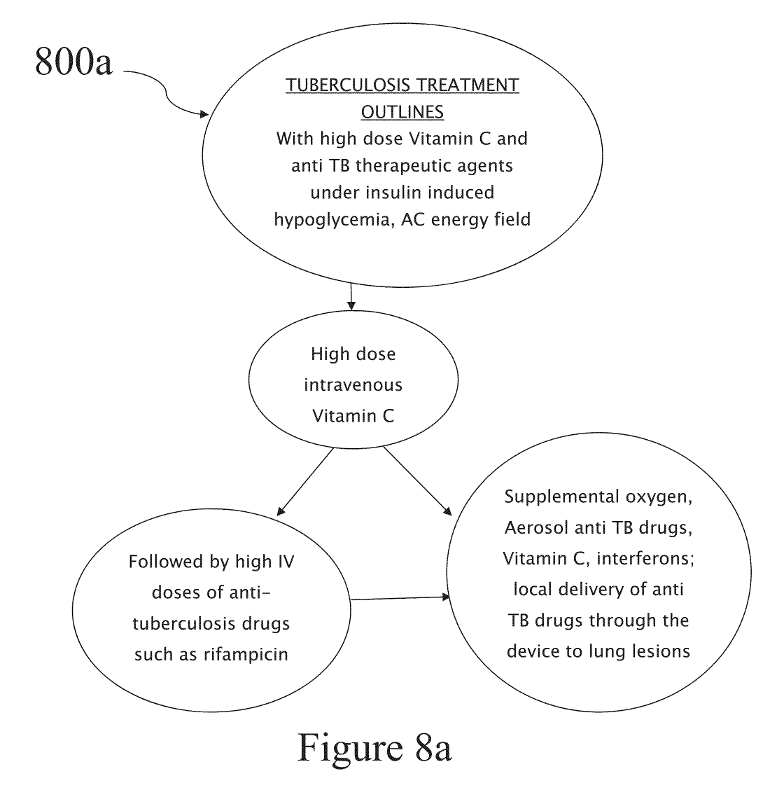

FIG. 8 is the diagrammatic presentation 800 of the flow chart as a result of tuberculosis bacterial lung infection of a person. It shows that once the tuberculosis bacteria infect the body, the person suffers from the serious consequences with increased morbidity and mortality. If untreated, 50% of the patients face death. The flow chart is self-explanatory.

FIG. 8a is the diagrammatic presentation 800a of flow chart showing the treatment outlines described in our invention. We start our treatment with daily intravenous infusion of high dose Vitamin C followed by administration of various anti-tuberculosis therapeutic agents with insulin. In addition, the patient is provided with supplemental oxygen to increase the ROS species killing effect by Vitamin C and anti TB drugs and these therapeutic agents are also delivered to the lungs by aerosol. We have described the invention of a device and method to deliver anti TB therapeutic agents in high doses to the site of lesion in the lungs and in to the cold abscesses after drainage through a delivery device. The flow chart is self-explanatory.

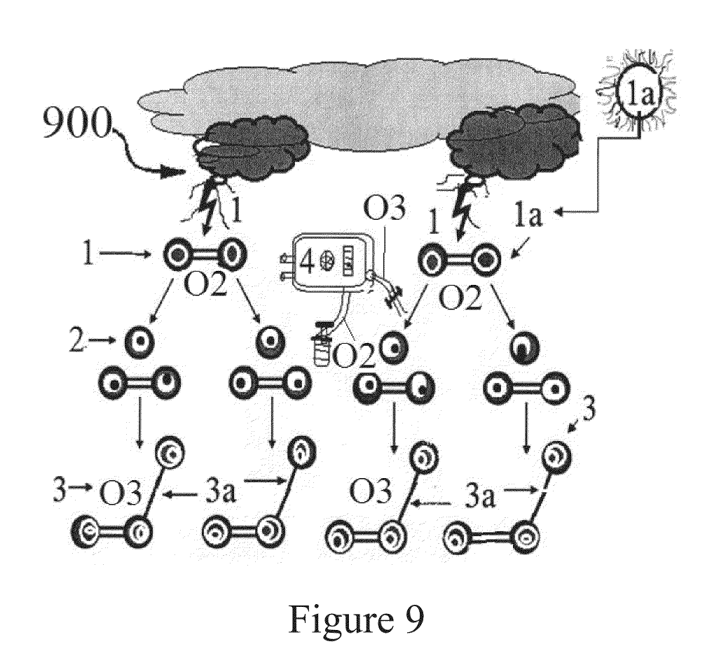

FIG. 9 is the diagrammatic presentation 900 showing mechanism of formation of ozone in nature 1, 1a and artificially by using oxygen 4. The electrical energy in nature by lightening 1, and UV radiation from the Sun 1a (or in the lab) can form the ozone. It is produced artificially 4 by electrically activating the medical grade 100% oxygen (Ozone Generator by corona-discharge) to form ozone. Electrical energy due to lightening 1 breaks the Oxygen (O2) molecule into two atoms of free —O1. The free oxygen 2 atoms unite with one O2 atom of oxygen to produce ozone 3(O3) bound by weak bond 3a, which rapidly breaks (within 20 minutes) liberating oxygen and singlet oxygen species. Thus produced (or laboratory produced) O3 is extremely unstable, and when it comes in contact with viruses, bacteria, fungus, molds, fats, cancer cells (cell membrane), oils, organic and inorganic molecules; damages their structure and inactivates them by producing reactive oxygen species (singlet oxygen, hydroxyls, hydrogen peroxide etc.). In viruses, bacteria, molds, other pathogens, and disease affected cell membrane (phagocytosed immune cells, cancer cells), it pokes holes destroying the offending agent and spilling their intra cellular contents needed for survival. In tuberculosis (also viruses and cancer cells), it makes holes and disrupts its integrity, spills its matrix content (FIG. 1#23) thus killing the organism whether M. tuberculosis bacteria is drug sensitive or resistant. It also pokes holes in the bacteria engulfed macrophages (monocytes) and exposes the microbes to ROS species molecules and other anti TB therapeutic agents that are circulating and come in contact. These denatured spilled out offending agents are picked up by the immune system, as antigens resulting in production of anti-bodies against the offending disease causing agents. It is ideal to use by mixing with autogenous blood with ozone and re-infusing it back to the veins of the patient for the treatment of TB. This process is called autohemotherapy. Rectal ozone therapy and IV injection of ozone are complex and needs physicians well trained in the procedure, who are few and far in-between. Ozone therapy is another adjuvant therapy in the treatment of tuberculosis to eliminate the M. tuberculosis infection rapidly besides others described herein. It needs to be used only by trained physicians in oxidative therapies and is not used by inhalation due to its direct damaging effect on the respiratory system. It can be instilled to the site of lesion through a delivery catheter (ozonated irrigation solutions or ozonized oxygen) described herein and into cold abscess after drainage.

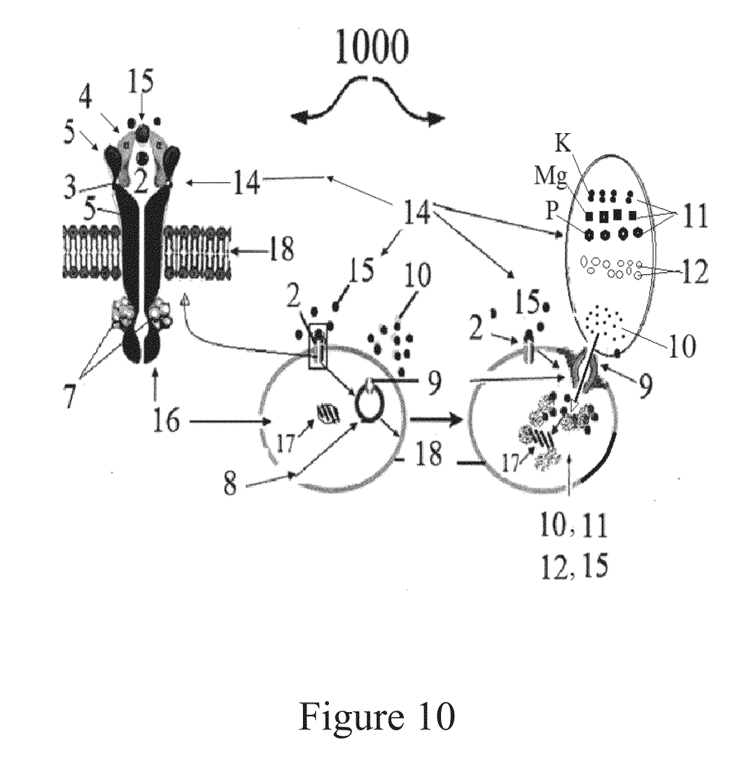

FIG. 10 is the diagrammatic presentation 1000 showing the effects of Vitamin C, insulin and anti-tuberculosis drugs on the cell membrane 18 in the extracellular fluid 14 in treatment of tuberculosis. The mechanism of action of insulin is explained simply, that the Insulin 15 binds to the insulin receptors 2 (a tyrosine kinase) on the cell wall 18, embedded in the plasma membrane 18. The insulin receptor is composed of two alpha subunits 4 and two beta subunits 5 linked by disulfide bonds 3. The alpha chains are entirely extracellular 14 and insulin 15 binding domains 4, while the linked beta chains 5 penetrate through the plasma membrane 18. Binding of insulin 15 to the alpha subunits 4 causes the beta subunits 5 to phosphorylate them (autophosphorylation), thus activating the catalytic activity of the receptor. The activated receptor 2 then phosphorylates a number of intracellular proteins 7, which in turn alters their activity, thereby generating a biological response such as Glut4 vesicles 8 to fuse with the plasma membrane 18. As the Glut4 9 from the vesicle separate and fuse with cell membrane 18, this allows the glucose 10 to enter the cell interior 16. GLUT4 is a glucose transporter that is stored in vesicles 8. A cascade of events that occurs upon insulin binding to a receptor in the plasma membrane 18 causes GLUT4-containing vesicles to fuse with the plasma membrane 18 so that glucose transported into the cell for energy needs. This action of Insulin also increases the permeability of cell membrane 18 to potassium (K), magnesium (Mg) and phosphate (P) ions 11, thus activates sodium-potassium ATPases inside the cells, causing a flux of potassium into cells. As this massive flux is taking place, the cell and bacterial membranes 18 become extra permeable to circulating chemotherapeutic agents and anti-tuberculosis drugs and Vitamin C 12 from extracellular space 14 to enter intracellular space 16 through the Glut4 opening 9, to kill the M. tuberculosis bacteria 17 (as well as other microorganisms). Insulin by itself increases the membrane permeability by altering cell membrane dynamics due to changes inside the cell. It is also Possible that the insulin biding to some of the anti-tuberculosis therapeutic agents also enhances their pharmacological activity many folds (Alabaster et. al., 1981), thus destroying the M. tuberculosis bacteria rapidly and effectively. Like cancer cells which develop multiple insulin receptors compared to normal cells due to their increased metabolic and energy need due to their rapid multiplication, likewise the M. tuberculosis bacteria infected macrophages may also develop multiple insulin receptors we believe, and hence subjected to the effects of insulin as described in this invention. Glucose transporter type 4, also known as GLUT4, is a protein that in humans is encoded by the GLUT4 gene. This explains our invention how the insulin (IHAT) increases the permeability to anti TB drugs into the phagocytes and its TB bacterial content to allow Vitamin C and anti-tuberculosis therapeutic agents for rapid curing of the tuberculosis. Our method of treating TB with insulin induced hypoglycemia activated therapy will augment the binary division of the TB bacteria as it induces mitosis in cells, at which time they become more vulnerable for the bactericidal effects of anti TB drugs.

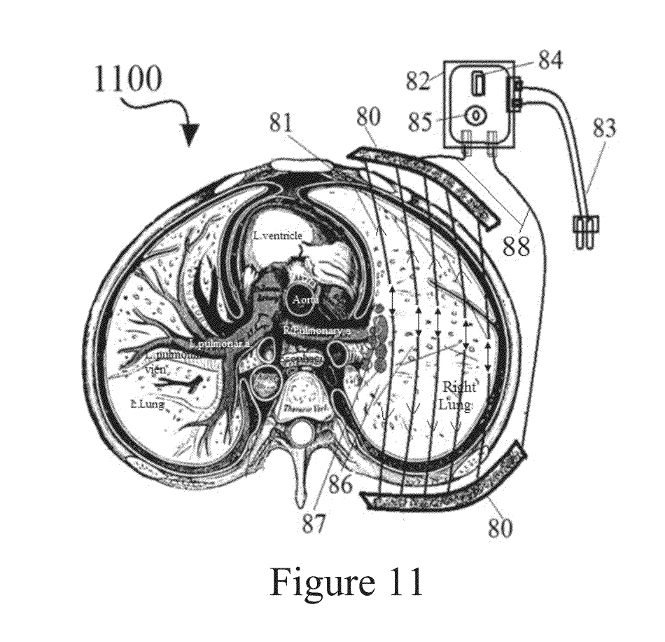

FIG. 11 showing the diagram 1100 of the coronal section of the thoracic cavity with lesions of M. tuberculosis infection of the right lungs 86 with lymph node enlargement 87. It shows the AC electric output generator manipulator 82 with on and off switch 84 and electrical power output adjuster 85 connected to the electrical outlet 83. Insulated electrical conductor 80 are attached to the anterior and posterior chest wall which generated electric field 81 that travel the entire lungs front and back over the lungs 86 with tuberculosis lesions. The alternate current generator 82 is connected by a pair of conductive leads 88. The device combines, and simultaneously delivers, frequency-modulated (FM) and amplitude-modulated (AM) electric cell membrane signaling currents in pulsed electromagnetic fields (EMFs) to kill TB bacteria and augment the healing of the affected site in the lungs. By using this method, we are combining physics and pharmacology work together to promote physiological environment that have killing of the offending TB bacteria and favor healing. The electromagnetic field created by AC reduces pain, causes relaxation of skeletal components, increases circulation, brings more therapeutic agents to the site of pathology, supplies more nutrients to improve the immune system response, reduces local and neuroinflammation by “wash out” of the metabolic products due to increased circulation; thus helps in the treatment of tuberculosis by clearing the area of infection for rapid healing.

The electrical field using AC is left on the chest wall applied for days and weeks or longer to inhibit the M. tuberculosis bacterial multiplication and augment the effect of the Vitamin C and anti-tuberculosis drugs. Water and chemical accumulation at the skin surface contact area can damage the superficial skin layers should be kept in mind when electrotherapy applied for long periods. The conductive gels used between the electrodes and skin. Appropriate safety mechanism be incorporated to turn off the power to the electrode if overheats as triggered by the sensor on the device. Studies show that when cells are subjected to relatively weak electric fields, by applying an AC voltage at high frequencies, having a frequency in the range of 50 KHz-500 KHz, which delivers a field strength between about 0.1 to 10V/cm at the target region (U.S. Pat. No. 8,170,684 B2), and have effect on the dividing microbes such as M. tuberculosis bacteria resulting in inhibition of the expansion and progression of the tuberculosis. These electrical fields have no effect on the non-dividing cells. Further, this method of creating electro-magnetic field combined with local anesthetic (in this case anti TB bacteria therapeutic agents) has proved effective in the treatment of pain (Odell, Sorgnard, and Milne. 2015). In the same fashion, using this method can alleviate the pain in the vertebral column and thoracic cage, besides inhibiting the multiplication of M. tuberculosis bacteria. In physics, electron behavior is referred to as “organized chaos.” This idea unites activities of electrical and pharmaceutical medicine, and thus ties disease and curative medicine together conceptually. We call this combined therapy as “Electro-chemo-antibiotic therapy”

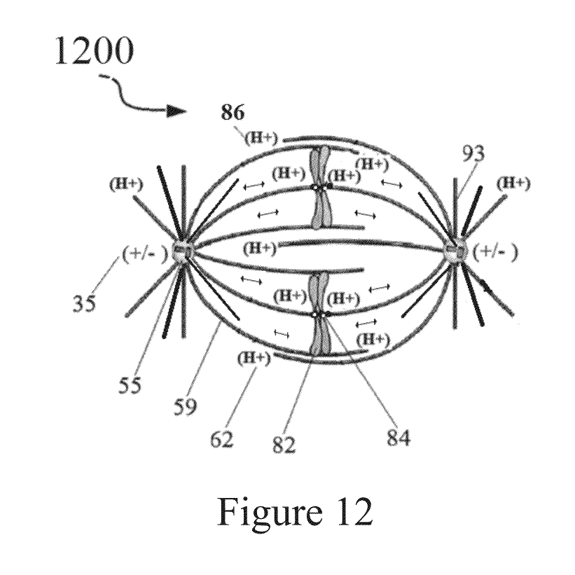

FIG. 12 diagram of eukaryote cell mitosis 1200 shows how the alternating (+/−=AC) electrical fields on the chest wall disrupts polar mitotic spindles 59 being pulled to the centrosome 55 for the effective cell division. Prokaryotes, which TB bacteria are, do not show all the classic histological features of mitosis as seen in eukaryote, they are affected same as eukaryote. The AC electrical therapy, induces change negative polarity at the centrosome replaced by the both positive and negative fields 35 created artificially by using alternating current which will prevent the organization of the mitotic spindles 59 for the cell to organize at the centrosome pole 55 for the cell to divide. Similar mechanism also plays a role in the M. tuberculosis bacteria and prevents their multiplication; though the dividing TB bacteria does not show classic histological feature of mitosis as seen in eukaryote (human body cells) with membrane bound organelle, and genetic material enclosed by nuclear envelope (Eukaryote). Further the alternating electrical field alters the concentration of H+ ions 85 which caused tissue acidity pH 6.9 or lower and reduces them to increase pH to 7.4, that is changed to alkaline media thus change the inflammation within the cell. The diagram show mitotic spindles 59 move towards polar centrosome 55 from the chromosome 82 and kinetochore 84, prevented by alternating current fields at the centrosome from forming astral mitotic tubule organization 93. It also shows the chromosome 82 and the kinetochore 84 in the middle of the chromosome spindle and kinetochore mitotic spindles 59. That is how effective applying the alternating electrical fields in the treatment of the tuberculosis and other infections of the lungs including cancers. The cells which are stuck at the middle of cell division, unable to complete the division are arrested from dividing. This arrest in mitotic stage increases the permeability of the cell membrane also. Hence these partly undivided TB bacteria are very vulnerable to the therapeutic agents against M. tuberculosis. As the bacteria are stuck in the middle of the cell division (mitosis by binary fission), the cell membrane becomes permeable anti TB therapeutic agents making them more vulnerable resulting in their death. Further AC electrical fields are shown to increase the number, size, and overall activity of mitochondria. Increased mitochondrial activity allows for substantially more energy for bio utilization and regeneration of cells. This physiologic benefit of electrical singling is important; because it increases production and use of energy are central to the healing process at the same time enhance the output of ROS by Vitamin C and anti-tuberculosis therapeutic agents which are destructive to the M. tuberculosis bacteria.

Anti-Bactericidal Activity of Vitamin C Against Mycobacterium tuberculosis and Other Viral and Bacterial Infections:

Decades back, Dr. Klenner and many others have treated using Vitamin C various infections both viral (polio, herpes, measles, pancarditis due to adenovirus), bacterial (pneumonia), parasitic (trichinosis), hyper cholesterimia; Shock (from toxalbumin, neurotoxin, proteotoxin, muscarine and formic acid); Epstein-Barr virus with Burkett lymphoma, hepatitis, tetanus, cancers (Cameron, Pauling and their associates, Casciari, Riordan, et al.), burn cases, and even mental conditions such as Schizophrenia—the list is not finite (Klenner, 1951-1974).

Vitamin C has the unique quality of entering all cells in the body-healthy or disease afflicted and the free floating pathogens. Any therapeutic agents that kill Mycobacterium tuberculosis rapidly as shown in the FIGS. 2-8, can shorten treatment time significantly and prevent the spread of infection to others individuals and other regions within. It is known that in Escherichia coli, a common mechanism of cell death by bactericidal antibiotics entails and requires the generation of highly reactive hydroxyl radicals (ROS) via the Fenton reaction. Similarly, studies by Vilcheze, C., T. Hartman, B. Weinrick W. R. Jacobs, Jr., (2013), have shown that vitamin C drives the Fenton reaction, sterilizes cultures of drug susceptible and drug-resistant mycobacterium tuberculosis, the infecting and causative agent of tuberculosis (FIGS. 3,4).

We subjected MRSA infected patients, cancer patients and others with persistent lung infection with high doses of intravenous Vitamin C plus antibiotics under insulin (optional) induced mild hypoglycemia, hyperbaric (optional) therapy which resulted in elimination of the resistant bacteria completely, thus curing the infection in many cases.

The same effect was seen when we administered high dose Vitamin C along with anti TB drugs in the patients infected by mycobacterium tuberculosis in one of our cancer patients. The bactericidal activity of vitamin C against mycobacterium tuberculosis is dependent on high ferrous ion levels and reactive oxygen species production (ROS), and causes a multiple effects, interfering with several biological processes leading to bactericidal and/or bacteriostatic effect (FIGS. 3, 4, 10). The studies Vilcheze et al., lead us to believe that when combined with other anti-mycobacterium tuberculosis therapeutic agents, the disease was curtailed and cured quickly, and will not take 6 to 24 months. This method of therapy was used to treat the latent mycobacterium tuberculosis infection. Further it also eliminates the possibility of developing tuberculosis from hidden Mycobacterium tuberculosis bacteria in the body macrophages and other cells, lodged in the lungs or other parts of the body away from the lungs, and thus prevent the activation and develop full blown TB.

Similarly, in Gram-positive and Gram-negative bacteria, a familiar mechanism of killing by bactericidal drugs, involves the generation of hydroxyl radicals by the somewhat controversial Fenton reaction or by protein mistranslation and stress-response to Hydroxyl radicals induce cell death via DNA damage, which is due in part to the oxidation of the guanine nucleotide pool (FIG. 3, 4). It is thought that the Hydroxyl radicals are produced by the combination of the Haber-Weiss cycle and the Fenton reaction. In these reactions, ferrous ion reacts with oxygen to produce superoxide (#1), which by dismutation leads to hydrogen peroxide formation (#2). Hydrogen peroxide then reacts with ferrous ions to form hydroxyl radicals via the Fenton reaction (3) (Fenton H. J. H. (1894). “Oxidation of tartaric acid in presence of iron”. J. Chem. Soc., Trans. 65 (65): 899-911). Fenton reaction is explained as follows (FIG. 3):

Fe2++O2→Fe3++O2.−

2O2.−+2H+→H₂O₂+O2

H₂O₂+Fe2+→Fe3++OH.+OH−

The Haber-Weiss reaction generates .OH (hydroxyl radicals) from H2O2 (hydrogen peroxide) and superoxide (.O2−). This reaction can occur in cells and is therefore a possible source for oxidative stress on the mycobacterium tuberculosis bacteria when Vitamin C is administered. The reaction is named after Fritz Haber and his student Joseph Joshua Weiss (Weiss J. (1932). “Über die Katalyse des Hydroperoxydes (On the catalysis of hydroperoxide)”. Naturwissenschaften 20 (51): 948-950; Koppenol, W. H. (2001). “The Haber-Weiss cycle—70 years later”. Redox Report 6 (4): 229-234). The reaction is very slow, but is catalyzed by iron. The first step of the catalytic cycle involves:

Reduction of ferric ion to ferrous:

Fe3++.O2−→Fe2++O2

The second step is the Fenton reaction:

Fe2++H₂O₂→Fe3++OH−+.OH

Net reaction:

.O2−+H₂O₂→.OH+OH−+O2

In the presence of above reductants, ferrous ions are produced by reduction of ferric ions (#2). One reductant known to drive the Fenton reaction is Vitamin C, also known as ascorbic acid. As a pro-oxidant, vitamin C can drive the Fenton reaction by reducing ferric ions to ferrous ions as shown in the above formula and FIG. 3. Most iron within the cell is bound within porphyrins and proteins, as ferric ions that are sparingly soluble and ferrous ions are very reactive as well as more soluble. To examine the role of iron concentration in vitamin C bactericidal activity, the concentrations of bound iron and reduced and oxidized free iron were measured in tuberculosis cultures treated with vitamin C. In vitamin C-treated samples, intracellular free iron levels were increased by 50 to 75% compared to the untreated cultures after three days while extracellular free iron concentrations were nearly four-fold higher than that of the untreated cultures according Jacob and his group of investigators (Vilcheze, C., T. Hartman, B. Weinrick, and WR. Jacobs, Jr. 2013). This increase in free iron levels was also the dose which peaked at day three. Experiments showed that the ferric iron oxidation was responsible for the tuberculosis bacteriostatic and bactericidal effect of Vitamin C. We treated a 45 year old female has traveled to Southeast Asia and was diagnosed with left breast cancer. It was an early case with discrete lymph node metastasis. She has cough with blood tinged sputum, night sweats, and weight loss. Her chest X ray showed right upper lung mass and her sputum came back as positive for mycobacterium tuberculosis bacilli. By the time we made the TB diagnosis she was treated with high dose Vitamin C 10 to 25 grams in 500 ml normal saline every day for a total of 10 doses as an adjuvant therapy for cancer. She underwent local injection of anti-breast cancer chemotherapy therapeutic agents with insulin and local heat application with magnetic heat waves and hyperbaric therapy using soft air HBO chamber. She was completely cured of tuberculosis and continues to be negative during the follow up.

The ability of vitamin C to reduce ferric ions to ferrous ions results in the generation of ROS (superoxide, hydrogen peroxide, and hydroxyl radicals-see FIG. 3) via the Harber-Weiss and Fenton reactions, which results in DNA damage. As described in this invention, insulin will increase the entry of Vitamin C into the mycobacterium tuberculosis, enhance the Fenton reaction and increases the production of ROS to kill the organism (FIG. 3). It also enhances and augments the therapeutic effectiveness of anti TB drugs inside the bacteria (Alabastor et. al. 1981). Analysis of transcription patterns revealed that the vitamin C exposed cells accumulated ferrous ions, which is consistent with the known bactericidal mechanism of vitamin C (Vilcheze et. al.). The vitamin C reduces ferric to ferrous iron, and the ferrous ions react with oxygen to produce hydroxyl radicals, which are a type of reactive oxygen species. The hydroxyl radicals damage guanine residues in DNA, causing cell death.

The data indicate that combinations of INH/Cyst (cysteine) and RIF/Cyst treatments lead to ROS production and DNA damage (WO 2014/176498. PCT/US2014/035448), so also Vitamin C. This might occur via the Harber-Weiss/Fenton reactions as described in FIG. 3, which generate ROS by reducing cupric or ferric ion to produce cuprous or ferrous ion. Ferrous ion (or cuprous ion) can then react with oxygen to form superoxide, which by dismutation will produce hydrogen peroxide. Hydrogen peroxide will next reduce ferrous ion (or cuprous ion) and form hydroxyl radical and ferric (or cupric) ion. The intracellular levels of ferrous ions were measured in tuberculosis treated with cysteine, INH, RIF, Vitamin C for 3 days and found to be up to ten times higher than in the untreated sample with killing of M. tuberculosis bacteria. The DFO (deferoxamine) treated cultures could reverse the killing of tuberculosis bacteria by a vitamin C-induced Fenton reaction (Vilcheze et al., 2013. WO 2014/176498). Thus Vitamin C and many of the anti-tuberculosis drugs act by producing ROS through Fenton reaction as shown in FIG. 3.

Vitamin C Produces ROS in M. tuberculosis:

The ability of Vitamin C to reduce ferric ions to ferrous ions is well known and is the basis of its pro-oxidant property. The production of ferrous ions leads to the generation of ROS (superoxide, hydrogen peroxide and hydroxyl radicals) via the Barber-Weiss and Fenton reactions, which can result in DNA damage. The production of ROS by Vitamin C was tested using flow cytometry. Up to threefold increase in total ROS was observed in M. tuberculosis treated with Vitamin C. The studies also showed ROS induced DNA damage it bacteria increased over time to reach 17-25% after 9 days, tenfold higher than in the untreated culture or in the culture treated with Vitamin E. That is why we want to continue high doses Vitamin C therapy for 4-6 weeks to cause maximum damage to the M. tuberculosis bacteria.

Monitor the TB patients with digital pulse Oxymeter to measure the arterial oxygen saturation of the blood. We subjected the TB patients to accelerated ROS production by Vitamin C (FIG. 3) by addition of supplemental oxygen using one of these methods described below:

Administer supplemental oxygen through the nasal canula or mask,

100% oxygen hyperbaric therapy (HBO) in a special chamber during the last hour of administration of Vitamin C to provide ferrous ion more tissue oxygen to produce more ROS including hydroxyl radicals to kill the mycobacterium tuberculosis,

Or place the patient in soft less expensive, atmospheric HBO therapy chamber which is easy to use, and transport, which will increase the oxygen level in the blood at 1.7 to 2.00 air atmospheric pressure and enhance the killing effect of Vitamin C and other therapeutic agents.

It is important to note in another study, researchers (Vilcheze, C., T. Hartman, B. Weinrick W. R. Jacobs, Jr., (2013) found that vitamin C killed TB bacteria growing in laboratory dishes, including the strains that are resistant to available anti-tuberculosis drug. That study was published May 21 in the journal Nature Communications (see the references). We recorded a case of complete cure of TB with high dose Vitamin C when treating cancer. Now we know why! This tells us that the resistant Mycobacterium tuberculosis has no place to hide and pester the infected individuals as latent and active tuberculosis year after year. It also shows that our method of treatment of MRSA and other resistant infection decades back had a scientific basis. The study in the laboratory on the tuberculosis culture of tuberculosis showed that the Vitamin C completely killed the bacteria (Vilcheze et. al.) once again substantiates our clinical finding in hum pulmonary tuberculosis.

Methicillin Resistant Staphylococcus Aureus (MSRA) Infection Treatment with High Dose Vitamin C and Antibiotics with Insulin

We treated a number of cases of MRSA (Methicillin resistant staphylococcus aureus—also named as superbug) bacterial infection that is hospital-acquired (HA-MRSA) or community-acquired (CA-MRSA) resistant to beta-lactam antibiotics treatment with broad spectrum antibiotics, and Vitamin C with insulin induced hypoglycemia and hyperbaric therapy with complete elimination of the infection. MRSA is contagious and can cause life-threatening infection. It spreads by coming in contact with an infected person or by exposure to an MRSA-contaminated object or surface that an infected person touches. About one in four healthy people are colonized by staphylococcus bacteria. Those who are colonized have the bacteria present in their skin and nasal passages, but the presence of the bacteria doesn't make the person ill. Most staph bacteria are sensitive to beta-lactam antibiotics, such as penicillin, methicillin, and ampicillin. Some strains of staph developed resistance to beta-lactam antibiotics. It is estimated that 2 percent of the population now carry a strain of staph that is resistant to beta-lactam antibiotics known as the super bug. Most often, it causes mild infections on the skin, like sores or boils. It can also cause more serious skin infections or infect surgical wounds, the bloodstream, the lungs, or the urinary tract resulting in untold morbidity and mortality. We successfully treated them with high dose (5-15-15-25 grams Vitamin C IV infusion, depending upon the severity and patient condition) with hyperbaric therapy (optional), and high doses of broad spectrum antibiotics after dropping blood sugar to ±40 mg % using fast acting insulin (IHAT) then reversing hypoglycemia by administering glucose within an hour or two at the end of the treatment if needed based on finger stick blood glucose level.

Physiological Functions of Vitamin C in the Body and its Implications in TB Treatment:

Vitamin C (also known as L-ascorbic acid or as sodium L-ascorbate) was isolated by Szent-Györgyi in 1928 and is easily synthesized. In our body physiology, it plays a key role in several biological functions including the biosynthesis of collagen. To maintain health, vitamin C, is required and essential for the biosynthesis of collagen, L-carnitine, as well as certain neurotransmitters. Collagen is an essential component of connective tissue, which plays a vital role in wounds healing and its deficiency leads to scurvy. Vitamin C also plays a role in immune function and improves the absorption of nonheme iron, the form of iron present in plant-based foods important to form red blood cells (RBC).

Vitamin C inhibits the release of creatine kinase, an indicator of muscle damage, compared with the placebo group. Thus Vitamin C can help to reduce the muscle loss and emaciation in TB patients. Studies also showed smokers who received the vitamin C showed 24% reduction in their plasma C reactive protein (CRP) concentrations, while neither of the other groups showed a significant change indicating that Vitamin C can play a role in reducing the CRP which affects the cardiovascular health of TB affected patients. Dutch researchers showed that 2,000 mg/day of vitamin C for two weeks reversed endothelial dysfunction caused by the abnormal migration of monocytes implicated in atherosclerosis (Stadler N, et al. 2007).

Besides Vitamin C's role in production of ROS in high doses (FIGS. 3, 10), it also prevents adhesion of monocytes, reduction of cytokines, and C reactive proteins, thus lowering the inflammatory processes at the site of TB bacteria. Thus it exposes the microbe for the action of anti TB drugs. Adding ibuprofen and aspirin for the treatment of tuberculosis augment the above effects of Vitamin C.

Above and beyond the above explanation of ROS production by Vitamin C (like a free thiol—a cysteine, dithiothreitol, penicillamine), and their bactericidal effect, it enhances respiration in the M. tuberculosis bacteria and thus enhances the uptake of anti-TB drugs which becomes effective in killing them in short duration. These aerobic respiration enhancer, inhibits expression of M. tuberculosis succinate dehydrogenase I or inhibits activity of M. tuberculosis succinate dehydrogenase I, thus enhance the killing of this resistant bacteria. Induction of moderate hypoglycemia as described in this invention will enhance M. tuberculosis bacterial respiration, increases the uptake of therapeutic agents, further augmenting the killing effect of all therapeutic agents and Vitamin C. Adding DNP described in this invention with Vitamin C will add further improvement in bactericidal effect of anti-TB drugs by increasing the M. tuberculosis bacterial respiration. The anti-tuberculosis medication comprises one or more of isoniazid, rifampicin, pyrazinamide, and ethambutol. In an embodiment of the composition, the enhancer of aerobic respiration inhibits expression of M. tuberculosis succinate dehydrogenase I or inhibits its activity of tuberculosis bacterial succinate dehydrogenase I (US2014/035448).

Humans and higher primates, as well as guinea pigs and small numbers of other animal species such as bats, carry a mutated and ineffective form of the enzyme L-gulonolactone oxidase, the fourth and last step in the ascorbate-producing machinery. This mutation likely occurred millions of years ago (in the anthropoid's lineage). The three surviving enzymes continue to produce the precursors to vitamin C, but the process is incomplete and the body then disassembles them. That is why human and many primates need to have supplement Vitamin C to counter the deficiency by oral supplemental intake of Vitamin C. Vitamin C helps to heal the injury caused by the tuberculosis lesion, facilitates to builds a capsule around the TB lesion by increasing the collagen output by fibroblasts (localize the lesion), and activates the phagocytosis of the dead or dying t bacteria and host structures at the site of the lesions by white blood cells and macrophages, and their destruction by lysosomal enzymes within the white blood cells.

What is the Dose of Vitamin C Needed to Treat Tuberculosis, Mode of Action, and its Route of Administration?

Our studies treating MSRA, and other infections with Vitamin C support that the mycobacterium tuberculosis is more amenable to Vitamin C and eliminated combining with other appropriate anti TB therapeutic agents as described in this invention. Studies suggest that high dose vitamin C given by mouth is poorly absorbed, hence less effective in treating TB. Blood levels “max out” at doses of 500 mg given several times during the day. But vitamin C given intravenously, delivered in a “drip,” achieves much higher concentrations of C in all tissues especially in lungs which has rich blood supply-literally the lungs are bathed in the blood media. At these higher concentrations, vitamin C bathes the lungs which lodges the infection. Hence giving high dose IV have different characteristics than if given orally. While oral vitamin C boosts immunity and assists tissue repair, it is too weak to kill or inhibit mycobacterium tuberculosis and other infecting agents. But at high doses delivered directly into the bloodstream, it acts to increase levels of hydrogen peroxide and ROS deep in the tissues where infected cells and mycobacterium tuberculosis lurk and kill them.

Laboratory research currently under way has shown that high effective amount, or effective concentrations of vitamin C kill the mycobacterium tuberculosis (Vilcheze, C., T. Hartman, B. Weinrick & W. R. Jacobs, Jr., 2013). Only intravenous administration of vitamin C can deliver the high doses to the site of mycobacterium tuberculosis lesion site in the alveoli of the lungs which has rich 600 miles of capillary network. Intravenous (IV) vitamin C, when administered by a trained, experienced physician, is safe and well-tolerated, even at doses from 5 to 50 grams or higher per day. In our extensive use of high dose Vitamin C, we never experienced any complications or untoward effects and none of the patients developed renal stones either—as reported complications of high dose Vitamin C. Vitamin C is one of the most important adjuvant therapeutic agents as described in this invention in the treatment of TB and other microorganisms including those which develop resistance to therapeutic agents. When Vitamin C is administered intravenously (IV), the first site it reaches is lungs before it spreads through systemic circulation, making it even more effective in the treatment of pulmonary tuberculosis.

At the normal low physiological concentrations (0.1 mM), vitamin C is an anti-oxidant that inactivates reactive oxygen species. However, at high pharmacologic concentrations (up to 20 mM) it was found to become a pro-oxidant generating reactive oxidative species (ROS), such as extracellular hydrogen peroxide, which is lethal to M. tuberculosis bacteria, viruses, and cancer cells. That is why we use high intravenous doses of Vitamin C for the killing the M. tuberculosis bacteria. Mitochondria are the major source of ROS, and specifically mitochondria-generated ROS, are involved in physiological signaling cascades regulating various cellular and organ functions, with H2O2 being a chief messenger molecule. Reactive oxygen species (ROS) are chemically reactive molecules containing oxygen. Examples include peroxides, superoxide (H2O2), hydroxyl radical (.OH and HOO.), and singlet oxygen (.O2). Peroxide is a compound containing an oxygen—oxygen single bond or the peroxide anion, O2— and simplest stable peroxide is hydrogen peroxide, superoxides, dioxygenyls, ozones and ozonides. ROS can punch a hole in the mitochondria leading to leaking of cytochrome C and death of the organism. The ROS are produced in normal cell metabolism also. It is known that the cochlear damage resulting in hearing loss resulting from anti-TB drugs is due to ROS production by these therapeutic agents. The ROS thus produced also causes lipid peroxidation that refers to the oxidative degradation of lipids. It is the process in which free radicals “steal” electrons from the lipids in cell membranes, resulting in cell damage. The ROS produced in the cells after injury play a role in leukocyte and platelet recruitment which is also important to fight the TB infection.

Superoxide dismutases (SOD) are a class of enzymes that catalyze the dismutation of superoxide into oxygen and hydrogen peroxide which is converted to water. As such, they are an important antioxidant defense in nearly all cells exposed to oxygen including M. tuberculosis bacteria. SODs are found in the cytoplasm (SOD1), mitochondria (SOD2), and extracellular (SOD3).

In our study, to treat TB, we want to use between 10-15-25 grams or more (depending on the severity of TB disease and condition of the patient) of Vitamin C by IV infusion given over the period of 3-4 hours multiple times a week. Infusion up to 50 grams are used in some cancer patients (by integrated medicine practicing physicians) with peak plasma concentration of 13.4 mM; while maximum tolerated dose of 3 g every 4 hours taken orally resulted in peak plasma concentrations of only 0.22 mM (Padayatty S. J, et. al. 2010). In phase I dose finding studies in cancer patients, the investigators used 1.5 g to 2 g intravenous vitamin C per kg body weight three to four times per week (Hoffer L J, et. al. 2008, Stephenson C M, et. al. 2013). In further clinical research, it is advised to start treatment with a lower dose (5 Grams IV), and, if no adverse events are observed, to gradually increase doses to their final level of 25 Grams or more. We want to follow the same guide lines in the treatment of tuberculosis. We use high dose Vitamin C intravenous infusion to achieve the desired bactericidal plasma concentration of Vitamin C, for achieving pharmacologic concentrations needed for killing the M. tuberculosis bacteria.

All studies in our clinic indicate that giving Vitamin C IV drip will achieve higher levels of Vitamin C in the blood compared to oral routes, to kill the mycobacterium tuberculosis bacteria and it is a safe method. Hydrogen Peroxide-mediated killing is one of the white blood cells' key mechanisms for fighting infection and so also the killing of mycobacterium tuberculosis by vitamin C which produces hydrogen peroxide and other ROS (FIG. 3) in high doses by Fenton reaction within the bacterial cell which are fatal to the tuberculosis bacteria.

Blood Levels of Iron to Produce ROS by Vitamin C to be Effective (FIG. 3):

Our body needs iron to build healthy red blood cells and hemoglobin, a blood protein that carries oxygen to every cell in our body. We know that Vitamin C kills the mycobacterium tuberculosis by Fenton reaction by reacting with cellular ferric ions, so also many antibiotics and therapeutic agents. For Fenton reaction to take place to kill mycobacterium tuberculosis, ferric ions are needed inside the bacteria, phagocytosed cells, and in the tissues surrounding the microbe. Iron is an important component in red blood cells that move oxygen to your muscles and organs. Do we need to be concerned about body irons levels to increase the mycobacterium tuberculosis bacterial levels of iron to make the Vitamin C therapy more effective? We do not have the answer for this. More experimental studies and lab tests are needed to expose the mycobacterium tuberculosis to ferritin from the surrounding media in which they are bathed, and then treat with Vitamin C and compare bactericidal effect. Until then we need to use common sense by maintaining the proper iron levels in the form of ferric ions. We do put patient on diet rich in iron and if needed supplemented with orally or parenteral iron administration as well as advise them to iron rich vegetarian diet such as: raisin bran (enriched), instant oatmeal, beans (kidney, lima, navy), tofu, lentils, molasses, spinach, whole wheat bread, peanut butter, brown rice, collards and non-vegetarian sources (heme iron) such as chicken liver, oysters, clams, beef liver, beef, turkey leg, tuna, eggs, shrimp, lamb. Further our studies show that the potency of the Vitamin C and other therapeutic agents to treat tuberculosis and other diseases is enhanced by insulin and insulin induced hypoglycemia which is labeled as insulin Potentiation therapy (IPT).

Precautions and Safeguards: During Treatment of Tuberculosis Using Our Invention:

G6PD, an enzyme in red blood cells needed to maintain red blood cell membrane integrity. High dose intravenous vitamin C is a strong pro-oxidant and giving a pro-oxidant to a G6PD-deficient patient can cause hemolysis of the red blood cells due to break down of RBC membrane by ROS. Those using high dose IV Vitamin C need to be cognizant of this adverse effect when treating tuberculosis, cancers and other afflictions. If hemolysis develops during the treatment, laboratory testing for G6PD is in order.

Renal insufficiency and dialysis add to the difficulty in the management of TB because anti-tuberculosis medications are cleared by the kidneys. Some of the anti-tuberculosis agents are removed via hemodialysis making the treatment less effective. Hence with kidney disease afflicted patients with a creatinine clearance of <30 ml/minute or those who are on renal dialysis, the alterations in dosing and frequency of anti TB drugs is adjusted. Patients on hemodialysis, medications should be given 3 times per week after dialysis.

Persons with chronic gastrointestinal disease resulting in malabsorption that encompasses defects occurring during the digestion and absorption of food by the gastrointestinal tract (e.g., Cohn's disease, various malabsorption syndromes, chronic diarrhea, steatorrhoea, regional ileitis, HIV-related diarrhea, Whipple's disease, intestinal TB, tropical sprue, enteropathy, parasitic infestation, radiation enteritis, collagen vascular diseases, short bowel syndrome, surgical structure changes, carcinoid syndrome, Addison's and other endocrine diseases including Zollinger-Ellison syndrome and other pancreatic insufficiencies, primary bile acid diarrhea, etc.) are at risk for drug treatment failure. In such cases serum drug levels are obtained to document the adequacy of medication delivery with malabsorption or those who fail to respond to TB treatment.

Anti-TNF alpha drugs (tumor necrosing factor alpha antagonists) are a new class of immunosuppressive drugs utilized for treatment of inflammatory conditions widely to treat a wide array of autoimmune diseases. The anti-TNF alpha drugs are associated with increased risk of TB disease and other infections including developing cancers (Keane J. et al. 2001). These agents include: infliximab (Remicade®), Etanercept (Enbrel®), and adalimumab (Humira®) and many under creation and production to treat wide array of diseases. Whenever clinically feasible, people with a history of a positive tuberculin skin test (TST>5 mm) should start treatment for latent tuberculosis also known as dormant, inactive mycobacterium tuberculosis infection before commencing TNF-α blocking agents. The preferred regimen is 9 months of isoniazid in such cases as prophylactic. Our invention cuts down this long protracted treatment. Consider postponing TNF-α antagonist therapy until the conclusion of treatment for Latent mycobacterium tuberculosis infection (LTBI) or in treating active TB patients. In future studies, we will evaluate the mode of applying our invention on a case by case basis. So far we have not encountered such cases in our study.

Hepatic Toxicity of the anti-mycobacterium tuberculosis agents is a concern. The liver injury can be caused by three of the first-line TB disease drugs, INH, RIF and PZA. Significant liver toxicity is indicated by liver enzyme changes such as AST≧3 to ≧5 times normal. If the AST and ALT are <5 times the upper limit of normal, toxicity can be considered mild; an AST or ALT of 5-10 times normal defines moderate toxicity; and >10 times normal indicates severe hepatic toxicity by the administered drugs. Bilirubin and alkaline phosphatase elevation also indicate hepatic toxicity. Hence, the dose and type of drug given is adjusted or changed if the patient develops hepatic toxicity indicated by liver functions tests.

Recurrence of fever in a patient who has been receiving therapy for several weeks suggest drug related fever, especially if the patient is showing improvement. One needs to know that the fever from TB may persist for as long as 2 months after therapy has been initiated due to any number of known and unknown etiologies.

Delivery of Vitamin C Directly to the Site of Tuberculosis Lesion to Treat Pulmonary TB by Inhalation Nebulizers and or Special Delivery Device Described Herein (FIGS. 5, 6, 7):

Studies show that introduction of Vitamin C in to M. tuberculosis bacteria culture in vitro results in their death. That being the case, we deliver Vitamin C locally by injection and/or inhalation by aerosol as described in this invention. A nebulizer is a machine that turns liquid medicine into a mist so that it can be inhaled with ease delivered deep into the lung tissue according to the American Thoracic Society. A nebulizer may be used instead of a spray bottle or nasal inhaler. There are many kinds of nebulizer devices in the market. Ultrasonic nebulizers' nebulise liquids very fast and very quietly using ultrasonic waves in the conversion of liquid medicine into a vapors-like fine mist called ultrasound atomization. They deliver very fine vapors/mist for effective absorption deep in the respiratory system-bronchioles and alveoli. Countless therapeutic agents both FDA approved and approved natural therapeutic agents have been administered by this method. Examples are: bronchodilator, antibiotics, silver nitrate, magnesium, hydrogen peroxide, vitamin B12, glutathione, new therapeutic agents are added nonstop and the list is endless. Any therapeutic agents used in the nebulizer should be nonirritating to the air passages and alveoli to prevent the irritation and its related cough reflex. It is important to note a portable ultrasonic nebulizer is effective, versatile; and can be used at home and during travel; for adults and children with some modification of delivery tubing.

We want to introduce the Vitamin C delivered by nebulizer for the treatment of pulmonary tuberculosis especially those cases whose sputum is positive for mycobacterium tuberculosis bacteria and those with miliary pulmonary tuberculosis to prevent the droplet spread of tuberculosis due to coughing. We have used it with Vitamin C and antibiotics-chemotherapeutic agents in the nebulizers to treat various pulmonary diseases with good results. We intend to use this method in the treatment of pulmonary tuberculosis along with our invention. To deliver Vitamin C through a nebulizer, the patient is made to stay recumbent position on a recliners or bed. If one is using a mask, position to deliver the mist, place it comfortably and securely on the face around the mouth. If a mouthpiece is used, place it between the teeth and seal the lips around it. Take slow, deep breaths through the mouth. Hold each breath for 5-10-15 seconds before breathing out. This allows the therapeutic agents droplets to settle into the airways and alveoli. Breathing from the diaphragm allows the mist with therapeutic agents fill and empty the lungs with the mist. Vitamin C and Various anti TB therapeutic agents can be delivered through the nebulizer to treat pulmonary TB and other afflictions of the respiratory system.

Cavitary TB (FIG. 5) with positive cultures at 2 months poses special problems in the treatment of the disease. It is known that greater than the normal or average relapse occur in patients who present with TB cavitation's on chest radiograph and whose sputum cultures can remain positive after 2 months of treatment. Sputum culture positive at two months, cavitation's on chest radiography, being underweight, and bilateral pulmonary involvement increases the risk of treatment failure and/or relapse (Tuberculosis Trials Consortium (2002) Treatment of drug-susceptible tuberculosis with a once weekly regimen of isoniazid and rifapentene in the continuation phase. Lancet 360: 528-534). We want to deliver to the TB cavity or lesion Vitamin C and anti TB drugs through our device positioned besides delivering by aerosol method (FIGS. 5, 6).

Dawson et al (2007) study using nebulized recombinant interferon-Y⁻1b to DOTS in a randomized, controlled clinical trial resulted in reduced bronchoalveolar lavage (BAL) cytokines, more rapid clearance of M. tuberculosis bacteria from the sputum, improved symptoms, and reduced inflammatory macrophage-neutrophil alveolitis. These findings suggest that nebulized recombinant interferon-Y⁻1b may have a role in adjunctive immune stimulation in patients with cavitary tuberculosis. We believe that these TB cavities in the lungs can be sterilized by direct infusion of Vitamin C, streptomycin, rifampicin, interferon Y⁻1 b, and other anti TB drugs as described above shown in the FIGS. 5,6,7 using our infusion device and nebulization. The same method of continuous infusion is adopted to treat cold abscess also after drainage, irrigation with Vitamin C, streptomycin, rifampicin and other anti TB drugs through an indwelling catheter.

Insertion of a continuous therapeutic agents delivery catheter (FIGS. 5,6,7) of our invention through trachea-bronchial tree directly through the cricothyroid membrane or subcricoid (Shantha T R: Retrograde intubation's using the sub-cricoid region, British Journal of Anesthesia 68:109-112 (1992) method positioned inside the tuberculosis cavitary and in the center of the tuberculosis lesion (FIGS. 5, 6 #42, 48) and direct delivery of Vitamin C and other anti-tuberculosis therapeutic agents to sterilize the cavitary by liquid injections or aerosol methods can contribute to prevention of spread of the disease if the sputum is positive for M. tuberculosis bacteria and assure early cure and prevent further destruction of the lungs.

Inhibition or Prevention of M. tuberculosis Bacterial Multiplication (Division) and Overcome the MDR with Artificially Induced Electrical Field Around the Chest Wall and Boney Lesions:

There are report the application of the cancer treatment modality using low-intensity, intermediate-frequency, alternating electric fields (Tumor-Treating Fields, TTF) for treatment of malignancies in human patients (Salzberg et. al. 2008). This feasibility study to treat tuberculosis has the goal to demonstrate the safety and absence of adverse effects of this simple method in patients. Weak electric AC currents generated employing conductive electrodes are known to increase the efficacy of antibiotics against bacterial biofilms, a phenomenon termed “the bioelectric effect.” We have used the battery operated pads, inserted electrodes, ultrasound and magnetic fields for the treatment of breast and lung cancers, infections, over two decades. We have used ultrasound waves to treat severe prostate hypertrophy and to enhance the immune system. Light sources of various types are being used for photodynamic therapy in dentistry, dermatology and treatment of cancers. We have used hypertheramia (Thermotherapy) to treat cancers, infections and many other diseases' in our practice. The sound waves and electrical fields are known to kill bacteria, and multiplying cells so the effect of using this method to treat tuberculosis (Carmen J C. Et. al. 2004) will be an added simple modality.

The effect of alternating electric field disrupts the cancer cell replication has been described before, so also its enhancing chemotherapy effects (Kirson et. al. 2004. 2007. Berg et. al. 1987. Janigro et. al. 2006). It has been used effectively to treat the neuropathic pain (Odell, Sorgnard 2008, 2011 Odell et. al. 2015). In our own treatment of cancers, we have used this and magnetic induction therapy combined with low dose chemotherapy with good results (Shantha-2001-2004 unpublished data). The proposed main biological basis of this therapy in treating tuberculosis is a added modality with therapeutic effect of the alternating electrical fields on the formation of the mitotic spindle in the multiplying M. tuberculosis bacteria (FIG. 12). A secondary effect is rupture of the membrane during mitosis, as observed in cultured cells and reported earlier and allowing the anti TB drugs enter the bacteria and kill it. Insulin will augment the mitosis and enhance the AC electrical field effect on the M. tuberculosis bacteria.

The energy of electrical fields created by this method (FIG. 11, 12) acts as electrical signaling of the cell wall and produces a hormone-like effect by triggering an electrical field induced change to the cell membrane G protein. This change influences the activity of adenylate cyclase, resulting in the formation of cAMP. cAMP induced repair processes are necessary to stabilize the cell membrane and inhibit continued leakage of acids known to trigger pain and inflammatory mediators. By promoting changes to G proteins in cell membranes, this method of treatment ultimately normalize cell function and neuropathic pathology (Odell et. al. 2015). Vasodilations promotes nutrient transfer to the area of inflammation especially in nerve tissue, and “wash out” of waste products (the products of inflammation) with pH normalization; the reduction in inflammation produces a concomitant reduction in edema and pain. Using the vinpocetine will augment this electromagnetic effect on vasodilation. But the application of AC fields for many days will prevent the healing and reformation of tuberculosis bacteria interfering with its division resulting in its destructions.

Organisms from microbes to elephants proliferate by cell division, a basic mechanism of survival, reproduction and propagation. The microorganisms such as bacteria, mycoplasma, yeast, protozoa, and other single-celled organisms, fungi, algae, plant cells, divide to reproduce, spread and survive. The process of eukaryotic cell division is called “mitosis” with or without chromosome separation (meiosis). TB bacteria also divide by chromosome separation but not like classic mitosis. The electric current and electrical fields have been used to simulate, destroy, propagate, and record (ECG, EEG, EMG, ERG,) and are used to cure and diagnose diseases. Use of TENS and acupuncture to treat pain is widely known and applied therapy even now. Chemotherapy, antibiotic, radiation, radiofrequency, electrical cautery have been used to kill the dividing and non-dividing cells. It has been shown that the steady field or fields that change at relatively slow rates, and alternating fields of low frequencies that induce corresponding electric currents in the tissues, and/or high frequency alternating fields above 1 MHz between 5 and 20 MHz or more applied to the body surface by positioning biocompatible field electrodes on either side of chest cavity by means of the conducting electrodes have killing effect on the dividing cells including M. tuberculosis bacteria and cancer cells. The electrical field created is strong enough to damage and kill the multiplying TB bacteria but not the area of skin contact, unless used for long periods of time.

Slow frequency nerve stimulation of 1-4 Hz may produce vasoconstriction. On the other hand the faster frequency greater than 10 Hz produce vasodilation. Hence we use this frequency or higher. It is known that the cells that are in the process division are vulnerable to damage by AC electric fields that have specific electrical frequency and fields. This selective destruction of rapidly dividing M. tuberculosis bacteria (cancer cells including) accomplished by intruding AC electric field in a target region for protracted periods of time. The multiplying TB bacteria in the applied field are damaged, sparing the lung parenchyma cells that do not divide rapidly. The vulnerability of the dividing cells is powerfully related to the alignment between the long axis of the dividing cells and the lines of force of the electric fields on the mitotic spindles, improved results can be obtained when the field is consecutively intruded in different directions. It is important to note here that the Insulin and vasodilatation we use will augment the cell division, thus have the bactericidal and/or bacteriostatic effect by inhibition of TB bacterial growth.

We want to use the high frequency alternating AC electrical fields around the chest wall (FIG. 11) during the Vitamin C and high dose anti TB drugs therapy (for example, high doses of rifampicin) to augment and expand the killing of the multiplying TB bacteria. The alternating current created electrical field due to high frequency alternating electrical current applied to the chest wall (FIGS. 11, 12) passing from front to back and side to side applied to the body by means of insulated electrodes acts by blocking the mitotic mechanism and changing the pH (changing from acid to neutral or alkaline pH) within the TB bacteria and macrophages, to curtail and cure tuberculosis. This method is safe and has no adverse effects. The treatment is applied by electrodes attached onto the shaved skin of the patient (FIG. 11) chest, and powered by a mobile generator that is carried by the patient over a time span for one week or more to be used 24 hours a day.

Artemisinin as Anti M. tuberculosis Bacteria Adjuvant Therapeutic Agents:

Artemisinin comes from Chinese Sweet Wormwood plant (Artemisia annua) or synthesized in the laboratory. Chemically, artemisinin is a sesquiterpene lactone containing an unusual peroxide bridge. This peroxide is believed to be responsible for the drug's mechanism of action. We administer oral Artemisinin to react with M. tuberculosis bacteria and macrophage containing the TB bacteria with intracellular iron to produce ROS to kill the infecting agent. We know that the Vitamin C and many anti-TB drugs kills the M. tuberculosis bacteria by producing ROS radical as described above. Artemisinin contains an endoperoxide moiety that can react with iron to form cytotoxic ROS free radicals. It is taken up by the digestive vacuoles (Phagosome) or forms its own vacuole by fusion of the cell membrane around Artemisinin in the cell and exerts their action from that site. We know that the TB bacteria also enter the macrophages in this manner with formation of phagosome.

Artemisinin has an endoperoxide bridge that is activated by intra-parasitic heme-iron to form free radicals, which kill malaria parasites by alkylating biomolecules so also in TB bacteria. In recent years, there are many reports of anticancer activities of artemisinins both in vitro and in vivo. Artemisinins also have inhibitory effects on cancer cell growth, including many drug- and radiation-resistant cancer cell lines. The cytotoxic effect of artemisinin is specific to cancer cells because most cancer cells express a high concentration of transferrin receptors on cell surface and have higher iron ion influx than normal cells via transferrin mechanism. In addition, some artemisinin analogs have been shown to have anti-angiogenesis activity. Artemisinin tagged to transferrin via carbohydrate chain has also been shown to have high potency and specificity against cancer cells (Lai H. et. al. 2005. Nakase I. et. al. 2008. Deng X R et. al. 2013). Since iron influx is high in cancer cells and TB bacteria, artemisinin and its analogs selectively kill M. tuberculosis bacteria with increased intracellular iron concentrations as it does cancer cells. Iron is required for its division, and it is well known that many cancer cell and also TB bacteria we believe selectively accumulate iron for this purpose. Most M. tuberculosis bacteria have large number of iron attracting transferring receptors on their cell surface. In laboratory studies of radiation resistant breast cancer cells that has high propensity for accumulating iron revealed that artemisinin has 75 percent cancer cell killing properties in an 8 hours and almost 100 percent killing properties within 24 hours when these cancer cells are “pre-loaded” with iron after incubation with holotransferrin. On the other hand, the normal cells remained virtually unharmed (Efferth T, Dunstan H, et. al. 2001). The same result can be expected on the M. tuberculosis bacteria and macrophage infected cells.

Artemisinin becomes cytotoxic to TB bacteria in the presence of iron molecules. The conjugation enables targeted delivery of artemisinin into bacteria and macrophage with M. tuberculosis bacteria. Here we discuss the anti-TB activities and mechanisms of action of artemisinins and the transferrin-conjugate by producing free radicals to kill the TB bacteria. We have used artemisinin during the treatment of almost every kind of cancers as one of the adjuvant therapeutic agent for decades with positive response and hardly any ill effects. We want to administer oral Artemisinin which is available as adjuvant therapeutic agents along with other therapeutic modalities. For malaria, there is no resistance nor did toxicity at the dosage of 3 grams, (about 50 mg/kg) administer over a 3 to 5 day period and especially useful in the treatment of drug resistant malaria. There is no Artemisinin available for parenteral administration, and it is likely to be developed with lapse of time with its wide use to treat many afflictions of the body including malaria, cancers and tuberculosis to improve its delivery, therapeutic effectiveness at the same time by pass the liver metabolism and its toxic effects. Artemisinin with induced hypoglycemia and glucose administration to reverse it will increase the uptake of this adjuvant free radical producing therapeutic agent and enhances the production of free ROS radicals within the bacteria and infected macrophages to kill the M. tuberculosis bacteria.

Artemisinin and its derivatives are well known anti-malaria drugs and particularly useful for the treatment of infection of Plasmodium falciparum malaria parasites resistant to traditional anti-malarials. The mechanism of action is debated; several lines of evidence indicate that artemisinins exert their antimalarial action by radical formation that depends on their endoperoxide bridge. When the parasite that causes malaria infects a red blood cell, it consumes hemoglobin within its digestive vacuole, a process that generates oxidative stress. In the primary theory of the mechanism of action, the iron of the heme directly reduces the peroxide bond in artemisinin, generating high valent iron-oxo species and resulting in a cascade of reactions that produce reactive oxygen radicals which damage the parasite and lead to its death (Cumming J N. Et. al. 1997. Haynes R K. Et. al. 2013. Shandilya A. et. al. 2013). We believe that it has the same mechanism of action on killing TB bacteria. Currently artemisinin-based combination therapy (ACT) is recommended for the treatment of P. falciparum malaria. Fast acting artemisinin-based compounds are combined with a drug from a different class. Companion drugs include lumefantrine, mefloquine, amodiaquine, sulfadoxine/pyrimethamine, piperaquine and chlorproguanil/dapsone. Artemisinin derivatives include dihydroartemisinin, artesunate and artemether. Effect of Artemisinin on tuberculosis bacterial culture on the growth of TB bacillus needs to be investigated.

Insulin to Increase the Membrane Permeability to Allow More Anti-Tuberculosis Therapeutic Agents to Enter the Infected Phagocytes and Bacteria; Augment Effectiveness of Vitamin C and Other Anti-Tuberculosis Drugs Against M. tuberculosis Bacteria (FIG. 10):

We name this therapy as insulin induced hypoglycemia activated augmentation therapy (IHAT). It has also been called insulin Potentiation therapy (IPT), insulin-potentiated targeted low dose therapy (IPTLD). We do induce hypoglycemia not below 40 mg %. I have done IHAT in hundreds of cases over the decade without a single complication. Proper knowledge of the method and monitoring during procedure are a must if one want to avoid complications. Effects of severe hypoglycemia and how to manage such episodes should be part of the protocol.

Insulin hormone produced in beta cells of pancreas is established as having actions that affect the trans-membrane transport of different substances, particularly glucose, into numerous different kinds of cells of the human body. Insulin is a large polypeptide molecule with a molecular weight of 5808. It consists of “A” Chain (consists of 21 amino acids) and “B” Chain (30 amino acids), connected by two disulfide bonds. Apart from its membrane transport of glucose, insulin also regulates transport of some amino acids, certain fatty acids, the minerals potassium and magnesium, and specific monosaccharide's. It is well known that glucose stimulates glycogenolysis, lipogenesis, proteogenesis, and nucleic acid synthesis. It also increases glucose oxidation and magnesium-activated sodium-potassium ATPases activity. These biological effects are due to the interaction of the hormone insulin with its specific cell receptor.

The insulin receptor consists of two alpha subunits, each of molecular weight 135,000 and two beta subunits, each having a molecular weight of 95,000, which are linked together by disulfide bonds. The alpha unit is predominantly located upon the outer surface of the cell membrane, and the insulin binding/linkage domain is located there. The Trans membrane beta unit contains tyrosine kinase activity on its cytoplasmic domain that results in rapid receptor autophosphorylation, that is, effective absorption of the beta subunit into the cell. Activation of the kinase toward exogenous substrates of the cell is, it appears, preceded by this insulin-dependent autophosphorylation reaction of the beta subunit. Action on other cellular substrates leads to the expression of the full range of insulin actions at the cellular level (Schnetzler, Rubin, and Pilch. 1986).

After insulin binds to the receptor with activation of the kinase, followed by receptor autophosphorylation, the insulin-receptor combination is endocytosed (absorbed) into the cell cytoplasm. This phenomena account for the down-regulation of insulin receptor activity within the blood that ensues following insulin stimulation. With this endocytosis, a variety of events may then take place. Insulin disassociates from the receptor and, following fusion of the endocytotic vesicle with cellular lysosomes, and finally it is degraded by lysosomal enzymes (Heidenreich K A, Olefsky J M. 1985). As the insulin receptors are endocytosed from the cell membrane, with insulin binding, they leave a microscopic nano pores in the cell membrane, that facilitate the transport of various anti TB drugs, and Vitamin C which become effective in killing the tuberculosis bacteria.

The recognized action of insulin is lowering blood glucose is a processes of facilitated diffusion across cell membrane. This is accomplished via a process of facilitated diffusion across cell membranes. It has been hypothesized that the mechanism of this facilitated diffusion involves the translocation of a glucose transport protein (GLUT4) from the cytoplasm out to the cell membrane (the exterior substrate). This translocation process involves the fusion of intra-cytoplasmic vesicles with the membrane of the cell. These vesicles contain the glucose transport protein (GLUT4) in their enclosing membranes. Once exteriorized on the cell surface, the transport proteins of the vesicles serve as channels for glucose to enter the cell (FIG. 10). This particular protein has been identified as a 40,000 molecular weight moiety that is associated with the Golgi apparatus (Burdett, et. al. 1987. Keno, 1984). We believe the same channels for glucose also acts as channels for passage of many therapeutic agents against TB. The above process of translocation is reversible via endocytosis of the membrane fragment containing said glucose transport proteins of GLUT4, thus reconstituting the intra cytoplasmic vesicles (FIG. 10).

It is known that insulin receptors (FIG. 10) are widely distributed in mammalian organisms, there being in the range of 100 to 100,000 receptors per cell in different tissue. Rarely do any cells have no receptors at all (Rosen. 1987). Malignant cells have much more insulin receptors (Wong and Holdaway. 1985) because cancer cells need more glucose for energy and growth (Cone, U.S. Pat. No. 4,935,450, Myal, et. al. 1984). A number of different cancers have been found to actually secrete their own insulin (Shamas, et. al., 1981, Popovic. 1981). Investigation of many of the actions of insulin upon insulin receptors in numerous species has demonstrated that the properties of insulin receptors in mammalian U.S. Pat. No. 2,145,869, discloses a composition including insulin and glucose for the treatment of syphilis. Further, U.S. Pat. No. 4,196,196 (1980) to Tiholiz discloses a composition of insulin, glucose and magnesium, dipotassium ethyline diamine tetraacetic acid (EDTA), to enhance tissue perfusion and to facilitate a divalent-monovalent cation gradient to maintain the functional integrity of the myocardium during open heart surgery. The general value and significance, in cancer treatment, of such a cation gradient, however facilitated, is recognized in U.S. Pat. No. 4,018,649 (1977) to Cone, entitled Process and Control of Cell Division. A further U.S. Pat. No. 4,277,465 (1981) to Kamada, teaches the use of an enamine derivative molecularly linked to insulin to facilitate its therapeutic absorption across the digestive tract. The importance of insulin activity messengers is set forth in U.S. Pat. No. 4,839,466 (1989) to Saltiel. The significance of insulin in the metabolism of malignant cells is recognized and discussed in U.S. Pat. No. 4,935,450 (1990) and on viral diseases in U.S. Pat. No. 4,971,951.

Our extensive studies in the treatment of every kind of disease, including one TB patient with cancer, have shown that insulin augments and amplifies the effect of therapeutic agents many fold against every kind of cancers, infection, neurodegenerative diseases including Parkinson's disease and Alzheimer's (US patent pub. No. US 2012/0323214 A1), uveitis (inflammation of the uvea which consists of the iris, choroid and ciliary body), and other eye afflictions including retinitis pigmentosa, hair loss, every kind of autoimmune disease including scleroderma, and the list is endless. It enhances the uptake and augments the therapeutic agent's effect locally or systemically. Further our clinical studies show that the potency of the Vitamin C and other therapeutic agents to treat tuberculosis and other diseases are enhanced by insulin and insulin induced hypoglycemia which is labeled as insulin induced hypoglycemia activated therapy (IHAT). It is based on the research work of (Alabator et. al., 1981) and other studies (U.S. Pat. Nos. 2,145,869, 4,971,951, 5,155,096, Shantha, T R. in US20110052678 age related macular degeneration, US20110217260 enhancing eye lashes growth, US20110294730 for the treatment of glaucoma, US20120003296 for the treatment of dry eye syndrome, US20120101033 retinitis pigmentosa, US20120128683 for Autism treatment, US20090304776 for diabetes, US20110020279 for rabies. Shantha, T R. Alzheimer's disease treatment with multiple therapeutic agents including insulin delivered to the olfactory region through a special delivery catheter and iontophoresis. US20120323214, US20140012182, WO 2015/013252 A1 for the treatment of Alzheimers, treatment of Parkinson's disease—patent submitted).

Mechanism of action of insulin is explained simply, that the Insulin binds to the insulin receptors (a tyrosine kinase, FIG. 10) on the cell wall, embedded in the plasma membrane and this receptor is activated by insulin, IGF-I, IGF-II as well, and belongs to the large class of tyrosine kinase receptors. It completely alters the pharmacokinetics of other therapeutic agents including anti TB drugs. The main activity of activation of the insulin receptor is inducing glucose uptake besides other anti-tuberculosis agents described here. The insulin receptor is composed of two alpha subunits and two beta subunits linked by disulfide bonds (FIG. 10). The alpha chains are entirely extracellular and house insulin binding domains, while the linked beta chains penetrate through the plasma membrane. Binding of insulin to the alpha subunits causes the beta subunits to phosphorylate themselves (autophosphorylation), thus activating the catalytic activity of the receptor. The activated receptor then phosphorylates a number of intracellular proteins, which in turn alters their activity, thereby generating a biological response such as GLUT 4 vesicles to fuse with the plasma membrane, makes more permeable which allows the glucose to enter the cell (Lee and Pilch 1994). GLUT4 is the insulin-regulated glucose transporter in cells (abundant in adipose tissues and striated muscle-skeletal and cardiac, liver, now found in CNS especially in hippocampus). This action of Insulin also increases the permeability of cells to potassium, magnesium and phosphate ions, thus activates sodium-potassium ATPases in cells, causing a flux of potassium into cells. As this massive flux is taking place, the cell and bacterial membranes become more permeable to circulating chemotherapeutic agents, Vitamin C, and anti-tuberculosis drugs to enter to destroy the M. tuberculosis bacteria as well as other microorganism.

Further, Alabastor et al. (1981) and our unpublished clinical studies discovered the augmented effect of insulin on therapeutic agents in theirs experimental and our clinical studies. Their studies showed that the insulin enhances the activity of therapeutic agents such as methotrexate 10,000 times. The insulin comes in contact with the circulating therapeutic agents and enhances their therapeutic effect hundreds of times as they enter the peaceable cell membrane activating by insulin binding to a unit of the insulin rectors on the cell membrane (FIG. 10). In our decades of studies (Shantha 2004), such an effect was found when insulin was used with other therapeutic agents to argument their therapeutic activity to treat cancers, chronic infections; Lyme disease, scleroderma, lupus, Psoriasis, other autoimmune diseases; Methicillin-resistant Staphylococcus aureus (MRSA) infection, and other afflictions locally or systemically as described many patent applications as listed below. We believe that adding insulin to TB culture with Vitamin C and other anti TB drugs will decimate the bacterial culture even if they are drug resistant. The pharmacological agents augmentative effects of insulin on therapeutic agents in the treatment of every known disease other than in diabetes (type 1, 2) are described in detail by Dr. T. R. Shantha in US20120323214, US20140012182, WO 2015/013252 A1 for the treatment of Alzheimers, US20110052678 age related macular degeneration, US20110217260 enhancing eye lashes growth, US20110294730 in the treatment of glaucoma, US20120003296 for the treatment of dry eye syndrome, US20120101033 retinitis pigmentosa, US20120128683 for Autism treatment, US20090304776 for diabetes, US20110020279 for rabies cure, simple method to curtail and cure Parkinson's disease (provisional patent submitted) and many such publication and the reader are referred to these publication about why we use the insulin in the treatment of tuberculosis,

We have used insulin delivered to the olfactory mucosa (with or without therapeutic agents) with monoclonal antibodies and other therapeutic agents for the treatment of many neurodegenerative diseases including the cases of reduced mental cognition, Alzheimer's disease and Parkinson's disease (www.wedgetherapeutics.com; Alzheimer's) with declining memory in the aged, Parkinson's with glutathione and dopamine, as well as for depression due to any number of reasons including PTSD, concussions (including football related), cancers, Lyme disease, strokes etc. It reduced the depression, improved the memory, and increased cognition in PTSD patients as it did in Alzheimer's, Parkinson's disease, senile brain atrophy and other neurodegenerative diseases conditions.

Insulin is incorporated and compounded to treat TB disease with Vitamin C and other therapeutic agents as described; to augment, supplement, and amplify the effects of therapeutic agents, as well as to enhance their uptake by increasing the cell membrane permeability (FIG. 10) against this acid fast mycobacterium tuberculosis bacillus and macrophages containing the bacillus. Insulin has a metabolic augmenting effect on the Mycobacterium tuberculosis and enhances their activity by increasing the metabolism making the cell membrane more permeable for entry of large doses of anti TB therapeutic agents to kill the mycobacterium tuberculosis. It also enhances the Fenton reaction in the bacillus by producing rapid and enhanced output of ROS with administration of Vitamin C and other therapeutic agent's action against the Mycobacterium tuberculosis (FIGS. 3, 4, 10) due to enhanced metabolisms. Insulin enhances the uptake of large doses Vitamin C and anti-tuberculosis therapeutic agents to enter the bacterial membrane and kill them. Further it also enhances the activity of ROS many fold, and thus helps to destroy the tuberculosis bacteria with administration of Vitamin C and anti TB therapeutic agents. It enhances the permeability of cells containing Mycobacterium tuberculosis and bacillus itself and destroys the cell and the bacterial within it.

It is important to note, cancer cells develop 3-10 times more insulin receptors to meet the energy (glucose) needs of the rapidly multiplying cells. Similarly M. tuberculosis bacteria, infected macrophages and other phagocytosed cell need more energy to maintain and survive this bacterial infection within and other offending agents they are exposed also develop 3-10 times more insulin receptors. This hypothesis of Dr. Shantha needs to be investigated. Such a phenomenon also includes cancers and immune system cells of the AIDS and other neurodegenerative diseases disease exposed microglia cells. Besides their own metabolic needs, the TB infected immune system cells such as macrophages need to fight the offending infection and support their energy needs also. Thus, we believe that the M. tuberculosis bacteria infected cells as well as other infected immune system cells develop more insulin receptors on their cell membrane as seen in cancer cells (Milazzo. et al., 1992). Hence the administration of insulin will get attached to these infected cells receptors, and open the pathways for entry of glucose. These opening of the pathways on the cell membranes also allow the entry of large amounts of insulin tagged therapeutic agents against infected latent and persister cells also and kill the TB bacteria within the cells and the cell itself (FIG. 10). Insulin has the same effect on the free floating M. tuberculosis bacteria, other offending microbial agents and free floating cancer cells about to metastasize.

As of now, no one has investigated the insulin receptors on the cell membrane of M. tuberculosis bacteria and M. tuberculosis bacteria containing macrophages as of now. May be it is important to explore between the normal TB bacteria and drug resistance bacteria and the immune defense cells such as macrophages which act as host for this bacteria to see whether these insulin receptors play a role in development of drug resistance and virulence of these bacteria and in the development of latent disease and persister cells.

Insulin promotes the glucose metabolism within the mitochondria and other intracellular organelle, which increases the ATP production aerobically. The ATP produced at the inner cell membrane of the M. tuberculosis bacteria augments the Fenton reaction in the cells of the mycobacterium tuberculosis with increased production of ROS. It enhances the activity of Bedaquiline therapeutic agents many fold to inhibit the ATP synthase activity in drug resistant tuberculosis bacteria (FIG. 4). Due to increased permeability of the phagocytes lodging the bacteria and bacterial cell wall, it will augment the uptake of TB therapeutic agents inside the tuberculosis bacteria. The hypoglycemia induced by the fast acting insulin will make the tuberculosis bacteria more permeable, allowing even more therapeutic agents to kill the organisms. Thus insulin in our invention alters the pharmacokinetics in the treatment of TB has the following effects in curtailing and curing the TB and eliminating the MDR bacteria:

Insulin make the bacterial and infected cell more permeable to therapeutic agents surrounding the bacteria (FIG. 10), allowing large dose of therapeutic agents to enter the bacteria and the cells containing the mycobacterium tuberculosis (phagocytes), and kill the bacteria and the cells which contain them.

Insulin induces hypoglycemic milieu which makes the bacteria to seek glucose for energy resulting in entry of the high doses of therapeutic agents with glucose taken up in elevated concentration.

These effects take place in both sensitive and drug resistant bacteria.

Due to this therapy, even the latent or hidden mycobacterium tuberculosis bacteria and infected persister cells are destroyed thus preventing the future development of the full blown disease and its spread to other human.

The combination of this therapy can be used on the caregivers and close family members of the open TB patients as prophylactic, and prevent the future acquiring the TB and developing drug resistant tuberculosis bacteria.

Supplemental oxygen and hyperbaric (as described below) therapy during treatment using Vitamin C and other therapeutic agents amplifies the production of ROS which kills the bacteria and infected cells due to enhanced metabolic activity by insulin.

Enhances the uptake and activity of Vitamin C and many anti-tuberculosis drugs, and their effect at ferric ions to produce rapid and more ROS due to insulin that kills the mycobacterium tuberculosis bacteria.

Other Adjuvant Therepies that can be Used Besides Vitamin C and Known Anti-Tuberculosis Therapeutic Agents to Treat Tuberculosis:

There are many additional adjuvant therapies to treat TB and eliminate this M. tuberculosis bacteria from the body. The important ones are:

Ibuprofen and aspirin as an adjuvant therapeutic agents for treating Mycobacterium tuberculosis inflammation

Interferons therapy to treat tuberculosis

Dinitrophenol as intracellular hyperthermia (heating) agent

Ozone autohemotherapy and local delivery in the irrigation

Hydrogen peroxide: IV and local irrigation of cold abscess

Ezetimibe to deny needed lipids to M. tuberculosis bacterial membranes

Clavulanic acid binding to TB bacteria

Supplemental oxygen and hyperbaric therapy (HBOT) to increase the production of ROS and other systemic effects

Application of AC electrical energy on the chest wall to stop division of M. tuberculosis bacteria as described above

Ibuprofen and Aspirin as an Adjuvant Therapeutic Agents for Treating Mycobacterium Tuberculosis Inflammation:

Acetylsalicylic acid (Aspirin) is a classical non-steroidal antipyretic, analgesic and anti-inflammatory drug and in the United States alone, 35,000 kg are consumed daily (Jack 1997). It has irreversible inhibition of cyclo-oxygenease 1 and 2 that are responsible for the prostaglandin synthesis and some autacoids. Autacoids or “autocoids” are biological factors which act like local hormones, act near the site of synthesis briefly and may thus have systemic effect by being transported via circulation. These regulating molecules are also metabolized locally. So the compounds are produced locally, they act locally and are metabolized locally. Autacoids can have many different biological actions including modulation of the activity of smooth muscles, glands, nerves, platelets and other tissues. The anti-thrombotic action of acetylsalicylic acid is mainly due to its anti-platelet action. In a study in mice, Byrne et al, (2006) used aspirin or ibuprofen alone at 20 mg/kg per day had little effect on tuberculosis infection. After one month of treatment combined with pyrazinamide at 150 mg/kg per day led to a reduction of about 1.5 log₁₀ cfu in the lung and 2 log₁₀ cfu in the spleen compared with the control. Simultaneous administration of either aspirin or ibuprofen with pyrazinamide resulted in a further decrease of TB lesion in the spleen compared with mice receiving pyrazinamide alone. Of mice treated with both aspirin and/or pyrazinamide, had culture-negative spleens (Vilapalana. et al. 2013. Ivanyi J, et al. 2013). They concluded that aspirin and ibuprofen enhance the effect of pyrazinamide during the initial phase of tuberculosis treatment in the mouse model. Studies show that acetylsalicylic in large doses, induce alterations on membrane of red blood cells (Amman et al. 2002). That is what may happen in mycobacterium tuberculosis membrane when aspirin and/or ibuprofen are administered allowing more anti TB drugs to enter the mycobacterium tuberculosis, and augments its killing. Studies by Canan et al. have shown a trend toward reduced inflammation in the old lungs. They concluded that “ibuprofen might work on specific pathways to lower inflammation, and that might help with control of TB.” Turner and colleagues of this study have extended the work to test whether ibuprofen affects the elderly mouse immune response to TB infection (Canan 2014). Mice infected with TB bacteria that were treated with ibuprofen lived longer than mice not treated with ibuprofen, according to the study published online May 3 in the Journal of Infectious Diseases.

Dr. Pere-Joan Cardona, the lead investigator on the ibuprofen study, said that his results suggest that treating patients with a combination of standard TB drugs and ibuprofen might reduce treatment time, and enhance outcomes for patients. “Nevertheless, we need a clinical trial to demonstrate this in humans, and officially put it in the TB guidelines,” said Cardona, who is a researcher at the Experimental Tuberculosis Unit, a research organization affiliated with the Autonomous University of Barcelona, in Spain. The same is true for vitamin C; the researchers suggest that further research should be done to explore the potential of using it in tuberculosis treatment. “At the very least, this work shows us a new mechanism that we can exploit to attack TB,” said Dr. William Jacobs, the lead investigator on that study, and our finding of the effectiveness in a TB case supports these findings.

Ibuprofen (NSAID) inhibits Cox I and Cox 2 enzyme which stops or reduces the production of prostaglandin hormone being released that cause inflammation and helps to decrease swelling, pain, or fever. COX-2 selective inhibitor is a form of non-steroidal anti-inflammatory drug (NSAID) that directly targets COX-2 also, an enzyme responsible for inflammation and pain. At present we are prescribing the Ibuprofen which has both Cox 1 and 2 inhibitor effects in the treatment of TB. The NSAIDs as adjuvant therapeutic agents are used in our invention in the treatment of TB, to reduce the inflammation, and swelling at the site of the TB lesion of the lungs. The results showed that for TB-infected mice, ibuprofen treatment reduced the number of TB bacteria detected in the organs, and increased the survival of the animals (Byrne 2006). The next step should be to examine the effects of ibuprofen in combination with standard TB drugs with high dose Vitamin C. How does it work in tuberculosis? The following are the possibilities:

It reduces the inflammation processes involved in the formation of tuberculosis inflammatory granuloma lesions with subsequent breakdown (caseation) causing pus filled lesions (cold abscess in the vertebral column), and cavities in the lungs.

In the tissue, the aspirin and ibuprofen create acid media around the TB lesion where the therapeutic agents like Pyrazinamide (a first-line anti-tuberculosis agent) become more effective in killing non-replicating or slow-growing bacilli under acid pH conditions created by NSAID. The studies show that the simultaneous administration of aspirin with pyrazinamide resulted in statistically significant fewer bacteria in the lungs. Ibuprofen also enhanced the activity of pyrazinamide, much like aspirin, resulting in lower unit counts in the lung. The difference in the lungs was more substantial, with lower counts in the ibuprofen and pyrazinamide combination group when compared with the pyrazinamide-only group.

It reduces bringing the polymorphonuclear white blood cells to the site of the Mycobacterium tuberculosis infection that produce caseous lesions and cavities due to reduced inflammatory cytokines such as bradykinins, histamine, arachnidic acid with reduced production cyclo-oxygenease enzymes due to mycobacterium tuberculosis bacteria.

It is also is anti angiogenic, meaning, there is less new blood vessel formation to supply the infected areas and the site of infection. Thus it reduces the rapid multiplication of tuberculosis bacteria, progression and spread of the lesions and the disease; to neighboring and distant regions of the body. This effect may reduce the osteoporosis in bone tuberculosis granuloma due to reduction of blood supply. Increased vascularity due at the TB granuloma site is blamed on the development of osteoporosis. This will prevent the collapse of the vertebrae and fractures.

There is possibility that aspirin and Ibuprofen such agents also makes the Mycobacterium tuberculosis cell wall more permeable besides creating acidic membrane to therapeutic agents; thus augments the penetration of various therapeutic agents including Vitamin C used against the mycobacterium tuberculosis.

We administer 200 mgs of ibuprofen orally 2-3 times a day in all our tuberculosis patients in addition who are under high dose Vitamin C and other anti TB drugs therapy. It can be administered intravenously (IV) also 200-400-800 mg over 30 minutes depending upon the desired effects and the condition of the patient. Do not use ibuprofen just before or after heart bypass surgery (coronary artery bypass graft, or CABG). This medicine may cause life-threatening heart or circulation problems such as heart attack or stroke, especially if you use it long term. This medicine may also cause serious effects on the stomach or intestines, including bleeding or perforations (forming of a hole) that can occur without warnings while the patient are taking ibuprofen, especially in older adults. Exercise caution if a person has:

a history of heart attack, stroke, or blood clot, congestive heart failure, high blood pressure;

a history of stomach ulcers or bleeding; bleeding or blood clotting disorder,

Pregnancy and breast feeding.

asthma; allergic to NSAIDs, polyps in the nose,

Liver or kidney disease;

Systemic lupus erythematosus (SLE).

Interferons Therapy to Treat Tuberculosis:

Tuberculosis bacteria with multiplication of the organism within macrophages and monocytes and control of the infection by cell-mediated immunity (CMI) orchestrated by T-cell-derived lymphokines and carried out by the effector cells, activated macrophages. In fact, the CMI generated in tuberculosis is so potent that 90% of the immunocompetent humans infected with M. tuberculosis are able to contain the infection and avoid progression to clinical disease during their lifetimes. This potency of CMI may be demonstrated experimentally: guinea pigs previously infected with M. tuberculosis react against cutaneous reinfection with live bacilli by rapidly forming a necrotic skin lesion that subsequently resolves spontaneously. This reaction, now known as the “Koch phenomenon,” suggests that the immune reaction mounted by infected animals is also particularly potent (Grosset, J. 2003).

An interferon is a small protein produced by the body's immune system (cell mediated immunity) in response to an infection made by cells such as leukocytes, T-cells, and fibroblasts. There are three types of interferon: Type I, Type II, and Type III. These types are divided by the types of cells they interact with, how they are produced, and what they do. Type I includes sub classifications known as alpha, beta, kappa, delta, epsilon, tau, omega, and zeta. The Type II category includes interferon-gamma, made by the T-cells, while the Type III category consists of several versions of interferon-lambda.

Interferons are secreted by cells in response to stimulation by viruses, bacteria, parasites, tumor or other foreign substances; they activate immune cells, by activating natural killer cells and macrophages, by up regulating the antigen presentation. They do not directly inhibit the viruses or bacteria multiplication but they stimulate the infected cells and those nearby to produce proteins that prevent the offending agent from replicating within them. When TB spreads out of lungs and within the lungs, interferon-Y⁻, and number of CD4+ cells increase compared to uninfected. After binding, they are drawn inside the cell's cytoplasm (FIG. 1a), where they cause a series of reactions that produce other proteins that fight and ward off disease. So far, scientists have identified over 30 disease-fighting proteins produced by interferons. We have used interferons for the treatment of lung, pancreatic and other types of cancers as well as in the treatment of viral hepatitis.

The interferons direct the immune system's attack on viruses, bacteria, tumors and other foreign substances that invade the body. Interferon-Y⁻ is a pleiotropic cytokine that has specific immune-modulating effects, e.g. activation of macrophages, enhanced release of oxygen radicals, microbial killing, enhanced expression of major histocompatibility complex (MHC— normally found only on antigen-presenting cells such as dendritic cells, mononuclear phagocytes, some endothelial cells, thymic epithelial cells, and B cells), Class II molecules (located on cell surface molecules on antigen presenting cells such as macrophages, B cells and dendritic cells), anti-viral effects, induction of the inducible nitric oxide synthase gene and release of NO, chemotactic factors to recruit and activate immune effector cells, own regulation of transferrin receptors limiting microbial access to iron necessary for survival of intracellular pathogens, and such acts. Because class II MHC is loaded with extracellular proteins, it is mainly concerned with presentation of extracellular pathogens.

Genetically engineered mice that lack interferon-Y or its receptor are very susceptible to mycobacterial infection. An open-label study of nebulized IFN-Y⁻ 1b in 5 patients with MDR-TB showing clearing of the sputum after 1 month, as well as a second study in 10 TB patients showing enhanced IFN-Y⁻ signaling after one month of therapy with nebulized IFN-Y⁻ 1 b was previously reported (Condos et al, L1997, 2003). Dawson et al (2009) describe the use of recombinant interferon-Y⁻ b adjuvant therapy plus Directly Observed Therapy (DOT) in cavitary pulmonary tuberculosis can reduce inflammatory cytokines at the site of disease, improve clearance of M. tuberculosis bacteria from the sputum, and improve constitutional symptoms. A method for treating tuberculosis in a subject comprising administering 2000-1000 μg of an aerosolized interferon-Y resulting in deposition of a therapeutically effective amount of interferon-Y⁻ in the lungs of the subject as described in U.S. Pat. No. 8,105,572 B2 by Rany Condos, and Gerald Smaldone. Use of our inventive device described here in to deliver this interferon will make sputum negative faster (infecting to non-infecting patients), and facilitate curing cavitary tuberculosis.

Bronchoalveolar lavage (BAL) of patients with pulmonary tuberculosis has shown increases in inflammatory cytokines and percent of CD4+ cells compared to uninfected controls. In advanced TB cases and cavitary tuberculosis markers of effective immunity are reduced. Nitric oxide (NO) has been identified as a mechanism of mycobacterial killing, while mycobacteria can also be eliminated by autophagy, apoptosis, cytotoxic CDS 8+ cells, alpha defensins from neutrophils, and phagosomal-lysosomal rupture with subsequent cytosolic demise (Martineau, et al. 2007, Vander Wei, et. al. 2007, Gutierez, et. al. 2004). Using the device described herein, it is easy to perform bronchoalveolar and cold abscess lavage. Autophagy is a defense mechanism inhibiting BCG and M. tuberculosis survival in infected macrophages (Maximiliano G.). However, mycobacteria have evolved virulence factors to persist in macrophages, probably disrupting the interferon-Y signaling pathways.

It is thought that once the M. tuberculosis bacteria is phagocytosed (FIG. 1a) by white blood cell (macrophages), the bacteria within the phagosome arrest the maturation of phagosome, and escape to the cytoplasm by piercing the phagosome membrane via ESAT-6 protein (FIG. 1a), then the pathogen modulates the host cell death to escape from the phagocytosed macrophage and spreads locally and to distant organs. M. tuberculosis actively modulates the autophagic pathway to avoid degradation. To avoid autophagic killing by infected cell, there is a complex interplay between the host defense mechanisms and M. tuberculosis machinery to avoid autophagic killing. It is obvious that the capacity of the pathogen to exploit and manipulate host cell pathways to its own benefit is critical for its survival and delicate balance exists between triggered immune defense responses and the host processes manipulated by the pathogen. As the immune response declines due to any number of conditions including HIV infection, the M. tuberculosis takes advantage, multiplies, emerges out of disabled or dead macrophage (apoptosis?), and spreads to produce active diseases locally, spread far and wide (FIG. 1a #64, 65, 66). This happens because the M. tuberculosis somehow escapes autophagic killing by the macrophages.

It is important to note that the interleukin-6 (IL-6) productions by M. tuberculosis infected macrophages selectively inhibit macrophage response to IFN-Y⁻. IL-6 is produced at the site of M. tuberculosis infection in the early stages of the disease. The IL-6 is also produced by a variety of cells including T cells, macrophages, endothelial cells, fibroblasts, B cell and various cancer cell lines. Elevated levels of IL-6 were found in pulmonary TB patients harboring drug sensitive or drug resistant M. tuberculosis bacteria. It has been shown that the recombinant IL-6 promotes growth of the TB bacteria and it has been also shown experimentally that IL-6 deficient mice are more susceptible to M. tuberculosis infection. Nagabhushanam et al (2003) showed that IL-6 produced by M. tuberculosis infected macrophages selectively inhibits macrophage response to IFN-Y⁻. The M. tuberculosis unregulated IL-6 production to inhibit IFN-Y induced autophagy formation, thus avoiding phagosome maturation and subsequent killing by lysosomal enzymes. It is known that the M. tuberculosis infected macrophages or monocytes secret pro-inflammatory cytokines such as TNF-alpha, IL-1, IL-6, and IL-12 as well as anti-inflammatory cytokines IL 10, and TGF β (Beltan E. et al. 2000). These cytokine play a critical role in the recruitment of monocytes and lymphocytes from the bloodstream to the infected sites, in control of inflammatory response, and subsequent granuloma formation and in the outcome of M. tuberculosis bacterial infection.

It is hypothesized that pharmacologic doses of rIFN-Y⁻ 1 b could augment the host immune response in TB, and to evaluate this we need to conduct randomized, controlled clinical trial with rIFN-Y⁻ 1 b bat 200-400 μg/day for three days/week with or without other therapies discussed here. Such a study of effect using rIFN-Y⁻ 1 b is reported in U.S. Pat. No. 8,105,572 B2 with positive results. The study reported rapid clearance of M. tuberculosis bacteria from the sputum, improved symptoms, and reduced inflammatory macrophage-neutrophil alveolitis. These findings suggest that nebulized or directly delivered (by using our device) recombinant interferon-Y⁻ 1b may have a role in adjunctive immune stimulation in patients with TB granuloma, miliary TB and cavitary tuberculosis. With our inventive device described herein, by direct infusion or by humidity aerosol produced by ultrasonic nebulizer, we can deliver this cytokine directly into the cavitary tuberculosis and/or to the site of alveolar TB infection in the treatment of tuberculosis. This will add to the therapy by high dose Vitamin C and other anti-tuberculosis therapeutic agents we plan to use in our invention.

Intracellular Hyperthermia by 2,4-Dinitorphenol (DNP) to Disorganize Mycobacterium Tuberculosis Metabolism and Produce More ROS to Kill the Bacteria:

Hyperthermia as a method of treating cancer has a long history, dating back to around 3000 B.C. The term hyperthermia is a combination of two Greek words: hyper (rise) and therme (heat) and refers to the increasing of body temperature or selected tissues in order to achieve a precise therapeutic effect. The first known use of heat treatment was carried out by an Egyptian aruspice named Imhotep (2655-2600 B.C.) to heat locally breast cancers. Hyperthermia treatments were also popular in ancient China and India. Early Greek and Roman physicians knew about hyperthermia therapy. For example, the Greek philosopher Parmenides (ca 540-ca. 470 B.C.) was deeply convinced of the effectiveness of hyperthermia as evidenced by the words: “Give me the power to produce fever and I will cure all diseases” This view was shared by Hippocrates (460-370 B.C.), a Greek philosopher and scientist who is considered the “father of medicine” He used heat to cure breast cancers like Egyptians. He claimed that the disease must be incurable, if it cannot be cured by using heat. Belief in the curative effect of fever was also shared by Celsus (ca. 25 B.C.-45 A.D.), a Roman author of the first systematic treatise on medicine “De Medicina” and Rufus of Ephesus, a Greek physician who lived at the turn of the 1st and 2nd century. Celsus described the hot baths as a tool in the treatment of various diseases.

The present author practices hyperthermia two to three times a month, since 1982, He believes in its disease preventing, health promoting, immune system enhancing, blood vessels cleansing, heat shock protein producing, brain shielding, heart protecting (from ASVD, M I, post MI, etc.), BV dilating, enzyme activating, infection fighting, cancer preventing, and untold number of other effects besides stimulating the proliferation of immunocompetent cells, enhances synthesis of immunoglobulin's, activates function of macrophages and increases sensitivity of microorganisms to phagocytosis, promotes the synthesis of biologically active substances such as interleukins, leukotrienes, and prostaglandins which is beneficial in reducing inflammation. The heated blood with rapid blood flow (due to increased cardiac output, vasodilatation, and ↑BP-chronotrophic and ionotrophic effect), the platelets, lipids and leukocytes sticking to the BV endothelium are washed out like hot water or steam cleaning the greasy engine and thus inhibits the formation and progression of ASVD of BV all over the body. My experiences suggest that hyperthermia I practice like moderate regular exercise attenuates oxidative stress. The mild oxidative stress possibly elicited by regular mild hyperthermia like exercise appears to manifest a hormesis-like effect (a biological phenomenon whereby a beneficial effect of improved health, stress tolerance, growth or longevity results) from exposure to low doses of an in non-muscular tissues, constituting beneficial mechanisms of hyperthermia and exercise by adaptively up regulating various antioxidant mechanisms, including antioxidative and repair-degradation enzymes for damaged molecules. Importantly, the adaptation induced by regular low hyperthermia and exercise were effective even if initiated late in life.

The first paper on hyperthermia was published in 1867 by a German surgeon Carl D. W. Busch (1867). He described the case of a 43-year-old woman with advanced sarcoma on her face. After the tumor was removed, the patient fell ill with erysipelas. The disease induced high temperature which led to tumor regression. Busch's discovery was fundamental because it was the first reported case showing that high temperature can selectively kill cancerous cells while not affecting the healthy. This event and other similar reports had led to increased interest in hyperthermia and make many trials to induce infectious fever in patients with cancer, for example, by applying dirty bandages or blood of people suffering from malaria into open wounds. Apart from the research of Busch, the relationship between infection and cancer regression was looked for by an American surgeon William B. Coley (1862-1936). His studies involve the injection of different types of bacterial pyrogens into tumors and observation of their behavior. In 1891 he developed a toxin that caused typical erysipelas with its typical fever and cured dozens of bone and soft tissue malignant tumors (Coley W B. 1891, 1893). For this reason, Coley is called the “father of immunotherapy” against cancer. So-called Coley's toxin had been used to treat various types of cancer for nearly a century. I have prescribed Coley's vaccine for advance renal cell carcinoma, and the patient—a practicing physician in US is still alive and free of cancer after a decade of treatment. Results of research conducted by Cooley's showed that the five year survival rate increased from 28 to 64% for patients with inoperable cancer. Dr. Coley as head of the Bone Tumor Service at Memorial Hospital in New York injected more than 1000 cancer patients with bacteria or bacterial products which became known as Coley's toxins. It produced hyperthermia and stimulated and/or enhanced the immune system attack on tumors. Coley's daughter tabulated every patient he treated and reviewed all his notes. She published 18 monographs and tabulated over 1000 of his cases and noticed that in 500 of these there was near-complete regression (Coley-Nauts H, McLaren J R. 1990). Radiologic clinic of North America, (Vol 27, Number 3 May 1989, guest editor Dr. Steeves A. R, published by W.B. Saunders) published an important monograph containing scholarly articles on Hyperthermia. Those who want to know more about history, biology, methods and types of hyperthermia are referred to this important monograph.

Increasing the temperature is known to have killing effect on micro-organisms and cancers besides other metabolic effects on the human body. Rise in body temperature after an infection is our bodies' a natural way of eliminating the infection by the immune system. Hyperthermia activates tumor necrosis factor (TNF), interferons, streptokinase and many other cytokines, all related to the immune system. Immune therapies are being developed for many cancers, and one of it is in the market for treating advanced prostate cancer (PROVENGE® is a prescription medicine that is used to treat certain patients with advanced prostate cancer and is made from your own immune cells). In fact vaccines are being developed for the treatment of numerous types of cancer, particularly colon cancer and melanoma. One form of immunotherapy which is consistently effective is the installation of BCG bacilli into the bladder with insulin (IHAT) to treat superficial bladder cancer which I have used successfully in 3 cases.

Hyperthermia is considered the “fourth leg” of cancer treatment so also for infections as we describe here with wide range of therapeutic effects. Even if the cancerous cells do not die outright, they may become more susceptible to ionizing radiation therapy or to certain chemotherapy drugs, immune system attack, so also M. tuberculosis bacteria and bacillus leprae. There is three forms of hyperthermia have been developed for treating cancers, infections and other body afflictions. They are:

Local:

Heating only the tumor or site of TB infection locally. This is the method we want to use to treat TB cervical lymphadenitis, cold abscess and lung lesions.

Regional:

Heating a sector of the body, including the area affected, such as extremities, lungs, or intestines etc.

Whole Body:

Heating the entire body to treat generalized diseases such as MRSA infections, AIDS and related infection, Leprosy, cancers and in some cases of tuberculosis.

Without going to details various forms of hyperthermia, we want to use DNP in small doses to induce Local hyperthermia by delivering to the lungs and cold abscess treatments with combination anti-tuberculosis drugs delivered through special delivery device described herein. The exact molecular and cellular mechanism by which heat kills (by DNP and other hyperthermia methods) or inactivates microorganisms and tumor cells is unknown. Publications and patents such as U.S. Pat. No. 7,635,722 BI to Bachynsky and Roy, WO 2015/031598 PCT/US2014/053127, WO 2015/031756, PCT/US2014/053406 to Shulman, Jamison and Spiegel; U.S. Pat. Nos. 4,724,230, 4,724,234, 4,935,450 to Cone Jr. Clarence D for the treatment of cancers; articles by Gerald 1. Flanagan et al. 1998, Tainter et. al. 1932-1986, Yanase, M. et. al. 1998; tell us some of the mechanisms of action of DNP and hyperthermia against diseases, and awakened great interest in this metabolic heat producing uncouplers. Patent to Shulman, Jamison and Spiegel (2014), describe on use of DNP for Non-alcoholic fatty liver disease (NAFLD) and other diseases in 117 page patent in great detail. They showed that DNP can be used safely, and the study was supported by US Grant and the patent assigned to Yale University. We have incorporated their finding by reference.

Body temperature is a critical factor in determining host susceptibility, location of lesions, and the natural history of many infectious diseases. Temperature has direct effects on the growth of all microorganisms, including those that are pathogenic. Almost all of the bacteria that cause diseases in humans grow optimally within the range of 33-41 degree C. and, their temperature growth characteristics are not easily altered in vitro. The temperature above that is both bacteriostatic and bactericidal. We successfully treated the massive peritoneal candida infection due to intestinal leak after surgery with Diflucan and peritoneal hyperthermia, saving the life of the patient (Shantha 2003).

To avoid the toxicity of DNP, Shulman et. al., described that, by altering the pharmacokinetics of DNP to promote a low sustained systemic release, the therapeutic window of DNP is increased by more than 500-fold according to their patent WO 2015/031756, Page 19 with no toxicity. Such a formulation compositions in therapeutic low dose, sustained release formulations of DNP, useful in treating a disease or disorder, such as but not limited to non-alcoholic fatty liver disease (NAFLD), non-alcoholic steatohepatitis (NASH), hepatic steatosis, type 2 diabetes (T2D), acquired lipodystrophy, lipodystrophy (inherited), partial lipodystrophy, hypertriglyceridemia, obesity, metabolic syndrome, Rett's syndrome, metabolic syndrome associated with aging, metabolic diseases associated with increased reactive oxygen species (ROS), Friedreich's ataxia, insulin resistance, hepatic fibrosis, liver cirrhosis and hepatocellular carcinoma, auto-immune disease, congenital muscular dystrophy, fatal infantile myopathy, “later-onset” myopathy, MELAS (mitochondrial encephalopathy, lactic acidosis, and stroke), MIDD (mitochondrial diabetes and deafness), MERRF (myoclonic epilepsy ragged red fiber syndrome), arthritis, NARP (Neuropathy; Ataxia; Retinitis Pigmentosa), MNGIE (Myopathy and external opthalmoplegia; Neuropathy; Gastro-Intestinal; Encephalopathy), LHON (Leber's; Hereditary; Optic; Neuropathy), Kearns-Sayre disease, Pearson's Syndrome, PEO (Progressive External Opthalmoplegia), Wolfram syndrome, DIDMOAD (Diabetes Insipidus, Diabetes Mellitus, Optic Atrophy, Deafness), ADPD (Alzheimer's disease; Parkinson's disease), AMFD (ataxia, myoclonus and deafness), CIPO (chronic intestinal pseudo obstruction; myopathy; opthalmoplegia), CPEO (chronic progressive external opthalmoplegia), maternally inherited deafness, aminoglycoside-induced deafness, DEMC HO (dementia; chorea), DMDF (diabetes mellitus; deafness), exercise intolerance, ESOC (epilepsy; strokes; optic atrophy; congenitive decline), FBSN (familial bilateral striatal necrosis), FICP (fatal infantile cardiomyopathy plus a MELAS-associated cardiomyopathy), GER (gastrointestinal reflux), LIMM (lethal infantile mitochondrial myopathy), LDYT (Leber's hereditary optic neuropathy and Dystonia), MDM (myopathy; diabetes mellitus), MEPR (myoclonic epilepsy; psychomotor regression), MERME (MERRF/MELAS overlap disease), MHCM (maternally inherited hypertrophic cardiomyopathy), MICM (maternally inherited cardiomyopathy), MILS (maternally inherited Leigh syndrome), mitochondrial encephalocardiomyopathy, mitochondrial encephalomyopathy, mitochondrial myopathy, MMC (maternal myopathy; cardiomyopathy), multisystem mitochondrial disorder (myopathy; encephalopathy; blindness; hearing loss; peripheral neuropathy), NIDDM (non-insulin dependent diabetes mellitus), PEM (progressive encephalopathy), PME (progressive myclonus epilepsy), Rett's syndrome, SIDS (sudden infant death syndrome), SNHL (sensorineural hearing loss), Leigh's Syndrome, dystonia, schizophrenia, and psoriasis (WO 2015/031756 Pages 18-20; and WO 2015/031598 A2). These patents were issued to treat non-alcoholic fatty liver metabolic disorder (NAFLD) using DNP. This inventive DNP study was made possible with US government support under DK085638 and DK040936 awarded by National Institutes of Health of US government which has certain rights in this invention.

Adding insulin and other therapeutic agents using insulin induced hypoglycemia activated therapy (IHAT) will enlarge and amplify the effectiveness of DNP (hyperthermia by other methods) even in small doses in treating various diseases including Alzheimer's disease. I propose using small doses of DNP (hyperthermia triggering dose) and then augment the hyperthermia effect by heating the body with Far Infrared Sauna such the one made by Smarty®. By this method we induce intracellular hyperthermia with nontoxic doses of DNP and augment its effect by external hyperthermia to cure and curtail diseases.

Leprosy Caused by Acid-Fast Bacillus Like M. tuberculosis Bacteria, with Use of Whole Body Hyperthermia Combined with Anti-Leprosy Drugs:

Unlike M. tuberculosis bacteria, leprosy organisms which are also acid fast bacilli proliferate and follow the coolest temperature gradients in the body, 25-33° C. That is why the lesions of Hansen's disease (leprosy) caused by Mycobacterium leprae, characteristically grow and destroy the most acral (peripheral body parts), coolest parts of the body such as fingers, toes, external ear, the air-stream cooled nasal alae and larynx. For adults the standard regimen is: Rifampicin: 600 mg once a month Dapsone: 100 mg daily, Clofazimine: 300 mg once a month and 50 mg daily for 12 months. Single Skin Lesion Paucibacillary leprosy in adults, the standard regimen is a single dose of: Rifampicin: 600 mg Ofloxacin: 400 mg Minocycline: 100 mg. These treatments become more effective combining hyperthermia with Vitamin C as described in this invention.

In animals, the leprae organisms can only be grown in the armadillo (cold blooded) or foot pads of mice where the “in situ” lesion temperatures are 27-30° C. Spontaneous improvement in leprosy lesions have been reported in patients following febrile illness. Fever therapy, hot baths and local heat therapy were formerly utilized in treating this disease. We propose Hyperthermia with FIR (far infrared) sauna hyperthermia (with or without using sustained release low dose DNP formulation of Shulman (2014), Vitamin C, with insulin and anti-leprosy therapeutic agents to cure this condition within days, instead of months and years. Whole body hyperthermia can be induced by FIR sauna (Smarty®) or hot water bath tub hyperthermia without DNP also, which we did use it to treat cancers, Lyme disease, autoimmune diseases, obesity, and MRSA infections. We are planning to use this method in treatment of Leprosy-both multibacillary (positive smears at any site) and paucibacillary (shows negative smears at any site) groups. By this method along with high dose rifampicin (high IV doses), high dose Vitamin C, with dapsone and clofazimine (multi drug therapy-MDT with or without Thalidomide [thalidomide contraindicated in pregnant woman] and prednisone, under IHAT, we can cure leprosy within days or weeks instead of months and years as it takes to cure and curtail this chronic ultimately debilitating disease. Thus eliminate this chronic ancient scourge or illness from the earth. We have used this method successfully for the treatment of Lyme disease, MRSA infection and cancers. All I need is an opportunity to try this safe method to cure leprosy within days instead of months and years.

Hyperthermia is also known to destroy Treponema pallidum (Julius Wagner-Jauregg was an Austrian physician, won the Nobel Prize in Medicine in 1927 for his discovery of the therapeutic value of malaria inoculation to induce fever (hyperthermia) in the treatment of dementia paralytica due to Syphilis spirochete infection-here the treatment was worse than the disease), the causative agent of syphilis, by heating five hours at 39° C., three hours at 40° C., two hours at 41.5° C. or one hour at 41.5° C. The spirochetes responsible for yaws, bejel, pinta and Lyme disease show similar temperature sensitivity. We have treated using far infrared method to induce hyperthermia for treating Lyme diseases, MRSA infection, and cancers successfully. Other bacteria that predominately cause lesions at cool sites and are susceptible to heat inactivation include, Neisseria gonorrhea, Hemophillus ducrei (chancroid), Mycobacterium ulcerans, Mycobacterium marinum (“swimming pool” granuloma), Diphtheria, etc. Further, hyperthermia has been reported to be synergistic with antibiotic and chemotherapy in the treatment of various bacterial diseases. Elevated body temperature potentiates the effect of penicillin on staphylococci and syphilis. Hyperthermia makes sulfadiazene bactericidal for streptococci. We treated MRSA infection with high dose IV antibiotics under IHAT therapy (insulin induced hypoglycemia augmentation activation therapy, also known as IPT, IPTLD), whole body hyperthermia, HBO successfully. Moreover, recent controlled studies show that when antipyretics are used in animals with severe experimentally induced infections, there is increased mortality. Nonetheless, systemic hyperthermia has generally been abandoned as a treatment for bacterial infections with the advent of antibiotics though it can be effectively used in local treatment of TB lesions with anti TB therapeutic agents as described here. The local use of DNP has very little or no effect on the rest of the body due to its low does deposited locally.

Effect of Heat on the Body and Microorganism Such as M. tuberculosis Bacteria:

Heating is known to cause conformational changes in proteins, high heat denatures or low heat activates enzymes, activates hormones, augment the effects of various therapeutic agents, produce heat shock protein, increase the blood flow by vascular dilation and increased cardiac output, increase the movement of immune system cells and humoral factors, increases the output of antibodies and cytokines, increase the production and circulation of lymphocytes and red blood cells, affect cell membrane fluidity and makes it more permeable, cause pathologic processes more amenable to treatment. Heat suppresses ribonucleic acid (RNA) protein synthesis systems. Without RNA synthesis, the TB bacteria cannot survive. This method can be applied in the treatment of leprosy and eliminate the infection rapidly instead of months of therapy. Heat induced by any methods including using DNP increases cell metabolism many folds. It causes anaerobic cell respiration and anaerobic sugar breakdown (glycolysis). This results in production of acids in the cell. These acids and heat increase lysosomal (a kind of incinerator within the cell) activity. The acid milieu also enhances the bactericidal effect of many anti TB drugs especially Pyrazinamide.

Hyperthermia by DNP or other methods (FIR methods or hot water tub immersion) increases the formation of oxygen free radicals wherein second medication's (anti TB drugs) effects increases due to an increase in free radical flux, including superoxide, hydroxyl, hydroperoxyl, hydrogen peroxide and lipid peroxides. These reactive oxygen species (ROS) react indiscriminately and oxidize many organic molecules causing DNA damage, protein denaturation, lipid peroxidation and other destructive chain reactions. Acid microenvironments, known to exist in microorganisms (and in tumors) with high rates of glycolysis (Embden-Meyerhof Pathway) and lactic acid production, favor protonation of the superoxide radical to form the highly reactive and toxic hydroperoxyl radical. Thus, thermal sensitivity of many bacteria and tumors increases with decreasing intracellular pH. As compared to normal cells, many bacteria including M. tuberculosis and malignant cells have a reduced total functional capacity to withstand the increase flux of oxygen free radicals produced by hyperthermia, resulting in their destruction. All these effects of DNP other Hyperthermia's augment the effects of various anti-tuberculosis therapeutic agents including Vitamin C.

Though DNP is inappropriately labeled as weed killer, there are patents and research publications describing its immense value in the treatment of cancers (U.S. Pat. Nos. 4,935,450, 4,724,234), weight loss (U.S. Pat. No. 4,673,691), Lyme disease, and neurodegenerative diseases such as Alzheimer's disease (Ferreira S T, De Felice F G. Neuroprotective actions of 2,4-dinitrophenol: Friend or foe? Dementia & Neuropsychologia 2007; 1(4):334-338. De Felice F G, Houzel J C, Garcia-Abreu J, et al. Inhibition of Alzheimer's disease beta-amyloid aggregation, neurotoxicity, and in vivo deposition by nitrophenols (DNP): implications for Alzheimer's therapy. FASEB J 2001; 15: 1297-1299. De Felice F G. Vieira M N, et. al. Targeting the neurotoxic species in Alzheimer's disease: inhibitors of Abeta oligomerization. FASEB J 2004; 18: 1366-1372. De Felice F G, Ferreira S T. Beta-amyloid production, aggregation, and clearance as targets for therapy in Alzheimer's disease. Cell Mol Neurobiology, 2002; 22:545-563). Thousands of people have taken DNP to control the weight and still taking. One has to bear in mind that it can cause premature cataract and even death if excesses dose taken indiscriminately unsupervised. One has to weigh one benefit against the other. We believe that olfactory mucosal deliveries of DNP in micro doses can reduce the production of beta amyloid and enhance its removal by intracellular hyperthermia. It is not toxic and easily passes blood brain barrier to reach the limbic system including entorhinal cortex and other Alzheimer's disease afflicted regions in the brain. Why not give a chance to this falsely tainted therapeutic agent?

The 2,4, dinitrophenol (DNP—HOC6H3(NO2)2 defeats the proton gradient across mitochondria like membrane (FIGS. 1, 3, 4) of the tuberculosis bacterial cell wall membranes, collapsing the proton motive force that the cell uses to produce most of its ATP biochemical energy (FIG. 1 #85, 2 #15, 17). Instead of producing ATP, the energy of the proton gradient is lost as heat. One of the best characterized mitochondrial uncoupling agents is 2, 4-dinitrophenol (DNP), a protonophore is that it shuttles protons across the mitochondrial membrane, dissipating the mitochondrial proton gradient and promoting heat dissipation of the energy derived from mitochondrial substrate oxidation. Further, the dinitrophenol uncouples oxidative phosphorylation, causes release of calcium from mitochondrial stores and prevents calcium re-uptake. This leads to free intracellular calcium and causes muscle contraction and hyperthermia induction in subjects taking it. Dantrolene, an antidote for malignant hyperthermia treatment (under anesthesia, heat stroke, or DNP induced-though we did not experience any such effect), inhibits calcium release from the sarcoplasmic reticulum in normal muscles, which reduces intracellular calcium. The resulting muscle relaxation allows heat dissipation.

At present we recommend use local DNP injection through the device described herein in measured doses (10-20 mg dissolved in DMSO and diluted in distilled water for injection or aerosolize), or delivering through the aerosol nebulizer methods to the lesions of tuberculosis in the lungs using the device described in this invention. In tuberculosis bacteria, due to its effects, there is lack of ATP energy needed for its survival and spasm of the microbe due to increased calcium levels resulting in its death. The cytotoxic effect of hyperthermia due to local injection or aerosol inhalation of DNP causes numerous complex changes and damage to multiple vital bacterial structures, and its host cell, resulting in their death. The large doses used to lose weight are not used which will eliminate the fear of whole body hyperthermia, tachycardia, diaphoresis, tachypnoea, which may lead severe morbidity and mortality (Grundlingh J. et. al. 2011). We will never see such complication with local injection and aerosol using less than 2-5% (±10 mg) of the dose used for weight loss (400 to 5000 mg per day), delivered locally under monitored conditions. If the obese patients use 50-100 mg of DNP followed by FIR sauna, can achieve the same results (weight reduction) without the any adverse reactions. One need to know that the effects of hyperthermia are global on the body: both on the healthy cell, microbes and any and all diseases of the body and their prevention and treatment.

We plan to use un-coupler of metabolism by using 2, 4, dinitrophenol (DNP), by local injection with a needle, or through the device described herein (FIGS. 5-7). DNP due to intracellular heat production within the cells due to uncoupling of oxidative phosphorylation by allowing the protons to leak across the inner mitochondrial like M. tuberculosis membrane; part of the energy that is normally produced from mycobacterium tuberculosis is lost. Cellular respiration is fatigued-protons exhausted as heat and ultimately the bacteria succumb due to lack of energy-lack of ATP and intracellular heat disrupting the metabolic function of the bacteria. Note that the bacteria inside the membranes contain bacterial matrix 83 (shown in FIG. 1 cytoplasm with various nucleic acids and messenger RNA) whose calcium is increased adding to the bactericidal effect of uncoupling agents. Uncoupling also causes release of calcium from bacterial stores and macrophages and prevents calcium re-uptake. This leads to free intracellular calcium elevation, and causes bacterial stiffness like skeletal muscle contraction and hyperthermia disturbing its metabolism, disturbing its membrane stability, ultimately the death of M. tuberculosis. There is no toxicity using±10 mg per dose. The complications reported in the literature for losing weight are due to taking 400-5000 mg a day (Grundlingh J et. al. 201) without proper medical supervision.

Coley's Toxins as Vaccine to Mount Immune System Attack on the TB Bacteria and as Inducer of Hypertheramia:

Coley's toxin is a mixture consisting of killed bacteria of species Streptococcus pyogenes and Serratia marcescens, named after William Coley (Coley W B. 1983. Thotathil Z, Jameson M B (2007). Dr. Coley was a surgical oncologist who developed the mixture in the late 19th century as a treatment for cancer who cured many cases of cancer. Boosting the immune system to attack cancer cells is called immunotherapy. Many researchers around the world are looking into immunotherapy and a lot of progress has been made in this field since Coley's time. It possible to use Coley's vaccine to induce fever at the same time stimulating the immune system to attack the TB bacteria tuberculosis bacteria infected phagocytes. No one has tried. We have used this vaccine in the treatment of cancers. The latest case I developed protocol was massive renal cell carcinoma with metastasis in the inferior vena cava. The patient is physician in US, still alive and free of cancer. Can we use this vaccine to treat tuberculosis? I do believe we can. It is easy to prepare, inexpensive, and it may act by boosting the immune system to attack TB bacteria and macrophages with the bacteria. It acts as immunotherapy and hyperthermia. Many researchers around the world are looking into immunotherapy and a lot of progress has been made in this field since Coley's time, it need to be tried in latent TB and if possible in active cases.

Ozone Therapy as Adjuvant Therapy in the Treatment of TB to Create More Reactive Oxygen Species (FIG. 9):

Another therapeutic agent that should be considered to treat tuberculosis is ozone therapy along with Vitamin C and anti-tuberculosis drugs. The German chemist Christian Friedrich Schönbein (1840), of the University of Basel in Switzerland is regarded as the Father of ozone therapy. When he passed an electrical discharge through water, a gas with strange smell was produced, which he called Ozone, derived from the Greek word ozein which means odor; the systematic names are 1Δ1,3Δ1-trioxidane and catena-trioxygen. In 1857 Joachim Hänsler, a German physicist-physician, along with German physician, Hans Wolff, developed the first ozone generator for medical use. Dr. C. Lender in 1870, for the first time applied Ozone into medical field. Dr. E. A. Fisch, a German dentist was said to be the first dentist to use ozone in his dental practice in Zurich in 1950 for many dental afflictions,

Ozone is inexpensive, easily produced (FIG. 9 #4) in the clinics and adopted to treat tuberculosis and other ailments of every kind. Ozone is a chemical compound consisting of three Oxygen atoms O3 (triatomic oxygen), a highly unstable, energetic form of normal (diatomic) atmosphere oxygen O2. At room temperature, O3 is a colorless gas with a characteristic odor. Besides after lightening, ozone can be produced artificially for medical use (FIG. 9 #4) and by UV rays of Sun (FIG. 9 #1a). Ozone protects living organisms by surrounding the earth at altitudes of 50,000-100,000 feet from the harmful rays of sun. One molecule of ozone is equal to 3000-10,000 molecules of chlorine and kills pathogenic organism's 3500 times faster (Filippia A. 2001).

In 1856, just 16 years after its discovery, ozone was first used in a health care setting to disinfect operating rooms and sterilize surgical instruments (Stoker, George 1916). By the end of the 19th century the use of ozone to disinfect drinking water of bacteria and viruses was well established in mainland Europe (Chemical Technology Encyclopedia; Barnes & Noble 1968 vol 1 pp 82-3. Suchkov B P June 1964). Study of the Ozonization of Drinking Water Containing Pathogenic Bacteria and Viruses, Gig Sanit (in Russian) 29: 22-9. PMID 14235449). In 1892 The Lancet published an article describing the administration of ozone for treatment of tuberculosis (Administration of Ozone in the Treatment of Phthisis, Lancet II: 1180-1181). Ozone was used during the First World War to disinfect wounds. The use of Ozonized fluids in dental care is impending. Extensive research has been carried out over the past 50 years into the use of Ozonized fluids for infection control and wound management and is proposed as an alternative oral antiseptic in dentistry (Srikanth et. al. 2013, Jyoti et. al. 2013). There are many known action of ozone on human body such as immunostimulating, analgesic, antihypoxic, detoxicating and antimicrobial properties. Hence it can be easily used in dental care and procedures for eliminating the infected and demineralized dental tissue and hence conserves and protects the tooth structure as described in excellent review article by Jyoti et. al. In the same fashion, it can be used in irrigating cold abscess. Ozone is used for every ailment we know of—dozens of them. Thousands of patients are treated with it and hundreds of traditional and alternative medical practice physicians continue to use it as a complementary health care therapy especially in Germany, Russia, Cuba, and some in US.

In the normal arrangement, oxygen (O2) has 2 atoms of oxygen; ozone is comprised of 3 atoms of oxygen (FIG. 9, O3). Ozone does not stay in that tri-atomic state long and unless held in check or bound by other molecular couplings, ozone breaks down from O3 to O2+O1 within 20 minutes (at atmospheric pressure) releasing —O₁ and ¹O₂ singlet oxygen atoms. Singlet oxygen is an inorganic chemical in an excited state of the dioxygen molecule with the chemical formula O2(a¹Δ_{g}), also written O₂*. Ozone has a short life, and is highly reactive with just about any substance that should NOT be in the human body including all pathogens (virus, bacteria, cancers, etc.) and synthetic compounds or their metabolites such as drugs and their metabolite residues.

Ozone has been administered to the rectum, intramuscular (in a muscle), into vagina, into the joints, ears, dental care (such as caries and periodontis), subcutaneously (under the skin), or intravenously (directly into veins) to treat an endless list of diseases. When infused into human blood, ozone produces reactive oxygen species (ROS) or free radicals (Bocci V, Valacchi G, Corradeschi F et al. 1998. Dodwad V, et. al. 2011). Ozone can be applied to the body in a number of ways. The Germans have a long history of applying ozone in a therapy known as autohemotherapy. We recommend the autohemotherapy with ozone for treating tuberculosis (and leprosy also). We have used it to treat cancers by autohemotherapy. We also have instilled inside the inside the prostatic urethra and bladder to treat prostate hypertrophy and cancers, prostitutes, bladder cancers, cystitis, and interstitial cystitis by insufflations through a catheter without complications (Shantha, Unpublished data 2005) with excellent therapeutic results.

It is important to note that an over-abundance or excess use of ozone is known to cause oxidative stress and cell damage. One should not become carried away with ozone therapy—the person should know how much to use, how long to use, how to deliver, when to use and the concentration of ozone delivered per ml. High levels of inhaled ozone are known to be toxic (Bocci, V. et. al. 1998, 2002, 2009). Serious complications reported from the improper use (Ernst E. 2001), hence we are against inhaling plain ozone with breathing. We have used ozone to treat various diseases without a single complication for more than a decade. The user should remember that it is not a cure all (panacea) universal therapy and needs knowledge to use it.

How does ozone work and its potency are not well established? Ozone improves the transportation of oxygen in blood, which results in change of cellular metabolism-activation of aerobic processes (glycolysis, Krebs cycle, 3-oxidation of fatty acids) and use of energetic resources. Ozone improves the metabolism of inflamed tissues by increasing their oxygenation and reducing total inflammatory processes (Seaverson K, et. al. 2010). As a response to this activation through ozone, the body's immune cells produce special messengers called cytokines (including important mediators such as interferons and interleukins). These inform other immune cells, setting off a cascade of positive change throughout the immune system, which is stimulated to resist diseases. This means that the application of medical ozone is extremely useful for immune activation in patients with a low immune status and/or immune deficit such as tuberculosis. Small quantities of ozone applied in what is called “major autohemotherapy” (intravenous treatment of the patient's blood) consequently activate the body's own antioxidants and free radical scavengers. Ozone is also said to amplify the effect of drugs and supplements—due to increased cellular absorption of these therapeutic agents.

Antibodies, Ozone and Hydrogen Peroxide for Treatment of Tuberculosis:

Antibodies are secreted proteins produced by varieties of immunity producing white blood cells that are designed to recognize and eliminate a wide range of foreign pathogens and substances. After a bacterium, virus, or other pathogen are denatured by ozone, the antigens enter the immune system and produce antibodies against the offending pathogen. The antibodies enter the bloodstream, target antigens—proteins, fat molecules, and other pieces of the pathogen—specific to that foreign invader. These antibodies then alert the immune system to the presence of the invaders and attract lethal “effector” immune cells to the site of infection. Antibodies do this by producing the oxidant hydrogen peroxide. Hydrogen peroxide is lethal to bacterial cells because it pokes holes in their cell walls (cell lysis), bursting the cells and killing them. Studies also suggest the antibodies also make Ozone which poke holes in the cell membrane.

All antibodies have the ability to produce hydrogen peroxide, but they need to first have available a molecule known as “singlet” oxygen—a highly reactive oxygen species—to use as a substrate. Ozone therapy provides this singlet oxygen. Singlet oxygen is an electrically excited form of oxygen that formed spontaneously during normal metabolic processes or when oxygen is subjected to visible or ultraviolet light in the presence of a sensitizer or ozone therapy. “Phagocytic” innate immune cells, like neutrophils, also produce singlet oxygen and are the most likely source of the substrate for antibodies, since during an immune response; antibodies will recruit neutrophils and other immune cells to the site of an infection. The neutrophils will engulf and destroy bacteria and other pathogens by blast with singlet oxygen and other oxidative molecules. The antibodies reduce singlet oxygen by combining it with water to produce hydrogen peroxide, producing ozone as well. A free radical is any atom or molecule which has one of its valences unsatisfied. The valence (or valency) of an element is a measure of its combining power with other atoms when it forms chemical compounds or molecules. This leaves it with an unpaired electron in its outer shell. In trying to get a partner for the lone electron, free radicals react avidly with any neighboring molecules, and so can theoretically do damage. Now we can see how the ozone therapy can be adopted to treat tuberculosis along with Vitamin C, hydrogen peroxide, and other anti TB therapeutic agents described here. There is a possibility that it can be effectively used by autohemotherapy and other such methods to curtail and cure Ebola infection according to premier scientist in this field, Dr. Robert Rowen of USA (Personal communication 2015). We advised the use of Vitamin C, hydrogen peroxide, and Ozone to treat Ebola virus infection at different intervals with insulin (Shantha T. R., Alternative Therapies to Cure and Curtail Ebola Viral Infection: 2015, under publication).

Ozone stimulates proliferation of immunocompetent cells, enhances synthesis of immunoglobulins, activates function of macrophages and increases sensitivity of microorganisms to phagocytosis. Ozone promotes the synthesis of biologically active substances such as interleukins, leukotrienes, and prostaglandins which is beneficial in reducing inflammation and wound healing especially in pulmonary TB.

It is important to note that singlet oxygen is highly reactive usually generated with a photo sensitizer pigment. The damaging effects of sunlight on many organic materials (polymers, etc.) are often attributed to the effects of singlet oxygen. In photodynamic therapy, singlet oxygen is produced to kill cancer cells. Various methods for the production of singlet oxygen exist besides Vitamin C, ozone, hydrogen peroxide such as the irradiation of normal oxygen gas in the presence of an organic dye as a sensitizer, such as Rose Bengal, methylene blue or porphyrins. For autohemotherapy (AHT): draw blood from the patient in a sterile IV glass bottle with anticoagulants (citrated or heparinzed blood), then mix (shaking gently up and down) it with a predetermined concentration (55-70 μg/ml) and quantity of ozone (25-50-100 ml), and then administer it through an intravenous catheter.

Contraindications for Ozone Therapy:

A 5.00 g oral dose of vitamin C will stop the effect of already administered ozone. As ozone counteracts the effects of Vitamin C, it should be used about 24-36-48 hours before or after the use of Vitamin C while treating tuberculosis or other diseases. Ozone cannot oxidize fluoride but can oxidize toxins—including mercury, allowing it to be sweated out through the skin pores. Direct inhalation of ozone through a mask is contraindicated because it can damage the alveoli. Though it cannot be used by mask to treat pulmonary tuberculosis, it can be used by insufflations through the delivery device described herein directly to the site of tuberculosis lesion (FIGS. 5, 6) avoiding the rest of the lungs. Other contraindications are: Pregnancy, active menses, recent internal bleeding, liver under stress, history of stroke, severe anemia, hyperthyroidism, patients on ACE inhibitors, thrombocytopenia, cardiovascular instability, acute alcohol intoxication, serious myasthenia, ozone allergy, and with fauvism (deficient G-6-PD enzyme). It can cause euphoria, rhinitis, cough, headache, nausea and vomiting, shortness of breath, blood vessel swelling, poor circulation, and at a times stroke. Since ozone's high oxidative power, all materials that come in contact with the gas must be ozone resistant, such as glass, silicon, and Teflon. We used only sterile glass bottle for autohemotherapy. Ozone intoxication the patient placed in the supine position, and treated with vitamin E and n-acetylcysteine.

Hydrogen Peroxide as Adjuvant Therapeutic Agents for the Treatment of Tuberculosis:

Hydrogen peroxide, chemically as H2O2, is a compound of hydrogen and oxygen. Hydrogen peroxide is converted to water and singlet oxygen (H2O2→H₂O+O−). This singlet oxygen located at the end of this reaction is a powerful oxidizing agent and is the active agent in hydrogen peroxide therapy. For IV therapy, hydrogen peroxide is infused into the circulatory system through a vein drip over a ninety-minute. Five cc (ml-milliliters) of pharmaceutical-grade, three-percent hydrogen peroxide are mixed with 500 cc five percent glucose solution. Two grams of magnesium chloride are added along with a small amount of manganese to prevent vein sclerosis. Taking hydrogen peroxide orally has a corrosive and tumorous effect on the GI tract and is advised against such route of use.

Hydrogen peroxide reacts with fatty acids, ascorbate and iron to form hydroxyl radicals (FIG. 3). It is a known fact that the TB bacterium has a fatty layer coating (acid fast) with which hydrogen peroxide can react. Hydroxyl free radicals are probably one of the major factors in many degenerative diseases, including cancer. Much of the body contains enzymes (catalase) that quickly break up hydrogen peroxide into water and oxygen. In the blood, H2O2 encounters enzymes catalase (that releases singlet oxygen immediately) and cytochrome-C. After forty minutes, the hydrogen peroxide that binds with cytochrome-C, begins to act like catalase and breaks down the hydrogen peroxide to water and singlet oxygen. By this time, the hydrogen peroxide/cytochrome-C complex has been spread throughout the body. In this way the benefits of hydrogen peroxide are made available to all cells.

The effect of singlet oxygen produced by like Vitamin C, ozone, anti TB drugs, hyperthermia by DNP, and hydrogen peroxide in the human body is twofold. It kills, or severely inhibits the growth of, bacteria, fungus, and viruses. (All viruses are anaerobic). The second effect of H2O2 and related therapies is that it provides singlet oxygen, which, in turn, transforms biological waste products and toxins into inert substances by oxidizing them. It doubles the rate of enzymatic metabolism in the mitochondria within each cell, thus enabling the body to cleanse itself of toxins. This increase in metabolism probably accounts for some of the antibacterial, antifungal, and antiviral effects of hydrogen peroxide. Many studies have shown that intravenous Hydrogen Peroxide therapy will kill bacteria, viruses, fungi, parasites and has also been shown to destroy certain tumors.

Intravenous Hydrogen Peroxide was first used by Dr. T. H. Oliver in 1920 successfully to treat patients during an epidemic of influenzal pneumonia which was published the results in the British Medical Journal, Lancet. 70 years later this old treatment was rediscovered by Charles Dr. Farr (1987) whose modern day research resulted in the scientific paper “Therapeutic Use of Intravenous Hydrogen Peroxide.” This paper is basis for the protocol used by most physicians who administer intravenous hydrogen peroxide without serious side effects. Hydrogen peroxide has been used by many alternative therapists on every disease known which the present patent presenter is opposed on many of them. Collins J 1989 is one of the crusaders for its use to treat various diseases.

Hydrogen peroxide is a part of normal metabolism and our bodies produces it constantly. Peroxisomes (also called microbodies) are organelles found in virtually all eukaryotic cell, said to be derived from endoplasmic reticulum, and contain oxidative enzymes, such as catalase, D-amino acid oxidase, and uric acid oxidase. These peroxisomes units in certain white blood cells produce H2O2. The white cells rich in peroxisomes engulf bacteria which cause disease and mix them together with these peroxisomes. The singlet oxygen from H2O2 produced by the leukocytes destroys the bacteria or virus. This happens naturally, without any help from outside sources of hydrogen peroxide. We believe that the use of hydrogen peroxide in the treatment of tuberculosis should be tried and careful protocol should be followed by those with proper knowledge and training in its use.

Ezetimibe Administration to Deny Needed Lipids to M. tuberculosis Bacteria:

Ezetimibe is (3R,4S)-1-(4-fluorophenyl)-3-[(3S)-3-(4-fluorophenyl)-3-hydroxypropyl]-4-(4-hydroxyphenyl) zetidin-2-one (Ezetimibe), an anti-hyperlipidemic medication which is used to lower cholesterol levels. It acts by decreasing cholesterol absorption in the intestine epithelial cells as well as in hepatocytes. U.S. Pat. No. 8,455,474 B2 by Lu and Tsai describe Ezetimibe, an anti-cholesterol drug capable of significantly inhibiting the survival and proliferation of Mycobacterium tuberculosis bacteria in the monocytes. Ezetimibe acts through stimulating CD13 leading to monocytes activation and thus bacterial killing of Mycobacterium tuberculosis, and partly through depleting the intracellular nutrition (lipids) necessary for the survival of Mycobacterium tuberculosis and it is in itself an anti-tuberculous antibiotic. Ezetimibe is capable of directly killing Mycobacterium tuberculosis outside cells according to this patent. We plan to use Ezetimibe in selected cases of tuberculosis in addition to the methods described herein as needed as we proceed with exploring new therapeutic approach.

During the past 3 years, monoclonal antibodies that inhibit proprotein convertase subtilisin-kexin type 9 (PCSK9) have emerged as a new class of drugs that very effectively lower LDL cholesterol levels (Sabatine M. S., et. al., NEJMoa1500858; Mar. 15, 2015D01: 10.1056). One of the members of this class is evolocumab, a human monoclonal antibody that achieves 60% reduction in LDL cholesterol levels on parenteral administration. This may have therapeutic implication in the treatment of tuberculosis akin to Ezetimibe with other therapeutic agents need to be explored when such agents are approved.

Clavulanic Acid Binding to TB Bacteria to Overcome the Resistance:

Clavulanic acid is a competitive inhibitor of β-lactamase, meaning that it ‘tricks’ the bacteria into binding to it instead of the ‘real’ antibiotic, thus allowing the antibiotic to overcome resistance. Such a possibility is explored by John S. Blanchard in US patent publication 2011/0190253 A1 for the treatment of M. tuberculosis. Despite the use of these combinations, some bacterial strains have emerged that are resistant to even such combinations. We plan to use IV infusion solutions of Augmentin which contain Clavulanic acid with other therapeutic agents to treat tuberculosis associated with or without other secondary infection.

Supplemental Oxygen and Hyperbaric Therapy (HBOT) to Increase the Production of ROS, Other Systemic Effects and Other Effects:

Many of the patients with lung affliction have low oxygen saturation. Hence we provide external source of oxygen by: Providing 100% oxygen is delivered by nasal canula or breathing or non-breathing face mask. It is simple to use and will raise the oxygen saturation of the blood as indicated by the pulse oxymeter. Further, the TB lung lesions are directly exposed to 100% oxygen instead of 21% oxygen from breathing room air facilitating the effectiveness of Vitamin C and other anti-tuberculosis drugs to kill the mycobacterium tuberculosis. It is simple to use by using external tank oxygen or through an oxygen generator.

The most efficient way to raise the tissue oxygen in the treatment of TB is hyperbaric oxygen therapy (HBOT). We are aware that the tuberculosis bacterium is an obligate aerobe and thrive where oxygen is high such as apex of the lungs, but high levels of oxygen are toxic to bacteria. It is breathing 100% oxygen while under increased atmospheric pressure in a pressurized closed chamber. The physics of HBOT lies within the ideal gas laws. Henry's law states that the amount of gas dissolved in a liquid is equal to the partial pressure of the gas exerted on the surface of the liquid. By increasing the atmospheric pressure in the chamber, more oxygen can be dissolved into the plasma (blood) than would be seen at surface pressure. By this method more oxygen rich blood is delivered to all the tissues including lungs which contain TB lesions. This will augment the effectiveness of Vitamin C and other therapeutic agents by using oxygen to produce reactive oxygen species (ROS) using the Fenton reaction which kills the mycobacterium tuberculosis (FIG. 3).

Benefits of hyperbaric and supplemental oxygen therapy are multiple such as Increased energy, stamina, and endurance of the emaciated TB patients; improves circulation, calms the nervous system and improves memory; accelerates healing of the TB lung lesion wounds. We have used HBOT to heal non healing post-surgery wounds and is used chronic non-healing wounds due to other injuries and diabetic ulcers. The caseous, cavitary granuloma and cold abscess of tuberculosis is nothing but a chronic wound that does not heal. Hence the HBO is very effective in the treatment of TB. It is used by deep sea divers and carbon monoxide poisoning routinely. I have used it to treat chronic leg wounds due to diabetes and trauma, multiple sclerosis, cancers and other chronic diseases with great successes. By this method we have saved many legs from amputation. Our treatment of cancers patient included HBOT under low dose chemotherapy in soft HBOT chamber with excellent results. Pressurized oxygen therapy also kills many infectious bacteria including tuberculosis, viruses, fungi and parasites. Hyperbaric oxygen therapy strengthens the immune system. The basic principal of hyperbaric oxygen therapy is to infuse red blood cells, plasma, lymph, cerebral spinal fluid and other tissues throughout the body with more oxygen and deliver to the site of tuberculosis infection. During Vitamin C therapy, at the middle of the infusion the patient placed in a multiplace chamber or in a monoplace or soft portable HBOT chambers. These soft vessels HBOT can be pressurized to 1.5-1.7 atmospheres absolute (ATA) and that is all we need in the treatment of TB with Vitamin C treatment instead of using steel-glass expensive immobile HBOT chamber. HBOT combined with Vitamin C and other anti-mycobacterium tuberculosis drugs can be effective in eradicating tuberculosis rapidly including the drug resistance TB.

Latent—Persister M. tuberculosis Bacterial Infection and TB Disease:

Not everyone infected with TB bacteria develops the disease and becomes sick. As a result, two TB-related conditions exist: latent TB infection and TB disease. With latent TB Infection, M. tuberculosis bacteria can live in the body without making the infected person sick. 90-95% of people who breathe in TB bacteria and become infected, the body is able to fight the bacteria to stop them from growing contributing to latent infection. People with latent TB infection do not feel sick, do not have any symptoms, and cannot spread TB bacteria to others. However, if TB bacteria become active in the body (due to any number of reasons including immune system dysfunction) and multiply, the person will go from having latent TB infection to being sick with active tuberculosis. Our method of treatment may eliminate this condition.

M. tuberculosis Bacterial (TB) Disease Presentation for Therapy:

M. tuberculosis bacteria become active when the mycobacterium tuberculosis enters the lungs (FIG. 8) from the other infected person, if the immune system can't stop them from growing and spreading. When M. tuberculosis bacteria grow in numbers and become active (multiplying in the body), this is called tuberculosis disease, producing symptoms such as cough with or without blood tinged sputum, fever, loss of appetite, emaciation, and night sweats with associated diagnostic signs and tests. They may also be able to spread the bacteria to people they spend time or come in close contact. 90 to 95% of the people who have latent TB infection never develop tuberculosis. Some people develop TB disease soon after becoming infected (within weeks) before their immune system can fight the TB bacteria or due to lack of mounting immune system response due to any number of reasons. For people whose immune systems are weak, especially those with HIV infection, malnutrition, and other debilitating diseases, the risk of developing tuberculosis from the latent or acute exposure to infection is much higher than for healthy people with normal immune systems.

It was discovered Mycobacterium tuberculosis secreted Cu, ZnSOD in. It has been found that antibodies which specifically bind this M. tuberculosis SOD are useful in detecting the presence of the bacterium. It has also been discovered that tuberculosis patients develop antibodies against the M. tuberculosis Cu, ZnSOD. Thus, a patient producing antibodies against M. tuberculosis Cu, ZnSOD is diagnostic for tuberculosis in that patient and also monoclonal antibodies can be used attack this bacteria which will bind specially to the M. tuberculosis SOD. Superoxide dismutases (SOD) have been classified based on the inorganic atoms they require for activity. Three SOD families have been identified: those requiring manganese (MnSOD), those requiring iron (FeSOD), and those requiring copper and zinc (Cu, ZnSOD). MnSODs have been found in mitochondria and rokaryotes, whereas FeSODs have been found in prokaryotes, primitive eukaryotes, and some plants. Cu, ZnSODs were originally found in eukaryotes and later found in several bacteria. Macrophages are an important arm of a vertebrate's immune system. Such cells can kill pathogens such as bacteria by engulfing the pathogen and bombarding it with superoxide radicals. Therefore, a secreted Cu, ZnSOD may play a role in the survival of bacterial pathogens, especially those known to survive and grow in macrophages (Wu et. al., 1998, U.S. Pat. No. 7,094,403 B2 by Lee and Wu).

Mycobacterium Tuberculosis Bacterial Infection Diagnostic Tests:

There are several tests available to diagnose tuberculosis, susceptible to anti TB drug treatment or are drug resistant. These are TB tests which can be used to determine if someone has: 1. Latent TB, which means that they are infected with TB bacteria. 2. Active TB. Even if a person has symptoms. The tests either have low sensitivity (the ability to correctly detect people with TB) and/or low specificity (the ability to correctly detect people who haven't got TB), If a TB test has low sensitivity, it means that there will be a significant number of “false negatives”, meaning that the test result is suggesting that a person has not got TB when they actually have. Similarly, a low specificity means that there will be a significant number of “false positives” suggesting that a person has TB when they actually haven't.

The Tuberculosis Skin Test:

Approximately 6 weeks after tuberculosis infection, the growth of tubercle bacilli to a certain extent all of a sudden ceases, the host becomes tuberculin positive and caseous necrosis take place. The interval from infection to tuberculin conversion is less than 8 weeks. The onset of caseous necrosis coincides with the development of acquired immune resistance or cell mediated immunity (CMI).

The TB skin test is a widely used diagnostic test and involves intradermal injection of tuberculin PPD (protein-purified derivative). The skin test involves injecting a small amount of fluid called tuberculin into the skin in the lower part of the arm. Antigen-specific T cell responses result in measurable induration at the injection site by 48-72 hours after injection, which indicates exposure to mycobacterial antigens. Then the person must return after 48 to 72 hours to have a trained health care worker look at their arm. The health care worker will look for a raised hard area or swelling, and measure its size. They will not include any general area of redness. TB skin test also cannot tell if the person has latent TB or active TB disease. Sensitivity and specificity have, is a problem with this test, because the individuals vaccinated with BCG cannot be distinguished from infected individuals because it is made up of attenuated M. bovis bacteria and takes about up to 12 weeks to heal at the site of injection. However, the safety and efficacy of BCG is a source of controversy and some countries, including the United States, do not vaccinate public.

The Mantoux TB test is the type of TB test most often used, although the Heaf and Tine tests are still used in some countries. None of these diagnostic TB tests will guarantee a correct result. Delayed cell mediated immunity to tuberculosis bacillus is measured by the Mantoux skin test. The test is done by using antigen “purified protein derivative” from media in which tuberculosis bacilli have grown. The skin test is positive if an area of induration and swelling at least 10 mm in diameter develops within 48 hours after the intradermal injection of the antigen. Patients develop this hypersensitivity reaction 2 to 4 weeks after infection.

Although a positive skin test indicates infection with tubercle bacilli, it is not necessarily associated with clinical disease. Most people with positive skin tests have immune systems that have confined and limited the infection. A positive skin test also develops after vaccination with Bacilli Calmette-Guerin (BCG). When BCG is used in an effort to control natural tuberculosis, the skin test is not reliable as an indicator of naturally acquired infection. Once positive, the person remains so for life.

The Interferon Gamma Release Assays (IGRAs):

They are a new type of more accurate TB test. In this context referring to an assay is simply a way of referring to a test or procedure. IGRAs blood tests that measure a person's immune response to the bacteria that cause TB. The immune system mounts a complex response to TB bacteria, and produces some special molecules called cytokines. These assays work by detecting a cytokine called the interferon gamma cytokine. They are performed in practice by taking a blood sample and mixing it with special substances to identify if the cytokine is present. Two IGRAs that have been approved by the U.S. Food and Drug Administration (FDA), and are commercially available in the U.S., are the QuantiFERON® TB Gold test, and the T-SPOT® TB test. The advantages of an IGRA TB test include the fact that it only requires a single patient visit to conduct the TB test, results can be available within 24 hours, and prior BCG vaccination does not cause a false positive result. Disadvantages include the fact that the blood sample must be processed fairly quickly, laboratory facilities are required, and the test is for latent TB. It is also thought that the IGRAs may not be as accurate in people who have HIV. In low prevalence resource rich settings, IGRAs are beginning to be used in place of the TB skin test. Serodiagnostic tests for TB are tests carried out on samples of blood, and they claim to be able to diagnose TB by detecting antibodies in the blood. However, testing for TB by looking for antibodies in the blood is very difficult and complicated and need special equipment and trained personal.

Sputum Smear Microscopy to Detect M. tuberculosis Bacteria:

It is a test for TB sputum smear stained using fluorescent acid fast stain and is being used to test for TB, (copyright CDC R W Smithwick). Sputum may be tinged with blood. Smear microscopy of sputum is often the first TB test to be used in countries with a high rate of TB infection. Sputum is a thick fluid that is produced in the lungs and the airways leading to the lungs, and a sample of sputum is usually collected by the person coughing.

Sputum smear microscopy is inexpensive and simple, and people can be trained to do it relatively quickly and easily. In addition the results are available within hours. The sensitivity though is only about 50-60%. For the diagnosis of TB several samples of sputum will normally be collected. Historically it has been recommended that three sputum specimens are collected on two consecutive days, but in 2007 the World Health Organization (WHO) recommended that just two specimens could be examined from consecutive days. Now it has been suggested that two specimens can be collected on the same day without any loss of accuracy. To do the TB test a very thin layer of the sample is placed on a glass slide, and this is called a smear. A series of special stains are then applied to the sample, and the stained slide is examined under a microscope for the presence of the TB bacteria.

Fluorescent Microscopy:

The use of fluorescent microscopy is a way of making sputum TB tests more accurate. With a fluorescent microscope the smear is illuminated with a quartz halogen or high pressure mercury vapor lamp, allowing a much larger area of the smear to be seen and resulting in more rapid examination of the specimen. In 2011 the World Health Organization issued a policy statement recommending that conventional fluorescence microscopy should be replaced by LED microscopy. It also recommended that in a phased way, that LED microscopy should replace conventional Ziehl-Neelsen light microscopy.

Mycobacterium tuberculosis Culture:

Using culture to test for TB Colonies of Mycobacterium tuberculosis growth on either solid media on culture plates (on Lowenstein-Jensen medium slant at 37° C. in a 10% CO2 humidified atmosphere) or bottles of liquid media (culture broths). Culture is much more complex and expensive than microscopy to perform as it requires specific equipment with enhanced laboratory facilities and can take weeks because of the slow growth of TB bacilli. We are developing a method to augment the growth of the M. tuberculosis bacteria in the culture much faster, and will be reported as the results are relevant to diagnose the disease rapidly.

TB Drug Susceptibility Tests:

Drug susceptibility testing means testing to find out which drugs the TB bacteria in a patient are susceptible to, and can therefore determine whether the person has got drug resistant TB. Some drug susceptibility tests, such as the Xpert TB test can be used to diagnose TB, as well as testing for some types of TB drug resistance.

Fluoroscopy and x ray of the chest are important tools to locate the site of TB lesion in lungs and bones and for early rapid diagnosis.

Standard Treatment of TB Using a Combination of Therapeutic Agents (FIG. 8a)

Standard treatment consists of four drugs given over six months. But for people infected with bacteria that have developed resistance to medication, treatment can take up to two years, and it may fail to cure or curtail the disease. As a general rule, the principles used for the treatment of pulmonary TB disease also apply to extrapulmonary forms of the disease. A 6-month treatment regimen is recommended for patients with extrapulmonary tuberculosis disease, unless the organisms are known or suspected to be resistant to the first-line drugs. Strains of drug-resistant TB bacteria appear faster than the time it takes to develop new drugs. With our inventive method, we can cut down the period of TB treatment to few weeks instead of months.

Drug-Resistant TB Disease can Develop in Two Different Ways:

Primary resistance disease occurs in persons who are initially exposed to and infected with resistant organisms.

Secondary resistance, or acquired resistance develops during TB therapy because of the patient being treated with an inadequate regimen, the patient not taking prescribed regimen appropriately, drug malabsorption, or drug to drug interactions leading to low serum levels.

“First-line” chemotherapeutic anti-tuberculosis agents used to treat M. tuberculosis infection that is not drug resistant includes isoniazid, rifampin, ethambutol, streptomycin and pyrazinamide.

“Second-line” chemotherapeutic anti-tuberculosis agents used to treat M. tuberculosis infection drug resistance to one or more “first-line” drugs include ofloxacin, ciprofloxacin, ethionamide, amino salicylic acid, Cycloserine, amikacin, kanamycin and capreomycin.

Standard treatment for TB usually includes a mix of four drugs (see tables below) over a period of six months and multi-drug resistant TB can take 18 to 24 months to treat. Since most of the disease is cleared in the first few months, people often do not finish their full regimen of TB drugs, which can lead to drug resistance bacteria development, making TB more dangerous and more difficult to treat. According to the World Health Organization, in some parts of the world, one in four people with TB has a form of the disease that can no longer be treated with standard drug cocktails.

If a person has latent tuberculosis, they may need to take just one type of TB drug for many months. Active tuberculosis, particularly if it's a drug-resistant strain, will require several drugs combination at once. The most common medications used to treat tuberculosis include: a. Rifampicin (Rifampin, Rifadin, Rimactane™); b. Isoniazid; c. Pyrazinamide; d. Ethambutol (Myambutolm™).

If the person has drug-resistant TB, a combination of antibiotics called fluoroquinolones and injectable medications, such as amikacin, kanamycin or capreomycin, are generally used for 20 to 30 months. Some types of TB are developing resistance to these medications as well. With our method of IV Vitamin C and with appropriate anti-tuberculosis therapeutic agents described below with insulin, even the resistance bacteria can be cleared in a short period, not 2 years. A number of new drugs are being looked at as add-on therapy to the current drug-resistant combination treatment including:

Bedaquiline

Delamanid

PA-824

Linezolid

Sutezolid

Management of Patients with Drug-Resistant (MDR) Tuberculosis is Based on the Following Guidelines:

A single new drug is not added to a failing regimen;

In patients with MDR organisms resistant to first-line drugs in addition to INH and RIF, regimens employing four to six drugs that are new to the patient and to which the isolate shows in vitro susceptibility appear to be associated with better results;

Patients with multidrug-resistant organisms should receive the highest priority for direct observation therapy (DOT), administered in the hospital, home, or other supervised clean facility;

The use of drugs to which there is demonstrated in vitro resistance is not prescribed,

Resistance to rifampicin is associated with cross-resistance to rifabutin and rifapentine (RPT) and be avoided;

There is no cross-resistance between streptomycin and the other injectable agents, amikacin, kanamycin, and capreomycin (although resistance to all may occur as independent events); cross-resistance between amikacin and kanamycin though rare can occur,

Resistance to Pyrazinamide (PZA) is uncommon. If mono-resistance (SRD) to PZA is observed, the TB is caused by M. bovis, not M. tuberculosis; Intermittent therapy should not be used in treating TB disease caused by drug-resistant organisms, except for injectable agents after the initiation phase (usually 2 to 3 months) of daily therapy.

With our inventive methods, we can attack the latent, persister, resistant, acute and any kind of mycobacterium tuberculosis in any part of the body.

Mode of Action of Commonly Used Anti-Tuberculosis Therapeutic Agents (FIG. 4) is as Follows:

Advancement toward the goal of shortening TB chemotherapy requires knowledge of the physiological state of drug tolerant bacilli. Though inhibition of mycolic acid biosynthesis is obvious within hours after the addition of INH, and RIF uptake occurs within minutes (Winder and Collins, 1970; Piddock, 2000); it is still not clear why a fraction of M. tuberculosis cells are not sterilized after eight weeks under these conditions in vivo (Wallis et al., 1999, Mitchison and Davies 2012). We hope our method of therapy described in this invention will sterilize TB bacteria which persist in spite anti-TB therapy. We believe that anti-tuberculosis medication and an enhancer of Mycobacteria tuberculosis respiration with DNP, Vitamin C with or without insulin, act as enhancer of respiration that can eliminate resistant cells which survive after 8 weeks in spite of therapy. It should be tested in vitro using DNP and Vitamin C with appropriate doses of anti-TB drugs. This will be one of the important research projects in finding the rapid cured for TB. The following are explanation of how the anti-TB drugs act to kill M. tuberculosis bacteria:

Streptomycin antibiotic stops bacterial growth by damaging cell membranes and inhibiting protein synthesis. Specifically, it binds to the (16S) 23S rRNA of the 30S subunit of the bacterial ribosome, which prevents the release of the growing protein (polypeptide chain) i.e. it inhibits the microbial protein synthesis. Humans have structurally different ribosomes than bacteria, thereby allowing the selectivity of this antibiotic for bacteria not for human cells. At low concentrations, it inhibits growth of the bacteria by inducing prokaryotic ribosomes to misread mRNA. Streptomycin also inhibits both Gram-positive and Gram-negative bacteria, and is therefore a useful broad-spectrum antibiotic. It is most likely that Kanamycin, Amikacin, and other derived from Streptomyces species exert their effect on the microbes similarly.

Rifampicin (RMP) is one of the most potent and broad spectrum antibiotics against bacterial pathogens and is a key component of the anti-TB therapy. Rifampicin diffuses into tissues, living cells, and bacteria, making extremely effective against intracellular pathogens like M. tuberculosis (Shinnick, 1996, Gumbo T. et. al., 2003, 2007, Rosenthal et. al., 2006). It acts by targeting and inactivating a bacterial enzyme called RNA-polymerase. The tuberculosis bacteria use RNA-polymerase to make essential proteins and to copy their own genetic information (DNA). Without this enzyme the bacteria cannot reproduce and they die. Rifampicin binds in a pocket of RNA polymerase β sub unit deep within the DNA/RNA channels, but more than 12 Å away from the active site. This indicates that this rifampicin inhibitors acts on RNA polymerase function by blocking the path of the elongating RNA when the transcript become 2 to 3 nucleotide in length. In nutshell Rifampicin inhibits bacterial DNA-dependent RNA synthesis by inhibiting bacterial DNA-dependent RNA polymerase. This drug can be safely used intravenously compared to other anti TB therapeutic agents; however the bacteria develops resistance to this drug with high frequency and we can reduce such occurrence by use of insulin induced hypoglycemia before administration. Using insulin induced hypoglycemia activated therapy (IHAT) can reduce or eliminate this resistance.

Isoniazid (INH):

The exact mechanism of action of Isoniazid is unknown, but it is thought to prevent tuberculosis bacteria from making substances called mycolic acids, which are needed to form the cell walls of the bacteria. INH is converted to an isonicotinyl-NAD adduct by the bacterial peroxidase katG. This adduct inhibits inhA, a key bacterial enzyme in the FAS II (fatty acidbiosynthesis) production of the cell wall mycolic acid. Resistance usually arises by mutation in katG, but less often in inhA, ahpC and ndh. INH is highly bactericidal against dividing bacteria. It also seems to combine with an enzyme that interferes with the cell metabolism of the bacteria. As a result of the disruption in its metabolism and without a cell wall, the bacteria die. The Thionamide may also act similarly.

Pyrazinamide (PZA) is an important sterilizing drug that shortens the duration of current tuberculosis (TB) therapy. However, the mechanism of action of Pyrazinamide is poorly understood because of its unusual properties of its requirement for an acid pH for activity and its sterilising activity. Pyrazinoic acid, the active moiety of Pyrazinamide, disrupted membrane energetics and inhibited membrane transport function in Mycobacterium tuberculosis by pyrazinoic acid and acid pH. Inhibitors of membrane energetics increased the anti-tuberculosis activity of pyrazinamide. When first discovered, it had activity in murine tuberculosis but no apparent in vitro activity, and its subsequent use in treatment depended largely on classic experiments at Cornell University, which showed its requirement for an acid pH for activity and its sterilising activity in the mouse. Recent studies have shown that PZA enters Mycobacterium tuberculosis by passive diffusion. We want to point out that insulin induced hypoglycemia described in this invention will enhance this diffusion, making it more effective anti mycobacterium tuberculosis drug. Once again the Vitamin C therapy creates acid media also, which will enhance the bactericidal effect of tuberculosis. Once it enters the bacteria, it is converted to pyrazinoic acid (POA) by nicotinamidase/pyrazinamidase (PZase) and is then excreted by a weak efflux pump. Protonated POA (HPOA) is reabsorbed into the bacilli under acid conditions (enhanced by Vitamin C and insulin described in this invention), and accumulates because the efflux pump is inefficient, causing cellular damage. Unlike other antibacterials, PZA has no defined target of action (Zhang and Mitchison 2003).

Fluoroquinolones:

The fluoroquinolones interfere with the action of the A subunit of bacterial topoisomerase which is responsible for supercoiling DNA and thus packing it within the cell useful in the treatment of MDR-TB.

Ethambutol (EMB-Myambutol™ and Servambutol™) is an inhibitor of cell-wall synthesis, and is a bacteriostatic against actively growing bacilli. Mycolic acids attach to the 5′-hydroxyl groups of D-arabinose residues of arabinogalactan and form mycolyl-arabinogalactan-peptidoglycan complex in the cell wall. It disrupts arabinogalactan synthesis by inhibiting the enzyme arabinosyl transferase. Disruption of the arabinogalactan synthesis inhibits the formation of this complex and leads to increased permeability of the cell wall. Thus kills it and also allows more anti TB drugs to enter the bacteria. It is useful in treatment of MDR-TB. There is a risk of developing optic neuritis during protracted continued treatment (Lim S A. 2006).

TMC207 (Bedaquiline, Sirturo™) demonstrated unique and specific anti-mycobacterial activity derives from inhibition of the proton pump of mycobacterial ATP synthase, a critical enzyme in the synthesis of ATP for M. tuberculosis energy needs. Binding of TMC207 to the oligomeric and proteolipic subunit C of mycobacterial ATP synthase (FIG. 4) leads to inhibition of ATP synthesis, which subsequently results in bacterial death. When this therapeutic agent is added to the triple therapy of isoniazid, rifampicin and pyrazinamide or substituted for any component of the triple therapy, R-207910 increased the effectiveness (Doggrell, 2005) of treatment in curtailing and curing the disease.

Cycloserine (4-amino-3-isoxazolidinone) is a drug sold under the brand name Seromycin™C. It is an antibiotic effective against Mycobacterium and acts by inhibiting cell-wall biosynthesis in bacteria (FIG. 4). As a cyclic analogue of D-alanine, cycloserine acts against two crucial enzymes important in the cytosolic stages of peptidoglycan synthesis: alanine racemase (Alr) and D-alanine:D-alanine ligase (Ddl). This antibiotic effectively leads to inhibition of peptidoglycan synthesis in the bacterial cell wall.

Injectable: Aminoglycosides and Capreomycin:

These injectable are particularly effective for treating MDR-TB disease. Aminoglycosides act to inhibit protein formation in the ribosomes of these, one is in the rpsL gene encoding the ribosomal protein S12 and another is in 16S rRNA, which interacts with S12.64. Capreomycin is a cyclic peptide antibiotic that binds across the face of 23S and 16S ribosomal fragments. They do not reach their possible potential, perhaps because their activity is greatly influenced by pH, being low in the mildly acid conditions of acute tuberculous inflammation and seems that it is less effective against intra-cellular bacilli than against extra-cellular bacilli.

RIFATER™ tablets contain three active ingredients, rifampicin, isoniazid and pyrazinamide. These are all antibiotics used to treat tuberculosis (TB) and work by killing the bacteria that cause the disease.

Two Drug Combination Taken Daily Orally

| | Milligrams (mg) | | milligrams (mg) |
|---|---|---|---|
| Isoniazid150 | + | Ethambutol | 400 |
| Rifampin60 | + | Isoniazid30 | |
| | 150 | + | 75 |
| | 300 | + | 150 |

Three Drug Combination Taken Daily Orally

| Rifampin in mg + | Isoniazid in mg + | Pyrazinamide in mg |
|---|---|---|
| 60 + | 30 + | 150 |
| 150 + | 75 + | 400 |
| 300 + | 150 + | 500 |

Four Drug Combination

| Rifampin + | Isoniazid + | Pyrazinamide + | Etambutol |
|---|---|---|---|
| 150 mg | 75 mg | 400 mg | 275 mg |

Below table shows the WHO recommended doses of the first-line and second line of anti-tuberculosis drugs.

Who Recommended Doses of the First-Lineanti-Tuberculosis Drugs

| | Daily Doses | | Thrice Weekly |
|---|---|---|---|
| Drugs | (Mg/kg) | Route | Dosage (mg/kg) |
| Isoniazid (H) | 5 (4-6) | Oral | 10 (8-12) |
| Rifampin (R) | 10 (8-12) | Oral/IV | 10 (8-12) |
| Ethambutol (E) | 15 (15-20) | Oral | 30 (25-35) |
| Pyrazinamide (Z) | 25 (25-30) | Oral | 35 (30-40) |
| Streptomycin (S) | 15 (12-18) | Parenteral | 15 (12-18) |

Who Recommended Doses of the Second-Line Anti-Tuberculosis Drugs

| | Daily Doses | | Maximum | |
|---|---|---|---|---|
| Drugs | (Mg/kg) | Route | Daily Dose | |
| Kanamycin (K) | 15 | IM | up to 1 | g |
| Amikacin (A) | 15 | IM | up to 1 | g |
| Ethionamide | 10-15 | Oral | up to 1 | g |
| Cycloserine (Cs) | 10 | Oral | up to 1 | g |
| Para amino salicylic acid (PAS) | 250 | Oral | up to 1 | g |
| Ofloxacin (Ofx) | 15-20 | Oral | 800-10000 | mg |
| Levofloxacin | 7.5-10 | Oral | 750-1000 | mg |
| Moxifloxacin | 7.5-10 | Oral | 400 | mg |

Pyrazinamide Usual Adult Dose for Active Tuberculosis:

The pyrazinamide works well under acid media and high dose Vitamin C provides such acid surrounding around the tuberculosis bacteria making it very effective anti-tuberculosis agent. It is given 15 to 30 mg/kg (up to 2 g) orally once a day in combination with three other antituberculous drugs for the initial 2 months of a 6-month or 9-month treatment regimen, until drug susceptibility tests are known. An alternate dosing regimen of 50 to 75 mg/kg (up to 3 g) orally twice a week may be used after 2 weeks of daily therapy to increase patient compliance. While administering Vitamin C mega dose, we advise the patient to take oral doses of Pyrazinamide double the indicated dose every other day if needed depending on the complications. This drug is preferred because the acid media provided by the large doses of Vitamin C make it more effective. Pyrazinamide is contraindicated in patients with severe hepatic disease and with acute gout. The CDC, The American Thoracic Society, and the Infectious Diseases Society of America suggest the following Pyrazinamide dosing based on lean body weight:

| Daily | Daily | Twice | Thrice |
|---|---|---|---|
| dosing: | dosing: | weekly dosing: | weekly dosing: |
| 40 to 45 kg: 1000 mg | 40 to 45 kg: 1000 mg | 40 to 55 kg: 2000 mg | 40 to 55 kg: 1500 mg |
| 56 to 75 kg: 1500 mg | 56 to 75 kg: 1500 mg | 56 to 75 kg: 3000 mg | 56 to 75 kg: 2500 mg |
| 76 to 90 kg: 2000 mg | 76 to 90 kg: 2000 mg | 76 to 90 kg: 4000 mg | 76 to 90 kg: 3000 mg |

Usual Adult Dose for Latent Tuberculosis:

A public health expert should be consulted prior to the use of the combination regimen with rifampin. 15 to 20 mg/kg, based on actual body weight (lean), orally once daily (maximum 2 g) for 2 months. Alternatively, a dosage of 50 mg/kg may be administered orally twice-weekly (maximum 4 g). Streptomycin is used as injectable daily for 5 days a week during the intensive phase of therapy with Vitamin C during first four to eight weeks of treatment. We gave 20 doses over the period of 4 weeks carefully monitoring its ototoxicity effects. Instead of streptomycin, in 50% of the cases rifampicin high dose IV administration conducted as described below combined with Vitamin C.

Treatment of Multi Drug Resistant (MDR) Tuberculosis:

An estimated 50 million people are presently infected with Mycobacterium tuberculosis resistant to both first-line drugs isoniazid and rifampicin (WHO, Fact sheet No. 104, March 2007. U.S. Pat. No. 8,912,329 B2). Ethionamide (2-ethylthioiso-nicotinamide, 2-ethylpyrimidine-4-carbothioamide), a structural analogue of isoniazid, is currently the last line of defense in the treatment of multi-drug-resistant tuberculosis (MDR-TB). Up to 1.00 gram a day dose is needed which is associated with severe side-effects including neurotoxicity and fatal hepatotoxicity. WO 2008/003861 describes compounds having a potentiating effect on the activity of ethionamide in the treatment of tuberculosis and related diseases. Such compound is 2-phenylethyl butyrate, a licensed food additive, and related compounds potentiate the activity of thioamides or thioureas, e.g. ethionamide, in the treatment of tuberculosis (WO 2009/080432). We want to use Insulin a natural biological hormone for potentiating on the resistant TB bacteria instead of food addictive's to augment the effect of ethionamide. The method we describe here in using Vitamin C, rifampicin and with insulin as described will overcome the MDR-TB without or minimum toxicity. Ethionamide is used as part of a South Africa's standard regimen to treat MDR TB. It has been proposed for use in combination with gatifloxacin (WHO Model List of Essential Medicines” (PDF). World Health Organization. October 2013. Retrieved 2 Feb. 2015).

Application of alternate electric current of different frequencies around the chest wall (as described above, FIG. 11, 12) will also facilitate in killing these multidrug drug resistant tuberculosis bacteria (MDR). By this method a biocompatible field guide is positioned between the surface of the chest wall and the target region beneath the skin surface. Electrodes are positioned on either side of the field guide, and an AC voltage with an appropriate frequency and amplitude is applied between the electrodes so that the field guide routes the electric field to the target region, in this case tuberculosis lesions in the lungs (FIG. 11) and extrapulmonary lesions.

Testing for Resistance:

To test drug sensitivity and drug resistance is very difficult due to waxy coat of the mycobacterium tuberculosis. The immune system “walls off” the TB bacilli which, protected by a thick waxy coat, can lie dormant for years as latent TB. In recent years drug resistant tuberculosis testing has shown a lot of progress. Some studies have found an in-house assay that could rapidly detect resistance to drugs involved in the definition of XDR-TB directly from smear-positive specimens. The assay is called Reverse Line Blot Hybridization Assay also known as RLBH. The study showed that the results of RLBH were as accurate as other drug susceptibility tests, but at the same time didn't take weeks to get results. RLBH testing only took 3 days to determine how resistant the strain of bacteria was. The current research has shown progress in the testing of drug resistance. A recent study found that a research technique known as direct nitrate reductase assay (D-NRA) showed efficient accuracy for the rapid and simultaneous detection of resistance to isoniazid (INH), rifampicin (RIF), kanamycin (KAN) and ofloxacin (OFL). D-NRA results were obtained in 16.9 days, comparably less than other drug susceptibility testing, a low-cost technology, easy to set up in clinical laboratories and suitable to be used for DST of M. tuberculosis in all smear-positive samples.

Antibiotics with Vitamin C Therapeutic Agents to Treat Multi Drug Resistant (MDR) Tuberculosis:

There are three other injectable used as second-line drugs (amikacin, kanamycin, or capreomycin) administered by injection to treat MDR TB. In our study with Vitamin C in MDR TB we used Kanamycin and Amikacin—a semi-synthetic, aminoglycosides antibiotic—can be given by IV at 15 mg/Kg diluted in normal saline or 5% dextrose.

Kanamycin is used as part of South Africa's standard regimen for treatment of MDR TB given 20 mg/kg up to 1000 mg by injection three times weekly up to 6 months if needed. We use it one gram given over a period of one hour intravenously to make it more effective in the treatment of TB of any kind after bringing down the blood sugar to 40 mg % with insulin injection. Amikacin can also be given IV at 15 mg/Kg diluted in normal saline or 5% dextrose can replace the streptomycin or kanamycin in the same fashion. Single daily dosing of aminoglycosides is feasible because of their rapid concentration-dependent killing and reduced post-antibiotic effect has the possibility for decreased toxicity. Hence the single daily dosing of aminoglycosides (examples: streptomycin, kanamycin, Amikacin, neomycin, netilmicin, dibekacin, tobramycin, gentamicin, sisomicin, and others) are safe, efficacious and cost effective.

Aminoglycosides like kanamycin “irreversibly” bind to specific 30S-subunit proteins and 16S rRNA. Specifically Kanamycin binds to four nucleotides of 16S rRNA and a single amino acid of protein S12. This interferes with decoding site in the vicinity of nucleotide 1400 in 16S rRNA of 30S subunit. This region interacts with the wobble base in the anti-codon of tRNA. This leads to interference with the initiation complex, misreading of mRNA so incorrect amino acids are inserted into the polypeptide leading to nonfunctional or toxic peptides and the breakup of polysomes into nonfunctional monosomes. Similarly, Amikacin (formerly Apothecon's, Amikin) an aminoglycoside antibiotic used to treat different types of bacterial infections works by binding to the bacterial 30S ribosomal subunit, causing misreading of mRNA and leaving the bacterium unable to synthesize proteins vital to its growth. Capreomycin (Capastat™) is a peptide antibiotic, commonly grouped with the aminoglycosides, which is given in combination with other antibiotics for MDR-tuberculosis. Adverse effects include nephrotoxicity and 8th cranial auditory vestibular nerve toxicity. The drug should not be given with streptomycin or other drugs that may damage the auditory vestibular nerve. Patients on this drug will often require audiology tests.

Sutezolid (PNU-100480, PF-02341272) is an oxazolidinone antibiotic currently in development as a treatment for highly drug-resistant tuberculosis in South Africa.

Capreomycin is a peptide antibiotic, commonly grouped with the aminoglycosides, which is given in combination with other antibiotics for MDR-tuberculosis. Adverse effects include nephrotoxicity and ototoxicity of cranial auditory vestibular nerves. Capreomycin is frequently used to treat Mycobacterium tuberculosis infections. Mycobacterium tuberculosis growth has been found to be inhibited at a concentration of 2.5 μg/mL It is administered by intramuscular injection into a muscle or infused into a vein over 1 hour once a day for 2 to 4 months then reduced to 2 or 3 times a week depending on the patient's condition and response to treatment, or use as directed by their doctor. Dosage is based on medical condition, kidney function, and response to treatment. The drug should not be given with streptomycin or other drugs that may damage the auditory vestibular nerve. Patients on this drug will often require audiology tests.

TMC 207: In patients who had newly diagnosed multidrug-resistant pulmonary tuberculosis to receive either TMC207 400 mg daily for 2 weeks, followed by 200 mg three times a week for 6 weeks. The primary efficacy end point was the conversion of sputum cultures, in liquid broth, from positive to negative with alleviation of TB signs and symptoms. It is important to note that the use of insulin augments the effectiveness of the therapeutic agents even more with rapid curtailing and cure of TB, drug resistant or not.

It is important to note that due to insulin induced mild hypoglycemia, the anti-tuberculosis therapeutic agents becomes hundreds of time more powerful than usual resulting in their destruction M. tuberculosis bacteria, no matter how resistant they are. That is why we are combining Ethionamide, a structural analogue of isoniazid, can be substituted for rifampicin with insulin, and Vitamin C.

Reduce Ototoxicity of Some of the Anti-Tuberculosis Drugs with Vinopocetine, Bioflavonoids and Insulin Ear Drops and Oral Administration:

This can result in bilateral sensorineural hearing loss, disequilibrium, or both; may be reversible or irreversible and over 200 medications cause it including streptomycin. We were able to reduce the effect on the nerve deafness by daily administration of Insulin compounded with bioflavonoid, folic acid, vinopocetine and riboflavin with EDTA compounded external ear drops on both the ears two to three times a day. Vinpocetine 2 mg/kg prevented hearing loss induced by the aminoglycoside antibiotic amikacin 450 mg/kg in guinea pigs (Sitges M, Nekrassov V. Vinpocetine prevents 4-aminopyridine-induced changes in the EEG, the auditory brainstem responses and hearing. Clin Neurophysiol. 2004; 115: 2711-2717). Clinical data Details of the study are limited, but Cavinton prevented neurosensory hypoacusis in 118 tuberculosis patients (17 to 63 years of age) who had normal hearing or hearing problems (Maliavina U S, Ovchinnikov I U M, Fasenko V P, Maliev B M, Kalinina M V, Dadasheva B B. Cavinton prevention of neurosensory hypoacousis in patients with different forms of tuberculosis [in Russian]. Vestn Otorinolaringol. 2003; (3):35-40). We have used the ear drops with vinopocetine compounded 3-4 times a day effectively to prevent the development ototoxicity. The combination method was effective in reducing the toxicity of therapeutic agents during the chemotherapy treatment of cancer patients. It did reduce the degree of Ototoxicity related deafness that can result from the use of this therapeutic agent. A number of streptomycin treated TB patients were found to develop irreversible cochlear and vestibular dysfunction by use of Streptomycin therapeutic agent. Other therapeutic agents that can have the same effect include kanamycin and aminoglycosides based antibiotics listed above, platinum-based antineoplastic agents, salicylates, quinine, and loop diuretics. It is bilateral sensorineural hearing loss with tinnitus. Start using the vinopocetine and flavonoid ear drops with insulin before the start of the therapy three times a day 24 hours before starting the therapy.

Monitoring Progression of Treatment of TB Patients Before, During, and after the Treatment:

All active TB diseases should be monitored at least monthly by a physician to evaluate the clinical response to therapy and to monitor side effects of medications. This includes baseline laboratory studies, TB medication regimens, imaging methods such as chest X rays, and monitoring of adverse reactions.

Test for Bacteriologic Sputum Conversion:

Patients with sputum cultures positive for M. tuberculosis should have 3 adequate morning sputum cultures obtained every month until sputum cultures convert to negative. A final sputum culture is obtained at the completion of successful treatment as a reference culture. Sputum cultures positive for M. tuberculosis after 2 months of drug treatment indicate ineffective therapy. Those failing to convert sputum cultures within 2 months, repeat drug sensitivities tested.

Chest X Ray Radiographic Monitoring:

CXRs should be obtained at baseline, at the completion of therapy, and during treatment (when clinically indicated). Patients with suspected pulmonary TB and negative sputum cultures at 2 months are tested by repeat CXR at that time. CXR improvement also points to us that the treatment effective and is indicative of culture-negative TB.

Monitor Drug-Induced Hepatitis:

Three of the first line TB therapeutic agents (INH, RIF, and PZA) can induce liver injury. Liver transaminases and other liver tests are in order. Symptoms of hepatitis (nausea, vomiting, abdominal pain, fatigue) are reviewed as they appear and therapeutic agents stopped if they occur. Monthly monitoring of liver enzymes is done on all patients. At any point liver transaminases are greater than 3 times normal (with symptoms) or greater than 5 times normal (without symptoms), hepatotoxic anti TB drugs should be stopped immediately and the patient evaluated carefully. Screening tests for HAV, HBV, and HCV infections should be obtained in non-immune patients. Once the liver enzymes return to normal, the person should be restarted with TB medications.

Monitoring TB Drug Toxicities:

Baseline complete blood count, platelets, and uric acid are obtained in addition to liver function tests. Thrombocytopenia can be associated with Rifampin; Elevated uric acid can occur with pyrazinamide, Ethambutol can result in visual acuity (Snellen) and red-green color vision (Isihara-color perception test) because of the risk of optic neuritis. For patients on prolonged treatment with ethambutol, optometry or ophthalmology evaluations are done three times monthly. Baseline and monthly creatinine and audiograms are indicated for inmates receiving streptomycin or other aminoglycosides, due to the risk of nephrotoxicity and ototoxicity.

Side Effects of Anti-Tuberculosis Therapeutic Agents:

Serious side effects of TB drugs aren't common but can be unsafe when they do occur. All anti-tuberculosis medications are toxic to the liver except Vitamin C. When taking these medications, call your doctor immediately if you experience any of the following signs and symptoms: nausea or vomiting, loss of appetite, a yellow color to your skin (jaundice), dark urine, fever that lasts three or more days, and has no obvious cause.

The following table labels some important adverse events of therapeutic agents and we watch for development of any of these adverse effects.

| DRUG | ADVERSE EVENT/CONTRAINDICATION |
|---|---|
| Isoniazid | Peripheral neuropathy |
| Pyrazinamide | Hepatitis |
| Rifampin | Hepatitis, GI reactions |
| Streptomycin | Deafness, Contraindicated during pregnancy |
| Thiacetazone | Severe skin rash among HIV +ve (not recommended) |

It is crucial to finish the full course of therapy and take the medications as prescribed. After a week or two, the patient will not be contagious and feel better. If the person stops the treatment too soon or skips doses, it can allow the bacteria that are still alive multiply and become resistant to those drugs, leading to TB that is difficult to treat. To help people stick with their treatment, a program called directly observed therapy (DOT) is recommended. In this approach, a health care worker administers medication so that the patient does not have to remember to take it on their own. With our therapy described in this invention, the patients become sputum negative within one to two weeks and cured of the disease in less than 6 weeks.

Methods of Preparation of the Patient, Compounding of Vitamin C and Administration of Anti-Tuberculosis Therapeutic Agents Based on Our Inventive Methods Describe Above:

Based on the above descriptions, we describe below the method of treating all kinds of tuberculosis infection, latent, active and resistant cases. We select the patients who are diagnosed with sputum test and X-ray examination. We will present the treatment protocol to the group of experts in the field (anti TB board) and/or the approval of intuitional review board (IRD). The following tests are performed before the treatment begins. The patients are admitted to outpatient facility or short stay hospital facility.

Explain to the patient about the treatment and obtain a consent form to proceed with the method of treatment.

Patient gets complete physical examination

Patient blood panel is drawn, and plasma iron levels measured. ESR increased with increased lymphocyte count in TB patients.

Patients complete blood work is done including liver and kidney function tests, liver enzymes,

Mountoux tuberculin skin test performed on all patients.

Patient sputum tested for Acid fast Mycobacterium tuberculosis bacteria by Ziehl-Neelsen staining,

ELISA (enzyme-linked immunosorbent assay) test for mycobacterium tuberculosis antibody (60-80% sensitive), PCR test (40% sensitivity).

Culture takes 4-6 weeks, only 40% positive.

IFN release assay (IGRAs) measures T-Cell release IFN in the response to stimulation with highly specific tuberculosis antigens ESAT6 & CFP₁₀

Chest x-Ray is taken both AP and Lateral views

If there is a boney lesion suspected, an x-ray, bone or body scan is performed at the areas of suspect (i.e. localized pain, lymph adenopathy, local swelling, suspected cold abscess, bone deformity, arthritic changes).

The patient comes to the clinic on an empty stomach.

An IV catheter in a vein and fluid containing normal saline or lactated ringer is started.

An EKG is hooked up and preliminary recording is done.

Patient's finger is hooked up to the pulse Oxymeter

Blood pressure is monitored every 30 minutes during the procedure.

Finger stick is done to measure the blood glucose levels The clinic will have facilities to check blood sugar levels through the finger sticks 15 minutes after insulin and after one hour or as needed depending upon the exhibited symptoms.

The clinic will have emergency CPR equipment and therapeutic agents to be administered during resuscitation if needed.

Supplemental oxygen delivery system provided with a mask, nasal canula or non-rebreathing mask during the therapy to increase alveolar-lung oxygen concentration to 100%. This will provide the extra oxygen and at the same time will facilitate increased output of ROS due to increased oxygen content of the lung alveoli with TB lesion by Vitamin C.

### Examples

### Promising Conjectural Cases Stories

The methods and therapeutic agents used in the treatment of tuberculosis in humans are described here and follow the examples of existing protocols with new protocol. There are 10 drugs currently approved by the U.S. Food and Drug Administration (FDA) for treating TB. Of the approved drugs, the first-line anti-TB agents that form the core of treatment regimens include the following combinations and doses. All the TB patients were put on oral Vitamin D3, 5000 IU a day besides multivitamin supplements.

### Example 1

A group of 6 patients were selected for the study. They are close to each other in age and the pulmonary TB condition. They were all treated identically and no one dropped out of the study.

Week 1.

These are 28, 35, 38, 45, 46 and 49 years old male and female patients (2 female and 4 male patients). Fairly healthy, not emaciated. Developed chronic cough, night sweats and wasting. Their physical examination showed lung pathology indicating possible tuberculosis lesion. Their sputum was positive for acid fast bacillus. Chest X ray showed apical lobe, or middle lobe granuloma TB lesion.

EKG, BP, and Oxymeter were hooked up. All their vital signs with blood pressure were taken and recorded.

Patients take the above prescribed therapeutic agents for TB as indicated.

IV infusion catheter was inserted and maintained a week at a time at each arm and kept open with 500 ml normal saline.

Inject one gram of streptomycin intramuscularly as soon all the vital signs are recorded. Rifampicin can be replaced for streptomycin.

Blood sugar was tested by using finger stick. We administered 5-10 units of homolog (rapid acting) insulin subcutaneously or IV slowly depending on the blood sugar levels. The glucose was checked 10 minutes later. It has dropped to ±40 mg %.

500 ml of ringers lactate was mixed with 25 grams of soluble Vitamin C will give us 5% solution (prepared in 500 ml normal saline, ringers lactate, Locke-ringers) Administer it intravenously slowly over a period of 4-6 hours.

Once the infusion of Vitamin C was completed, continue the IV infusion of normal saline.

Test the blood sugar by finger stick, and if it low (below 70 mg %) administer 50-100 ml of 15% dextrose. Ten minutes after, test the blood sugar level, and make sure it is normal.

Once the vital signs are normal, the patient is discharged with instructions to come next day.

Continue the therapy for five days and give rest for two days.

Week 2

The patients were asked to collect the sputum in a sterile plastic cup and asked to bring it in. It was prepared for acid fast stain and examined under microscope for the mycobacterium tuberculosis. Examination showed reduced number of bacteria in the sputum. They were not rod shaped as seen in the first stain. They were deformed indicating they are having the effect of the Vitamin C therapy and other therapeutic agents. The sputum was sent to the pathology lab to culture. Routine vital signs were taken and therapy as described above started and continued to the end of the procedure. It was repeated for another 5 days.

Week 3

The patients came in the morning with sputum collected in a sterile container. It was stained and looked at under a microscope for any bacteria present. On careful examination of more than half a dozen slides, we could see possible presence of mycobacterium tuberculosis the slides. The bacteria were deformed and did not look as we saw them before starting the treatment. These are the reported cases of culturing the sputum:

All smear-positive specimens that grow M. tuberculosis are culture positive within two weeks of incubation, regardless of specimen type. We got the report that no mycobacterium tuberculosis could be grown at the end of the two weeks of incubation.

It is known that >95% of smear-negative respiratory specimens that grow M. tuberculosis are culture positive within three weeks of incubation.>95% of smear-negative non-respiratory specimens (bone, joint, CSF, skin, etc.) that grow M. tuberculosis are culture positive within four weeks of incubation. Specimen is unlikely to grow M. tuberculosis, but not that it will be negative for mycobacteria, by taking into account the duration of incubation and smear grade. One needs to consider that some mycobacterium that grow more slowly than M. tuberculosis include M. avium-intracellulare and M. xenopi. The patient treated for TB with a positive sputum smear is very unlikely to grow M. tuberculosis from the sputum after two weeks of incubation. That was the case in our study. A smear negative patient not treated for TB has less than a 5% chance of growing M. tuberculosis from sputum specimen is culture negative after three weeks of incubation.

Week 4:

All smear-positive specimens that grow M. tuberculosis are culture positive within two weeks of incubation, regardless of specimen type. All the protocol described in week 1 is followed. The sputum test showed lack of any TB bacteria. It did show numerous inflammatory white blood cells. The same regimen followed as done during week one.

Week 5 and 6:

The patient was given same treatment as described up to six weeks. Complete rest and no treatment after completion of six weeks of therapy. His sputum and chest X-ray were performed. The sputum was negative for TB bacillus and the chest X-ray showed the dissolution of the lesion.

Week 7—

24: The patients were tested for sputum, which consistently showed negative. The patient did not suffer from weight loss, cough, night sweats and gained weight. At the end of 8 weeks we declared the patient is cured of the disease. Chest X ray at the end of the 6 months showed complete resolution of the TB lesion from the lungs with occasional fibrosis in the lungs parenchyma probably due to tuberculosis inflammation. For patients with positive cultures at diagnosis, a repeat chest radiograph after 2 months of treatment is useful. A chest radiograph at completion of treatment and its comparison before the start of the treatment indicates the effectiveness and outcome of the therapy.

These groups of 6 patients were treated using this method with combination of high dose Vitamin C with appropriate anti TB therapeutic agents cure the tuberculosis within 6 weeks. Those with extensive pulmonary lesions need a much longer period of therapy than early cases. We are for providing oral administration of two anti-tuberculosis therapeutic agents for up to 4 months.

### Example 2

This is 25 year old young female patient present with all the signs and symptoms of TB. The preliminary test revealed the presence of mycobacterium tuberculosis bacteria in the sputum in large numbers. The chest X ray showed miliary TB affecting both lungs—in all the lobes (FIG. 5C #48). This patient was diagnosed as a patient with miliary tuberculosis. Miliary tuberculosis is the disseminated form of tuberculosis and is caused by seeding of the bacilli through lymphatic's and blood vessels to produce minute due to opening of the TB lesion to the BV mostly. They are presented as yellow-white lesions resembling millet seeds (hence miliary). She was isolated from the rest of the family members till the sputum became negative. Patient was admitted to the isolation ward in the hospital. The testing and monitoring is the same as described in example 1. The patient got one gram of streptomycin every day

The patient received the following combination of anti TB therapeutic agents for 3 weeks, the following protocol was followed:

Four Drug Combination

| Rifampin + | Isoniazid + | Pyrazinamide + | Etambutol |
|---|---|---|---|
| 300 mg | 150 mg | 500 mg | 275 mg |

The patient received 10% Vitamin C infusion in 500 ml of ringers lactate every day for 3-4 hours, following the induction of moderate hypoglycemia with use of rapid acting insulin.

On day 5, the IV Vitamin C was reduced to 7.5% and was continued for 4 more days,

On third week, the IV Vitamin C was reduced to 5% and was continued for 4 more days,

This method of IV Vitamin C was continued for next 3 weeks with other anti TB therapeutic agents.

The rifampicin given intravenously in high doses up to 600 to1200 mgs that eliminates the TB bacteria early from the sputum and lung lesions. This regimen was followed every day for 2 weeks and then every other day for the rest of her stay. This was given after reducing the blood sugar to 40 mg % to enhance its bactericidal effectiveness.

The patient's sputum became negative at the end of the first week of treatment. At the end of the 4th week, the chest x ray showed all the miliary TB lesions have disappeared. Night sweats, cough, chest pain, and fever were alleviated. The patient started gaining strength and weight.

The patient was discharged at the end of 6 weeks as cured. She was followed every 4 weeks for a total of six months. The health care professionals visited the family in their house setting every two weeks. They examined the rest of the family for any spread of the disease from this patient. Her sputum was tested every two weeks by slide preparation and staining.

The patient continues to receive oral 4 combination of anti TB therapeutic agents for six weeks along with streptomycin injections of 1 gram, five days week for 6 weeks during high dose administration of Vitamin C. She was put on oral two regimens anti-tuberculosis drugs for 4 months.

### Example 3

This 45 year old female has traveled to Southeast Asia and was diagnosed with left breast cancer. It was an early case with discrete lymph node metastasis. She has cough with blood tinged sputum, night sweats, and weight loss. Her chest X ray showed right upper lung mass and her sputum came back as positive for mycobacterium tuberculosis bacilli. By the time we made the TB diagnosis she was treated with high dose Vitamin C 10 to 25 grams in 500 ml normal saline every day for a total of 10 doses as an adjuvant therapy for cancer. She underwent local injection of anti-breast cancer chemotherapy therapeutic agents with insulin and local heat application with magnetic heat waves and hyperbaric therapy using soft air HBO chamber.

Her cough was completely disappeared by second week. After the lab came with mycobacterium tuberculosis, we wanted to see why the cough has gone with no sputum. Her chest X ray on third week showed disappearance of the lesion from the right lugs. We sent the sputum again, and came negative for mycobacterium tuberculosis after culture. This also made us to diagnose that the lesion in the lungs was not a metastatic breast cancer. The patient was treated with chemotherapy, local injection of breast mass and lymph nodes with high dose Vitamin C chemotherapy therapeutic agents. After completion of therapy, she was sent to surgery for the removal of the remaining lesion mass in the breast which had shrunk considerably, and axillary lymph node resection. She was seen 3 months after surgery. Her chest X ray was negative and so also sputum M. tuberculosis bacteria. She was free of cancer and tuberculosis.

We were surprised her TB lesion was gone and was cured of the condition—she was free of tuberculosis infection. We knew that Vitamin C produces hydrogen peroxide, along with ROS free radicals production by Fenton reaction, and this is one of the mechanisms of killing cancer cells. We deduced that the mycobacterium tuberculosis was killed by high dose IV Vitamin C due to production hydrogen peroxide along with ROS production in the TB lesion and its surrounding pulmonary tissue. This is the first reported case of tuberculosis treated and cured using high dose Vitamin C. The latest culture studies on the anti M. tuberculosis effects of Vitamin C have been recently reported supporting our clinical findings (Vilcheze, et. al. 2013).

### Example 4

In this group of 6 patients we plan to use high dose Vitamin C and rifampicin because of the safety margin of these therapeutic agents. Protease inhibitors used to treat AIDS interfere with rifampicin. We already know that Vitamin C is very safe in high doses from 25 grams up to 100 grams IV infusion as we have seen being used to treat cancer patients at various centers and in our clinic also.

How do the Rifamycins Achieve Selective Toxicity to M. tuberculosis and not to Human Cells?

RNA polymerase (RNAP), also known as DNA-dependent RNA polymerase, is an enzyme that produces primary transcript RNA. In cells, RNAP is necessary for constructing RNA chains using DNA genes as templates, a process called transcription. RNA polymerase enzymes are essential to life and are found in all organisms and many viruses including tuberculosis bacteria. Let us see how rifampicin attacks the RNAP in the M. tuberculosis bacteria, not normal cells? Fortunately, rifamycins do not bind to eukaryotic (healthy cells of our body) RNA polymerases, so our own cells can continue to transcribe genes normally even when we are taking these antibiotics which inhibit selectively the bacterial DNA dependent RNA polymerase in tuberculosis bacteria—a prokaryocyte (organisms without a cell nucleus (=karyon), or any other membrane-bound organelles). This is because the enzymes that carry out this reaction are bit different in eukaryocyte and Prokaryocyte, thus rifampicin do not attack our eukaryotes (organisms whose cells are organized into complex structures by internal membranes bound organelles such as mitochondria, ER, Golgi complex, nucleus, and a cytoskeleton) in our body.

Another advantage of using mega doses of rifampicin with insulin is that unlike some antibiotics that rifampicin can penetrate into all cells and tissues with ease including infected macrophages and M. tuberculosis bacteria within. The rifamycins can always cross cell membranes and get in and gain access to their target enzyme—in this case RNA polymerase in the tuberculosis bacterial cytoplasm-matrix (FIGS. 1, 4). The insulin augments and enhances the pharmacologic therapeutic effectiveness of rifampicin hundreds of times (Alabaster. et al. 1981, Shantha 2003). This antibiotic molecule is thought to bind to the polymerase in such a way that it creates a wall that prevents the chain of RNA from elongating to replicate. In the presence of rifamycins, bacteria can't transcribe any genes that they need to carry out their normal functions and multiply resulting in their death. Hence rifamycins are effective bactericidal anti-tuberculosis antibiotic.

Table below shows the Plasma Concentrations based on published studies (mean±standard deviation, mcg/mL) rifampicin mg/ml of plasma at different doses administered. We want to administer the maximum dose depending upon the condition of the patient and sputum positive or negative to kill the M. tuberculosis bacteria after inducing hypoglycemia with insulin.

Rifampin

| Dosage | | | | | |
|---|---|---|---|---|---|
| IV | 30 min | 1 hr to 2 hr | 4 hr | 8 hr | 12 hr |
| 300 | 8.9 ± 2.9 | 4.9 ± 1.3 | 2.5 ± 1.0 | 1 ± 0.6 | <0.4 |
| mg | | | | | |
| 600 | 17.4 ± 5.1 | 11.7 ± 2.8 | 6.4 ± 1.7 | 3.5 ± 1.4 | 1.2 ± |
| mg | | | | | 0.6 |
| 1200 | 34 ± 8.0 | 24 ± 5.0 | 12 ± 3.0 | 7 ± 2.5 | 2.5 ± |
| mg* | | | | | 1.0 |

(Approximate* adjusted)

A 53 year old male patient presented with all the signs and symptoms of TB. The preliminary test revealed the presence of mycobacterium tuberculosis bacteria in the sputum. The chest x ray showed TB lesion affecting right lung, upper lobe. This patient was diagnosed as patient with tuberculosis granuloma with possibility of cavitary formation in progress. The testing and monitoring is the same as described in example 1.

The patient was induced hypoglycemia to 40 mg-60 mg % by injection of fast acting insulin, dose decided based on initial blood sugar levels and condition of the patient.

Then the infusion of 1200 mg of rifampicin diluted in 500 ml of normal saline was started.

The patient was put on 200 mgs of ibuprofen twice a day, and received supplemental oxygen through the non-rebreeding mask. Nasal canula can also be used.

The infusion was completed at the end of 3 hours.

All the vital signs were stable.

At the end of the infusion blood glucose level was tested. If it was not greater than 80 mg %, 25% glucose was administered intravenously, and then rechecked to achieve the normal desired level of glucose.

Then the patient was rested for 2-4 hours.

At the end of the rest, the patient was infused 20 grams of Vitamin C diluted in normal saline for 4 hours.

The patient rested and continues to monitor.

The above table shows the possible plasma concentration reached by rifampicin.

We continued this therapy regimen for 5 days with two days' week end rest.

The patient was tested for sputum for presence of M. tuberculosis bacteria. The results were negative. The chest x ray revealed the shrinking of the TB lesion in the lungs. The treatment was continued for 6 weeks to make sure all the TB bacteria were eliminated, possibly in latent or hidden bacillus (in persisters cells) anywhere. The cough was reduced to the minimum and was hardly productive. He begins to gain weight. The patient was checked every month for first 3 months then tested every 6 months. The test showed that the patient was cured of the pulmonary tuberculosis. The patient was prescribed ibuprofen 200 mg twice daily to inhibit the inflammatory processes in the lungs and make TB bacteria more receptive and vulnerable to therapy. Similarly 6 other patients were treated resulting in complete cure of the tuberculosis within 6 weeks of intense therapy as described above. All these patients were monitored for liver function test.

Granuloma and Cold Abscess Treatment Due to Mycobacterium Tuberculosis Infection Outside the Pulmonary System Using Our Method and Device Described in this Invention:

The mycobacterium tuberculosis induces lung lesions and cavitary tuberculosis (FIGS. 2, 3, 6, 7). From this site, the infecting organisms spread to other parts of the body through hematologic and lymphatic spread. They are localized TB infection other than the lungs resulting in abscess formation, what is named as “cold abscess.” (FIG. 8) Abscess is a localized collection of pus in a cavitary formed by disintegration of tissues due to inflammatory reaction by immune system cells and the local tissue reaction to M. tuberculosis bacterial infection. It is called cold abscess in TB due to lack of classic signs and symptoms of localized infection (heat, redness, edema, swelling, pain).

Extrapulmonary TB accounts for roughly 15% of TB cases among immunocompetent hosts (Peto, HM, Pratt, R H, et. al. 2009), and for 50% to 70% of cases that occur in the context of conjunctivitis-infection with HIV. Up to 40% of extrapulmonary TB cases are attributable to tuberculous lymphadenitis. In low-incidence countries, immigrants from endemic countries are much more likely to present with extrapulmonary TB As a rule, TB can present in any organ system; therefore, vigilance and examination for extrapulmonary disease are important for all persons being evaluated for TB infection.

Other than mycobacterium tuberculosis of lungs; an estimated 2 million people have spinal TB all over the world and up to 3% of all TB involve skeletal system. Bone and Joint TB develops about 2 to 3 years after primary focus of infection. This abscess was first described by Sir Percival Pott in 1779 hence the name Potts disease. Bones and joints are the 4th commonest site of extrapulmonary TB, affects weight bearing joint commonly (Ex. spine 40%, Hips 13%, knees 10%). TB spine is always secondary and estimated 2 million people have active spinal TB the developing world. Cold abscesses (FIG. 7) are by and large associated with tuberculosis bacterial infections commonly seen at neck, axilla, groin, back of the spines, side of the chest wall, ribs, posterior mediastinal lymph nodes and other unusual sites (view the informative and excellent power point presentations by Drs. Vishal Sankpal, Mohan Krishna, Shadid Latheef, Hardik Pawar, Sudheer Kumar, Khaled Abdeen et. al, T. V. Rao (outstanding overview on tuberculosis student up date, TB basics and others), Dave Jay S. Manriquez and others on Tuberculosis of lungs, spine and bones-ww.slideshare.net/drmkreddy78/tuberculosis-of-spine-Jul. 20, 2013). Within the tubercle lesion, small patches of caseous necrosis develop (FIG. 1a, 2, 7). The TB granuloma with necrosis coalesce into a larger yellowish cheese like mass (caseous material) or the center may breakdown leading to cold abscess made up of liquefaction of granuloma and caseous center surrounded by reactive exudates composed of serum, leucocytes, and M. tuberculosis bacilli (FIGS. 2, 7).

In some cases, a cold abscess can form without a tuberculosis infection, such as when skin abscesses form as a result of a staph infection. Persistent cold abscesses from staph infections are known as MRSA abscesses. Cold abscesses can also form in the psoas region in patients with inflammatory bowel disease, or in patients suffering from specific types of gunshot wounds.

While some of these abscesses enlarge, bulge and fade on their own, the majority of cold abscesses (FIG. 2, 7) in advanced state (large swellings) may travel on and along the muscle and facial planes. They are diagnosed by MRI scans, x rays and looking for primary source of infection. They require drainage via per-cutaneous needle—catheter or surgical drainage through a small incision if the caseous material cannot be aspirated as shown in FIG. 7. Our experience in treating various local and regions lesions both cancerous and non-cancerous teaches us that the cold abscess be effectively treated by:

Under local anesthesia, draining the pus completely using a large bore needle and/or after small incision and curettage of the cold abscess and cheesy caseous material.

Irrigate the abscess with specific anti TB drugs mixed in lactated ringer, normal saline, or Locke solution. Ozonized normal saline or 3% hydrogen peroxide solution and/or Ozonized solutions be used for irritation. The irrigation solution temperature be raised to 41 to 43(106.80, to 109.4 Fahrenheit) degree centigrade (Shantha 2003) to kill the persistor M. tuberculosis bacteria in the wall of the abscesses. Irrigate the abscess lesion over a period of 5-10 minutes depending on the size of the abscess and location till the exiting irrigating fluid is clear.

The irrigation fluid contained Vitamin C (5-10 grams), mixed with high doses of anti TB drugs (rifampicin and streptomycin) are used to irrigate once the necrotic material is evacuated and then deposited high concentrations of Vitamin C and rifampicin at the end of the drainage and irrigation.

Repeat the procedure every day up to six days or more depending upon the response and circumstances and as the drainage from the cold abscess decrease or dries out. The he irrigation canula is withdrawn, inject high doses of Vitamin C mixed with anti TB drugs (streptomycin with or without rifampicin) and insulin (4-10 IU) and then seal the irritation site after the procedure to prevent the leaking of the final injectate and prevent secondary infection.

We have injected locally, hundreds of primary as well as metastasis lesions of cancer or non-operable localized cancers including those inside the bladder, interstitial cystitis, and MRSA resistant abscesses by appropriate chemotherapy—antibiotics, vitamin C with insulin and warmed or room temperature solutions (apply external heat through external heat delivery system after injections can also instituted). Many of the non-operable tumors locally spread or otherwise disappeared or become smaller which were surgically excised later. The MRSA abscesses melted away. We believe that that the same method of treatment of tuberculosis cold abscess can be easily adopted. Untreated peritoneal infection by massive Candida albicans after stomach surgery, not responding any treatment, literally on death bed, was treated by us with peritoneal hyperthermia with Diflucan for 4 hours with continuous irrigation (Shantha 2003). The patient recovered completely and walked out of the hospital without infection.

Treatment of Enlarged, Palpable and I or Accessible Tuberculosis Bacteria Infected Cervical, Supra-Clavicular, Axillary, Inguinal Lymph Nodes and Other Such Lesions by Local Injection of Anti TB Therapeutic Agents Rifampicin, and I or Streptomycin Including Vitamin C and Insulin:

Up to 40% of extrapulmonary TB cases are attributable to tuberculous lymphadenitis (Peto, H M, et. al. 2009). We also injected neck and supra-clavicular enlarged lymph nodes suspected of tuberculosis bacterial infection (some lesions confirmed by biopsy) with insulin, rifampicin and Vitamin C combination. The injection was repeated every day or every other day for a week and then every three days once. Wet heat compressors applied after injection to augment the therapeutic effects of injectate. The lymph nodes became smaller within two weeks indicating that the M. tuberculosis bacteria were killed by this method and cleared of the inflammatory reaction. Later biopsy revealed absence of M. tuberculosis bacteria. This is one of the best methods to treat local, easily accessible TB bacterial lesions instead of surgical excision. If the patient has enlarged nodes in the neck, above the clavicle and other areas, if it is concluded that their enlargements were due to M. tuberculosis bacterial infection. If the lymph node swelling is huge, shrink the swelling by direct injection before or after draining its caseous material from the node.

Signs and Symptoms of the Effectiveness of the Anti-Tuberculosis Therapy:

They are measured by lessening in tiredness, a reduction in the occurrence of chills and night sweats of, no more chest pain and late noon fever, weight gain, an improvement in appetite, decrease in the frequency or duration of coughs, diminution production of sputum after coughing, with a reduction in dyspnea with feeling of wellbeing with improvement or negative sing by physical examination of the lungs. In other embodiments, the improved symptoms may be measured physiologically such as one or more of an increase in the number of lymphocytes present in biological samples obtained by bronchoalveolar lavage, a decrease in the number of neutrophils present in biological samples obtained by Bronchoalveolar lavage, or a reduction in the amount of inflammatory cytokines present in biological samples obtained by bronchoalveolar lavage, reduction of blood ESR (nonspecific measure of inflammation), and conversion of M. tuberculosis bacteria positive to negative sputum, indicator of the decrease or elimination of the tuberculosis pathology as indicated by X rays, and Scans.

Experimental results suggest that drugs that target the succinic dehydrogenase I or enhance the tuberculosis bacterial respiration would shorten the tuberculosis chemotherapy. That is what we describe in this invention. Our method of induction of hyperthermia, supplemental oxygen with large doses of Vitamin C and anti-tuberculosis drugs with insulin enhances the respiration in the TB bacteria, thus killing it rapidly. This invention provides a method of enhancing efficacy of an anti-tuberculosis medication in treating tuberculosis in a subject or in treating resistant and/or dormant Mycobacteria tuberculosis infection in a subject, comprising: administering to the subject who has, is or will be receiving the anti-tuberculosis medication of an amount of an enhancer of Mycobacteria tuberculosis respiration effective to enhance the efficacy of anti-tuberculosis medication treating tuberculosis or resistant and/or dormant Mycobacteria tuberculosis inflection.

Numerous modifications, adjuvant, alternative arrangements of steps explained and examples given herein may be devised by those skilled in the description and art without departing from the spirit and scope of this invention and the appended claims are intended to cover such modifications and arrangements. While the preferred embodiment of the present invention has been described, it should be understood that various changes, adaptations, and modifications may be made thereto. It should be understood, for that reason, that the invention is not limited to details of the illustrated invention described above and claims declared below. Therefore, this invention shall include embodiments falling within the scope of the appended claims. For that reason, the specification and figures are to be regarded in an illustrative rather than a restrictive sense, and all such modifications are intended to be included within the scope of present invention. The invention is defined solely by the appended claims including any amendments made during the pendency of this application and all equivalents of those claims as issued.

Although the instant invention has been described in relation to particular embodiments thereof, many other variations and modifications and other uses will become apparent to those skilled in the art.

1. Method for treating Mycobacterium tuberculosis bacterial infection in a subject to achieve rapid cure by intravenous administration of a therapeutically effective high dose Vitamin C combined with insulin and selected anti M. tuberculosis bacterial therapeutic agents comprising the steps of:
a) administering a medically effective dose of insulin parenterally delivered to a patient wherein blood sugar levels are lowered;
b) preparing a Vitamin C solution by mixing a selected amount of Vitamin C in 500 ml of ringers lactate or normal saline to produce a Vitamin C solution;
c) administering intravenously to said patient said Vitamin C solution over a period of 3 hours; and
d) administering to said patient a medically effective dose of an anti-tuberculosis drug.

2. (canceled)

3. The method as claimed in claim 1, wherein said medically effective dose of an anti-tuberculosis drug is administered intravenously.

4. The method as claimed in claim 1, wherein said medically effective dose of an anti-tuberculosis drug is administered orally.

5. The method as claimed in claim 1, wherein said Vitamin C solution is administered intravenously

6. The method as claimed in claim 1, wherein said Vitamin C solution is selected to be in a range of from 5% to 25% Vitamin C.

7. The method as claimed in claim 3, wherein said medically effective dose of an anti-tuberculosis drug is rifampicin selected to be in a range from 600 mgs to 1800 mgs.

8. The method as claimed in claim 3, wherein said medically effective dose of an anti-tuberculosis drug is Artemisinin.

9. The method as claimed in claim 3, wherein said medically effective dose of an anti-tuberculosis drug is Ethionamide.

10. The method as claimed in claim 1, further comprising the step of administering one gram of streptomycin parentally 5 days a week for 4 weeks.

11. The method as claimed in claim 4, wherein said medically effective dose of an anti-tuberculosis drug is a combination of Isoniazid and Ethambutol administered daily.

12. The method as claimed in claim 1, wherein said medically effective dose of an anti-tuberculosis drug is Rifampin administered intravenously and Ethambutol administered orally.

13. The method as claimed in claim 1, wherein said medically effective dose of an anti-tuberculosis drug is Rifampin administered intravenously and a combination of Isoniazid and Pyrazinamide administered orally.

14. The method as claimed in claim 1, wherein said medically effective dose of an anti-tuberculosis drug is Rifampin administered intravenously and a combination of Isoniazid, Etambutol and Pyrazinamide administered orally.

15. The method as claimed in claim 1, wherein said medically effective dose of an anti-tuberculosis drug is Rifampin administered intravenously and a combination of Isoniazid, Etambutol, Pyrazinamide, and TMC207 (Bedaquiline, SIRTURO®) orally,

16. The method as claimed in claim 1, wherein said medically effective dose of an anti-tuberculosis drug is at least one of the drugs selected from the group consisting of isoniazid, rifampicin, pyrazinamide, ethambutol, fluoroquinolones (CIPRO™ Levaquin, Avelox or generics ciprofloxacin, Levofloxacin), amikacin, capreomycin and kanamycin.

17. The method as claimed in claim 1, wherein said medically effective dose of an anti-tuberculosis drug is at least one of the drugs selected from the group consisting of Ethionamide (2-ethylthioiso-nicotinamide, 2-ethylpyrimidine-4-carbothioamide), Kanamycin, Amikacin, Cycloserine, Para amino salicylic acid (PAS), Ofloxacin, Levofloxacin, and moxifloxacin.

18. The method as claimed in claim 1, further comprising the step of administering an anti-inflammatory adjuvant mycobacterium tuberculosis therapeutic agent.

19. The method as claimed in claim 1, wherein said Vitamin C solution and said medically effective dose of an anti-tuberculosis drug are delivered to the lungs.

20. The method as claimed in claim 1, wherein said Vitamin C solution and said medically effective dose of an anti-tuberculosis drug are delivered directly to a tuberculosis lesion through a catheter inserted in said lesion.

21. The method as claimed in claim 1, for treating pulmonary and extrapulmonary tuberculosis and tuberculosis resistant to anti TB drugs by creating electrical fields using AC currents in the range of 10 KHz to 500 KHz which passes from anterior to posterior and right to left side around the chest wall traversing through the infected lung's granuloma and cavitary TB with M. tuberculosis lesions; wherein said electrical fields will have a bactericidal effect on the dividing M. tuberculosis bacteria in the infected lungs and hilar lymph nodes by inhibiting and preventing the multiplication of the bacteria.
